(19) United States
(12) Patent Application Publication
(10) Pub. No.: US 2022/0041606 A1
(43) Pub. Date: Feb. 10, 2022

(54) POSITIVE ALLOSTERIC MODULATORS OF THE MUSCARINIC ACETYLCHOLINE RECEPTOR M1

(71) Applicant: Vanderbilt University, Nashville, TN (US)

(72) Inventors: Craig W. Lindsley, Brentwood, TN (US); P. Jeffrey Conn, Nashville, TN (US); Julie L. Engers, Brentwood, TN (US); Alison R. Gregro, Mount Juliet, TN (US); Madeline F. Long, Nashville, TN (US)

(21) Appl. No.: 17/386,876

(22) Filed: Jul. 28, 2021

Related U.S. Application Data
(60) Provisional application No. 63/057,393, filed on Jul. 28, 2020.

(51) Int. Cl. C07D 487/04; C07D 471/04; C07D 491/048; C07D 495/04

(52) U.S. Cl. CPC C07D 487/04; C07D 495/04; C07D 491/048; C07D 471/04

(57) ABSTRACT

Described are substituted fused bicyclic pyridinone and pyrimidinone carboxamide positive allosteric modulators of muscarinic acetylcholine receptor M₁ (mAChR M₁), pharmaceutical compositions including the compounds, and methods of using the compounds and compositions for treating neurological disorders, psychiatric disorders, or a combination thereof.

### RELATED APPLICATIONS

This application claims priority to U.S. Provisional Application No. 63/057,393, filed Jul. 28, 2020, which is hereby incorporated by reference in its entirety.

### STATEMENT OF GOVERNMENT INTEREST

This invention was made with government support under Grant number MH106839 awarded by the National Institute of Health (NIH). The government has certain rights in the invention.

### TECHNICAL FIELD

The present disclosure relates to compounds, compositions, and methods for treating muscarinic acetylcholine receptor M₁ related diseases and/or disorders, such as neurological and psychiatric disorders.

### BACKGROUND

Positive allosteric modulators are compounds that bind to a site distinct from that of the orthosteric agonist binding site of a target protein. These modulators enhance the affinity or efficacy of an orthosteric agonist. For example, a selective muscarinic M₁ positive allosteric modulator would result in an increased affinity at the orthosteric binding site for acetylcholine (ACh), the endogenous agonist for the muscarinic M₁ receptor, or an increase in the efficacy induced by ACh. In some systems, the compound may also have an intrinsic activity to activate the receptor in the absence of orthosteric ligand. Positive allosteric modulation (potentiation), therefore, can be an attractive mechanism for enhancing appropriate physiological receptor activation.

Cholinergic neurotransmission involves the activation of nicotinic acetylcholine receptors (nAChRs) or the muscarinic acetylcholine receptors (mAChRs) by the binding of the endogenous orthosteric agonist ACh. Acetylcholinesterase (AChE) inhibitors, which inhibit the hydrolysis of ACh, have been approved in the United States for use in the palliative, but not disease-modifying, treatment of the cognitive deficits in Alzheimer's disease (AD) patients.

mAChRs are members of the family A GPCRs, and include five subtypes, designated M₁, M₂, M₃, M₄, and M₅. M₁, M₃ and M₅ mainly couple to G_{i/o} and activate phospholipase C, whereas M₂ and M₄ mainly couple to Guo and associated effector systems. These five distinct mAChR subtypes have been identified in the mammalian central nervous system where they are prevalent and differentially expressed. M₁-M₅ mAChRs have varying roles in cognitive, sensory, motor and autonomic functions. Activation of various muscarinic receptors, particularly the M₁ subtype, has been proposed as a mechanism to enhance cognition in disorders such as AD and schizophrenia (as well as negative symptoms). Thus, selective positive allosteric modulators of mAChR subtypes that regulate processes involved in cognitive function could prove superior to AChE inhibitors for treatment of AD and related disorders as these compounds may exhibit improved selectivity for specific mAChRs.

Efforts to create selective M₁ agonists have been largely unsuccessful, in part due to the high conservation of the orthosteric ACh binding site. As a result, mAChR agonists in clinical studies induce the same adverse effects of AChE inhibitors by activation of peripheral mAChRs. To fully understand the physiological roles of individual mAChR subtypes and to further explore the therapeutic utility of mACh receptors in AD, schizophrenia and other disorders, there exists a need to develop compounds that are highly selective modulators of M₁ and other individual mAChR subtypes. Accordingly, allosteric modulation may be an advantageous pathway because allosteric sites on mAChRs are less highly conserved.

Despite advances in muscarinic receptor (mAChR) research, there remains a scarcity of compounds that are potent, efficacious and selective positive allosteric modulators of the M₁ mAChR that are also effective in the treatment of neurological and psychiatric disorders associated with cholinergic activity, or other neurologic diseases in which the muscarinic M₁ receptor may be involved.

### SUMMARY

In one aspect, disclosed are compounds of formula (I), or pharmaceutically acceptable salts thereof,

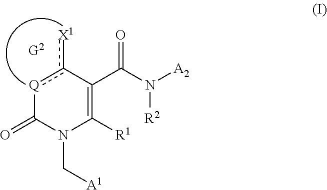

wherein
Q is C or N;
G² is a 5- to 6-membered monocyclic heteroarene containing X¹ and, in addition, 0, 1, or 2 nitrogen atoms, the heteroarene being unsubstituted or substituted with 1-3 substituents independently selected from the group consisting of halogen, C₁₋₄alkyl, and C₁₋₄haloalkyl;
X¹ is an unsubstituted heteroatom selected from the group consisting of nitrogen, oxygen, and sulfur;
A¹ is

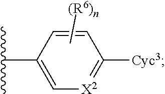

X² is N or CR^{6a};
Cyc³ is a phenyl or 5- to 12-membered heteroaryl, wherein the phenyl and 5- to 12-membered heteroaryl are unsubstituted or substituted with 1-5 substituents independently selected from the group consisting of halogen, C₁₋₄alkyl, C₁₋₄haloalkyl, —OC₁₋₄alkyl, —OC₁₋₄haloalkyl, —OC₃₋₆cycloalkyl, —O—C₁₋₃alkylene-C₃₋₆cycloalkyl, —OH, oxo, cyano, C₃₋₆cycloalkyl, and —C₁₋₃alkylene-C₃₋₆cycloalkyl, wherein each cycloalkyl is unsubstituted or substituted with 1-4 substituents independently selected from the group consisting of halogen and C₁₋₄alkyl;
A² is C₁₋₆alkyl, C₁₋₆haloalkyl, or -L¹-G¹, wherein the C₁₋₆alkyl and C₁₋₆haloalkyl are unsubstituted or substituted with 1-2 substituents independently selected from the group consisting of cyano, oxo, —OH, and —OC₁₋₄alkyl;
L¹ is a bond, C₂₋₆alkenylene, or C₁₋₆alkylene, wherein the C₂₋₆alkenylene and C₁₋₆alkylene are unsubstituted or substituted with 1-4 substituents independently selected from the group consisting of halogen, cyano, —OH, oxo, —OC₁₋₄alkyl, and C₃₋₆cycloalkyl;
G¹ is C₃₋₁₂cycloalkyl or 4- to 12-membered heterocyclyl, wherein G¹ is unsubstituted or substituted with 1-4 substituents independently selected from the group consisting of C₁₋₄alkyl, C₁₋₄haloalkyl, —OH, —OC₁₋₄alkyl, cyano, oxo, C₃₋₆cycloalkyl, —C₁₋₃alkylene-OH, and —C₁₋₃alkylene-OC₁₋₄alkyl;
R¹ is hydrogen, halogen, cyano, C₁₋₄alkyl, C₁₋₄haloalkyl, —OC₁₋₄alkyl, or —C₁₋₃alkylene-OC₁₋₄alkyl;
R² is hydrogen, C₁₋₆alkyl, or C₁₋₆haloalkyl;
R⁶, at each occurrence, is independently selected from the group consisting of halogen, C₁₋₄alkyl, C₁₋₄haloalkyl, —OC₁₋₄alkyl, —OC₁₋₄haloalkyl, —OH, cyano, C₃₋₆cycloalkyl, and —C₁₋₃alkylene-C₃₋₆cycloalkyl;
R^{6a} is selected from the group consisting of hydrogen, halogen, C₁₋₄alkyl, C₁₋₄haloalkyl, —OC₁₋₄ alkyl, —OC₁₋₄haloalkyl, —OH, cyano, C₃₋₆cycloalkyl, and —C₁₋₃alkylene-C₃₋₆cycloalkyl; and
n is 0, 1, 2, or 3.

In another aspect, the invention provides a pharmaceutical composition comprising a compound of formula (I), or a pharmaceutically acceptable salt thereof, and a pharmaceutically acceptable carrier.

In another aspect, the invention provides a method for the treatment of a disorder associated with muscarinic acetylcholine receptor activity in a mammal, comprising administering to the mammal an effective amount of a compound of formula (I), or a pharmaceutically acceptable salt or composition thereof.

In another aspect, the invention provides a compound of formula (I), or a pharmaceutically acceptable salt or composition thereof, for use in the treatment of a disorder associated with muscarinic acetylcholine receptor activity.

In another aspect, the invention provides the use of a compound of formula (I), or a pharmaceutically acceptable salt or composition thereof, in the manufacture of a medicament for the treatment of a disorder associated with muscarinic acetylcholine recepter activity.

In another aspect, the invention provides a kit comprising a compound of formula (I), or a pharmaceutically acceptable salt or composition thereof, and instructions for use.

Also disclosed are methods of making the compounds, and methods of using the compounds for treatment of M₁ muscarinic acetylcholine receptor related diseases and/or disorders.

In another aspect, the invention provides a compound of formula (I), or a pharmaceutically acceptable salt or composition thereof, with minimal to substantially no M₁ agonist activity compared to acetylcholine. The relative absence of M₁ agonist activity is expected to avoid or reduce cholinergic adverse effect liability at therapeutic concentrations and/or doses.

### DETAILED DESCRIPTION

Disclosed herein are positive allosteric modulators of the M₁ muscarinic acetylcholine receptor M₁ (mAChR M₁). The modulators can have the structure of formula (I). Compounds of formula (I) exhibit high affinity for mAChR M₁, and can also exhibit selectivity over other muscarinic acetylcholine receptors. Compounds of formula (I) can be used to treat or prevent diseases and disorders associated with mAChR M₁ by modulating mAChR M₁ activity. mAChR M₁ has been implicated in a number of different diseases and disorders including, but not limited to, neurological and psychiatric disorders.

Since the orthosteric binding sites of the mAChR isoforms are highly conserved, selective modulators of the mAChRs that bind at the orthosteric site remain elusive. One strategy to selectively bind and modulate the mAChRs includes identifying allosteric sites which may be amenable to modulation by a small molecule. In particular, positive allosteric modulation of mAChR M₁ can result in potentiation of the mAChR M₁ receptor and provide therapeutic benefits for disorders associated with mAChR M₁ dysfunction.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain saturated hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkylene,” as used herein, refers to a divalent group derived from a straight or branched saturated chain hydrocarbon, for example, of 1 to 6 carbon atoms. Representative examples of alkylene include, but are not limited to, —CH₂—, —CH(CH₃)—, —C(CH₃)₂—, —CH₂CH₂—, —CH(CH₃)CH₂—, —C(CH₃)₂CH₂—, —CH₂CH₂CH₂—, —CH(CH₃)CH₂CH₂—, —C(CH₃)₂CH₂CH₂—, —CH₂C(CH₃)₂CH₂—, —CH₂CH₂CH₂CH₂—, and —CH₂CH₂CH₂CH₂CH₂—.

The term “alkenylene,” as used herein, refers to a divalent group derived from a straight or branched chain hydrocarbon having at least one carbon-carbon double bond.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), a bridged cycloalkyl in which two non-adjacent atoms of a ring are linked by an alkylene bridge of 1, 2, 3, or 4 carbon atoms (e.g., bicyclo[2.2.1]heptanyl), or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkene,” as used herein, means a non-aromatic monocyclic or multicyclic all-carbon ring system containing at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl.

The term “fluoroalkyl,” as used herein, means an alkyl group, as defined herein, in which one, two, three, four, five, six, seven or eight hydrogen atoms are replaced by a fluoro group. Representative examples of fluoroalkyl include CH₂F, CHF₂, CF₃, and CH₂CF₃.

The term “halogen” or “halo,” as used herein, means C₁, Br, I, or F.

The term “haloalkyl,” as used herein, means an alkyl group, as defined herein, in which one, two, three, four, five, six, seven or eight hydrogen atoms are replaced by a halogen.

The term “heteroaryl,” as used herein, refers to an aromatic monocyclic heteroatom-containing ring (monocyclic heteroaryl) or a bicyclic ring system containing at least one monocyclic heteroaryl (bicyclic heteroaryl). The term “heteroaryl” is used herein to refer to a heteroarene when present as a substituent, the term “heteroarene” being used in cases of ring fusion. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl is an 8- to 12-membered ring system and includes a fused bicyclic heteroaromatic ring system (i.e., “fully aromatic” 10π electron system) such as a monocyclic heteroaryl ring fused to a 6-membered arene (e.g., quinolin-4-yl, indol-1-yl), a monocyclic heteroaryl ring fused to a monocyclic 5- to 6-membered heteroarene (e.g., naphthyridinyl), and a phenyl fused to a monocyclic 5- to 6-membered heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, and 3-oxaspiro[5.5]undecanyl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. As used herein, “Cₘ to Cₙ,” “Cₘ-Cₙ” or “Cₘ₋ₙ” in which “m” and “n” are integers refers to the number of carbon atoms in the relevant group. That is, the group can contain from “m” to “n” inclusive, carbon atoms. Thus, for example, a “C₁ to C₆ alkyl” group refers to all alkyl groups having from 1 to 6 carbons, that is, CH₃—, CH₃CH₂—, CH₃CH₂CH₂—, (CH₃)₂CH—, CH₃CH₂CH₂CH₂—, CH₃CH₂CH(CH₃)—, CH₃CH(CH)₃CH₂—, CH₃CH(CH)₃CH₂— and (CH₃)₃C—. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched). If no “m” and “n” are designated with regard to a group, the broadest range described in these definitions is to be assumed.

A dashed bond, [symbol] , represents an optional unsaturation between the atoms forming the bond. This bond may be unsaturated (e.g. C═C, C±N, C═O) or saturated (e.g. C—C, C—N, C—O). When a dashed bond is present in a ring system it may form part of an aromatic ring system.

The term “substituted” refers to a group that may be further substituted with one or more non-hydrogen substituent groups. Substituent groups may include, for example, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

As used herein, an “optional substituent” is a substituent that may or may not be present on another molecular group, such as a ring (e.g., phenyl) or chain (e.g., alkyl). A group that is “optionally substituted” with a substituent means the group is either unsubstituted or substituted with the substituent.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Pharmaceutically acceptable solvates and hydrates are complexes of a compound with one or more solvent or water molecules, or 1 to about 100, or 1 to about 10, or one to about 2, 3 or 4, solvent or water molecules.

As used herein, a “prodrug” refers to a compound that may not be pharmaceutically active but that is converted into an active drug upon in vivo administration. The prodrug may be designed to alter the metabolic stability or the transport characteristics of a drug, to mask side effects or toxicity, to improve the flavor of a drug or to alter other characteristics or properties of a drug. Prodrugs are often useful because they may be easier to administer than the parent drug. They may, for example, be bioavailable by oral administration whereas the parent drug is not. The prodrug may also have better solubility than the active parent drug in pharmaceutical compositions. By virtue of knowledge of pharmacodynamic processes and drug metabolism in vivo, those skilled in the art, once a pharmaceutically active compound is known, can design prodrugs of the compound (see, e.g. Nogrady (1985) Medicinal Chemistry A Biochemical Approach, Oxford University Press, New York, pages 388-392).

The terms, “polymorphs” and “polymorphic forms” refer to crystalline forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice. Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation. Techniques for characterizing polymorphs include, but are not limited to, differential scanning calorimetry (DSC), X-ray powder diffractometry (XRPD), single crystal X-ray diffractometry, vibrational spectroscopy, e.g., IR and Raman spectroscopy, solid state NMIR, hot stage optical microscopy, scanning electron microscopy (SEM), electron crystallography and quantitative analysis, particle size analysis (PSA), surface area analysis, solubility studies and dissolution studies.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Compounds

A first aspect of the invention provides compounds of formula (I), wherein R¹, R², A¹, A², and G²

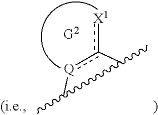

are as defined herein. The embodiments of formula (I) provided herein include any combinations of the variables R¹, R², A¹, A², and G², as these variables are described herein, including with sub-variables such as X¹, X², Cyc³, R⁶, etc.

Throughout the embodiments and description of the compounds of the invention, all instances of haloalkyl may be fluoroalkyl (e.g., any C₁₋₄haloalkyl may be C₁₋₄fluoroalkyl).

Unsubstituted or substituted rings such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

In the following, embodiments of the invention are disclosed. The first embodiment is denoted E1, the second embodiment is denoted E2 and so forth. In the numbered embodiments, the reference to a range of preceding embodiments in multiple dependent format (e.g., “the compound of any of E1-E15”) is a reference, in the alternative, to each embodiment sequentially listed herein in the recited range.

E1. A compound of formula (I), or a pharmaceutically acceptable salt thereof,

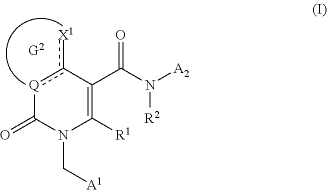

wherein
Q is C or N;
G² is a 5- to 6-membered monocyclic heteroarene containing X¹ and, in addition, 0, 1, or 2 nitrogen atoms, the heteroarene being unsubstituted or substituted with 1-3 substituents independently selected from the group consisting of halogen, C₁₋₄alkyl, and C₁₋₄haloalkyl;
X¹ is an unsubstituted heteroatom selected from the group consisting of nitrogen, oxygen, and sulfur;
A1 is

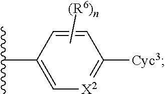

X² is N or CR^{6a}.
Cyc³ is a phenyl or 5- to 12-membered heteroaryl, wherein the phenyl and 5- to 12-membered heteroaryl are unsubstituted or substituted with 1-5 substituents independently selected from the group consisting of halogen, C₁₋₄alkyl, C₁₋₄haloalkyl, —OC₁₋₄alkyl, —OC₁₋₄haloalkyl, —OC₃₋₆cycloalkyl, —O—C₁₋₃alkylene-C₃₋₆cycloalkyl, —OH, oxo, cyano, C₃₋₆cycloalkyl, and —C₁₋₃alkylene-C₃₋₆cycloalkyl, wherein each cycloalkyl is unsubstituted or substituted with 1-4 substituents independently selected from the group consisting of halogen and C₁₋₄alkyl;
A² is C₁₋₆alkyl, C₁₋₆haloalkyl, or -L¹-G¹, wherein the C₁₋₆alkyl and C₁₋₆haloalkyl are unsubstituted or substituted with 1-2 substituents independently selected from the group consisting of cyano, oxo, —OH, and —OC₁₋₄alkyl;
L¹ is a bond, C₂₋₆alkenylene, or C₁₋₆alkylene, wherein the C₂₋₆alkenylene and C₁₋₆alkylene are unsubstituted or substituted with 1-4 substituents independently selected from the group consisting of halogen, cyano, —OH, oxo, —OC₁₋₄alkyl, and C₃₋₆cycloalkyl;
G¹ is C₃₋₁₂cycloalkyl or 4- to 12-membered heterocyclyl, wherein G¹ is unsubstituted or substituted with 1-4 substituents independently selected from the group consisting of C₁₋₄alkyl, C₁₋₄haloalkyl, —OH, —OC₁₋₄alkyl, cyano, oxo, C₃₋₆cycloalkyl, —C₁₋₃alkylene-OH, and —C₁₋₃alkylene-OC₁₋₄alkyl;
R¹ is hydrogen, halogen, cyano, C₁₋₄alkyl, C₁₋₄haloalkyl, —OC₁₋₄alkyl, or —C₁₋₃alkylene-OC₁-4alkyl;
R² is hydrogen, C₁₋₆alkyl, or C₁₋₆haloalkyl;
R⁶, at each occurrence, is independently selected from the group consisting of halogen, C₁₋₄alkyl, C₁₋₄haloalkyl, —OC₁₋₄alkyl, —OC₁₋₄haloalkyl, —OH, cyano, C₃₋₆cycloalkyl, and —C₁₋₃alkylene-C₃₋₆cycloalkyl;
R^{6a} is selected from the group consisting of hydrogen, halogen, C₁₋₄alkyl, C₁₋₄haloalkyl, —OC₁₋₄ alkyl, —OC₁₋₄haloalkyl, —OH, cyano, C₃₋₆cycloalkyl, and —C₁₋₃alkylene-C₃₋₆cycloalkyl; and
n is 0, 1, 2, or 3.

In ring G², “ [symbol] ” represents a single or double bond of an aromatic ring, wherein the electrons in ring G² are part of a delocalized aromatic system. Compounds of formula (I) may alternately be represented by formula (I-A)

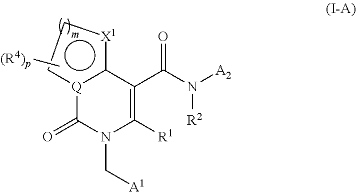

wherein R⁴, at each occurrence, is independently halogen, C₁₋₄alkyl, or C₁₋₄haloalkyl; p is 0, 1, 2, or 3; m is 1 or 2 (i.e., 1 or 2 aromatic carbon atoms); and Q, X¹, R¹, R², A¹, and A² are as defined in any of E1-E41.

In ring G², the 0, 1, or 2 nitrogen atoms are in addition to heteroatom X¹. Atom Q may be one of the 0, 1, or 2 nitrogen atoms in addition to heteroatom X¹. Thus, X¹ may be unsubstituted nitrogen and G² may contain 0, 1, or 2 additional nitrogen atoms, such that G² may have up to 3 total nitrogen ring atoms.

E1.1 The compound of E1, or a pharmaceutically acceptable salt thereof, wherein ring G² contains 1 optional nitrogen atom (i.e., G² contains X¹ and a second heteroatom that is nitrogen).

E2. The compound of E1-E1.1, or a pharmaceutically acceptable salt thereof, wherein G² is the 5-membered monocyclic heteroarene, wherein X¹ is unsubstituted nitrogen.

E3. The compound of E2 of formula (I-B), or a pharmaceutically acceptable salt thereof,

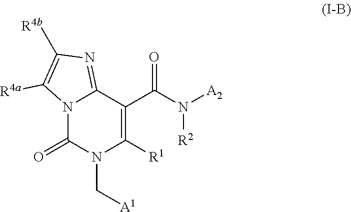

wherein R^{4a} and R^{4b} are independently selected from the group consisting of hydrogen, halogen, C₁₋₄alkyl, and C₁₋₄haloalkyl.

E4. The compound of E3, or a pharmaceutically acceptable salt thereof, wherein R^{4a} is hydrogen and R^{4b} is hydrogen or methyl.

E4.1 The compound of E4, or a pharmaceutically acceptable salt thereof, wherein R^{4a} is hydrogen and R^{4b} is hydrogen.

E4.2 The compound of E4, or a pharmaceutically acceptable salt thereof, wherein R^{4a} is hydrogen and R^{4b} is methyl.

E5. The compound of E2 of formula (I-C), or a pharmaceutically acceptable salt thereof,

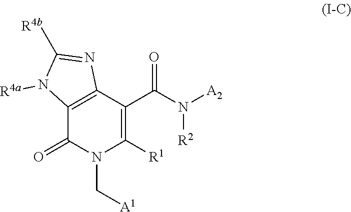

wherein R^{4a} and R^{4b} are independently selected from the group consisting of hydrogen, C₁₋₄alkyl, and C₁₋₄haloalkyl.

E5.1 The compound of E5, or a pharmaceutically acceptable salt thereof, wherein R^{4a} is C₁₋₄alkyl and R^{4b} is hydrogen or C₁₋₄alkyl.

E6. The compound of E5 or E5.1, or a pharmaceutically acceptable salt thereof, wherein R^{4a} is methyl and R^{4b} is hydrogen.

E7. The compound of any of E1-E1.1, or a pharmaceutically acceptable salt thereof, wherein G² is the 5-membered monocyclic heteroarene, wherein X¹ is oxygen or unsubstituted sulfur; and Q is C.

E8. The compound of E7 of formula (I-D), or a pharmaceutically acceptable salt thereof,

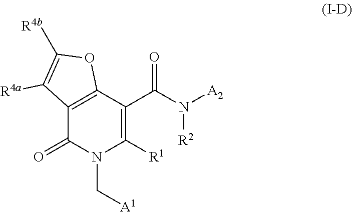

wherein R^{4a} and R^{4b} are independently selected from the group consisting of hydrogen, halogen, C₁₋₄alkyl, and C₁₋₄haloalkyl.

E9. The compound of E7 of formula (I-E), or a pharmaceutically acceptable salt thereof,

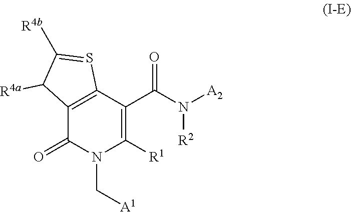

wherein R^{4a} and R^{4b} are independently selected from the group consisting of hydrogen, halogen, C₁₋₄alkyl, and C₁₋₄haloalkyl.

E10 The compound of E8 or E9, or a pharmaceutically acceptable salt thereof, wherein R^{4a} is hydrogen and R^{4b} is hydrogen.

E11. The compound of any of E1-E1.1, or a pharmaceutically acceptable salt thereof, wherein G² is the 6-membered heteroarene; X¹ is unsubstituted nitrogen; and Q is C.

E12. The compound of E11 of formula (I-F), or a pharmaceutically acceptable salt thereof,

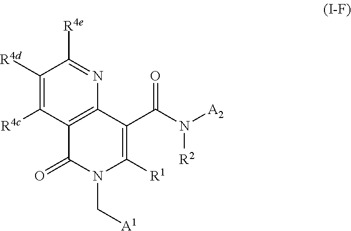

wherein R^{4c}, R^{4d} and R^{4e} are independently selected from the group consisting of hydrogen, halogen, C₁₋₄alkyl, and C₁₋₄haloalkyl.

E12.1. The compound of E12, or a pharmaceutically acceptable salt thereof, wherein R^{4c}, R^{4d} and R^{4e} are hydrogen.

E13. The compound of E11 of formula (I-G), or a pharmaceutically acceptable salt thereof,

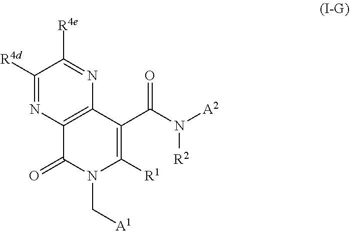

wherein R^{4d} and R^{4e} are independently selected from the group consisting of hydrogen, halogen, C₁₋₄alkyl, and C₁₋₄haloalkyl.

E13.1 The compound of E13, or a pharmaceutically acceptable salt thereof, wherein R^{4d} and R^{4e} are hydrogen.

E14. The compound of E11 of formula (I-H), or a pharmaceutically acceptable salt thereof,

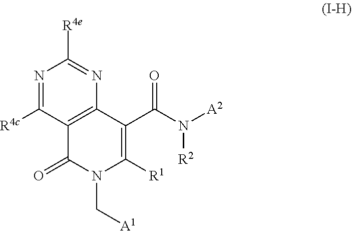

wherein R^{4c} and R^{4e} are independently selected from the group consisting of hydrogen, halogen, C₁₋₄alkyl, and C₁₋₄haloalkyl.

E14.1. The compound of E14, or a pharmaceutically acceptable salt thereof, wherein R⁴⁰ and R^{4e} are hydrogen.

E15. The compound of any of E12-E14.1, or a pharmaceutically acceptable salt thereof, wherein R^{4c}, R^{4d}, and R^{4e} are hydrogen.

E16. The compound of any of E1-E15, or a pharmaceutically acceptable salt thereof, wherein X² is CR^{6a}.

E17. The compound of any of E1-E16, or a pharmaceutically acceptable salt thereof, wherein when X² is CR^{6a} then R^{6a} is hydrogen.

E18. The compound of any of E1-E15, or a pharmaceutically acceptable salt thereof, wherein X² is N.

E19. The compound of any of E1-E18, or a pharmaceutically acceptable salt thereof, wherein n is 0, 1, or 2.

E19.1. The compound of E19, or a pharmaceutically acceptable salt thereof, wherein n is 0.

E20. The compound of any of E1-E19.1, or a pharmaceutically acceptable salt thereof, wherein R⁶ is halogen; and when X² is CR^{6a} then R^{6a} is hydrogen or halogen.

E20.1. The compound of E20, or a pharmaceutically acceptable salt thereof, wherein R⁶ is fluoro; and when X² is CR^{6a} then R^{6a} is hydrogen or fluoro.

E21. The compound of any of E1-E20.1, or a pharmaceutically acceptable salt halogen thereof, wherein A¹ is

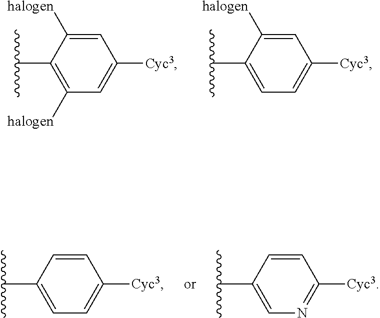

E22. The compound of E21, or a pharmaceutically acceptable salt thereof, wherein A¹ is

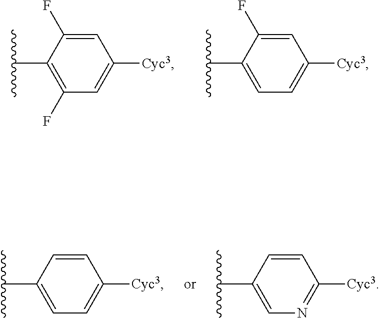

E23. The compound of any of E1-E22, or a pharmaceutically acceptable salt thereof, wherein Cyc³ is the unsubstituted or substituted 5- to 12-membered heteroaryl.

E24. The compound of any of E1-E23, or a pharmaceutically acceptable salt thereof, wherein the ring system of the unsubstituted or substituted 5- to 12-membered heteroaryl of Cyc³ is a 5- to 6-membered monocyclic heteroaryl containing 1-3 heteroatoms independently selected from the group consisting of O, N, and S.

E25. The compound of E24, or a pharmaceutically acceptable salt thereof, wherein the ring system of the unsubstituted or substituted 5- to 6-membered heteroaryl of Cyc³ is pyrazolyl (e.g., pyrazol-1-yl, pyrazol-3-yl, pyrazol-4-yl), oxazolyl (e.g., oxazol-4-yl), thiazolyl (e.g., thiazol-4-yl, thiazol-5-yl), isothiazolyl (e.g., isothiazol-5-yl), or pyridinyl (e.g., pyridin-3-yl, pyridin-4-yl).

E26. The compound of any of E1-E23, or a pharmaceutically acceptable salt thereof, wherein the ring system of the unsubstituted or substituted 5- to 12-membered heteroaryl of Cyc³ is a 9- to 10-membered fused bicyclic heteroaryl containing 1-3 nitrogen atoms.

E27. The compound of E26, or a pharmaceutically acceptable salt thereof, wherein the ring system of the unsubstituted or substituted 9- to 10-membered fused bicyclic heteroaryl is a phenyl fused to a 5-membered heteroarene containing 1-3 nitrogen atoms.

E28. The compound of E26 or E27, or a pharmaceutically acceptable salt thereof, wherein the ring sytem of the unsubstituted or substituted 9- to 10-membered fused bicyclic heteroaryl of Cyc³ is indazolyl.

E29. The compound of any of E1-E28, or a pharmaceutically acceptable salt thereof, wherein Cyc³ is unsubstituted or substituted with 1-3 substituents independently selected from the group consisting of C₁₋₄alkyl and C₃₋₄cycloalkyl.

E30. The compound of any of E1-E25, or a pharmaceutically acceptable salt thereof, wherein Cyc³ is

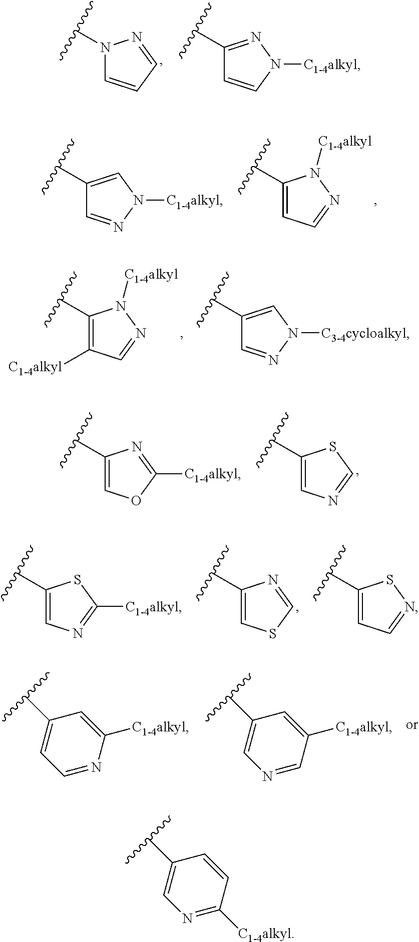

E31. The compound of E30, or a pharmaceutically acceptable salt thereof, wherein Cyc³ is

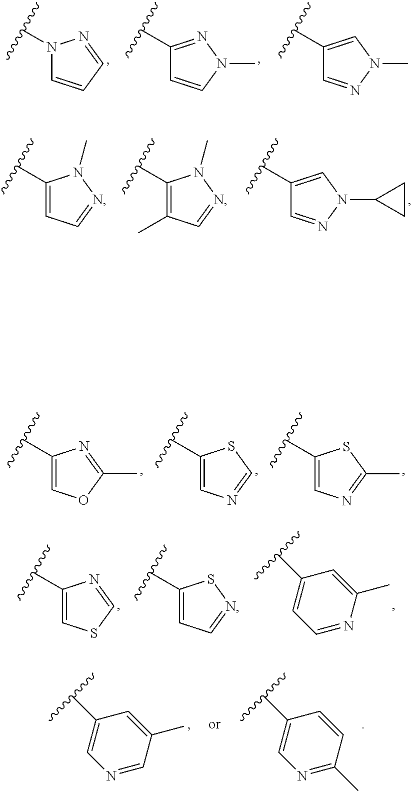

E32. The compound of E28, or a pharmaceutically acceptable salt thereof, wherein C₁₋₄alkyl Cyc³ is

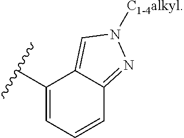

E33. The compound of E32, or a pharmaceutically acceptable salt thereof, wherein Cyc³ is

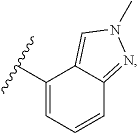 [SMILES: *c1cccc2nn(C)cc12]

E34. The compound of any of E1-E22, or a pharmaceutically acceptable salt thereof, wherein Cyc³ is the unsubstituted or substituted phenyl.

E35. The compound of E34, or a pharmaceutically acceptable salt thereof, wherein Cyc³ is phenyl unsubstituted or substituted with 1-3 substituents independently selected from the group consisting of C₁₋₄alkyl, halogen, and —OC₁₋₄alkyl.

E36. The compound of E35, or a pharmaceutically acceptable salt thereof, wherein Cyc³ is

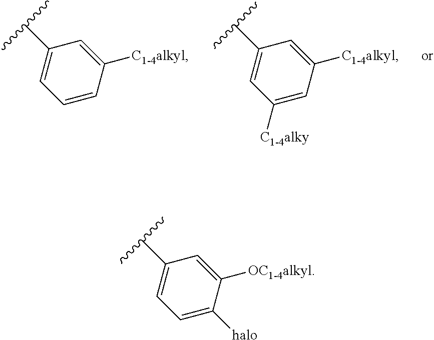

E37. The compound of E36, or a pharmaceutically acceptable salt thereof, wherein Cyc³ is

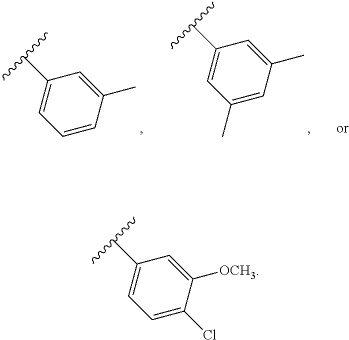

E38. The compound of any of E1-E15, or a pharmaceutically acceptable salt thereof, wherein A¹ is

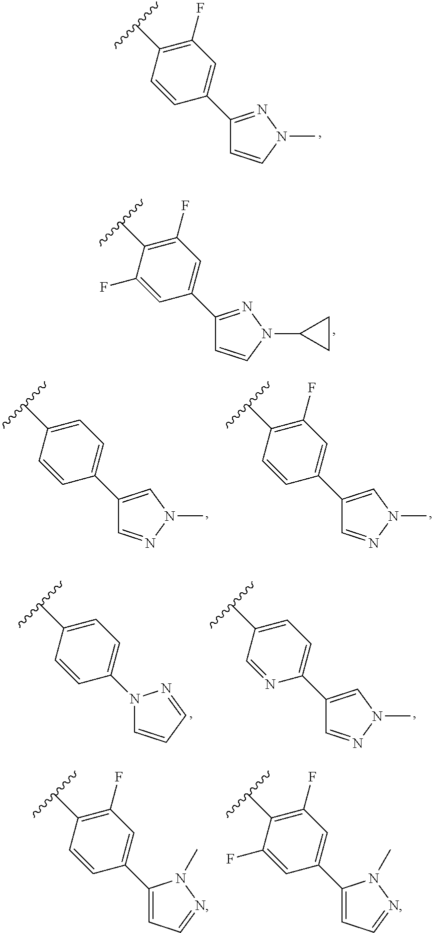

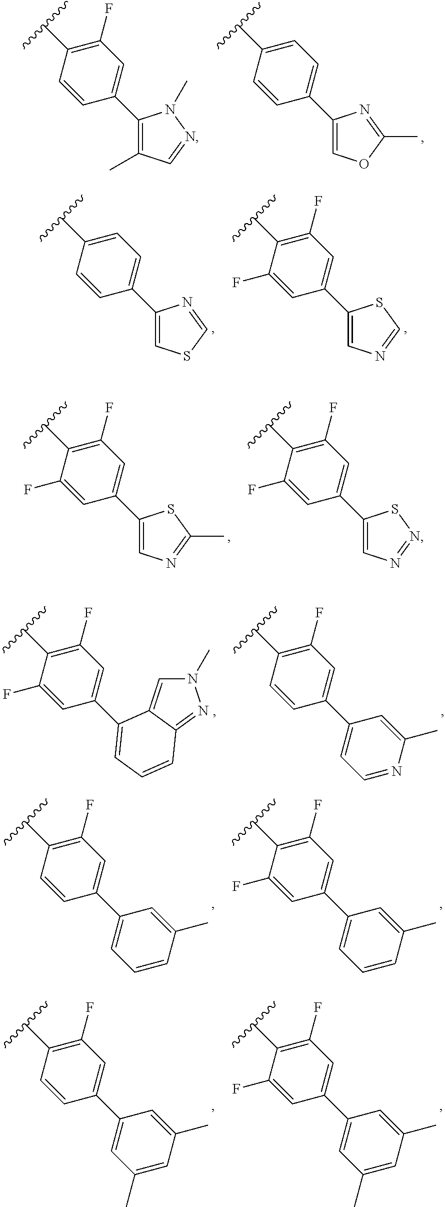

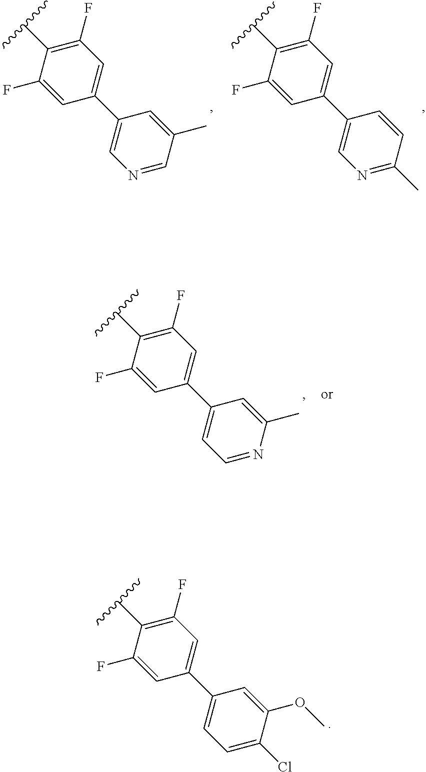

E39. The compound of any of E1-E38, or a pharmaceutically acceptable salt thereof, wherein A2 is -Li-Gi.

E40. The compound of any of E1-E39, or a pharmaceutically acceptable salt thereof, wherein the 4- to 12-membered heterocyclyl of G¹ contains one oxygen ring atom.

E41. The compound of any of E1-E40, or a pharmaceutically acceptable salt thereof, wherein G¹ is a monocyclic C₃₋₈cycloalkyl, a monocyclic 4- to 8-membered heterocyclyl containing one oxygen atom, or a 7- to 12-membered spirocyclic heterocyclyl containing one oxygen atom, wherein G¹ is unsubstituted or substituted with 1-2 substituents selected from the group consisting of —OH, —C₁₋₃alkylene-OH, and C₁₋₄alkyl.

E42. The compound of any of E1-E41, or a pharmaceutically acceptable salt thereof, wherein -L¹-G¹ is

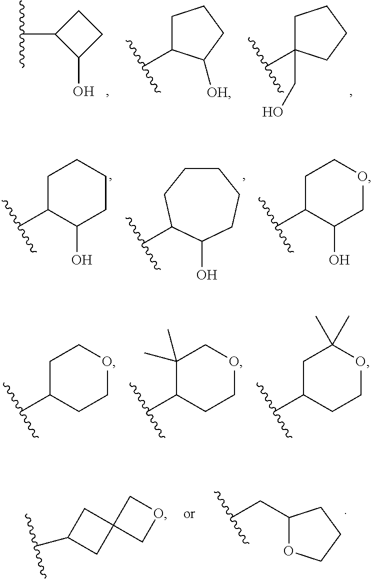

E43. The compound of any of E1-E42, or a pharmaceutically acceptable salt thereof, wherein L¹ is a bond.

E44. The compound of any of E1-E42, or a pharmaceutically acceptable salt thereof, wherein L¹ is a —CH₂—.

E45. The compound of any of E1-E38, or a pharmaceutically acceptable salt thereof, wherein A² is C₁₋₆alkyl optionally substituted with OH.

E45.1. The compound of E45, or a pharmaceutically acceptable salt thereof, wherein A² is

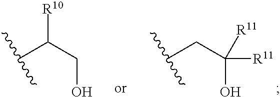

R¹⁰ is C₁₋₄alkyl; and each R¹¹ is independently C₁₋₂alkyl.

E45.2. The compound of E45 or E45.1, or a pharmaceutically acceptable salt thereof, wherein A² is

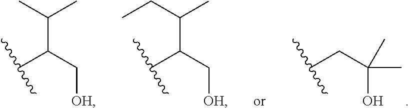

For example A² may be

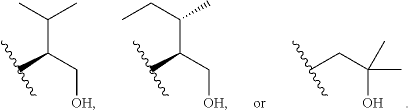

E46. The compound of any of E1-E38, or a pharmaceutically acceptable salt thereof, wherein A² is C₁₋₆haloalkyl optionally substituted with OH.

E46.1. The compound of E46, or a pharmaceutically acceptable salt thereof, wherein A² is

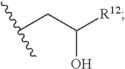

and R¹² is C₁₋₄haloalkyl.

E46.2. The compound of E46 or E46.1, or a pharmaceutically acceptable salt thereof, wherein A² is

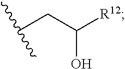

and R¹² is C₁fluoroalkyl. For example, A² may be

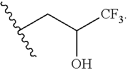 [SMILES: *CC(O)C(F)(F)F]

E47. The compound of any of E1-E46.2, or a pharmaceutically acceptable salt thereof, wherein R¹ is hydrogen.

E48. The compound of any of E1-E47, or a pharmaceutically acceptable salt thereof, wherein R² is hydrogen.

E49. Representative compounds of formula (I) include, but are not limited to the compounds listed in Tables A, B, C, and D, and their pharmaceutically acceptable salts.

Compounds may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. In the compounds of formula (I) when no specific configuration is indicated at a stereogenic center (e.g., carbon), the compounds include all possible stereoisomers.

Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compound may possess tautomeric forms, as well as geometric isomers, and that these also constitute embodiments of the disclosure.

In the compounds of formula (I), and any subformulas, any “hydrogen” or “H,” whether explicitly recited or implicit in the structure, encompasses hydrogen isotopes ¹H (protium) and ²H (deuterium).

The present disclosure also includes an isotopically-labeled compound (e.g., deuterium labeled), where an atom in the isotopically-labeled compound is specified as a particular isotope of the atom. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### 3. Pharmaceutical Compositions

The disclosed compounds may be incorporated into pharmaceutical compositions suitable for administration to a subject (such as a patient, which may be a human or non-human). The disclosed compounds may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

a. Spray-Dried Dispersion Formulations

The disclosed compounds may be formulated as a spray-dried dispersion (SDD). An SDD is a single-phase, amorphous molecular dispersion of a drug in a polymer matrix. It is a solid solution with the compound molecularly “dissolved” in a solid matrix. SDDs are obtained by dissolving drug and a polymer in an organic solvent and then spray-drying the solution. The use of spray drying for pharmaceutical applications can result in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

Thus, in one embodiment, the disclosure may provide a spray-dried dispersion formulation comprising a compound of formula (I).

### 4. Therapeutic Uses and Methods

The disclosed compounds are positive allosteric modulators of mAChR M₁. Thus, by positive allosteric modulation, the compounds indirectly activate the muscarinic receptor subtype M₁. In one aspect, the disclosed compounds potentiate the agonist response (e.g., acetylcholine) of mAChR M₁. In a further aspect, the disclosed compounds increase mAChR M₁ response to non-maximal concentrations of agonist in the presence of compound compared to the response to agonist in the absence of compound. The potentiation of mAChR M₁ activity can be demonstrated by methodology known in the art. For example, activation of mAChR M₁ activity can be determined by measurement of calcium flux in response to agonist, e.g. acetylcholine, in cells loaded with a Ca²⁺-sensitive fluorescent dye (e.g., Fluo-4). In a further aspect, the calcium flux was measured as an increase in fluorescent static ratio. In a yet further aspect, positive allosteric modulator activity was analyzed as a concentration-dependent increase in the EC₂₀ acetylcholine response (i.e. the response of mAChR M₁ at a concentration of acetylcholine that yields 20% of the maximal response).

In an embodiment, the disclosed compounds may activate mAChR M₁ response as an increase in calcium fluorescence in mAChR M₁-transfected CHO-K1 cells in the presence of the compound, compared to the response of equivalent CHO-K1 cells in the absence of the compound. For example, a disclosed compound may have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM. In an embodiment, the mAChR M₁-transfected CHO-K1 cells are transfected with human mAChR M₁. In another embodiment, the mAChR M₁-transfected CHO-K1 cells are transfected with rat mAChR M₁.

In an embodiment, the disclosed compounds exhibit weak or substantially no agonist activation of mAChR M₁ response (i.e., lack of activation in the absence of a known agonist such as acetylcholine). Lack of agonist activity may be measured as weak or no increase in calcium fluorescence in mAChR M₁-transfected CHO-K1 cells in the presence of the compound, compared to the response of equivalent CHO-K1 cells in the absence of the compound. Lack of mAChR M₁ agonist activity may be determined as a percent response relative to acetylcholine. For example, a disclosed compound may have less than or equal to 30%, 25%, 20%, 15%, 10%, 5%, or 1% mAChR M₁ agonist activity relative to acetylcholine. A disclosed compound may have substantially no mAChR M₁ agonist activity. In further embodiments, the disclosed compounds exhibit positive allosteric modulation of mAChR M₁ response to acetylcholine, as described herein, at concentrations that have weak or substantially no agonist activity, as described herein. The absence of mAChR M₁ agonist activity may contribute to the avoidance of cholinergic adverse effect liability.

In an embodiment, the disclosed compounds exhibit potentiation of mAChR M₁ response to acetylcholine as an increase in response to non-maximal concentrations of acetylcholine in CHO-K1 cells transfected with a mammalian mAChR M₁ in the presence of the compound, compared to the response to acetylcholine in the absence of the compound. For example, CHO-K1 cells can be transfected with human mAChR M₁. For example, CHO-K1 cells can be transfected with rat mAChR M₁. For example, a compound can exhibit positive allosteric modulation of mAChR M₁ with an EC₅₀ of less than or equal to 10 μM, less than or equal to 5 μM, less than or equal to 2.5 μM, less than or equal to 1 μM, less than or equal to 500 nM, less than or equal to 250 nM, or less than or equal to 100 nM. Alternatively, the disclosed compounds exhibit potentiation of mAChR M₁ response to acetylcholine as an increase in response to non-maximal concentrations of acetylcholine in CHO-K1 cells transfected with human mAChR M₁ in the presence of the compound, compared to the response to acetylcholine in the absence of the compound. For example, a compound can exhibit positive allosteric modulation of mAChR M₁ with an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM.

In an embodiment, the disclosed compounds exhibit positive allosteric modulation of mAChR M₁ response to acetylcholine as an increase in response to non-maximal concentrations of acetylcholine in CHO-K1 cells transfected with a mAChR M₁ in the presence of the compound, compared to the response to acetylcholine in the absence of the compound. For example, the disclosed compounds may exhibit positive allosteric modulation of the mAChR M₁ response to acetylcholine with an EC₅₀ of less than or equal to 10 μM, less than or equal to 5 μM, less than or equal to 2.5 μM, less than or equal to 1 μM, less than or equal to 500 nM, less than or equal to 250 nM, or less than or equal to 100 nM. In an embodiment, the EC₅₀ for positive allosteric modulation is determined in CHO-K1 cells are transfected with a mAChR M₁. In another embodiment, the CHO-K1 cells are transfected with a human mAChR M₁. In another embodiment, the CHO-K1 cells are transfected with a rat mAChR M₁.

In an embodiment, the compounds activate mAChR M₁ response in mAChR M₁-transfected CHO-K1 cells with an EC₅₀ less than the EC₅₀ for one or more of mAChR M₂, mAChR M₃, mAChR M₄, or mAChR M₅ response in mAChR M₂, M₃, M₄ or M₅-transfected CHO-K1 cells. That is, the disclosed compounds can have selectivity for the mAChR M₁ receptor vis-à-vis one or more of the mAChR M₂, M₃, M₄ or M₅ receptors. For example, the disclosed compounds can activate mAChR M₁ response with an EC₅₀ of at least 5-fold less than that for mAChR M₂, at least 10-fold less than that for mAChR M₂, at least 20-fold less than that for mAChR M₂, at least 30-fold less than that for mAChR M₂, at least 50-fold less than that for mAChR M₂, or at least 100-fold less than that for mAChR M₂. In another embodiment, the disclosed compounds can activate mAChR M₁ response with an EC₅₀ of at least 5-fold less than that for mAChR M₃, at least 10-fold less than that for mAChR M₃, at least 20-fold less than that for M₃, at least 30-fold less than that for mAChR M₃, at least 50-fold less than that for mAChR M₃, or at least 100-fold less than that for mAChR M₃. In another embodiment, the disclosed compounds can activate mAChR M₁ response with an EC₅₀ of at least 5-fold less than that for mAChR M₄, at least 10-fold less than that for mAChR M₄, at least 20-fold less than that for M₄, at least 30-fold less than that for mAChR M₄, at least 50-fold less than that for mAChR M₄, or at least 100-fold less than that for mAChR M₄. In another embodiment, the disclosed compounds can activate mAChR M₁ response with an EC₅₀ of at least 5-fold less than that for mAChR M₅, at least 10-fold less than that for mAChR M₅, at least 20-fold less than that for mAChR M₅, at least 30-fold less than that for mAChR M₅, at least 50-fold less than that for mAChR M₅, or at least 100-fold less than that for mAChR M₅. In another embodiment, the disclosed compounds can activate mAChR M₁ response with an EC₅₀ of at least 5-fold less than that for the mAChR M₂, M₃, M₄ or M₅ receptors, at least 10-fold less than that for the mAChR M₂, M₃, M₄ or M₅ receptors, at least 20-fold less than that for the mAChR M₂, M₃, M₄ or M₅ receptors, at least 30-fold less than that for the mAChR M₂, M₃, M₄ or M₅ receptors, at least 50-fold less than that for the mAChR M₂, M₃, M₄ or M₅ receptors, or at least 100-fold less than that for the mAChR M₂, M₃, M₄ or M₅ receptors. In another embodiment, the compound activates mAChR M₁ response in mAChR M₁-transfected CHO-K1 cells and is inactive for one or more of mAChR M₁, mAChR M₃, mAChR M₄, or mAChR M₅ response in mAChR M₂, M₃, M₄ or M₅-transfected CHO-K1 cells.

In an embodiment, the compounds activate mAChR M₁ response in M₁-transfected CHO-K1 cells with an EC₅₀ of less than or equal to 10 μM and exhibits a selectivity for the M₁ receptor vis-à-vis one or more of the mAChR M₂, M₃, M₄ or M₅ receptors. For example, the compounds can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for mAChR M₂, at least 10-fold less than that for mAChR M₂, at least 20-fold less than that for mAChR M₂, at least 30-fold less than that for mAChR M₂, or at least 50-fold less than that for mAChR M₂. In another embodiment, the compounds can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for mAChR M₃, at least 10-fold less than that for mAChR M₃, at least 20-fold less than that for mAChR M₃, at least 30-fold less than that for mAChR M₃, or at least 50-fold less than that for mAChR M₃. In another embodiment, the compounds can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for mAChR M₄, of at least 10-fold less than that for mAChR M₄, of at least 20-fold less than that for mAChR M₄, of at least 30-fold less than that for mAChR M₄, or at least 50-fold less than that for mAChR M₄. In another embodiment, the compound can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for mAChR M₅, of at least 10-fold less than that for mAChR M₅, of at least 20-fold less than that for mAChR M₅, of at least 30-fold less than that for mAChR M₅, or at least 50-fold less than that for mAChR M₅. In another embodiment, the compounds can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for the mAChR M₂, M₃, M₄ or M₅ receptors, at least 10-fold less than that for the mAChR M₂, M₃, M₄ or M₅ receptors, at least 20-fold less than that for the mAChR M₂, M₃, M₄ or M₅ receptors, at least 30-fold less than that for the mAChR M₂, M₃, M₄ or M₅ receptors, or at least 50-fold less than that for the mAChR M₂, M₃, M₄ or M₅ receptors.

The disclosed compounds may be used in methods for treatment of mAChR M₁ related medical disorders and/or diseases. The methods of treatment may comprise administering to a subject in need of such treatment a composition comprising a therapeutically effective amount of the compound of formula (I).

The compounds can be administered to a subject in need thereof to modulate mAChR M₁, for a variety of diverse biological processes. The present disclosure is directed to methods for administering the composition to potentiate mAChR M₁, a GPCR whose dysfunction is associated with neurological and psychiatric disorders, for example.

The compounds may be useful for treating and preventing certain diseases and disorders in humans and animals related to mAChR M₁ dysfunction. Treatment or prevention of such diseases and disorders can be effected by modulating mAChR M₁ in a subject, by administering a compound or composition of the disclosure, either alone or in combination with another active agent as part of a therapeutic regimen to a subject in need thereof.

In combination therapy, the other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

In an embodiment, the compounds can be coadministered with anti-Alzheimer's agents, beta-secretase inhibitors, gamma-secretase inhibitors, orthosteric muscarinic agonists, muscarinic potentiators, cholinesterase inhibitors, HMG-CoA reductase inhibitors, NSAIDs and anti-amyloid antibodies. In a further aspect, the compounds can be administered in combination with sedatives, hypnotics, anxiolytics, antipsychotics (typical and atypical), selective serotonin reuptake inhibitors (SSRIs), monoamine oxidase inhibitors (MAOIs), 5-HT2 antagonists, GlyT1 inhibitors and the like such as, but not limited to: risperidone, clozapine, haloperidol, fluoxetine, prazepam, xanomeline, lithium, phenobarbitol, and salts thereof and combinations thereof.

The compounds may be useful for treating a disease or disorder associated with dysfunction of mAChR M₁, wherein the disease or disorder is selected from at least one of Alzheimer's disease, a sleep disorder, a pain disorder, a cognitive disorder, psychosis, schizophrenia, conduct disorder, disruptive behavior disorder, bipolar disorder, psychotic episodes of anxiety, anxiety associated with psychosis, psychotic mood disorders, severe major depressive disorder, mood disorders associated with psychotic disorders, acute mania, depression associated with bipolar disorder, mood disorders associated with schizophrenia, behavioral manifestations of mental retardation, conduct disorder, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

The compounds may be useful for treating a pain disorder, wherein the pain disorder is neuropathic pain, central pain syndrome, postsurgical pain syndrome, bone and joint pain, repetitive motion pain, dental pain, cancer pain, myofascial pain, perioperative pain, chronic pain, dysmennorhea, inflammatory pain, headache, migraine headache, cluster headache, headache, primary hyperalgesia, secondary hyperalgesis, primary allodynia, secondary allodynia, or a combination thereof.

The compounds disclosed herein are useful for treating, preventing, ameliorating, controlling or reducing the risk of a variety of disorders wherein the patient or subject would benefit from selective positive allosteric modulation of the M₁ receptor. In one aspect, a treatment can include selective M₁ receptor modulation to an extent effective to affect cholinergic activity. Thus, a disorder can be associated with cholinergic activity, for example cholinergic hypofunction. In one aspect, provided is a method of treating or preventing a disorder in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more disorders, for which muscarinic receptor activation is predicted to be beneficial, in a subject comprising the step of administering to the subject at least one disclosed compound; at least one disclosed pharmaceutical composition; and/or at least one disclosed product in a dosage and amount effective to treat the disorder in the subject.

The disclosure is directed to the use of described chemical compositions to treat diseases or disorders in patients (preferably human) wherein muscarinic receptor activation would be predicted to have a therapeutic effect, such as Alzheimer's disease (both palliative cognitive and disease-modifying), cognitive impairment, schizophrenia, pain disorders (including acute pain, neuropathic pain and inflammatory pain), and sleep disorders, by administering one or more disclosed compounds or products.

Also provided is a method for the treatment of a disorder in a mammal comprising the step of administering to the mammal at least one disclosed compound, composition, or medicament.

a. Neurological and Psychiatric Disorders

The disclosed compounds have utility in treating a variety of neurological and psychiatric disorders, including one or more of the following conditions or diseases: schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthetics, amphetamine and other psychostimulants and cocaine) psychosis psychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age-related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; NMDA receptor-related disorders such as autism, depression, benign forgetfulness, childhood learning disorders and closed head injury; movement disorders, including akinesias and akinetic-rigid syndromes (including Parkinson's disease, drug-induced parkinsonism, post-encephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de La Tourette's syndrome, epilepsy, muscular spasms and disorders associated with muscular spasticity or weakness including tremors; dyskinesias including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics), and dystonia (including generalized dystonia such as idiopathic dystonia, drug-induced dystonia, symptomatic dystonia and paroxysmal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

1. Cognitive Disorders

The present disclosure provides a method for treating cognitive disorders, comprising: administering to a patient in need thereof an effective amount of a compound of the present disclosure. Particular cognitive disorders are dementia, delirium, amnestic disorders and age-related cognitive decline. The text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

2. Anxiety Disorders

The present disclosure provides a method for treating anxiety disorders, comprising: administering to a patient in need thereof an effective amount of a compound of the present disclosure. Particular anxiety disorders are generalized anxiety disorder, obsessive-compulsive disorder and panic attack. The text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

3. Alzheimer's Disease

Alzheimer's disease (AD) is a neurodegenerative disease affecting the elderly, which results in progressive impairment of memory, language skills and severe behavioral deficits. Hallmarks of the disease include degeneration of cholinergic neurons in the cerebral cortex, hippocampus, basal forebrain and other regions of the brain important for memory and cognition. Other hallmarks of AD include neurofibrillary tangles composed of hyperphosphorylated tau and accumulation of amyloid β peptide (Aβ). Aβ is a 39-43 amino acid peptide produced in the brain by proteolytic processing of β-amyloid precursor protein (APP) by the β-amyloid cleaving enzyme (BACE) and gamma secretase which leads to accumulation of Aβ in the brain, where Aβ 1-40 and 1-42 are the principal aggregate-forming species of Aβ.

Activation of various muscarinic receptors, particularly the M₁ subtype, has been proposed as a mechanism to enhance cognition in disorders such as AD. Thus, without wishing to be bound by theory, it is believed that selective positive allosteric modulators of mAChR subtypes that regulate processes involved in cognitive function could prove superior to AChE inhibitors for treatment of AD and related disorders as it is postulated that these compounds would exhibit improved selectivity for specific mAChRs.

Phase III clinical trials have shown that orthosteric mAChR activators can have efficacy in improving cognitive performance in AD patients. Moreover, data indicate that administration of M₁ activators decreases behavioral disturbances, including delusions, hallucinations, outbursts, and other symptoms in patients suffering from neurodegenerative diseases such as Alzheimer's disease. However, dose limiting adverse effects that may be due to lack of mAChR M₁ selectivity led to failed launches of previous M₁ agonists. In some cases, evidence suggests that mAChR activation also has the potential to be disease-modifying in that these agents may lower Aβ in AD patients. The M₁-selective allosteric agonist TBPB was found to display effects on the processing of APP toward the non-amyloidogenic pathway and decrease Aβ1-40 and 1-42 production in vitro. These data suggest that selective activation of M₁ may provide a novel approach for both symptomatic and disease modifying the treatment of Alzheimer's disease.

4. Schizophrenia

Schizophrenia is a debilitating psychiatric disorder characterized by a combination of negative (blunted affect, withdrawal, anhedonia) and positive (paranoia, hallucinations, delusions) symptoms as well as marked cognitive deficits. While schizophrenia remains an idiopathic disorder, it appears to be produced by a complex interaction of biological, environmental, and genetic factors. Over 40 years ago it was found that phencyclidine (PCP) induces a psychotic state in humans that is very similar to that observed in schizophrenic patients. The finding that the main mode of action of PCP is that of a non-competitive antagonist of the N-methyl-D-aspartate (NMDA) subtype of ionotropic glutamate receptor stimulated a series of studies that have led to the development of the NMDA receptor hypofunction model of schizophrenia.

The present disclosure provides a method for treating schizophrenia or psychosis comprising: administering to a patient in need thereof an effective amount of a compound of the present disclosure. Particular schizophrenia or psychosis pathologies are paranoid, disorganized, catatonic or undifferentiated schizophrenia and substance-induced psychotic disorder. The text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder.

NMDA receptor function can be modulated by activation of G Protein-Coupled Receptors (GPCRs) that are known to physically and/or functionally interact with the NMDA receptor. The NMDA receptor hypofunction hypothesis is a proposal to explain the underlying cause of schizophrenia. According to this hypothesis, any agent that can potentiate NMDA receptor currents, either directly by action on modulatory sites on the NMDA receptor (e.g., the glycine co-agonist binding site) or indirectly by activation of GPCRs known to potentiate NMDA receptor function (e.g. the mAChR M₁), has the potential to ameliorate the symptoms of schizophrenia. In both preclinical and in clinical studies, xanomeline, an M₁/M₄ preferring orthosteric agonist has proved efficacious with regard to positive, negative and cognitive symptoms, indicating that M₁ activation is a reasonable approach to the treatment of schizophrenia. More recently, the selective M₁ allosteric agonist TBPB demonstrated efficacy in multiple preclinical models of schizophrenia.

As used herein, the term “schizophrenia or psychosis” includes treatment of those mental disorders as described in DSM-W-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

5. Substance-Related Disorders and Addictive Behaviors

The present disclosure provides a method for treating substance-related disorders and addictive behaviors, comprising: administering to a patient in need thereof an effective amount of a compound of the present disclosure. Particular substance-related disorders and addictive behaviors are persisting dementia, persisting amnestic disorder, psychotic disorder or anxiety disorder induced by substance abuse; and tolerance of, dependence on or withdrawal from substances of abuse. The text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

6. Pain

In another aspect, the present disclosure provides a method for treating pain, comprising: administering to a patient in need thereof an effective amount of a compound of the present disclosure. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

7. Obesity and Eating Disorders

The present disclosure provides a method for treating obesity or eating disorders associated with excessive food intake and complications associated therewith, comprising: administering to a patient in need thereof an effective amount of a compound of the present disclosure. Obesity is included in the tenth edition of the International Classification of Diseases and Related Health Problems (ICD-10) (1992 World Health Organization) as a general medical condition. The text revision of the fourth edition of the Diagnostic and Statistical Manual of Mental Disorders (DSM-IV-TR) (2000, American Psychiatric Association, Washington D.C.) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-W-TR. The skilled artisan will recognize that there are alternative nomenclatures, nosologies and classification systems for general medical conditions, and that these systems evolve with medical and scientific progress. Thus, the term “obesity or eating disorders associated with excessive food intake” is intended to include like conditions and disorders that are described in other diagnostic sources.

The compounds are further useful in a method for the prevention, treatment, control, amelioration, or reduction of risk of the diseases, disorders and conditions noted herein. The compounds are further useful in a method for the prevention, treatment, control, amelioration, or reduction of risk of the aforementioned diseases, disorders and conditions in combination with other agents.

The present disclosure is further directed to administration of a selective M₁ receptor modulator for improving treatment outcomes in the context of cognitive or behavioral therapy. That is, in one aspect, the disclosure relates to a cotherapeutic method comprising the step of administering to a mammal an effective amount and dosage of at least one compound of the disclosure in connection with cognitive or behavioral therapy.

In another aspect, administration improves treatment outcomes in the context of cognitive or behavioral therapy. Administration in connection with cognitive or behavioral therapy can be continuous or intermittent. Administration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As another example, cognitive or behavioral therapy can be provided before or after administration within a period of time of 1, 2, 3, 4, 5, 6, 7, 8, 9, or 10 half-lives of the administered compound.

b. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

c. Combination Therapies

In one aspect, the disclosed compounds can be used in combination with one or more other drugs in the treatment, prevention, control, amelioration, or reduction of risk of diseases or conditions for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present disclosure. When a compound of the present disclosure is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present disclosure.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present disclosure. When a compound of the present disclosure is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present disclosure.

The weight ratio of a disclosed compound to the second active ingredient can be varied and will depend upon the effective dose of each ingredient. Generally, an effective dose of each will be used. Thus, for example, when a compound of the present disclosure is combined with another agent, the weight ratio of a disclosed compound to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used.

In such combinations disclosed compounds and other active agents can be administered separately or in conjunction. In addition, the administration of one element can be prior to, concurrent to, or subsequent to the administration of other agent(s).

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In an embodiment, the disclosed compounds can be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies. In another embodiment, the subject compound can be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, buspirone, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, Zolpidem, and salts thereof, and combinations thereof, and the like, or the subject compound can be administered in conjunction with the use of physical methods such as with light therapy or electrical stimulation.

In an embodiment, the disclosed compounds can be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In an embodiment, the disclosed compounds can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound can be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In an embodiment, the disclosed compounds can be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts. thereof.

### 5. Kits

In one aspect, the disclosure provides kits comprising at least one disclosed compound or a pharmaceutically acceptable salt thereof, and one or more of:

(a) at least one agent known to increase mAChR M₁ activity;

(b) at least one agent known to decrease mAChR M₁ activity;

(c) at least one agent known to treat a disorder associated with cholinergic activity;

(d) instructions for treating a disorder associated with cholinergic activity;

(e) instructions for treating a disorder associated with M₁ receptor activity; or

(f) instructions for administering the compound in connection with cognitive or behavioral therapy.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

That the disclosed kits can be employed in connection with disclosed methods of use.

The kits may further comprise information, instructions, or both that use of the kit will provide treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of treating or preventing medical conditions in mammals (e.g., humans).

The compounds and processes of the invention will be better understood by reference to the following examples, which are intended as an illustration of and not a limitation upon the scope of the invention.

### 6. Chemical Synthesis

Compounds of formula (I) may be prepared by synthetic processes or by metabolic processes. Preparation of the compounds by metabolic processes includes those occurring in the human or animal body (in vivo) or processes occurring in vitro.

Compounds of formula (I) may be synthesized as shown in the Schemes and Examples set forth below.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th}, ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

All NMR spectra were recorded on a 400 MHz AMX Bruker NMR spectrometer. ¹H chemical shifts are reported in δ values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration. Reversed-phase LCMS analysis was performed using an Agilent 1200 system comprised of a binary pump with degasser, high-performance autosampler, thermostatted column compartment, C18 column, diode-array detector (DAD) and an Agilent 6150 MSD with the following parameters. The gradient conditions were 5% to 95% acetonitrile with the aqueous phase 0.1% TFA in water over 1.4 minutes. Samples were separated on a Waters Acquity UPLC BEH C18 column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The M₅ detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

The following abbreviations may be used herein:
aq aqueous
BTF benzotrifluoride
conc. concentrated
Cpd compound
DCC N,N′-dicyclohexylcarbodiimide
DCM dichloromethane
DIPEA/DIEA diisopropylethylamine
DMF N,N-dimethylformamide
DMF⋅DMA N,N-dimethylformamide dimethyl acetal
DMSO dimethyl sulfoxide
dppf 1,1′-bis(diphenylphosphino)ferrocene
dppp 1,3-bis(diphenylphosphino)propane
EDC 1-Ethyl-3-(3-dimethylaminopropyl)carbodiimide
ES-MS electrospray mass spectrometry
EtOAc ethyl acetate
EtOH ethanol
Et3N triethylamine
eq/equiv equivalents
h or hr hour
HATU 1-[Bis(dimethylamino)methylene]-1H-1,2,3-triazolo[4,5-b]pyridinium 3oxide hexafluorophosphate
HBTU N,N,N′,N′-Tetramethyl-O-(1H-benzotriazol-1-yl)uronium hexafluorophosphate
HPLC high performance liquid chromatography
iPA isopropyl alcohol
KOAc potassium acetate
LCMS liquid chromatography-mass spectrometry
Me methyl
MeCN acetonitrile
MeOH methanol
mCPBA meta-chloroperoxybenzoic acid
min minutes
mw microwave
NBS N-bromosuccinimide
Pd(OAc)₂ palladium(II)acetate
Pd2(dba)₃ Tris(dibenzylideneacetone)dipalladium(O)
Pd(dppf)Cl₂ (1,1′-Bis(diphenylphosphino)ferrocene)palladium(II) dichloride
Ph phenyl
ppm parts per million
PyBOP (Benzotriazol-1-yloxy)tripyrrolidinophosphonium hexafluorophosphate
PyClU chlorodipyrrolidinocarbenium hexafluorophosphate
RP reverse phase
sat saturated
soln solution
r.t./rt/RT room temperature
TBTU 2-(1H-Benzotriazole-1-yl)-1,1,3,3-tetramethylaminium tetrafluoroborate
TFA trifluoroacetic acid
TFAA trifluoroacetic anhydride
THF tetrahydrofuran

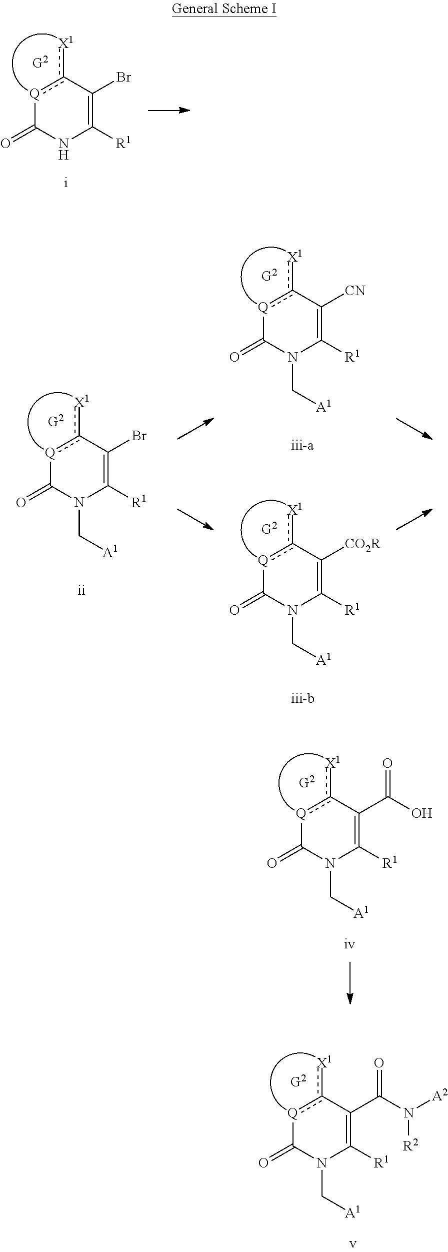

General Scheme I shows a method for preparing compounds of the invention. Starting bromo compound (i) may be reacted with suitable benzyl halides to provide bromo intermediates (ii). Typical palladium-catalyzed cross coupling of bromo intermediate (ii) with zinc cyanide may provide cyano intermediates (iii-a). Alternatively, palladium-catalyzed CO insertion of (ii) in the presence of an alcohol ROH (e.g., methanol) may provide the ester intermediate (iii-b). Reacting the cyano intermediates (iii-a) with concentrated acid (e.g., HCl) at elevated temperature may hydrolyze the nitrile to the corresponding acid (iv). Reacting the ester intermediates (iii-b) with base (e.g., LiGH in MeOH/THF) may hydrolyze the ester to the corresponding acid (iv). Reaction of acid (iv) with an amine under standard amide bond forming conditions known in the art may provide compounds of formula (v). Typical coupling conditions include a base (e.g., DIPEA) and a coupling reagent such as HATU, EDC, PyBOP, DCC, HBTU, or TBTU.

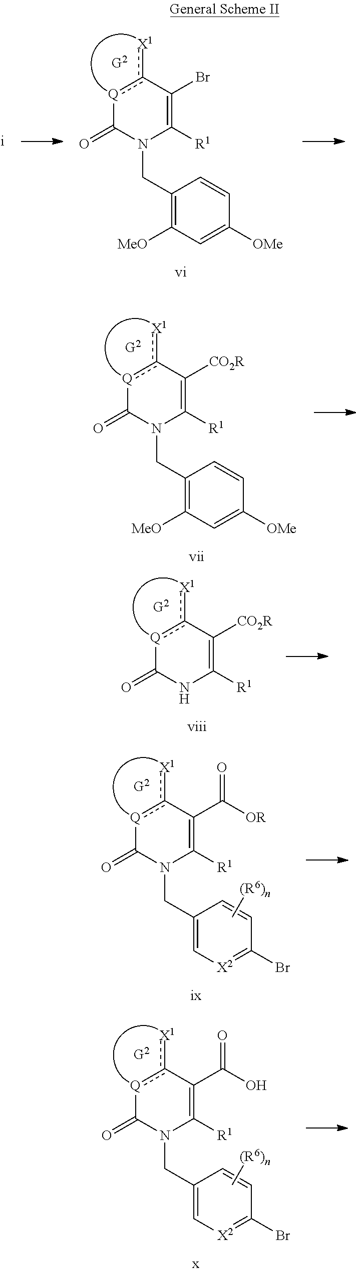

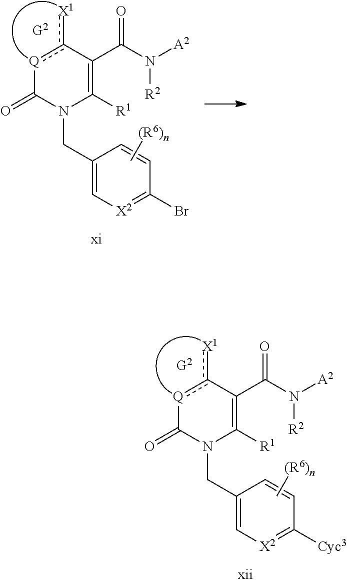

General Scheme II shows a method for preparing compounds of the invention of formula (xii). Protection of starting material (i) with a dimethoxybenzyl group by reaction with 1-(chloromethyl)-2,4-dimethoxybenzene in the presence of a base (e.g., potassium carbonate) in an organic solvent (e.g., DMF) may provide intermediate (vi). Reaction of (vi) using CO insertion chemistry described in General Scheme I may provide intermediate (vii). Deprotection of (vii) by reaction with trifluoroacetic acid with heating and optionally microwave irradiation may provide intermediate (viii). Benzylation of (viii) with an appropriate brominated reagent such as

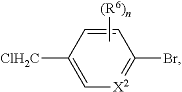

in the presence of base (e.g., potassium carbonate) in an organic solvent (e.g., DMF) may provide intermediate (ix). Reacting the ester intermediates (ix) with base (e.g., LiOH in MeOH/THF) may hydrolyze the ester to the corresponding acid (x). Reaction of acid (x) with an amine under standard amide bond forming conditions known in the art may provide compounds of formula (xi). Typical coupling conditions include a base (e.g., DIPEA) and a coupling reagent such as HATU, EDC, PyBOP, DCC, HBTU, or TBTU. Compounds of formula (xi) may be converted to compounds of formula (xii) using typical Suzuki reaction coupling conditions well known in the art, such as reaction with an aryl boronic acid or ester (e.g., pinacol boronate) in the presence of a catalytic Pd species, such as Pd(dppf)₂Cl₂ and a base such as K₂PO₄, Na₂CO₃, Cs₂CO₃, K₂CO₃, Ba(OH)₂ or Et3N.

### Example 1. 6-(2-Fluoro-4-(1-methyl-1H-pyrazol-3-yl)benzyl)-N-((3R,4S)-3-hydroxytetrahydro-2H-pyran-4-yl)-5-oxo-5,6-dihydro-1,6-naphthyridine-8-carboxamide (Compound A1)

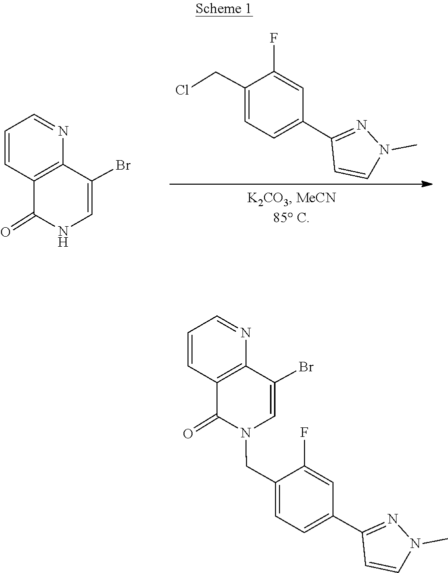 [SMILES: O=c1[nH]cc(Br)c2ncccc12 ; Cn1ccc(-c2ccc(CCl)c(F)c2)n1 ; Cn1ccc(-c2ccc(Cn3cc(Br)c4ncccc4c3=O)c(F)c2)n1]

8-Bromo-6-(2-fluoro-4-(1-methyl-1H-pyrazol-3-yl)benzyl)-1,6-naphthyridin-5(6H)-one. A reaction mixture of 8-bromo-1,6-naphthyridin-5(6H)-one (CAS #155057-97-9, 281 mg, 1.25 mmol, 1.25 eq.), 3-(4-(chloromethyl)-3-fluorophenyl)-1-methyl-1H-pyrazole (225 mg, 1.0 mmol, 1.0 eq.), and potassium carbonate (245 mg, 1.75 mmol, 1.75 eq.) in MeCN (5.0 mL, 0.2 M) was allowed to stir at 85° C. for 16 h. After cooling to rt, the reaction mixture was concentrated under reduced pressure. The crude material was re-dissolved in DMF/DMSO (1:1 v/v) and filtered to remove insoluble salts. Purification using reverse phase HPLC (30-70% MeCN in 0.05% NH₄OH aqueous solution) afforded the title compound as a white powder (348 mg, 84%). ¹H-NMR (400 MHz, DMSO-d₆) δ 9.05 (dd, J=6.3, 2.8 Hz, 1H), 8.57 (dd, J=8.0, 1.7 Hz, 1H), 8.34 (s, 1H), 7.73 (d, J=2.2 Hz, 1H), 7.64 (dd, J=8.0, 4.6 Hz, 1H), 7.59 (dd, J=5.8, 4.3 Hz, 1H), 7.57 (dd, J=1.9, 1.9 Hz, 1H), 7.27 (dd, J=8.1, 8.1 Hz, 1H), 6.73 (d, J=2.3 Hz, 1H), 5.26 (s, 2H), 3.87 (s, 3H); ES-MS [M+H]+=413.2/415.2.

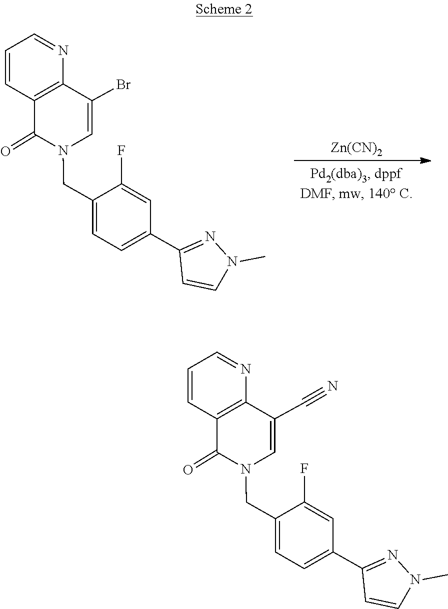 [SMILES: Cn1ccc(-c2ccc(Cn3cc(Br)c4ncccc4c3=O)c(F)c2)n1 ; "Zn(CN)2" ; Cn1ccc(-c2ccc(Cn3cc(C#N)c4ncccc4c3=O)c(F)c2)n1]

6-(2-Fluoro-4-(1-methyl-1H-pyrazol-3-yl)benzyl)-5-oxo-5,6-dihydro-1,6-naphthyridine-8-carbonitrile. 8-Bromo-6-(2-fluoro-4-(1-methyl-1H-pyrazol-3-yl)benzyl)-1,6-naphthyridin-5(6H)-one (207 mg, 0.5 mmol, 1.0 eq.), zinc cyanide (73 mg, 0.625 mmol, 1.25 eq.), tris(dibenzylideneacetone)dipalladium(0) (69 mg, 0.075 mmol, 0.15 eq.), 1,1′-bis(diphenylphosphino)ferrocene (42 mg, 0.075 mmol, 0.15 eq.) were combined into a microwave vial. Degassed DMF (5.0 mL, 0.2 M) was added. The reaction mixture was evacuated and filled with nitrogen and subjected to a microwave reactor for 30 min at 140° C. After cooling to rt, the reaction mixture was filtered to remove any insoluble salts. The crude material was purified using reverse phase HPLC (20-55% MeCN in 0.05% NH₄OH aqueous solution) to provide the title compound (135 mg, 75%). ¹H-NMR (400 MHz, DMSO-d₆) δ 9.06 (dd, J=4.6, 1.7 Hz, 1H), 8.90 (s, 1H), 8.57 (dd, J=8.1, 1.8 Hz, 1H), 7.74 (d, J=2.2 Hz, 1H), 7.68 (dd, J=8.1, 4.6 Hz, 1H), 7.60 (dd, J=6.3, 1.4 Hz, 1H), 7.57 (dd, J=1.9, 1.9 Hz, 1H), 7.34 (dd, J=7.9, 7.9 Hz, 1H), 6.74 (d, J=2.3 Hz, 1H), 5.26 (s, 2H), 3.87 (s, 3H); ES-MS [M+H]+=360.4.

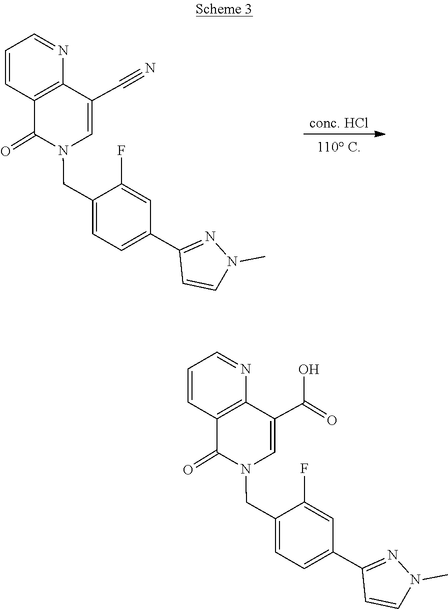 [SMILES: Cn1ccc(-c2ccc(Cn3cc(C#N)c4ncccc4c3=O)c(F)c2)n1 ; "conc. HCl" ; Cn1ccc(-c2ccc(Cn3cc(C(=O)O)c4ncccc4c3=O)c(F)c2)n1]

6-(2-Fluoro-4-(1-methyl-1H-pyrazol-3-yl)benzyl)-5-oxo-5,6-dihydro-1,6-naphthyridine-8-carboxylic acid. A mixture of 6-(2-fluoro-4-(1-methyl-1H-pyrazol-3-yl)benzyl)-5-oxo-5,6-dihydro-1,6-naphthyridine-8-carbonitrile (135 mg, 0.38 mmol, 1 eq.) and concentrated hydrochloric acid solution (37%, 2.0 mL) was stirred at 110° C. After 3 h, the mixture was concentrated and purified using reverse phase HPLC (5-45% MeCN in 0.1% TFA aqueous solution) to provide the title compound as a white powder (165 mg). ¹H-NMR (400 MHz, DMSO-d₆) δ 9.08 (dd, J=4.8, 1.7 Hz, 1H), 8.85 (s, 1H), 8.73 (dd, J=8.1, 1.7 Hz, 1H), 7.76 (dd, J=8.1, 4.6 Hz, 1H), 7.74 (d, J=2.2 Hz, 1H), 7.60 (dd, J=6.3, 1.6 Hz, 1H), 7.58 (s, 1H), 7.36 (dd, J=7.9, 7.9 Hz, 1H), 6.74 (d, J=2.3 Hz, 1H), 5.40 (s, 2H), 3.87 (s, 3H); ES-MS [M+H]+=379.3.

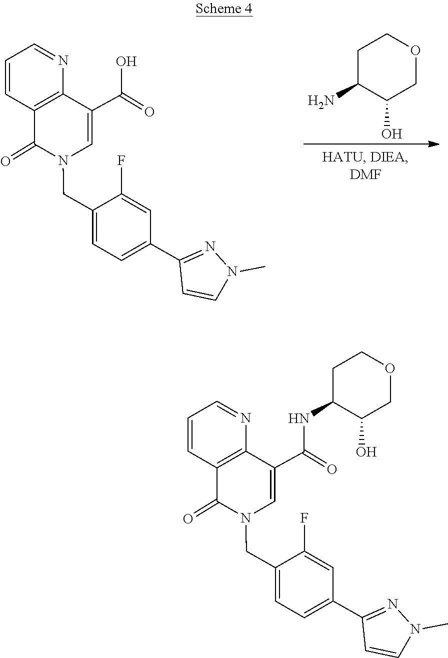 [SMILES: Cn1ccc(-c2ccc(Cn3cc(C(=O)O)c4ncccc4c3=O)c(F)c2)n1 ; N[C@H]1CCOC[C@@H]1O ; Cn1ccc(-c2ccc(Cn3cc(C(=O)N[C@H]4CCOC[C@@H]4O)c4ncccc4c3=O)c(F)c2)n1]

6-(2-Fluoro-4-(1-methyl-1H-pyrazol-3-yl)benzyl)-N-((3R,4S)-3-hydroxytetrahydro-2H-pyran-4-yl)-5-oxo-5,6-dihydro-1,6-naphthyridine-8-carboxamide (Compound A1). To a solution of 6-(2-fluoro-4-(1-methyl-1H-pyrazol-3-yl)benzyl)-5-oxo-5,6-dihydro-1,6-naphthyridine-8-carboxylic acid (as TFA salt, 15 mg, 0.030 mmol, 1 eq.) in DMF (1 mL) was added N,N-diisopropylethylamine (32 μL, 0.183 mmol, 6 eq.) and (3R,4S)-4-aminotetrahydropyran-3-ol (as L-tyrosine salt, 15 mg, 0.046 mmol, 1.5 eq.). After 3 min of stirring, HATU (23 mg, 0.061 mmol, 2.0 eq.) was added and the reaction mixture was allowed to stir for 20 min. Purification by reverse phase HPLC afforded the title compound (6.4 mg, 44%). ¹H-NMR (400 MHz, DMSO-d₆) δ 10.6 (d, J=7.3 Hz, 1H), 9.05 (dd, J=4.6, 1.8 Hz, 1H), 8.69 (s, 1H), 8.67 (dd, J=8.1, 1.8 Hz, 1H), 7.74 (d, J=2.2 Hz, 1H), 7.66 (dd, J=8.1, 4.6 Hz, 1H), 7.60 (s, 1H), 7.58 (dd, J=3.0, 1.6 Hz, 1H), 7.33 (dd, J=7.9, 7.9 Hz, 1H), 6.73 (d, J=2.3 Hz, 1H), 5.38 (s, 2H), 5.14 (d, J=5.3 Hz, 1H), 3.87 (s, 3H), 3.92-3.70 (m, 3H), 3.54-3.47 (m, 1H), 3.40 (ddd, J=11.3, 2.2, 2.2 Hz, 1H), 3.13 (dd, J=11.1, 9.2 Hz, 1H), 2.10-2.06 (m, 1H), 1.58-1.48 (m, 1H); ES-MS [M+H]+=478.4.

### Example 2. 6-(2-Fluoro-4-(1-methyl-1H-pyrazol-3-yl)benzyl)-5-oxo-N-(2-oxaspiro[3.3]heptan-6-yl)-5,6-dihydro-1,6-naphthyridine-8-carboxamide (Compound A5)

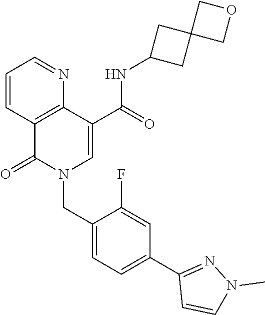 [SMILES: Cn1ccc(-c2ccc(Cn3cc(C(=O)NC4CC5(COC5)C4)c4ncccc4c3=O)c(F)c2)n1]

Compound 5 is prepared in a similar manner as Compound 1 as a white solid (8 mg, 45%). ¹H-NMR (400 MHz, DMSO-d₆) δ 10.6 (d, J=7.2 Hz, 1H), 9.06 (dd, J=4.6, 1.8 Hz, 1H), 8.66 (dd, J=8.1, 1.8 Hz, 1H), 8.65 (s, 1H), 7.74 (d, J=2.2 Hz, 1H), 7.66 (dd, J=8.1, 4.6 Hz, 1H), 7.60 (s, 1H), 7.58 (dd, J=3.0, 1.6 Hz, 1H), 7.32 (dd, J=7.9, 7.9 Hz, 1H), 6.73 (d, J=2.3 Hz, 1H), 5.36 (s, 2H), 4.64 (s, 2H), 4.54 (s, 2H), 4.31-4.22 (m, 2H), 3.87 (s, 3H), 2.67-2.62 (m, 2H), 2.26-2.21 (m, 2H); ES-MS [M+H]+=474.4.

### Example 3. 6-(2-Fluoro-4-(2-methylpyridin-4-yl)benzyl)-N-((1S,2S)-2-hydroxycyclohexyl)-5-oxo-5,6-dihydro-1,6-naphthyridine-8-carboxamide (Compound A27)

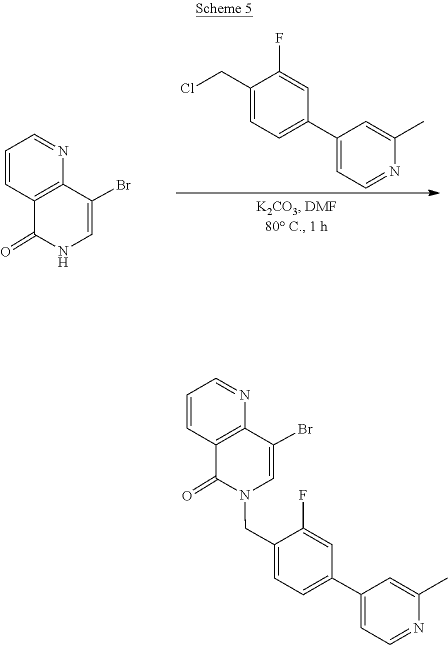 [SMILES: O=c1[nH]cc(Br)c2ncccc12 ; Cc1cc(-c2ccc(CCl)c(F)c2)ccn1 ; Cc1cc(-c2ccc(Cn3cc(Br)c4ncccc4c3=O)c(F)c2)ccn1]

8-Bromo-6-(2-fluoro-4-(2-methylpyridin-4-yl)benzyl)-1,6-naphthyridin-5(6H)-one. A mixture of 8-bromo-1,6-naphthyridin-5(6H)-one (74 mg, 0.40 mmol, 1.1 eq.), 4-(4-(chloromethyl)-3-fluorophenyl)-2-methylpyridine (70 mg, 0.3 mmol, 1.0 eq.) and potassium carbonate (73.4 mg, 0.52 mmol, 1.75 eq.) in DMF (3 mL, 0.1 M) was allowed to stir at 80° C. After 1 h, the reaction mixture was passed through a Whatman® syringe filter to remove any insoluble salts. Purification using reverse phase HPLC (35-65% MeCN in 0.05% NH₄OH aqueous solution) afforded the title compound (61 mg, 48%). ¹H-NMR (400 MHz, DMSO-d₆) δ 9.06 (dd, J=4.6, 1.7 Hz, 1H), 8.57 (dd, J=8.0, 1.7 Hz, 1H), 8.49 (dd, J=5.2, 0.8 Hz, 1H), 8.39 (d, J=0.7 Hz, 1H), 7.70 (dd, J=11.6, 1.8 Hz, 1H), 7.65 (dd, J=8.1, 4.6 Hz, 1H), 7.62-7.56 (m, 2H), 7.50 (dd, J=5.3, 1.8 Hz, 1H), 7.38 (t, J=8.0 Hz, 1H), 5.31 (s, 2H), 2.52 (s, 3H); ES-MS [M+H]+=424.0/426.0.

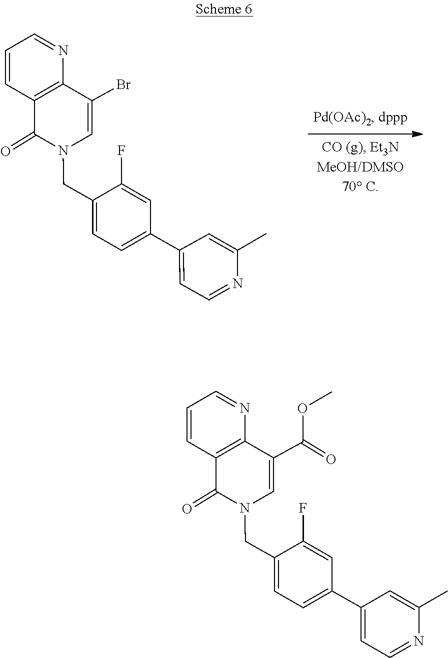 [SMILES: Cc1cc(-c2ccc(Cn3cc(Br)c4ncccc4c3=O)c(F)c2)ccn1 ; COC(=O)c1cn(Cc2ccc(-c3ccnc(C)c3)cc2F)c(=O)c2cccnc12]

Methyl 6-(2-fluoro-4-(2-methylpyridin-4-yl)benzyl)-5-oxo-5,6-dihydro-1,6-naphthyridine-8-carboxylate. A suspension of 8-bromo-6-(2-fluoro-4-(2-methylpyridin-4-yl)benzyl)-1,6-naphthyridin-5(6H)-one (61 mg, 0.14 mmol, 1.0 eq.), triethylamine (0.197 mL, 1.41 mmol, 10.0 eq.) in MeOH (2.0 mL) and DMSO (2.0 mL) was degassed thoroughly with nitrogen. Palladium(II) acetate (11.2 mg, 0.05 mmol, 0.35 eq.) and 1,3-bis(diphenylphosphino)propane (20.4 mg, 0.05 mmol, 0.35 eq.) were added. The reaction mixture was stirred at 70° C. under CO (1 atm). After 16 h, the reaction mixture was filtered through a pad of Celite® which was rinsed thoroughly with MeOH and DCM. Volatiles were removed under reduced pressure. The remaining DMSO solution was passed through a Whatman® syringe filter to remove any insoluble salts. Purification using reverse phase HPLC (35-65% MeCN in 0.05% NH₄OH aqueous solution) afforded the title compound as an off white powder (30 mg, 48%). ¹H-NMR (400 MHz, DMSO-d₆) δ 9.03 (dd, J=4.5, 1.9 Hz, 1H), 8.58 (s, 1H), 8.56 (dd, J=8.1, 1.9, 1H), 8.50 (dd, J=5.3, 0.7 Hz, 1H), 7.71 (dd, J=11.6, 1.8 Hz, 1H), 7.62-7.58 (m, 3H), 7.50 (dd, J=5.4, 1.9 Hz, 1H), 7.40 (t, J=8.0 Hz, 1H), 5.38 (s, 2H), 3.85 (s, 3H), 2.52 (s, 3H); ES-MS [M+H]⁺=404.4.

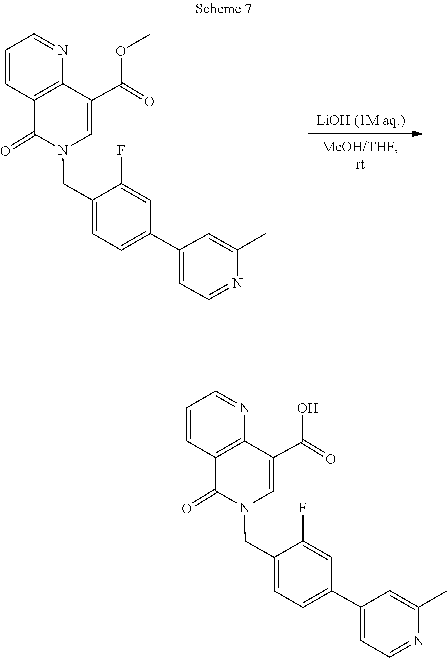 [SMILES: COC(=O)c1cn(Cc2ccc(-c3ccnc(C)c3)cc2F)c(=O)c2cccnc12 ; Cc1cc(-c2ccc(Cn3cc(C(=O)O)c4ncccc4c3=O)c(F)c2)ccn1]

6-(2-Fluoro-4-(2-methylpyridin-4-yl)benzyl)-5-oxo-5,6-dihydro-1,6-naphthyridine-8-carboxylic acid. To a suspension of methyl 6-(2-fluoro-4-(2-methylpyridin-4-yl)benzyl)-5-oxo-5,6-dihydro-1,6-naphthyridine-8-carboxylate (30 mg, 0.074 mmol, 1.0 eq.) in MeOH (0.5 mL) was added lithium hydroxide (1M aq. soln, 0.37 mL, 0.37 mmol, 5.0 eq.). The reaction mixture was stirred at 35° C. for 2 h. MeOH was partially removed under reduced pressure. The aqueous mixture was adjusted to a pH of 3-4 using HCl (2M aq. soln) and concentrated under reduced pressure. The title compound was obtained, assumed quantitative yield and carried forward without further purification. ¹H-NMR (400 MHz, DMSO-d₆) δ 9.10 (dd, J=4.9, 1.8 Hz, 1H), 8.93 (s, 1H), 8.80 (d, J=6.3 Hz, 1H), 8.73 (dd, J=8.2, 1.7 Hz, 1H), 8.34 (d, J=1.9 Hz, 1H), 8.21 (dd, J=6.3, 2.0 Hz, 1H), 7.98 (dd, J=11.5, 1.8 Hz, 1H), 7.83 (dd, J=8.1, 1.9 Hz, 1H), 7.78 (dd, J=8.2, 4.8 Hz, 1H), 7.56 (t, J=8.0 Hz, 1H), 5.49 (s, 2H), 2.79 (s, 3H), proton of carboxylic acid exchanged in solvent and not observable; ES-MS [M+H]⁺=390.3.

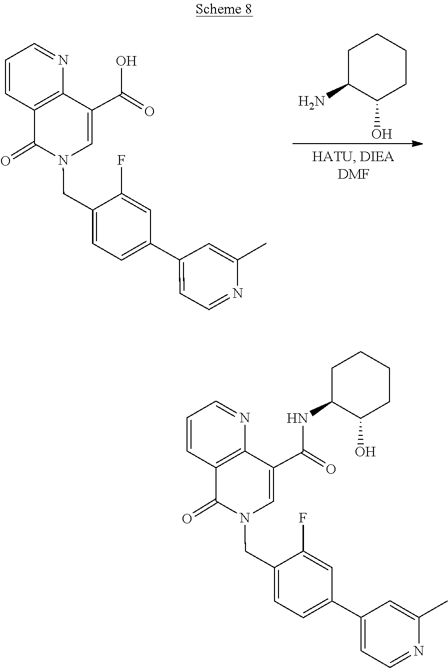 [SMILES: Cc1cc(-c2ccc(Cn3cc(C(=O)O)c4ncccc4c3=O)c(F)c2)ccn1 ; N[C@H]1CCCC[C@@H]1O ; Cc1cc(-c2ccc(Cn3cc(C(=O)N[C@H]4CCCC[C@@H]4O)c4ncccc4c3=O)c(F)c2)ccn1]

6-(2-Fluoro-4-(2-methylpyridin-4-yl)benzyl)-N-((1S,2S)-2-hydroxycyclohexyl)-5-oxo-5,6-dihydro-1,6-naphthyridine-8-carboxamide (Compound A27). A mixture of 6-(2-fluoro-4-(2-methylpyridin-4-yl)benzyl)-5-oxo-5,6-dihydro-1,6-naphthyridine-8-carboxylic acid (17 mg, 0.02 mmol, 1 eq.), (1S,2S)-2-aminocyclohexanol (5.8 mg, 0.05 mmol, 2.5 eq.) and N,N-diisopropylethylamine (35 μL, 0.20 mmol, 10 eq.) in DMF (1.0 mL) was stirred for 2 min. HATU (15.2 mg, 0.03 mmol, 2.0 eq.) was added. The reaction mixture was stirred at rt for 20 min. Purification using reverse phase HPLC (30-60% MeCN in 0.05% NH₄OH aqueous solution) afforded the title compound (3.8 mg, 39% 2 steps). ¹H-NMR (400 MHz, DMSO-d₆) δ 10.54 (d, J=7.5 Hz, 1H), 9.04 (dd, J=4.6, 1.9 Hz, 1H), 8.71 (d, J=0.9 Hz, 1H), 8.67 (dd, J=8.1, 1.9 Hz, 1H), 8.50 (dd, J=5.3, 0.7 Hz, 1H), 7.71 (dd, J=11.6, 1.8 Hz, 1H), 7.66 (dd, J=8.1, 4.6 Hz, 1H), 7.63-7.59 (m, 2H), 7.51 (dd, J=5.4, 1.8 Hz, 1H), 7.43 (t, J=8.0 Hz, 1H), 5.44 (s, 2H), 4.80 (d, J=4.8 Hz, 1H), 3.76-3.68 (m, 1H), 2.51 (s, 3H), 2.09-2.00 (m, 1H), 1.92-1.84 (m, 1H), 1.72-1.58 (m, 2H), 1.41-1.21 (m, 4H); ES-MS [M+H]⁺=487.4.

### Example 4. 6-(2,6-Difluoro-4-(thiazol-5-yl)benzyl)-N-((3R,4S)-3-hydroxytetrahydro-2H-pyran-4-yl)-5-oxo-5,6-dihydro-1,6-naphthyridine-8-carboxamide (Compound A49)

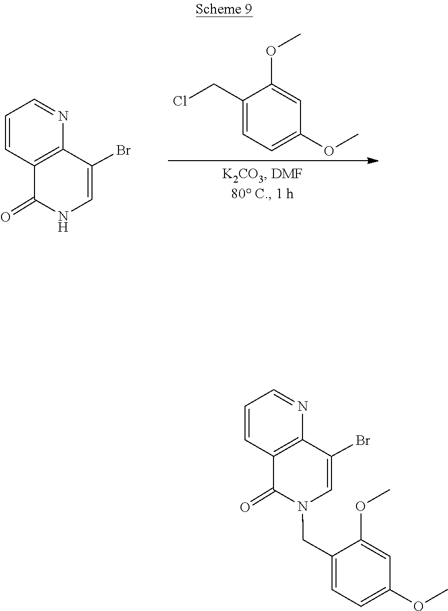 [SMILES: O=c1[nH]cc(Br)c2ncccc12 ; COc1ccc(CCl)c(OC)c1 ; COc1ccc(Cn2cc(Br)c3ncccc3c2=O)c(OC)c1]

8-Bromo-6-(2,4-dimethoxybenzyl)-1,6-naphthyridin-5(6H)-one. A mixture of 8-bromo-1,6-naphthyridin-5(6H)-one (675 mg, 3.0 mmol, 1.0 eq.), 1-(chloromethyl)-2,4-dimethoxybenzene (364 mg, 6.0 mmol, 2.0 eq.) and potassium carbonate (736 mg, 5.25 mmol, 1.75 eq.) in DMF (15 mL, 0.2 M) was allowed to stir at 80° C. After 1 h, the reaction mixture was passed through a Whatman® syringe filter and after the solvent was removed under vacuo, the crude material was purified using reverse phase HPLC (35-75% MeCN in 0.05% NH₄OH aqueous solution) to afford the title compound (650 mg, 88%). ¹H-NMR (400 MHz, DMSO-d₆) ¹H NMR (400 MHz, DMSO) δ 9.02 (dd, J=4.6, 1.7 Hz, 1H), 8.56 (dd, J=8.0, 1.8 Hz, 1H), 8.11 (s, 1H), 7.62 (dd, J=8.1, 4.6 Hz, 1H), 7.07 (d, J=8.4 Hz, 1H), 6.59 (d, J=2.3 Hz, 1H), 6.48 (dd, J=8.4, 2.4 Hz, 1H), 5.06 (s, 2H), 3.81 (s, 3H), 3.74 (s, 4H); ES-MS [M+H]⁺=375.0/377.0.

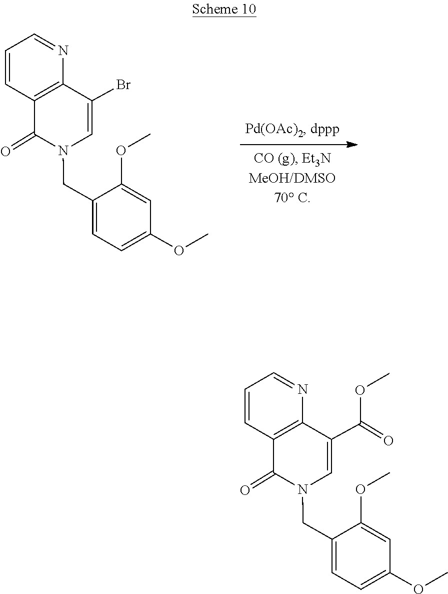 [SMILES: COc1ccc(Cn2cc(Br)c3ncccc3c2=O)c(OC)c1 ; COC(=O)c1cn(Cc2ccc(OC)cc2OC)c(=O)c2cccnc12]

Methyl 6-(2,4-dimethoxybenzyl)-5-oxo-5,6-dihydro-1,6-naphthyridine-8-carboxylate. A vial with a solution of 8-bromo-6-(2,4-dimethoxybenzyl)-1,6-naphthyridin-5(6H)-one (650 mg, 1.73 mmol, 1.0 eq.), triethylamine (2.41 mL, 17.3 mmol, 10.0 eq.) in MeOH (8.7 mL) and DMSO (8.7 mL) was evacuated under vacuo and purged with nitrogen (3×). Palladium(II) acetate (136 mg, 0.61 mmol, 0.35 eq.) and 1,3-bis(diphenylphosphino)propane (250 mg, 0.61 mmol, 0.35 eq.) were added. The reaction mixture was stirred at 70° C. under CO (1 atm). After 16 h, the reaction mixture was filtered through a pad of Celite® which was rinsed with MeOH and DCM. Volatiles were removed under reduced pressure. The remaining DMSO solution was passed through a Whatman® syringe filter and purified using reverse phase HPLC (30-70% MeCN in 0.05% NH₄OH aqueous solution) to afford the title compound as an off-white powder (528 mg, 86%). ES-MS [M+H]⁺=335.4.

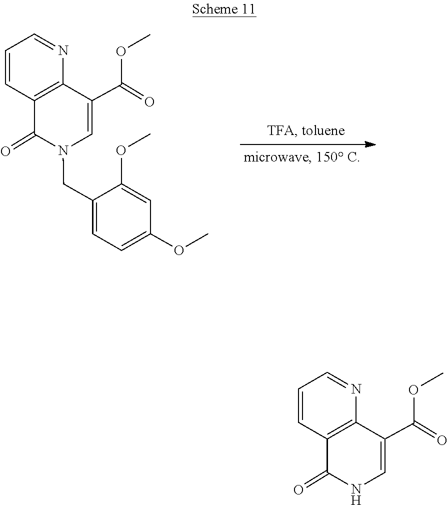 [SMILES: COC(=O)c1cn(Cc2ccc(OC)cc2OC)c(=O)c2cccnc12 ; COC(=O)c1c[nH]c(=O)c2cccnc12]

Methyl 5-oxo-5,6-dihydro-1,6-naphthyridine-8-carboxylate. To a suspension of methyl 6-(2,4-dimethoxybenzyl)-5-oxo-5,6-dihydro-1,6-naphthyridine-8-carboxylate (528 mg, 1.49 mmol, 1.0 eq.) in toluene (10 mL, 0.145 M) was added trifluoroacetic acid (1.14 mL, 14.9 mmol, 10.0 eq.). The mixture was subjected to microwave irradiation at 150° C. for 1 h. After being concentrated under reduced pressure, the title compound was obtained as a TFA salt, assumed quantitative yield and carried forward without further purification. ES-MS [M+H]⁺=205.4.

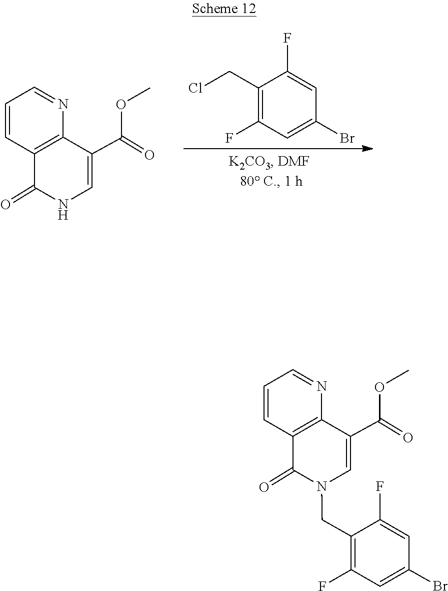 [SMILES: COC(=O)c1c[nH]c(=O)c2cccnc12 ; Fc1cc(Br)cc(F)c1CCl ; COC(=O)c1cn(Cc2c(F)cc(Br)cc2F)c(=O)c2cccnc12]

Methyl 6-(4-bromo-2,6-difluorobenzyl)-5-oxo-5,6-dihydro-1,6-naphthyridine-8-carboxylate. A mixture of methyl 5-oxo-5,6-dihydro-1,6-naphthyridine-8-carboxylate (447 mg, 1.40 mmol, 1.0 eq.), 5-bromo-2-(chloromethyl)-1,3-difluorobenzene (509 mg, 2.10 mmol, 1.5 eq.) and potassium carbonate (1.38 g, 4.21 mmol, 3.0 eq.) in DMF (7 mL, 0.2 M) was allowed to stir at 80° C. After 1 h, the reaction mixture was passed through a Whatman® syringe filter and after concentrating under reduced pressure, the crude material was purified using reverse phase HPLC (50-90% MeCN in 0.05% NH₄OH aqueous solution) to afford the title compound (470 mg, 82% over 2 steps). ES-MS [M+H]⁺=408.2/411.2.

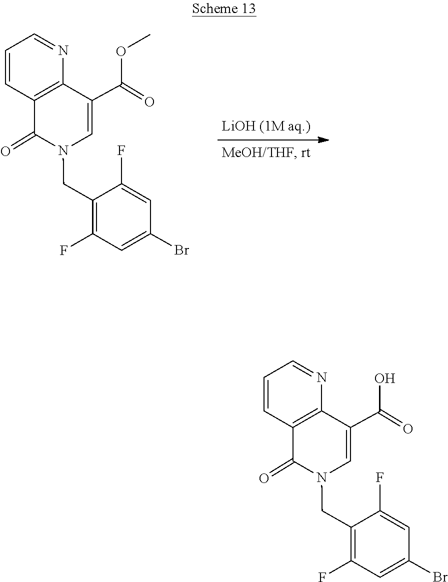 [SMILES: COC(=O)c1cn(Cc2c(F)cc(Br)cc2F)c(=O)c2cccnc12 ; O=C(O)c1cn(Cc2c(F)cc(Br)cc2F)c(=O)c2cccnc12]

6-(4-Bromo-2,6-difluorobenzyl)-5-oxo-5,6-dihydro-1,6-naphthyridine-8-carboxylic acid. To a suspension of methyl 6-(4-bromo-2,6-difluorobenzyl)-5-oxo-5,6-dihydro-1,6-naphthyridine-8-carboxylate (81.8 mg, 0.2 mmol, 1.0 eq.) in MeOH (4.0 mL, 0.05 M) was added lithium hydroxide (1M aq. soln, 1.2 mL, 1.2 mmol, 6.0 eq.). The reaction mixture was stirred at 40° C. for 2 h. MeOH was partially removed under reduced pressure. The aqueous mixture was adjusted to a pH of 3-4 using HCl (2M aq. soln) and concentrated under reduced pressure. The title compound was obtained, assumed quantitative yield and carried forward without further purification. ¹H-NMR (400 MHz, DMSO-d₆)¹H NMR (400 MHz, DMSO) δ 9.07 (dd, J=4.8, 1.7 Hz, 1H), 8.90 (s, 1H), 8.68 (dd, J=8.2, 1.7 Hz, 1H), 7.75 (dd, J=8.2, 4.8 Hz, 1H), 7.55-7.45 (m, 2H), 5.37 (s, 2H); ES-MS [M+H]⁺=395.2/397.2.

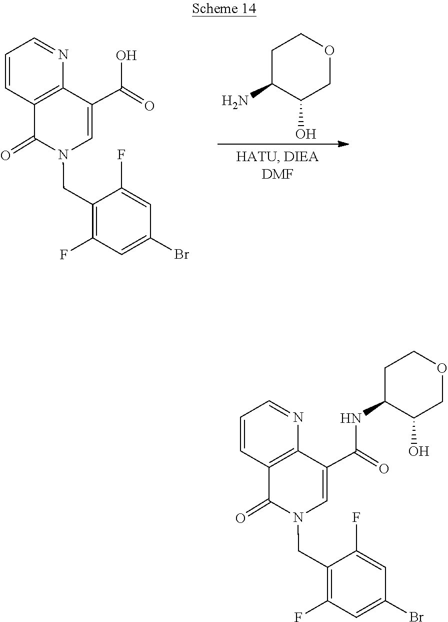 [SMILES: O=C(O)c1cn(Cc2c(F)cc(Br)cc2F)c(=O)c2cccnc12 ; N[C@H]1CCOC[C@@H]1O ; O=C(N[C@H]1CCOC[C@@H]1O)c1cn(Cc2c(F)cc(Br)cc2F)c(=O)c2cccnc12]

6-(4-Bromo-2,6-difluorobenzyl)-N-((3R,4S)-3-hydroxytetrahydro-2H-pyran-4-yl)-5-oxo-5,6-dihydro-1,6-naphthyridine-8-carboxamide. To 6-(4-bromo-2,6-difluorobenzyl)-5-oxo-5,6-dihydro-1,6-naphthyridine-8-carboxylic acid (120 mg, 0.199 mmol, 1 eq.) in DMF (4.0 mL) was added N,N-diisopropylethylamine (173 μL, 0.994 mmol, 5.0 eq.) and (3R,4S)-4-aminotetrahydropyran-3-ol (L-tyrosine salt, 77.4 mg, 0.240 mmol, 1.2 eq.). After 3 min, HATU (151 mg, 0.395 mmol, 2.0 eq.) was added and the reaction mixture was allowed to stir for 20 min. Purification using reverse phase HPLC (25-55% MeCN in 0.05% NH₄OH aqueous solution) afforded the title compound (70 mg, 71% over two steps). ¹H-NMR (400 MHz, DMSO-d₆) δ 10.62 (d, J=7.3 Hz, 1H), 9.04 (dd, J=4.6, 1.9 Hz, 1H), 8.73 (d, J=1.3 Hz, 1H), 8.61 (dd, J=8.1, 1.9 Hz, 1H), 7.64 (dd, J=8.1, 4.6 Hz, 1H), 7.55-7.46 (m, 2H), 5.35 (s, 2H), 5.15 (d, J=5.5 Hz, 1H), 3.90 (td, J=9.7, 5.3 Hz, 1H), 3.81 (td, J=10.7, 4.2 Hz, 2H), 3.54-3.47 (m, 1H), 3.44-3.38 (m, 1H), 3.13 (dd, J=11.2, 9.3 Hz, 1H), 2.13-2.05 (m, 1H), 1.61-1.46 (m, 1H); ES-MS [M+H]⁺=494.4/496.2.

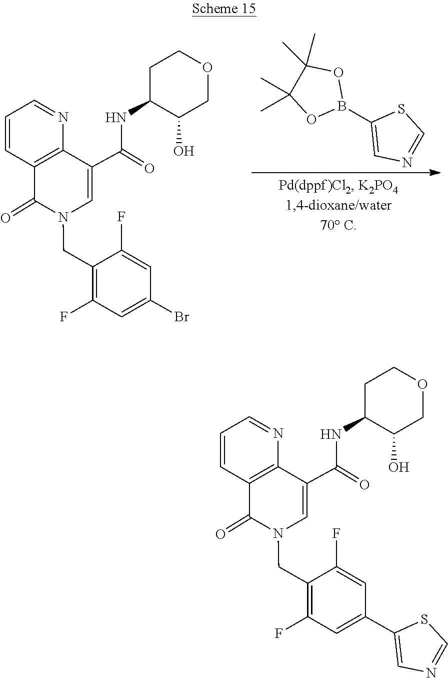 [SMILES: O=C(N[C@H]1CCOC[C@@H]1O)c1cn(Cc2c(F)cc(Br)cc2F)c(=O)c2cccnc12 ; CC1(C)OB(c2cncs2)OC1(C)C ; O=C(N[C@H]1CCOC[C@@H]1O)c1cn(Cc2c(F)cc(-c3cncs3)cc2F)c(=O)c2cccnc12]

6-(2,6-Difluoro-4-(thiazol-5-yl)benzyl)-N-((3R,4S)-3-hydroxytetrahydro-2H-pyran-4-yl)-5-oxo-5,6-dihydro-1,6-naphthyridine-8-carboxamide (Compound A49): 6-(4-Bromo-2,6-difluorobenzyl)-N-((3R,4S)-3-hydroxytetrahydro-2H-pyran-4-yl)-5-oxo-5,6-dihydro-1,6-naphthyridine-8-carboxamide (14.8 mg, 0.03 mmol, 1.0 eq.), 5-(4,4,5,5-tetramethyl-1,3,2-dioxaborolan-2-yl)thiazole (9.5 mg, 0.045 mmol, 1.5 eq.), 1,1′-bis(diphenylphosphino)ferrocene]dichloropalladium(II) (7.4 mg, 0.01 mmol, 0.3 eq.), and potassium phosphate tribasic (19.4 mg, 0.09 mmol, 3.0 eq.) in 1,4-dioxane (1.0 mL) and water (0.2 mL) were charged into a reaction vial and stirred under inert atmosphere at 70° C. for 1 h. The reaction mixture was filtered through a pad of Celite® which was rinsed with DCM:MeOH (9:1, v/v). The filtrate was concentrated under reduced pressure. The resulting material was re-dissolved in DMSO and filtered using a Whatman® syringe filter and the residue was purified using reverse phase HPLC (25-55% MeCN in 0.05% NH₄OH aqueous solution) to afford the title compound ¹H-NMR (400 MHz, DMSO-d₆) δ 10.63 (d, J=7.3 Hz, 1H), 9.16 (d, J=0.7 Hz, 1H), 9.04 (dd, J=4.6, 1.9 Hz, 1H), 8.75 (s, 1H), 8.62 (dd, J=8.1, 1.9 Hz, 1H), 8.46 (d, J=0.8 Hz, 1H), 7.64 (dd, J=8.1, 4.6 Hz, 1H), 7.56-7.50 (m, 2H), 5.40 (s, 2H), 5.15 (bs, 1H), 3.96-3.76 (m, 2H), 3.55-3.47 (m, 1H), 3.44-3.37 (m, 1H), 3.13 (dd, J=11.2, 9.4 Hz, 1H), 2.13-2.04 (m, 1H), 1.61-1.47 (m, 1H); ES-MS [M+H]⁺=499.3.

### Example 5. 6-(2,6-Difluoro-4-(2-methyl-2H-indazol-4-yl)benzyl)-N-((1S,2S)-2-hydroxycyclohexyl)-5-oxo-5,6-dihydropyrido[3,4-b]pyrazine-8-carboxamide (Compound A52)

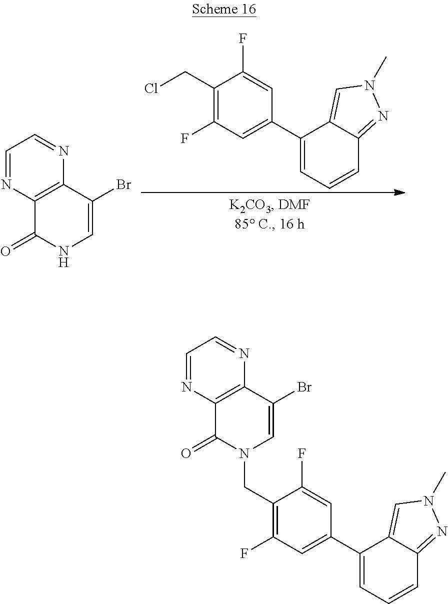 [SMILES: O=c1[nH]cc(Br)c2nccnc12 ; Cn1cc2c(-c3cc(F)c(CCl)c(F)c3)cccc2n1 ; Cn1cc2c(-c3cc(F)c(Cn4cc(Br)c5nccnc5c4=O)c(F)c3)cccc2n1]

8-Bromo-6-(2,6-difluoro-4-(2-methyl-2H-indazol-4-yl)benzyl)pyrido[3,4-b]pyrazin-5(6H)-one (Intermediate A). A mixture of 8-bromopyrido[3,4-b]pyrazin-5(6H)-one (226 mg, 1.0 mmol, 1.0 eq.), 4-(4-(chloromethyl)-3,5-difluorophenyl)-2-methyl-2H-indazole (351 mg, 1.2 mmol, 1.2 eq.) and potassium carbonate (280 mg, 2.0 mmol, 2.0 eq.) in DMF (5 mL, 0.2 M) was allowed to stir at 85° C. After 16 h, the reaction mixture was passed through a Whatman® syringe filter and purification using reverse phase HPLC (10-60% MeCN in 0.05% NH₄OH aqueous solution) afforded the title compound (398 mg, 83%). ¹H-NMR (400 MHz, CDCl₃) δ 8.97 (d, J=2.0 Hz, 1H), 8.85 (d, J=2.0 Hz, 1H), 8.04 (s, 1H), 7.83 (s, 1H), 7.74 (d, J=8.7 Hz, 1H), 7.34 (dd, J=7.0, 1.7, 1H), 7.28 (s, 1H), 7.26 (s, 1H), 7.14 (d, J=6.7 Hz, 1H), 5.40 (s, 2H), 4.24 (s, 3H); ES-MS [M+H]⁺=482.2/484.2.

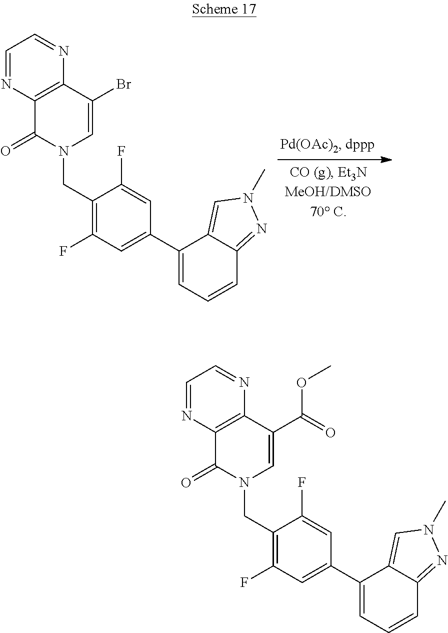 [SMILES: Cn1cc2c(-c3cc(F)c(Cn4cc(Br)c5nccnc5c4=O)c(F)c3)cccc2n1 ; COC(=O)c1cn(Cc2c(F)cc(-c3cccc4nn(C)cc34)cc2F)c(=O)c2nccnc12]

Methyl 6-(2,6-difluoro-4-(2-methyl-2H-indazol-4-yl)benzyl)-5-oxo-5,6-dihydropyrido[3,4-b]pyrazine-8-carboxylate (Intermediate B). A suspension of 8-bromo-6-(2,6-difluoro-4-(2-methyl-2H-indazol-4-yl)benzyl)pyrido[3,4-b]pyrazin-5(6H)-one (398 mg, 0.83 mmol, 1.0 eq.), triethylamine (1.15 mL, 8.30 mmol, 10.0 eq.) in MeOH (2.5 mL) and DMSO (3.9 mL) was evacuated and purged with nitrogen (3×). Palladium(II) acetate (46 mg, 0.21 mmol, 0.25 eq.) and 1,3-bis(diphenylphosphino)propane (85 mg, 0.21 mmol, 0.25 eq.) were added. The reaction mixture was stirred at 70° C. under CO (1 atm). After 16 h, the reaction mixture was filtered through a pad of Celite® which was rinsed with MeOH and DCM. The solvent was removed under reduced pressure, the remaining DMSO solution was passed through a Whatman® syringe filter and purification using reverse phase HPLC (45-85% MeCN in 0.05% NH₄OH aqueous solution) afforded the title compound (309 mg, 75%). ¹H-NMR (400 MHz, CDCl₃) δ 9.02 (d, J=2.1 Hz, 1H), 8.83 (d, J=2.0 Hz, 1H), 8.46 (s, 1H), 8.03 (s, 1H), 7.73 (d, J=8.7 Hz, 1H), 7.34 (dd, J=7.0, 1.7 Hz, 1H), 7.27-7.23 (m, 2H), 7.14 (d, J=6.4 Hz, 1H), 5.43 (s, 2H), 4.24 (s, 3H), 3.97 (s, 3H); ES-MS [M+H]⁺=462.2.

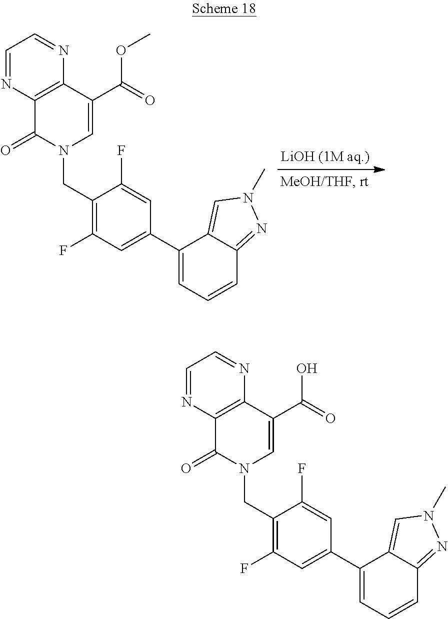 [SMILES: COC(=O)c1cn(Cc2c(F)cc(-c3cccc4nn(C)cc34)cc2F)c(=O)c2nccnc12 ; Cn1cc2c(-c3cc(F)c(Cn4cc(C(=O)O)c5nccnc5c4=O)c(F)c3)cccc2n1]

6-(2,6-Difluoro-4-(2-methyl-2H-indazol-4-yl)benzyl)-5-oxo-5,6-dihydropyrido[3,4-b]pyrazine-8-carboxylic acid (Intermediate C). To a suspension of methyl 6-(2,6-difluoro-4-(2-methyl-2H-indazol-4-yl)benzyl)-5-oxo-5,6-dihydropyrido[3,4-b]pyrazine-8-carboxylate (309 mg, 0.67 mmol, 1.0 eq.) in THF (1.7 mL) and MeOH (1.7 mL) was added lithium hydroxide (1M aq. soln, 3.35 mL, 3.35 mmol, 5.0 eq.). The reaction mixture was stirred at rt. The organic solvents were removed under reduced pressure. The aqueous mixture was adjusted to a pH of 3-4 using HCl (2M aq. soln) and concentrated under reduced pressure. The title compound was obtained and carried forward without further purification. ¹H-NMR (400 MHz, DMSO-d₆) δ 9.09 (d, J=2.2 Hz, 1H), 8.99 (d, J=2.2 Hz, 1H), 8.94 (s, 1H), 8.62 (s, 1H), 7.66 (d, J=8.5 Hz, 1H), 7.52-7.46 (m, 2H), 7.34-7.31 (m, 1H), 7.27-7.25 (m, 1H), 5.50 (s, 2H), 4.18 (s, 3H); ES-MS [M+H]⁺=448.2

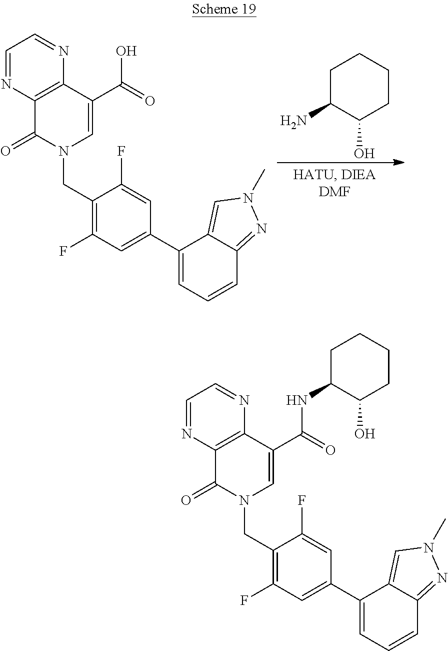 [SMILES: Cn1cc2c(-c3cc(F)c(Cn4cc(C(=O)O)c5nccnc5c4=O)c(F)c3)cccc2n1 ; N[C@H]1CCCC[C@@H]1O ; Cn1cc2c(-c3cc(F)c(Cn4cc(C(=O)N[C@H]5CCCC[C@@H]5O)c5nccnc5c4=O)c(F)c3)cccc2n1]

6-(2,6-Difluoro-4-(2-methyl-2H-indazol-4-yl)benzyl)-N-((1S,2S)-2-hydroxycyclohexyl)-5-oxo-5,6-dihydropyrido[3,4-b]pyrazine-8-carboxamide (Compound A52). A mixture of 6-(2,6-difluoro-4-(2-methyl-2H-indazol-4-yl)benzyl)-5-oxo-5,6-dihydropyrido[3,4-b]pyrazine-8-carboxylic acid (17 mg, 0.035 mmol, 1 eq.), HATU (26.4 mg, 0.07 mmol, 2.0 eq.) and N,N-diisopropylethylamine (30 μL, 0.173 mmol, 5 eq.) in DMF (1.0 mL) was stirred for 2 min. (1S,2S)-2-Aminocyclohexanol (6.0 mg, 0.05 mmol, 1.5 eq.) was added. The reaction mixture was stirred at 40° C. Purification using reverse phase HPLC (10-60% MeCN in 0.1% TFA aqueous solution) afforded the title compound (3.0 mg, 14% over 2 steps). ¹H-NMR (400 MHz, CDCl₃) δ 9.91 (d, J=7.0 Hz, 1H), 8.90 (d, J=2.1 Hz, 1H), 8.85 (d, J=2.1 Hz, 1H), 8.79 (s, 1H), 8.04 (s, 1H), 7.72 (d, J=8.8 Hz, 1H), 7.33 (dd, J=7.0, 1.7 Hz, 1H), 7.22 (s, 1H), 7.13 (d, J=6.8 Hz, 1H), 5.46 (s, 2H), 4.24 (s, 3H), 3.93-3.86 (m, 1H), 3.69 (s, 1H), 3.54-3.48 (m, 1H), 2.12-2.06 (m, 2H), 1.78-1.76 (m, 2H), 1.50-1.36 (m, 2H), 1.35-1.24 (m, 2H); ES-MS [M+H]⁺=545.3.

### Example 6. 6-(2,6-Difluoro-4-(2-methyl-2H-indazol-4-yl)benzyl)-N-((1S,2S)-2-hydroxycyclohexyl)-5-oxo-5,6-dihydropyrido[4,3-d]pyrimidine-8-carboxamide (Compound A88)

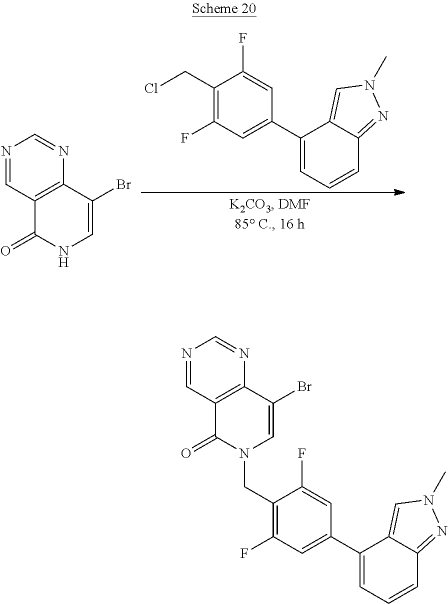 [SMILES: O=c1[nH]cc(Br)c2ncncc12 ; Cn1cc2c(-c3cc(F)c(CCl)c(F)c3)cccc2n1 ; Cn1cc2c(-c3cc(F)c(Cn4cc(Br)c5ncncc5c4=O)c(F)c3)cccc2n1]

8-Bromo-6-(2,6-difluoro-4-(2-methyl-2H-indazol-4-yl)benzyl)pyrido[4,3-d]pyrimidin-5(6H)-one is prepared in a similar manner as Intermediate A. ¹H-NMR (400 MHz, CDCl₃) δ 9.66 (s, 1H), 9.49 (s, 1H), 8.04 (s, 1H), 7.97 (s, 1H), 7.74 (d, J=8.7 Hz, 1H), 7.35 (dd, J=7.0, 1.8 Hz, 1H), 7.29 (s, 1H), 7.27 (s, 1H), 7.15 (d, J=6.8 Hz, 1H), 5.32 (s, 2H), 4.24 (s, 3H); ES-MS [M+H]⁺=482.0/484.0.

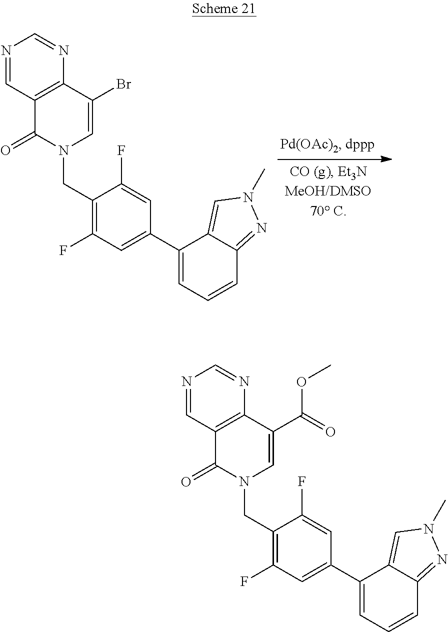 [SMILES: Cn1cc2c(-c3cc(F)c(Cn4cc(Br)c5ncncc5c4=O)c(F)c3)cccc2n1 ; COC(=O)c1cn(Cc2c(F)cc(-c3cccc4nn(C)cc34)cc2F)c(=O)c2cncnc12]

Methyl 6-(2,6-difluoro-4-(2-methyl-2H-indazol-4-yl)benzyl)-5-oxo-5,6-dihydropyrido[4,3-d]pyrimidine-8-carboxylate is prepared in a similar manner as Intermediate B. ¹H-NMR (400 MHz, CDCl₃) δ 9.68 (s, 1H), 9.53 (s, 1H), 8.61 (s, 1H), 8.03 (s, 1H), 7.74 (d, J=8.7 Hz, 1H), 7.35 (dd, J=7.0, 1.7 Hz, 1H), 7.29 (s, 1H), 7.27 (s, 1H), 7.15 (d, J=6.9 Hz, 1H), 5.36 (s, 2H), 4.24 (s, 3H), 3.99 (s, 3H); ES-MS [M+H]⁺=462.2.

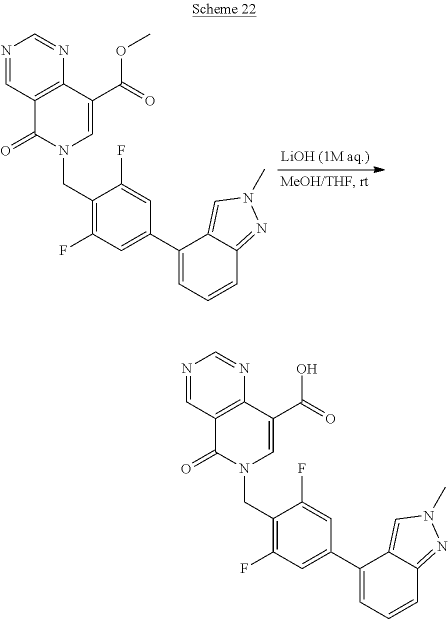 [SMILES: COC(=O)c1cn(Cc2c(F)cc(-c3cccc4nn(C)cc34)cc2F)c(=O)c2cncnc12 ; Cn1cc2c(-c3cc(F)c(Cn4cc(C(=O)O)c5ncncc5c4=O)c(F)c3)cccc2n1]

6-(2,6-Difluoro-4-(2-methyl-2H-indazol-4-yl)benzyl)-5-oxo-5,6-dihydropyrido[4,3-d]pyrimidine-8-carboxylic acid is prepared in the similar manner as Intermediate C. ¹H NMR (400 MHz, DMSO) δ 9.53 (s, 1H), 9.33 (s, 1H), 8.92 (s, 1H), 8.63 (s, 1H), 7.66 (d, J=8.5 Hz, 1H), 7.49 (d, J=8.8 Hz, 2H), 7.37-7.24 (m, 2H), 5.43 (s, 2H), 4.18 (s, 3H); ES-MS [M+H]⁺=448.0.

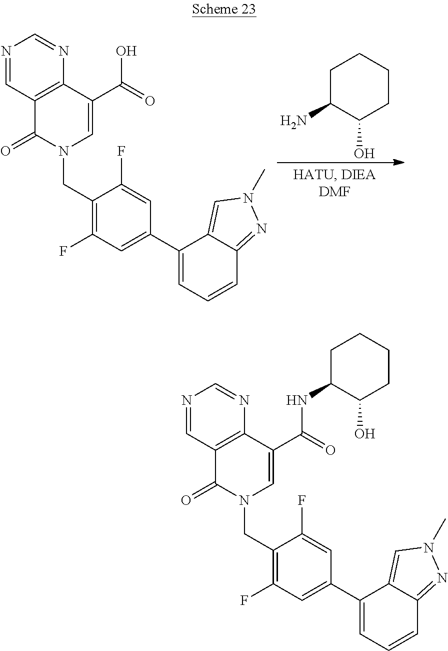 [SMILES: Cn1cc2c(-c3cc(F)c(Cn4cc(C(=O)O)c5ncncc5c4=O)c(F)c3)cccc2n1 ; N[C@H]1CCCC[C@@H]1O ; Cn1cc2c(-c3cc(F)c(Cn4cc(C(=O)N[C@H]5CCCC[C@@H]5O)c5ncncc5c4=O)c(F)c3)cccc2n1]

6-(2,6-Difluoro-4-(2-methyl-2H-indazol-4-yl)benzyl)-N-((1S,2S)-2-hydroxycyclohexyl)-5-oxo-5,6-dihydropyrido[4,3-d]pyrimidine-8-carboxamide (Compound A88) is prepared in a similar manner as Compound A1. ¹H NMR (400 MHz, DMSO) δ 9.97 (d, J=7.6 Hz, 1H), 9.54 (s, 1H), 9.49 (s, 1H), 9.01 (d, J=1.3 Hz, 1H), 8.62 (d, J=1.0 Hz, 1H), 7.67-7.65 (m, 1H), 7.54-7.43 (m, 2H), 7.33 (dd, J=8.5, 7.0 Hz, 1H), 7.27 (dd, J=7.0, 0.9 Hz, 1H), 5.49 (s, 2H), 4.83 (d, J=5.2 Hz, 1H), 4.18 (s, 3H), 3.79-3.72 (m, 1H), 3.48-3.43 (m, 1H), 2.07-2.05 (m, 1H), 1.92-1.89 (m, 1H), 1.69-1.62 (m, 2H), 1.32-1.24 (m, 4H); ES-MS [M+H]⁺=545.4.

### Example 7. 4-[4-(Chloromethyl)-3,5-difluoro-phenyl]-2-methyl-indazole

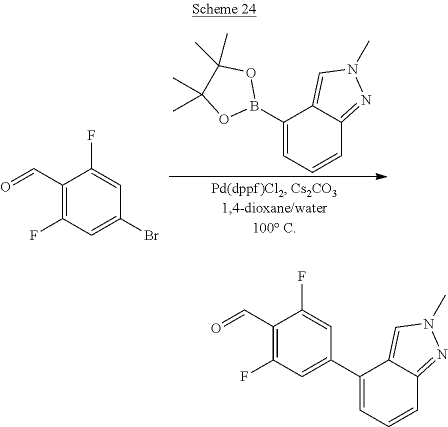

2,6-Difluoro-4-(2-methylindazol-4-yl)benzaldehyde: To a round-bottomed flask, 4-bromo-2,6-difluorobenzaldehyde (5.0 g, 23 mmol), 2-methyl-2H-indazole-4-boronic acid pinacol ester-(6.4 g, 25 mmol), [1,1′-bis(diphenylphosphino)ferrocene]dichloropalladium(II) (0.83 g, 1.1 mmol), and Cs₂CO₃ (15 g, 45 mmol) in 1,4-dioxane (42 mL) and water (42 mL) were added and allowed to stir under inert atmosphere at 100° C. for 30 min. The solution was cooled, diluted with 10:1 DCM:MeOH, washed with H₂O and brine, dried over Na₂SO₄ and concentrated. The solids were suspended in EtOAc (5 mL) (with sonication) then the solids were collected by vacuum filtration, washed with cold EtOAc and dried to give 2,6-difluoro-4-(2-methylindazol-4-yl)benzaldehyde (3 g, 48% yield) as a yellow solid. ¹H-NMR (400 MHz, DMSO) δ 10.3 (s, 1H), 8.70 (s, 1H), 7.74 (d, J=7.4, 1H), 7.63 (d, J=10, 2H), 7.40-7.37 (m, 2H), 4.22 (s, 3H). ES-MS [M+H]⁺=273.4.

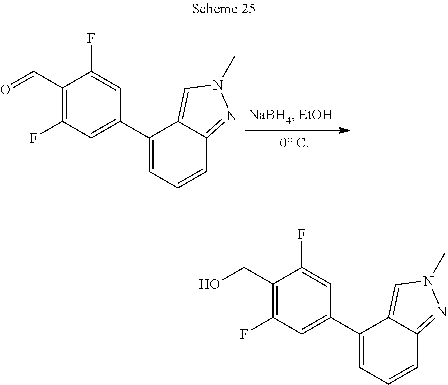 [SMILES: Cn1cc2c(-c3cc(F)c(C=O)c(F)c3)cccc2n1 ; Cn1cc2c(-c3cc(F)c(CO)c(F)c3)cccc2n1]

[2,6-Difluoro-4-(2-methylindazol-4-yl)phenyl]methanol: 2,6-Difluoro-4-(2-methylindazol-4-yl)benzaldehyde (3.0 g, 11 mmol) in ethanol (75 mL) was cooled to 0° C. and then sodium borohydride (0.5 g, 13 mmol) was added. The ice bath was removed and the reaction was allowed to stir at rt. After 18 h, the reaction was diluted with EtOAc and washed with water (2×), the collected organic layers were dried with MgSO₄ and concentrated to produce the desired product (3 g, 96% yield) as a white solid. ¹H-NMR (400 MHz, DMSO) δ 8.61 (s, 1H), 7.65 (d, J=8.5, 1H), 7.43 (d, J=8.5, 2H), 7.33 (dd, J=8.5, 1.5, 1H), 7.26 (dd, J=6.3, 0.6, 1H), 5.1 (t, J=4.7, 1H) 4.56 (d, J=3.0, 3H). ES-MS [M+H]⁺=275.4.

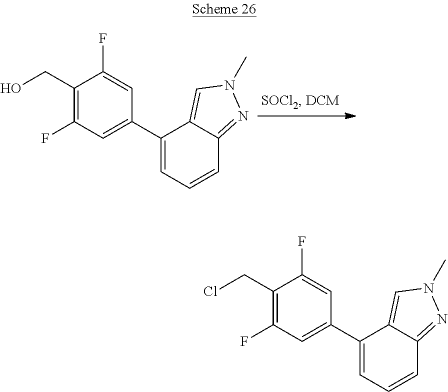 [SMILES: Cn1cc2c(-c3cc(F)c(CO)c(F)c3)cccc2n1 ; Cn1cc2c(-c3cc(F)c(CCl)c(F)c3)cccc2n1]

4-[4-(Chloromethyl)-3,5-difluoro-phenyl]-2-methyl-indazole: To a heterogeneous solution of [2,6-difluoro-4-(2-methylindazol-4-yl)phenyl]methanol (2.9 g, 11 mmol) in DCM was added thionyl chloride (1.2 mL, 16 mmol) at rt. After 3 h, to the reaction was added dropwise sat. NaHCO₃(aq) and the mixture was extracted with DCM (3×), dried with NaSO₄, filtered and concentrated. The crude product was purified using Teledyne ISCO Combi-Flash® system (330 G column, 0-100% MeOH/DCM) to afford the desired product (3 g, 99%) as a light yellow solid. ¹H-NMR (400 MHz, DMSO) δ 8.66 (s, 1H), 7.68 (d, J=8.4, 1H), 7.54 (d, J=8.9, 2H), 7.34 (dd, J=7.0, 1.4, 1H), 7.30 (dd, J=7.9, 0.9, 1H), 4.86 (s, 2H) 4.20 (s, 3H). ES-MS [M+H]⁺=293.2.

### Example 8. 6-(4-(1H-Pyrazol-1-yl)benzyl)-N-((3R,4S)-3-hydroxytetrahydro-2H-pyran-4-yl)-5-oxo-5,6-dihydroimidazo[1,2-c]pyrimidine-8-carboxamide (Compound B12)

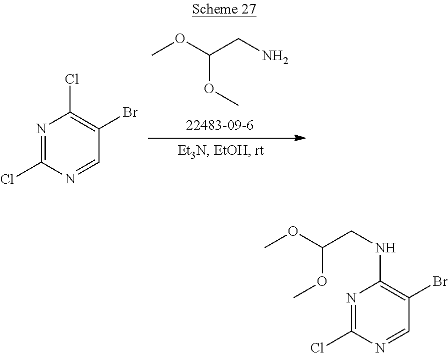

5-Bromo-2-chloro-N-(2,2-dimethoxyethyl)pyrimidin-4-amine. To a solution of 5-bromo-2,4-dichloropyrimidine (CAS #36082-50-5, 5.01 g, 22.0 mmol, 1.0.eq.) in ethanol (100 mL) was added triethylamine (3.68 mL, 26.4 mmol, 1.2 eq.) and aminoacetaldehyde dimethyl acetal (CAS #22483-09-6, 2.64 mL, 24.2 mmol, 1.1 eq.). After 24 h at rt, the reaction mixture was concentrated under reduced pressure. The residue was diluted with EtOAc, washed with water and brine, dried over Na₂SO₄, filtered and concentrated. Purification using normal phase flash chromatography on silica gel (20% EtOAc/hexanes) provided the title compound (2.5 g, 38%). ¹H-NMR (400 MHz, DMSO-d₆) δ 8.27 (s, 1H), 7.65 (t, J=5.8 Hz, 1H), 4.58 (t, J=5.4 Hz, 1H), 3.47 (dd, J=5.8, 5.8 Hz, 2H), 3.28 (s, 6H); ES-MS [M+H]⁺=296.2/298.2.

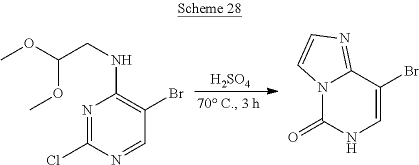

8-Bromo-6H-imidazo[1,2-c]pyrimidin-5-one. To a concentrated solution of sulfuric acid (2.0 mL) at 0° C. was added 5-bromo-2-chloro-N-(2,2-dimethoxyethyl)pyrimidin-4-amine (1.0 g, 3.37 mmol). The resulting suspension was allowed to stir at 70° C. for 3 h. After cooling to rt, the reaction mixture was poured onto an ice/water mixture and the pH was adjusted to 6-7 using 2N NaOH solution. The resulting precipitate was filtered using a Buchner funnel, washed with ice-cold water and dried under vacuum to provide the title compound (540 mg, 75%). ¹H-NMR (400 MHz, DMSO-d₆) δ 10.27 (broad s, 1H), 7.88 (d, J=1.5 Hz, 1H), 7.63 (s, 1H), 7.42 (d, J=1.5 Hz, 1H); ES-MS [M+H]+=214.0/216.0.

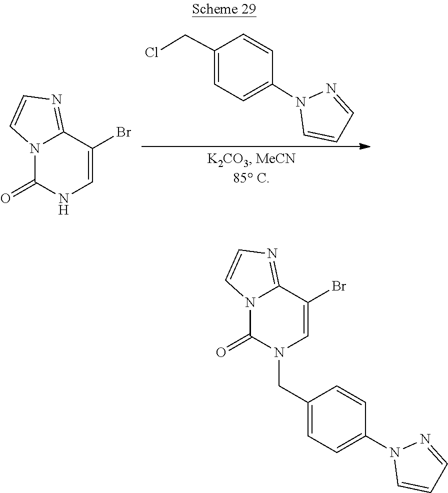 [SMILES: O=c1[nH]cc(Br)c2nccn12 ; ClCc1ccc(-n2cccn2)cc1 ; O=c1n(Cc2ccc(-n3cccn3)cc2)cc(Br)c2nccn12]

6-(4-(1H-Pyrazol-1-yl)benzyl)-8-bromoimidazo[1,2-c]pyrimidin-5(6H)-one. A reaction mixture of 8-bromo-6H-imidazo[1,2-c]pyrimidin-5-one (235 mg, 1.1 mmol, 1.1 eq.), 4-(1-pyrazolyl)benzyl chloride (CAS #143426-52-2, 193 mg, 1.0 mmol, 1.0 eq.), and potassium carbonate (245 mg, 1.75 mmol, 1.75 eq.) in DMF (5.0 mL, 0.2 M) was allowed to stir at 85° C. for 6 h. After cooling to rt, the reaction mixture was diluted with DMSO and filtered to remove insoluble salts. Purification using reverse phase HPLC (25-55% MeCN in 0.05% NH₄OH aqueous solution) provided the title compound as a powder (200 mg, 54%). ¹H-NMR (400 MHz, DMSO-d₆) δ 8.47 (d, J=2.4 Hz, 1H), 8.13 (s, 1H), 7.94 (d, J=1.6 Hz, 1H), 7.83-7.80 (m, 2H), 7.73 (d, J=1.6 Hz, 1H), 7.53-7.50 (m, 2H), 7.45 (d, J=1.6 Hz, 1H), 6.53 (dd, J=2.3, 1.9, 1H), 5.16 (s, 2H); ES-MS [M+H]+=370.2/372.3.

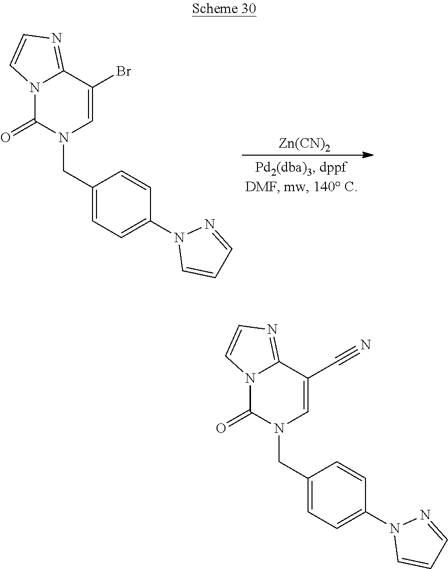 [SMILES: O=c1n(Cc2ccc(-n3cccn3)cc2)cc(Br)c2nccn12 ; "Zn(CN)2" ; N#Cc1cn(Cc2ccc(-n3cccn3)cc2)c(=O)n2ccnc12]

6-(4-(1H-Pyrazol-1-yl)benzyl)-5-oxo-5,6-dihydroimidazo[1,2-c]pyrimidine-8-carbonitrile. 6-(4-(1H-Pyrazol-1-yl)benzyl)-8-bromoimidazo[1,2-c]pyrimidin-5(6H)-one (200 mg, 0.54 mmol, 1.0 eq.), zinc cyanide (127 mg, 1.08 mmol, 2 eq.), tris(dibenzylideneacetone)dipalladium(0) (99 mg, 0.11 mmol, 0.15 eq.), 1,1′-bis(diphenylphosphino)ferrocene (60 mg, 0.108 mmol, 0.15 eq.) were combined into a microwave vial. Degassed DMF (5.4 mL, 0.2 M) was added. The reaction mixture was evacuated and filled with nitrogen and subjected to a microwave reactor for 30 min at 140° C. After cooling to rt, the reaction mixture was filtered to remove any insoluble salts. Purification using reverse phase HPLC (20-55% MeCN in 0.05% NH₄OH aqueous solution) provided the title compound (78 mg, 46%). ¹H-NMR (400 MHz, DMSO-d₆) δ 8.78 (s, 1H), 8.49 (d, J=2.5 Hz, 1H), 7.93 (d, J=1.6 Hz, 1H), 7.84-7.81 (m, 2H), 7.74 (d, J=1.6 Hz, 1H), 7.56-7.54 (m, 2H), 7.49 (d, J=1.6 Hz, 1H), 6.54 (dd, J=2.3, 1.9, 1H), 5.19 (s, 2H); ES-MS [M+H]+=317.4.

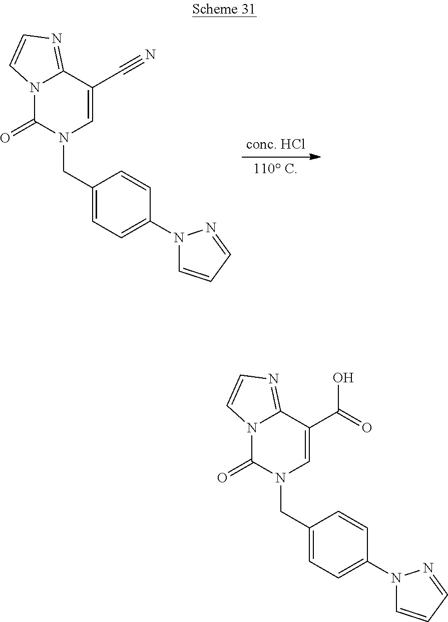 [SMILES: N#Cc1cn(Cc2ccc(-n3cccn3)cc2)c(=O)n2ccnc12 ; O=C(O)c1cn(Cc2ccc(-n3cccn3)cc2)c(=O)n2ccnc12]

6-(4-(1H-Pyrazol-1-yl)benzyl)-5-oxo-5,6-dihydroimidazo[1,2-c]pyrimidine-8-carboxylic acid. A mixture of 6-(4-(1H-pyrazol-1-yl)benzyl)-5-oxo-5,6-dihydroimidazo[1,2-c]pyrimidine-8-carbonitrile (78 mg, 0.25 mmol, 1 eq.) and concentrated hydrochloric acid solution (37%, 2.0 mL) was allowed to stir at 110° C. Upon completion, the reaction mixture was concentrated under reduced pressure. The product was purified using reverse phase HPLC (10-50% MeCN in 0.1% TFA aqueous solution) to afford the title compound (52 mg, 47%). ¹H-NMR (400 MHz, DMSO-d₆) δ 8.57 (s, 1H), 8.48 (d, J=2.4 Hz, 1H), 7.94 (s, 1H), 7.84-7.81 (m, 2H), 7.73 (d, J=1.6 Hz, 1H), 7.55-7.52 (m, 3H), 6.54 (dd, J=2.3, 1.9, 1H), 5.31 (s, 2H); ES-MS [M+H]+=336.3.

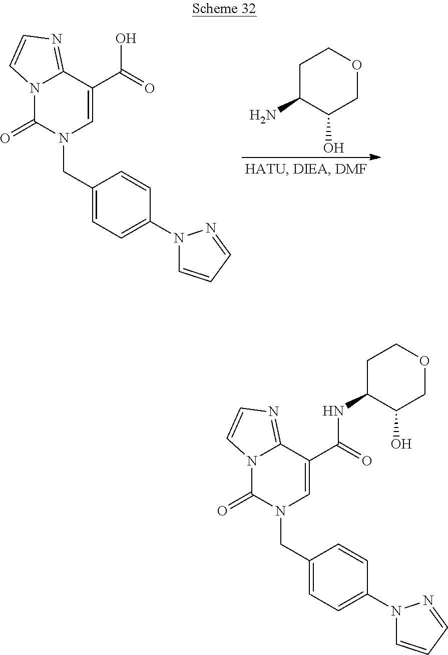 [SMILES: O=C(O)c1cn(Cc2ccc(-n3cccn3)cc2)c(=O)n2ccnc12 ; N[C@H]1CCOC[C@@H]1O ; O=C(N[C@H]1CCOC[C@@H]1O)c1cn(Cc2ccc(-n3cccn3)cc2)c(=O)n2ccnc12]

6-(4-(1H-Pyrazol-1-yl)benzyl)-N-((3R,4S)-3-hydroxytetrahydro-2H-pyran-4-yl)-5-oxo-5,6-dihydroimidazo[1,2-c]pyrimidine-8-carboxamide (Compound B12). To a solution of 6-(4-(1H-pyrazol-1-yl)benzyl)-5-oxo-5,6-dihydroimidazo[1,2-c]pyrimidine-8-carboxylic acid (as TFA salt, 10 mg, 0.022 mmol, 1 eq.) in DMF (1.0 mL) was added N,N-diisopropylethylamine (23 μL, 0.134 mmol, 6 eq.) and (3R,4S)-4-aminotetrahydropyran-3-ol (as L-tyrosine salt, 14 mg, 0.044 mmol, 2.0 eq.). After 3 min of stirring, HATU (17 mg, 0.044 mmol, 2.0 eq.) was added and the reaction mixture was allowed to stir at rt for 20 min. Purification using reverse phase HPLC afforded the title compound (5.9 mg, 61%). ¹H-NMR (400 MHz, DMSO-d₆) δ 9.39 (d, J=7.5 Hz, 1H), 8.47 (d, J=2.5 Hz, 1H), 8.39 (s, 1H), 7.95 (d, J=1.6 Hz, 1H), 7.84-7.81 (m, 2H), 7.73 (d, J=1.6 Hz, 1H), 7.54 (d, J=1.6, 1H), 7.53-7.50 (m, 2H), 6.53 (dd, J=2.3, 1.9, 1H), 5.33 (s, 2H), 5.13 (d, J=5.6 Hz, 1H), 3.89-3.76 (m, 3H), 3.47-3.36 (m, 2H), 3.10 (dd, J=11.3, 9.3 Hz, 1H), 2.06-2.02 (m, 1H), 1.53-1.43 (m, 1H); ES-MS [M+H]+=434.4.

### Example 9. 6-(2-Fluoro-4-(1-methyl-1H-pyrazol-4-yl)benzyl)-N-((3R,4S)-3-hydroxytetrahydro-2H-pyran-4-yl)-2-methyl-5-oxo-5,6-dihydroimidazo[1,2-c]pyrimidine-8-carboxamide (Compound B23)

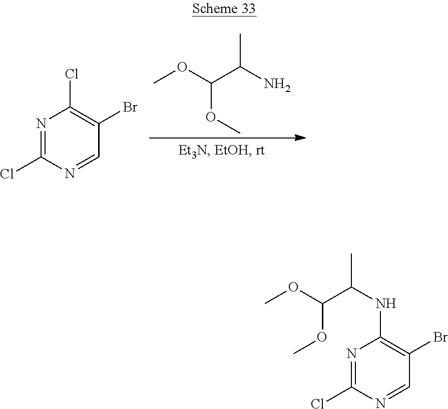

5-Bromo-2-chloro-N-(1,1-dimethoxypropan-2-yl)pyrimidin-4-amine. To a solution of 5-bromo-2,4-dichloropyrimidine (CAS #36082-50-5, 1.40 g, 5 mmol, 1.0.eq.) in ethanol (23 mL, 0.22 M) was added triethylamine (0.84 mL, 6.0 mmol, 1.2 eq.) and 1,1-dimethoxypropan-2-amine (CAS #57390-38-2, 0.68 mL, 5.5 mmol, 1.1 eq.). After 24 h at rt, the reaction mixture was concentrated under reduced pressure. The residue was diluted with EtOAc, washed with water and brine, dried over Na₂SO₄, filtered and concentrated. Purification using normal phase flash chromatography on silica gel (0-50% EtOAc/hexanes) provided the title compound (1.48 g, 95%). ¹H-NMR (400 MHz, DMSO-d₆) δ 8.28 (s, 1H), 7.17 (d, J=8.7 Hz, 1H), 4.45 (d, J=6.1 Hz, 1H), 4.38-4.26 (m, 1H), 3.33 (s, 3H), 3.28 (s, 3H), 1.16 (d, J=6.7 Hz, 3H); ES-MS [M+H]⁺=310.3/312.3.

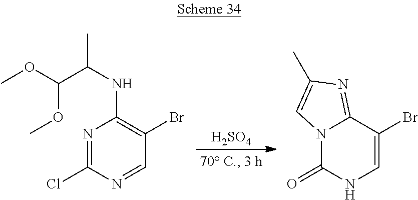

8-Bromo-2-methylimidazo[1,2-c]pyrimidin-5(6H)-one. To a concentrated solution of sulfuric acid (1.0 mL) at 0° C. was added 5-bromo-2-chloro-N-(2,2-dimethoxyethyl)pyrimidin-4-amine (1.48 g, 4.76 mmol, 1.0 eq). The resulting suspension was allowed to stir at 70° C. for 3 h. After cooling to rt, the reaction mixture was poured onto an ice/water mixture and the pH was adjusted to 6-7 using 2N NaOH solution. The precipitate was collected using a Buchner funnel, washed with ice-cold water and dried under vacuum to provide the title compound as a powder (988 mg, 91%). ¹H-NMR (400 MHz, DMSO-d₆) δ 11.42 (s, 1H), 7.61 (d, J=1.0 Hz, 1H), 7.59 (s, 1H), 2.27 (d, J=1.1 Hz, 3H); ES-MS [M+H]+=228.3/230.2.

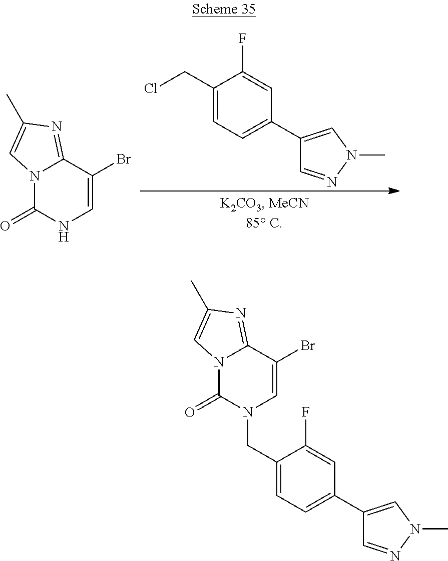 [SMILES: Cc1cn2c(=O)[nH]cc(Br)c2n1 ; Cn1cc(-c2ccc(CCl)c(F)c2)cn1 ; Cc1cn2c(=O)n(Cc3ccc(-c4cnn(C)c4)cc3F)cc(Br)c2n1]

8-Bromo-6-(2-fluoro-4-(1-methyl-1H-pyrazol-4-yl)benzyl)-2-methylimidazo[1,2-c]pyrimidin-5(6H)-one. A reaction mixture of 8-bromo-2-methylimidazo[1,2-c]pyrimidin-5(6H)-one (112.5 mg, 0.5 mmol, 1.0 eq.), 4-(4-(chloromethyl)-3-fluorophenyl)-1-methyl-1H-pyrazole (112.3 mg, 0.5 mmol, 1.0 eq.) and potassium carbonate (122.7 mg, 0.875 mmol, 1.75 eq.) in DMF (5.0 mL, 0.1 M) was allowed to stir at 80° C. for 1 h. After cooling to rt, the reaction mixture was diluted with DMSO and filtered. Purification using reverse phase HPLC (25-55% MeCN in 0.05% NH₄OH aqueous solution) provided the title compound (155 mg, 74%). ¹H-NMR (400 MHz, DMSO-d₆) δ 8.18 (d, J=0.8 Hz, 1H), 7.97 (d, J=0.7 Hz, 1H), 7.90 (d, J=0.8 Hz, 1H), 7.64 (d, J=1.1 Hz, 1H), 7.45 (dd, J=11.8, 1.7 Hz, 1H), 7.36 (dd, J=8.0, 1.7 Hz, 1H), 7.29 (t, J=8.0 Hz, 1H), 5.15 (s, 2H), 3.85 (s, 3H), 2.26 (d, J=1.1 Hz, 3H); ES-MS [M+H]+=416.2/418.2.

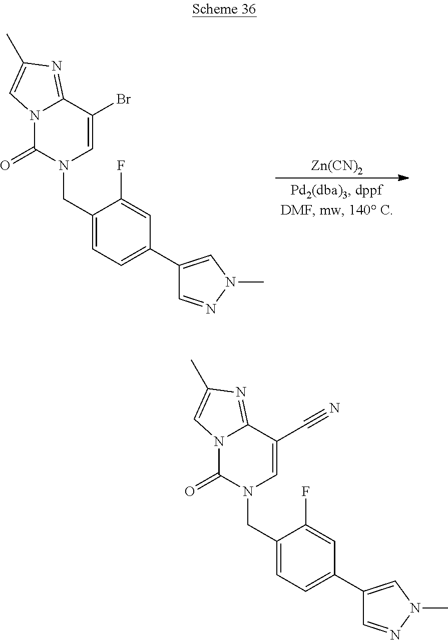 [SMILES: Cc1cn2c(=O)n(Cc3ccc(-c4cnn(C)c4)cc3F)cc(Br)c2n1 ; "Zn(CN)2" ; Cc1cn2c(=O)n(Cc3ccc(-c4cnn(C)c4)cc3F)cc(C#N)c2n1]

6-(2-Fluoro-4-(1-methyl-1H-pyrazol-4-yl)benzyl)-2-methyl-5-oxo-5,6-dihydroimidazo[1,2-c]pyrimidine-8-carbonitrile. 8-Bromo-6-(2-fluoro-4-(1-methyl-1H-pyrazol-4-yl)benzyl)-2-methylimidazo[1,2-c]pyrimidin-5(6H)-one (155 mg, 0.37 mmol, 1.0 eq.), zinc cyanide (87 mg, 0.75 mmol, 2 eq.), tris(dibenzylideneacetone)dipalladium(0) (68 mg, 0.075 mmol, 0.2 eq.), 1,1′-bis(diphenylphosphino)ferrocene (41 mg, 0.075 mmol, 0.2 eq.) were combined into a microwave vial. Anhydrous DMF (3.7 mL, 0.1 M) was added. The reaction mixture was evacuated and filled with nitrogen and subjected to a microwave reactor for 30 min at 140° C. After cooling to rt, the reaction mixture was filtered and purified using reverse HPLC (20-55% MeCN in 0.05% NH₄OH aqueous solution) to afford the title compound (92 mg, 68%). ¹H-NMR (400 MHz, DMSO-d₆) δ 8.65 (d, J=0.7 Hz, 1H), 8.21 (d, J=0.8 Hz, 1H), 7.92 (d, J=0.8 Hz, 1H), 7.64 (d, J=1.2 Hz, 1H), 7.46 (d, J=12 Hz, 1H), 7.43-7.33 (m, 2H), 5.18 (s, 2H), 3.86 (s, 3H), 2.28 (d, J=1.1 Hz, 3H); ES-MS [M+H]+=363.3.

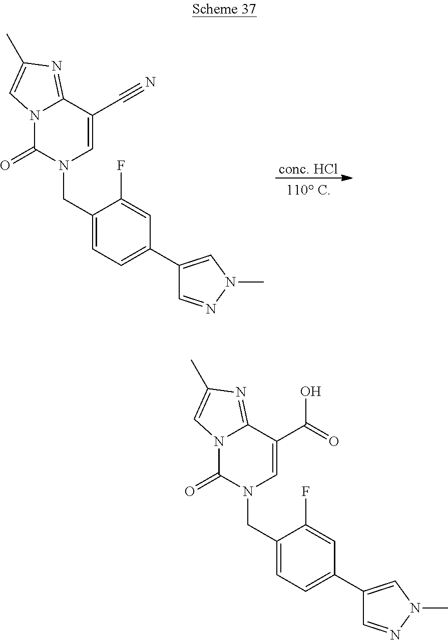 [SMILES: Cc1cn2c(=O)n(Cc3ccc(-c4cnn(C)c4)cc3F)cc(C#N)c2n1 ; "conc. HCl" ; Cc1cn2c(=O)n(Cc3ccc(-c4cnn(C)c4)cc3F)cc(C(=O)O)c2n1]

6-(2-Fluoro-4-(1-methyl-1H-pyrazol-4-yl)benzyl)-2-methyl-5-oxo-5,6-dihydroimidazo[1,2-c]pyrimidine-8-carboxylic acid. A mixture of 6-(2-fluoro-4-(1-methyl-1H-pyrazol-4-yl)benzyl)-2-methyl-5-oxo-5,6-dihydroimidazo[1,2-c]pyrimidine-8-carbonitrile (92 mg, 0.25 mmol, 1.0 eq.) and concentrated hydrochloric acid solution (37%, 2.0 mL) was allowed to stir at 110° C. After 3 h, the reaction mixture was concentrated under reduced pressure. The crude material was purified using reverse phase HPLC (5-45% MeCN in 0.1% TFA aqueous solution) to provide the title compound (112 mg, 89%). ¹H-NMR (400 MHz, DMSO-d₆) δ 8.72 (s, 1H), 8.21 (d, J=0.8 Hz, 1H), 7.92 (d, J=0.8 Hz, 1H), 7.86 (s, 1H), 7.46 (d, J=12.0 Hz, 1H), 7.44-7.36 (m, 2H), 5.34 (s, 2H), 3.86 (s, 3H), 2.35 (d, J=1.2 Hz, 3H), proton of carboxylic acid exchanged in solvent and not observable; ES-MS [M+H]+=382.3.

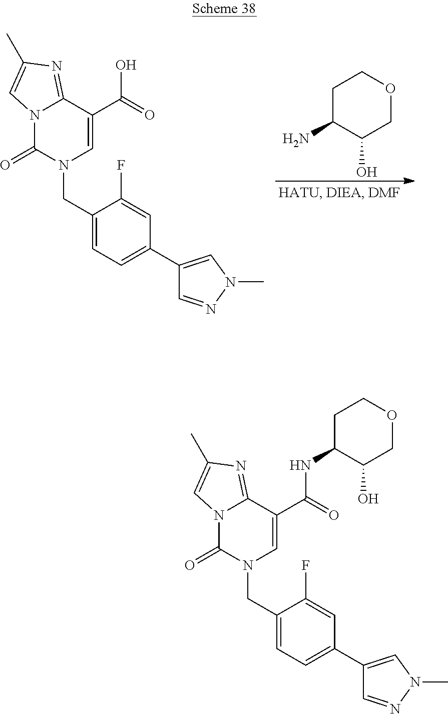 [SMILES: Cc1cn2c(=O)n(Cc3ccc(-c4cnn(C)c4)cc3F)cc(C(=O)O)c2n1 ; N[C@H]1CCOC[C@@H]1O ; Cc1cn2c(=O)n(Cc3ccc(-c4cnn(C)c4)cc3F)cc(C(=O)N[C@H]3CCOC[C@@H]3O)c2n1]

6-(2-Fluoro-4-(1-methyl-1H-pyrazol-4-yl)benzyl)-N-((3R,4S)-3-hydroxytetrahydro-2H-pyran-4-yl)-2-methyl-5-oxo-5,6-dihydroimidazo[1,2-c]pyrimidine-8-carboxamide (Compound B23). To a solution of 6-(2-fluoro-4-(1-methyl-1H-pyrazol-4-yl)benzyl)-2-methyl-5-oxo-5,6-dihydroimidazo[1,2-c]pyrimidine-8-carboxylic acid (12.4 mg, 0.025 mmol, 1.0 eq.) in DMF (1.0 mL) was added N,N-diisopropylethylamine (43.5 μL, 0.25 mmol, 10 eq.) and (3R,4S)-4-aminotetrahydropyran-3-ol (as L-tyrosine salt, 12 mg, 0.037 mmol, 1.5 eq.). After 3 min, HATU (19 mg, 0.05 mmol, 2.0 eq.) was added and the reaction mixture was allowed to stir at rt for 20 min. Purification using reverse phase HPLC afforded the title compound (6.0 mg, 50%). ¹H-NMR (400 MHz, DMSO-d₆) δ 9.39 (d, J=7.6 Hz, 1H), 8.28 (s, 1H), 8.20 (s, 1H), 7.91 (d, J=0.8 Hz, 1H), 7.64 (d, J=1.2 Hz, 1H), 7.46 (dd, J=11.8, 1.6 Hz, 1H), 7.40-7.32 (m, 2H), 5.30 (s, 2H), 5.15 (bs, 1H), 3.85 (s, 3H), 3.93-3.77 (m, 3H), 3.50-3.36 (m, 2H), 3.20-3.08 (m, 1H), 2.32 (d, J=1.1 Hz, 3H), 2.09-2.00 (m, 1H), 1.55-1.46 (m, 1H); ES-MS [M+H]+=481.4.

### Example 10. 5-(2-Fluoro-4-(1-methyl-1H-pyrazol-3-yl)benzyl)-N-((1S,2S)-2-hydroxycyclohexyl)-3-methyl-4-oxo-4,5-dihydro-3H-imidazo[4,5-c]pyridine-7-carboxamide (Compound C1)

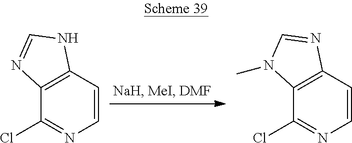

4-Chloro-3-methyl-3H-imidazo[4,5-c]pyridine. To a solution of 4-chloro-1H-imidazo[4,5-c]pyridine (3.07 g, 20.0 mmol, 1.0 eq.) in DMF (75 mL, 0.27 M) at 0° C. was added sodium hydride (2.4 g, 60.0 mmol, 3.0 eq.) portionwise. The resulting mixture was stirred at 0° C. for 30 min and iodomethane (3.74 mL, 60.0 mmol) was added dropwise. After 2 h at rt., sat. soln. of NH₄Cl was added. The reaction mixture was extracted with EtOAc (3×100 mL). The combined organic extracts were washed with water (3×50 mL), dried over Na₂SO₄, filtered and concentrated. Purification using reverse phase HPLC (0-50% MeCN in 0.05% NH₄OH aqueous solution) provided the title compound (1.2 g, 36%). ¹H-NMR (400 MHz, CDCl₃) δ 8.19 (d, J=5.6 Hz, 1H), 7.95 (s, 1H), 7.63 (d, J=5.6 Hz, 1H), 4.17 (s, 3H); ES-MS [M+H]+=168.0.

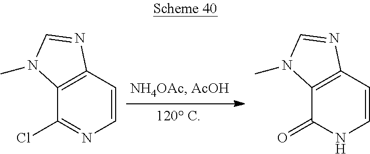

3-Methyl-3,5-dihydro-4H-imidazo[4,5-c]pyridin-4-one. A mixture of 4-chloro-3-methyl-3H-imidazo[4,5-c]pyridine (335 mg, 2.0 mmol, 1.0 eq.) and ammonium acetate (3.55 g, 46.0 mmol, 23.0 eq.) in acetic acid (4.5 mL) was stirred at 120° C. After 16 h, the mixture was adjusted to a pH of 6-7 with sat. soln. of NaHCO₃ and extracted with iPA/CHCl₃ (1:3). The combined organic extracts were dried over MgSO₄, filtered and concentrated to provide the title compound (298 mg, 99%). ¹H-NMR (400 MHz, DMSO-d₆) δ 11.22 (s, 1H), 8.11 (s, 1H), 7.05 (d, J=6.7 Hz, 1H), 6.52 (d, J=7.1 Hz, 1H), 4.00 (s, 3H); ES-MS [M+H]+=150.4.

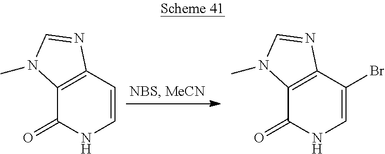

7-Bromo-3-methyl-3,5-dihydro-4H-imidazo[4,5-c]pyridin-4-one. 3-Methyl-3,5-dihydro-4H-imidazo[4,5-c]pyridin-4-one (298 mg, 2.0 mmol, 1.0 eq) was suspended in MeCN (5.0 mL, 0.4 M) and N-bromosuccinimide (426 mg, 2.4 mmol, 1.2 eq.) was added. After 1 h, the precipate was collected using a Buchner funnel and washed with EtOAc (2×2 mL), dried under vacumn to provide the title compound (305 mg, 67%). ¹H-NMR (400 MHz, DMSO-d₆) δ 11.50 (s, 1H), 8.18 (s, 1H), 7.33 (s, 1H), 4.00 (s, 3H); ES-MS [M+H]+=228.3/230.3.

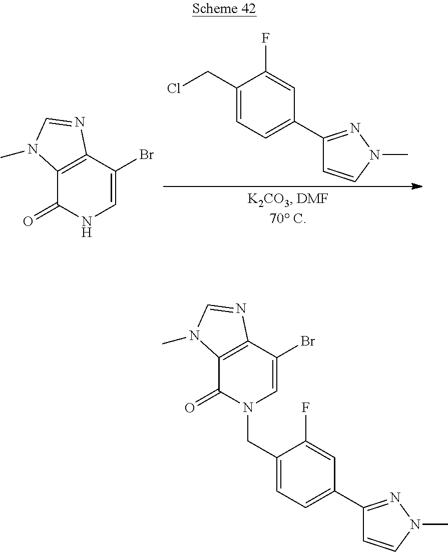

7-Bromo-5-(2-fluoro-4-(1-methyl-1H-pyrazol-3-yl)benzyl)-3-methyl-3,5-dihydro-4H-imidazo[4,5-c]pyridin-4-one. A reaction mixture of 7-bromo-3-methyl-3,5-dihydro-4H-imidazo[4,5-c]pyridin-4-one (100 mg, 0.44 mmol, 1.0 eq.), 3-(4-(chloromethyl)-3-fluorophenyl)-1-methyl-1H-pyrazole (118 mg, 0.48 mmol, 1.1 eq.) and potassium carbonate (111 mg, 0.79 mmol, 1.8 eq.) in DMF (3.3 mL, 0.13 M) was allowed to stir at 70° C. After 1 h, the reaction mixture was diluted with DMSO and filtered using a Whatman® syringe filter. After the solvent was reduced under pressure, purification using reverse phase HPLC (25-55% MeCN in 0.05% NH₄OH aqueous solution) provided the title compound (144 mg, 79%). ¹H-NMR (400 MHz, DMSO-d₆) ¹H NMR (400 MHz, DMSO) δ 8.23 (s, 1H), 7.87 (s, 1H), 7.74 (d, J=2.2 Hz, 1H), 7.59 (dd, J=5.8, 3.1 Hz, 1H), 7.54 (s, 1H), 7.17 (t, J=8.2 Hz, 1H), 6.73 (d, J=2.3 Hz, 1H), 5.23 (s, 2H), 4.01 (s, 3H), 3.88 (s, 3H); ES-MS [M+H]+=416.2/418.2.

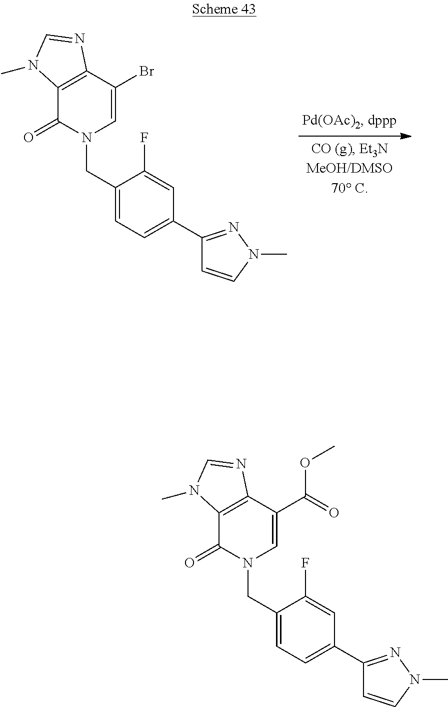

Methyl 5-(2-fluoro-4-(1-methyl-1H-pyrazol-3-yl)benzyl)-3-methyl-4-oxo-4,5-dihydro-3H-imidazo[4,5-c]pyridine-7-carboxylate. A vial with a suspension of 7-bromo-5-(2-fluoro-4-(1-methyl-1H-pyrazol-3-yl)benzyl)-3-methyl-3,5-dihydro-4H-imidazo[4,5-c]pyridin-4-one (144 mg, 0.35 mmol, 1.0 eq.), triethylamine (0.48 mL, 3.45 mmol, 10.0 eq.) in MeOH (1.5 mL) and DMSO (2.1 mL) was evacuated under vacuo and purged with nitrogen (3×). Palladium(II) acetate (27 mg, 0.12 mmol, 0.35 eq.) and 1,3-bis(diphenylphosphino)propane (49.9 mg, 0.12 mmol, 0.35 eq.) were added. The reaction mixture was stirred at 70° C. under CO (1 atm). After 16 h, the reaction mixture was filtered through a pad of Celite® which was rinsed with MeOH and DCM. Volatiles were removed under reduced pressure. The remaining DMSO solution was passed through a Whatman® syringe filter and after the solvent was removed under reduced pressure, the crude residue was purified using reverse phase HPLC (30-70% MeCN in 0.05% NH₄OH aqueous solution) to afford the title compound (116 mg, 85%). ¹H-NMR (400 MHz, CDCl₃) δ 8.37 (s, 1H), 8.21 (s, 1H), 7.74 (d, J=2.2 Hz, 1H), 7.59 (dd, J=3.3, 1.3 Hz, 1H), 7.54 (s, 1H), 7.22 (t, J=7.9 Hz, 1H), 6.73 (d, J=2.3 Hz, 1H), 5.34 (s, 2H), 4.00 (s, 3H), 3.88 (s, 3H), 3.83 (s, 3H); ES-MS [M+H]⁺=396.4.

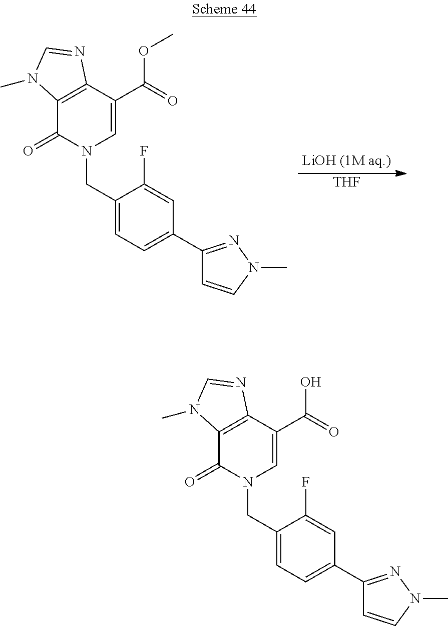

5-(2-Fluoro-4-(1-methyl-1H-pyrazol-3-yl)benzyl)-3-methyl-4-oxo-4,5-dihydro-3H-imidazo[4,5-c]pyridine-7-carboxylic acid. To a suspension of methyl 5-(2-fluoro-4-(1-methyl-1H-pyrazol-3-yl)benzyl)-3-methyl-4-oxo-4,5-dihydro-3H-imidazo[4,5-c]pyridine-7-carboxylate (116 mg, 0.116 mmol, 1.0 eq.) in THE (4.0 mL) was added lithium hydroxide (1M aq. soln, 1.47 mL, 1.47 mmol, 5.0 eq.). After 18 h at rt, the reaction mixture was adjusted to a pH of 3-4 using HCl (2M aq. soln) and concentrated under reduced pressure. The title compound was obtained and carried forward without further purification. ¹H-NMR (400 MHz, DMSO-d₆) δ 9.08 (s, 1H), 8.56 (s, 1H), 7.75 (d, J=2.3 Hz, 1H), 7.60 (s, 1H), 7.57 (dd, J=3.3, 1.2 Hz, 1H), 7.30 (t, J=7.9 Hz, 1H), 6.74 (d, J=2.3 Hz, 1H), 5.38 (s, 2H), 4.08 (s, 3H), 3.88 (s, 3H); ES-MS [M+H]⁺=382.4.

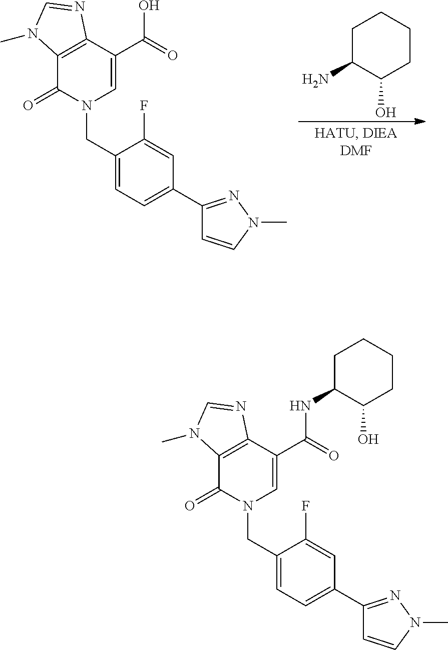

5-(2-Fluoro-4-(1-methyl-1H-pyrazol-3-yl)benzyl)-N-((1S,2S)-2-hydroxycyclohexyl)-3-methyl-4-oxo-4,5-dihydro-3H-imidazo[4,5-c]pyridine-7-carboxamide (Compound C1). A mixture of 5-(2-fluoro-4-(1-methyl-1H-pyrazol-3-yl)benzyl)-3-methyl-4-oxo-4,5-dihydro-3H-imidazo[4,5-c]pyridine-7-carboxylic acid (21 mg, 0.03 mmol, 1 eq.), (1S,2S)-2-aminocyclohexanol (14 mg, 0.09 mmol, 3.0 eq.) and N,N-diisopropylethylamine (30 μL, 0.173 mmol, 5 eq.) in DMF (1.0 mL) was stirred for 2 min. HATU (26 mg, 0.07 mmol, 2.0 eq.) was added. After 20 min at rt, the crude reaction mixture was purified using reverse phase HPLC (10-60% MeCN in 0.05% NH₄OH aqueous solution) to afford the title compound (7.4 mg, 52% over two steps). ¹H-NMR (400 MHz, DMSO-d₆) δ 9.11 (d, J=7.6 Hz, 1H), 8.35 (s, 1H), 8.21 (s, 1H), 7.74 (d, J=2.3 Hz, 1H), 7.62-7.58 (m, 1H), 7.56 (s, 1H), 7.22 (t, J=7.9 Hz, 1H), 6.73 (d, J=2.3 Hz, 1H), 5.35 (s, 2H), 4.77 (d, J=4.9 Hz, 1H), 4.04 (s, 3H), 3.88 (s, 3H), 3.73-3.66 (m, 1H), 3.44-3.34 (m, 1H), 2.04-1.96 (m, 1H), 1.89-1.81 (m, 1H), 1.69-1.57 (m, 2H), 1.36-1.16 (m, 4H); ES-MS [M+H]⁺=479.3.

### Example 11. 5-(2,6-Difluoro-4-(2-methyl-2H-indazol-4-yl)benzyl)-N-((3R,4S)-3-hydroxytetrahydro-2H-pyran-4-yl)-4-oxo-4,5-dihydrofuro[3,2-c]pyridine-7-carboxamide (Compound D18)

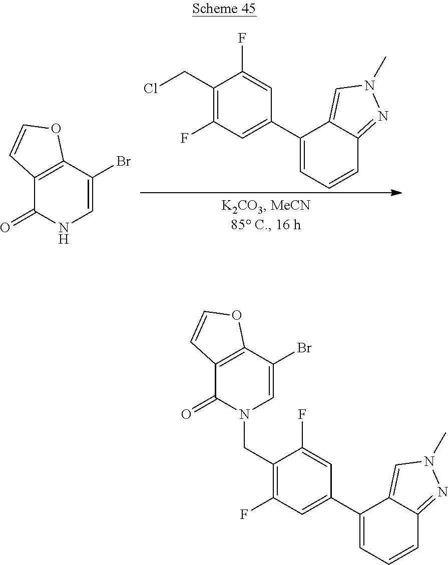 [SMILES: O=c1[nH]cc(Br)c2occc12 ; Cn1cc2c(-c3cc(F)c(CCl)c(F)c3)cccc2n1 ; Cn1cc2c(-c3cc(F)c(Cn4cc(Br)c5occc5c4=O)c(F)c3)cccc2n1]

7-Bromo-5-(2,6-difluoro-4-(2-methyl-2H-indazol-4-yl)benzyl)furo[3,2-c]pyridin-4(5H)-one (Intermediate D). A mixture of 7-bromofuro[3,2-c]pyridin-4(5H)-one (235.4 mg, 1.10 mmol, 1.1 eq.), 4-(4-(chloromethyl)-3,5-difluorophenyl)-2-methyl-2H-indazole (292.7 mg, 1.0 mmol, 1.0 eq.), and potassium carbonate (245.4 mg, 1.75 mmol, 1.75 eq.) in MeCN (10 mL, 0.1 M) was allowed to stir at 85° C. After 16 h, the reaction mixture was concentrated under reduced pressure. The remaining residue was re-dissolved in DMF/DMSO (1:1, v/v) and syringe filtered to remove insoluble salts. Purification using reverse phase HPLC (30-70% MeCN in 0.05% NH₄OH aqueous solution) provided the title compound (352 mg, 75%). ¹H-NMR (400 MHz, DMSO-d₆) δ 8.62 (s, 1H), 8.20 (s, 1H), 8.02 (d, J=2.1, 1H), 7.65 (d, J=8.6, 1H), 7.47-7.41 (m, 2H), 7.32 (dd, J=8.5, 7.0 Hz, 1H), 7.25 (dd, J=7.0, 0.7, 1H), 7.04 (d, J=2.1, 1H), 5.31 (s, 2H), 4.17 (s, 3H); ES-MS [M+H]⁺=470.2/472.3.

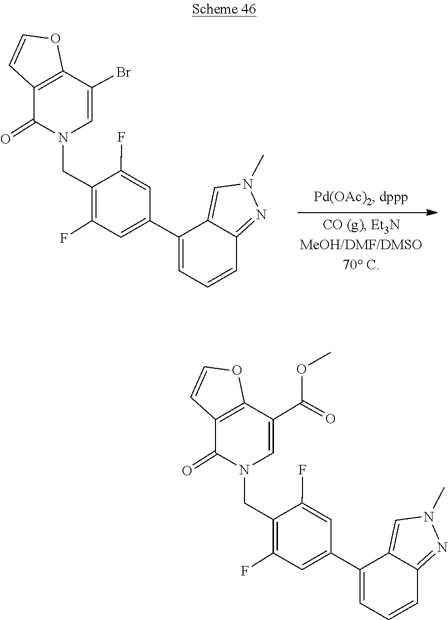 [SMILES: Cn1cc2c(-c3cc(F)c(Cn4cc(Br)c5occc5c4=O)c(F)c3)cccc2n1 ; COC(=O)c1cn(Cc2c(F)cc(-c3cccc4nn(C)cc34)cc2F)c(=O)c2ccoc12]

Methyl 5-(2,6-difluoro-4-(2-methyl-2H-indazol-4-yl)benzyl)-4-oxo-4,5-dihydrofuro[3,2-c]pyridine-7-carboxylate (Intermediate E). A solution mixture of 7-bromo-5-(2,6-difluoro-4-(2-methyl-2H-indazol-4-yl)benzyl)furo[3,2-c]pyridin-4(5H)-one (352 mg, 0.75 mmol, 1.0 eq.), triethylamine (1.04 mL, 7.49 mmol, 10.0 eq.) in MeOH (6 mL), DMSO (6 mL) and DMF (3.0 mL) was evacuated under vacuo and purged with nitrogen (3×). Palladium(II) acetate (58.8 mg, 0.26 mmol, 0.35 eq.) and 1,3-bis(diphenylphosphino)propane (108 mg, 0.26 mmol, 0.35 eq.) were added. The reaction mixture was stirred at 70° C. under CO (1 atm). After 16 h, the reaction mixture was filtered through a pad of Celite® which was rinsed with MeOH and DCM. Volatiles were removed under reduced pressure. The remaining DMF/DMSO solution was syringe filtered to remove insoluble salts and purified using reverse phase HPLC (35-75% MeCN in 0.05% NH₄OH aqueous solution). The title compound was obtained as an off-white powder (304 mg, 90%). ¹H-NMR (400 MHz, DMSO-d₆) δ 8.67 (s, 1H), 8.61 (s, 1H), 8.03 (d, J=2.1, 1H), 7.65 (d, J=8.5, 1H), 7.48-7.43 (m, 2H), 7.32 (dd, J=8.5, 7.0 Hz, 1H), 7.25 (dd, J=7.0, 0.7, 1H), 6.99 (d, J=2.1, 1H), 5.43 (s, 2H), 4.17 (s, 3H), 3.89 (s, 3H); ES-MS [M+H]⁺=450.2.

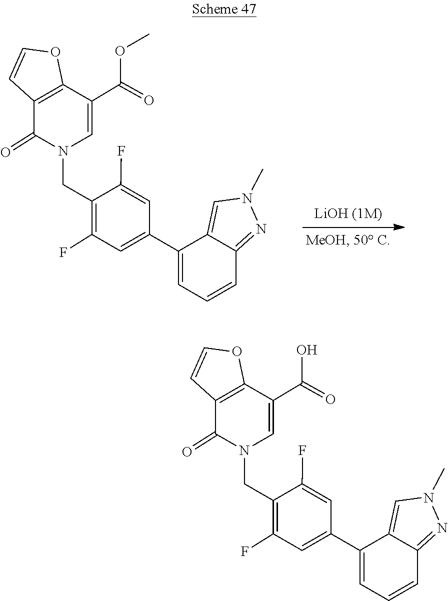 [SMILES: COC(=O)c1cn(Cc2c(F)cc(-c3cccc4nn(C)cc34)cc2F)c(=O)c2ccoc12 ; Cn1cc2c(-c3cc(F)c(Cn4cc(C(=O)O)c5occc5c4=O)c(F)c3)cccc2n1]

5-(2,6-Difluoro-4-(2-methyl-2H-indazol-4-yl)benzyl)-4-oxo-4,5-dihydrofuro[3,2-c]pyridine-7-carboxylic acid (Intermediate F). To a suspension of methyl 5-(2,6-difluoro-4-(2-methyl-2H-indazol-4-yl)benzyl)-4-oxo-4,5-dihydrofuro[3,2-c]pyridine-7-carboxylate (304 mg, 0.68 mmol, 1.0 eq.) in MeOH (4.5 mL) was added lithium hydroxide (1M aq. soln, 3.38 mL, 3.38 mmol, 5.0 eq.). The reaction mixture was stirred at 50° C. for 2 h. MeOH was partially removed under reduced pressure. The aqueous mixture was adjusted to a pH of 3-4 using HCl (2M aq. soln) and concentrated under reduced pressure. The title compound was obtained, assumed quantitative yield and carried forward without further purification. ¹H-NMR (400 MHz, DMSO-d₆) δ 8.62 (s, 1H), 8.57 (s, 1H), 7.99 (d, J=2.1, 1H), 7.65 (d, J=8.5, 1H), 7.48-7.43 (m, 2H), 7.32 (dd, J=8.5, 7.0 Hz, 1H), 7.25 (dd, J=7.0, 0.7, 1H), 6.96 (d, J=2.1, 1H), 5.43 (s, 2H), 4.17 (s, 3H); ES-MS [M+H]⁺=436.3.

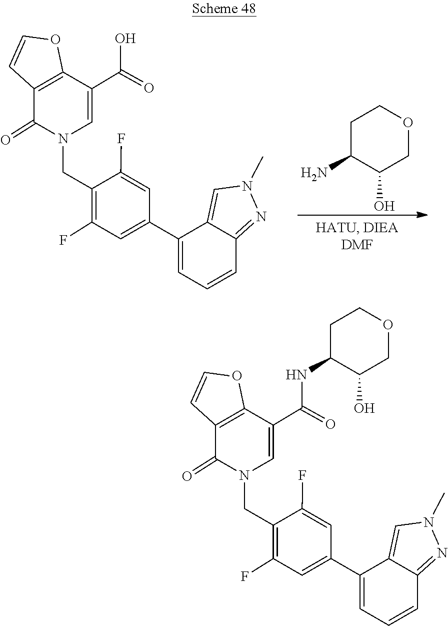 [SMILES: Cn1cc2c(-c3cc(F)c(Cn4cc(C(=O)O)c5occc5c4=O)c(F)c3)cccc2n1 ; N[C@H]1CCOC[C@@H]1O ; Cn1cc2c(-c3cc(F)c(Cn4cc(C(=O)N[C@H]5CCOC[C@@H]5O)c5occc5c4=O)c(F)c3)cccc2n1]

5-(2,6-Difluoro-4-(2-methyl-2H-indazol-4-yl)benzyl)-N-((3R,4S)-3-hydroxytetrahydro-2H-pyran-4-yl)-4-oxo-4,5-dihydrofuro[3,2-c]pyridine-7-carboxamide (Compound D18). To 5-(2,6-difluoro-4-(2-methyl-2H-indazol-4-yl)benzyl)-4-oxo-4,5-dihydrofuro[3,2-c]pyridine-7-carboxylic acid (LiCl salt, 66% purity, 15 mg, 0.021 mmol, 1 eq.) in DMF (1.0 mL) was added N,N-diisopropylethylamine (22 μL, 0.124 mmol, 6 eq.) and (3R,4S)-4-aminotetrahydropyran-3-ol (L-tyrosine salt, 10.8 mg, 0.031 mmol, 1.5 eq.). After 3-5 min, HATU (15 mg, 0.041 mmol, 2.0 eq.) was added and the reaction mixture was allowed to stir for 20 min. Purification using reverse phase HPLC (25-55% MeCN in 0.05% NH₄OH aqueous solution) afforded the title compound (6.0 mg, 54%). ¹H-NMR (400 MHz, DMSO-d₆) δ 8.62 (s, 1H), 8.40 (s, 1H), 8.04 (d, J=2.1, 1H), 7.70 (d, J=7.6, 1H), 7.65 (d, J=8.5, 1H), 7.48-7.43 (m, 2H), 7.32 (dd, J=8.5, 7.0 Hz, 1H), 7.26 (dd, J=7.0, 0.7, 1H), 7.02 (d, J=2.1, 1H), 5.41 (s, 2H), 5.08 (d, J=4.0 Hz, 1H), 4.17 (s, 3H); 3.92-3.73 (m, 4H), 3.54-3.47 (m, 1H), 3.08 (dd, J=10.7, 9.2 Hz, 1H), 1.97-1.92 (m, 1H), 1.64-1.54 (m, 1H); ES-MS [M+H]⁺=535.3.

### Example 12. 5-(2-Fluoro-4-(1-methyl-1H-pyrazol-3-yl)benzyl)-N-((1S,2S)-2-hydroxycyclopentyl)-4-oxo-4,5-dihydrofuro[3,2-c]pyridine-7-carboxamide (Compound D8)

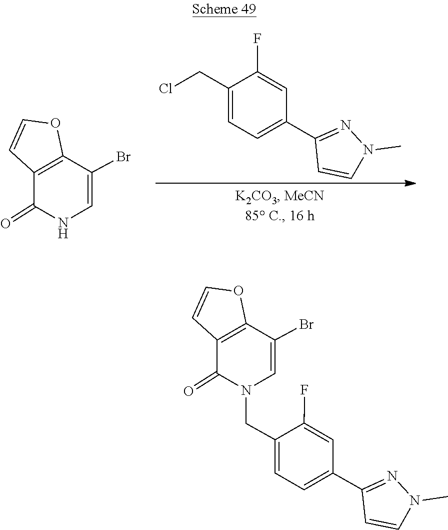 [SMILES: O=c1[nH]cc(Br)c2occc12 ; Cn1ccc(-c2ccc(CCl)c(F)c2)n1 ; Cn1ccc(-c2ccc(Cn3cc(Br)c4occc4c3=O)c(F)c2)n1]

7-Bromo-5-(2-fluoro-4-(1-methyl-1H-pyrazol-3-yl)benzyl)furo[3,2-c]pyridin-4(5H)-one is prepared in the similar manner as Intermediate D as a white powder. ¹H-NMR (400 MHz, DMSO-d₆) δ 8.12 (s, 1H), 8.04 (d, J=2.1, 1H), 7.73 (d, J=2.2, 1H), 7.59-7.55 (m, 2H), 7.15 (dd, J=8.1, 8.1, 1H), 7.09 (d, J=2.1 Hz, 1H), 6.72 (d, J=2.3, 1H), 5.23 (s, 2H), 3.87 (s, 3H); ES-MS [M+H]⁺=402.4/404.3.

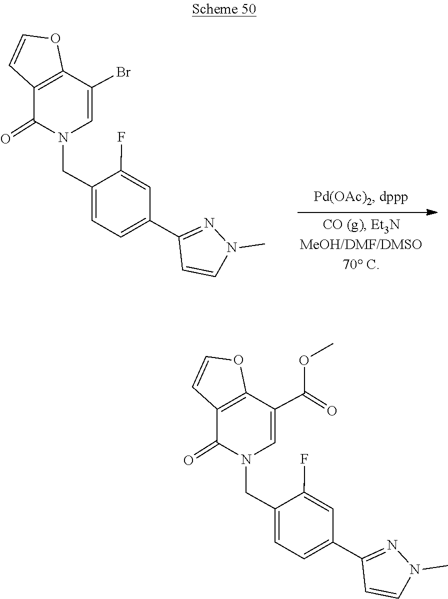 [SMILES: Cn1ccc(-c2ccc(Cn3cc(Br)c4occc4c3=O)c(F)c2)n1 ; COC(=O)c1cn(Cc2ccc(-c3ccn(C)n3)cc2F)c(=O)c2ccoc12]

Methyl 5-(2-fluoro-4-(1-methyl-1H-pyrazol-3-yl)benzyl)-4-oxo-4,5-dihydrofuro[3,2-c]pyridine-7-carboxylate is prepared in the similar manner as Intermediate E as a white powder. ¹H-NMR (400 MHz, DMSO-d₆) δ 8.56 (s, 1H), 8.04 (d, J=2.1, 1H), 7.74 (d, J=2.2, 1H), 7.58 (dd, J=5.0, 1.3, 1H), 7.56 (s, 1H), 7.21 (dd, J=8.1, 8.1, 1H), 7.03 (d, J=2.1 Hz, 1H), 6.73 (d, J=2.3, 1H), 5.36 (s, 2H), 3.87 (s, 3H), 3.86 (s, 3H); ES-MS [M+H]⁺=382.2.

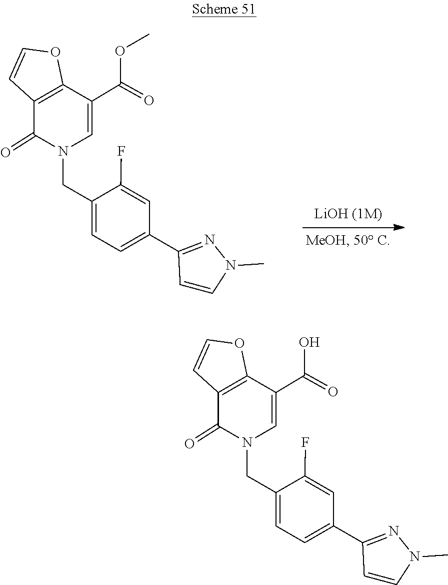

5-(2-Fluoro-4-(1-methyl-1H-pyrazol-3-yl)benzyl)-4-oxo-4,5-dihydrofuro[3,2-c]pyridine-7-carboxylic acid is prepared in the similar manner as Intermediate F as a white powder. ¹H-NMR (400 MHz, DMSO-d₆) δ 8.47 (s, 1H), 8.01 (d, J=2.1, 1H), 7.74 (d, J=2.2, 1H), 7.58 (dd, J=5.0, 1.3, 1H), 7.56 (s, 1H), 7.21 (dd, J=8.1, 8.1, 1H), 7.00 (d, J=2.1 Hz, 1H), 6.73 (d, J=2.3, 1H), 5.34 (s, 2H), 3.87 (s, 3H), proton of carboxylic acid exchanged in solvent and not observable; ES-MS [M+H]⁺=368.2.

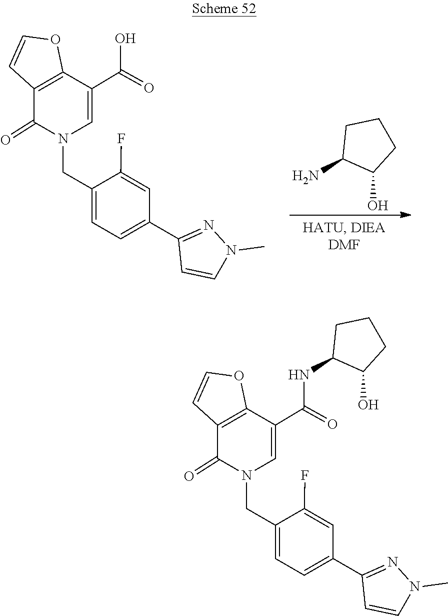 [SMILES: Cn1ccc(-c2ccc(Cn3cc(C(=O)O)c4occc4c3=O)c(F)c2)n1 ; N[C@H]1CCC[C@@H]1O ; Cn1ccc(-c2ccc(Cn3cc(C(=O)N[C@H]4CCC[C@@H]4O)c4occc4c3=O)c(F)c2)n1]

5-(2-Fluoro-4-(1-methyl-1H-pyrazol-3-yl)benzyl)-N-((1S,2S)-2-hydroxycyclopentyl)-4-oxo-4,5-dihydrofuro[3,2-c]pyridine-7-carboxamide (Compound D8) is prepared in the similar manner as Compound D18 as a white powder. ¹H-NMR (400 MHz, DMSO-d₆) δ 8.25 (s, 1H), 8.04 (d, J=2.1, 1H), 7.74 (d, J=2.2, 1H), 7.71 (d, J=2.2, 1H), 7.58 (dd, J=5.2, 1.3, 1H), 7.56 (s, 1H), 7.17 (dd, J=8.1, 8.1, 1H), 7.04 (d, J=2.1 Hz, 1H), 6.73 (d, J=2.3, 1H), 5.31 (s, 2H), 4.84 (d, J=3.6, 1H), 3.87 (s, 3H), 4.03-3.95 (m, 2H), 2.08-1.99 (m, 1H), 1.90-1.82 (m, 2H), 1.72-1.60 (m, 2H), ES-MS [M+H]+=451.3.

### Example 13. 5-(2,6-Difluoro-4-(2-methyl-2H-indazol-4-yl)benzyl)-N-((3R,4S)-3-hydroxytetrahydro-2H-pyran-4-yl)-4-oxo-4,5-dihydrothieno[3,2-c]pyridine-7-carboxamide (Compound D30)

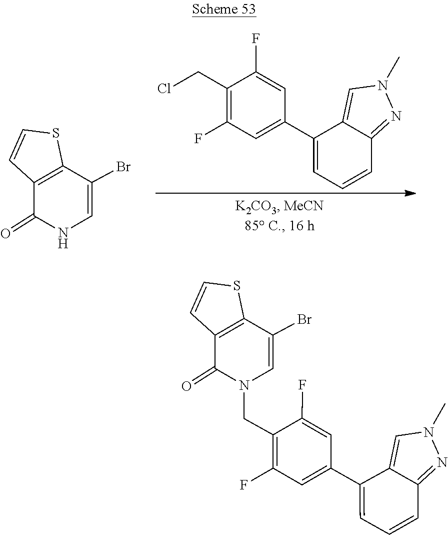 [SMILES: O=c1[nH]cc(Br)c2sccc12 ; Cn1cc2c(-c3cc(F)c(CCl)c(F)c3)cccc2n1 ; Cn1cc2c(-c3cc(F)c(Cn4cc(Br)c5sccc5c4=O)c(F)c3)cccc2n1]

7-Bromo-5-(2,6-difluoro-4-(2-methyl-2H-indazol-4-yl)benzyl)thieno[3,2-c]pyridin-4(5H)-one is prepared in the similar manner as Intermediate D. ¹H-NMR (400 MHz, DMSO-d₆) δ 8.62 (s, 1H), 8.13 (s, 1H), 7.75 (d, J=5.3, 1H), 7.65 (d, J=8.6, 1H), 7.59 (d, J=5.3, 1H), 7.46-7.40 (m, 2H), 7.32 (dd, J=8.5, 7.0 Hz, 1H), 7.25 (dd, J=7.0, 0.7, 1H), 5.31 (s, 2H), 4.17 (s, 3H); ES-MS [M+H]+=486.2/488.0.

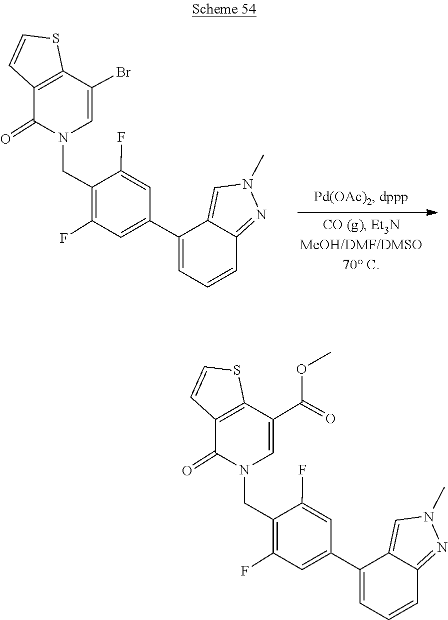 [SMILES: Cn1cc2c(-c3cc(F)c(Cn4cc(Br)c5sccc5c4=O)c(F)c3)cccc2n1 ; COC(=O)c1cn(Cc2c(F)cc(-c3cccc4nn(C)cc34)cc2F)c(=O)c2ccsc12]

Methyl 5-(2,6-difluoro-4-(2-methyl-2H-indazol-4-yl)benzyl)-4-oxo-4,5-dihydrothieno[3,2-c]pyridine-7-carboxylate is prepared in the similar manner as Intermediate E. ¹H-NMR (400 MHz, DMSO-d₆) δ 8.72 (s, 1H), 8.61 (s, 1H), 7.74 (d, J=5.4, 1H), 7.65 (d, J=8.6, 1H), 7.49 (d, J=5.3, 1H), 7.46-7.40 (m, 2H), 7.32 (dd, J=8.5, 7.0 Hz, 1H), 7.24 (d, J=6.8, 1H), 5.75 (s, 2H), 4.17 (s, 3H), 3.92 (s, 3H); ES-MS [M+H]⁺=466.4.

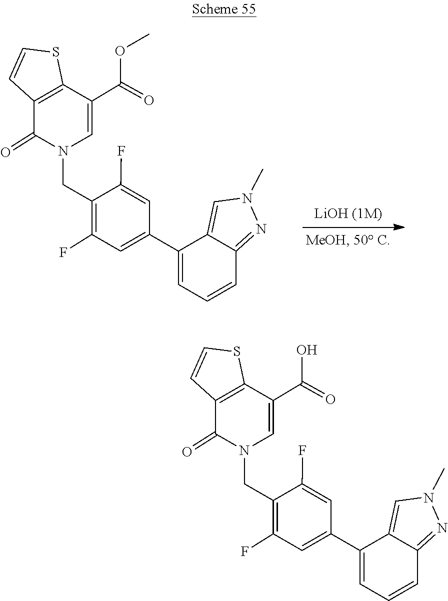 [SMILES: COC(=O)c1cn(Cc2c(F)cc(-c3cccc4nn(C)cc34)cc2F)c(=O)c2ccsc12 ; Cn1cc2c(-c3cc(F)c(Cn4cc(C(=O)O)c5sccc5c4=O)c(F)c3)cccc2n1]

5-(2,6-Difluoro-4-(2-methyl-2H-indazol-4-yl)benzyl)-4-oxo-4,5-dihydrothieno[3,2-c]pyridine-7-carboxylic acid is prepared in the similar manner as Intermediate F. ¹H-NMR (400 MHz, DMSO-d₆) δ 8.72 (s, 2H), 7.71 (d, J=5.4, 1H), 7.64 (d, J=8.6, 1H), 7.48-7.42 (m, 3H), 7.32 (dd, J=8.5, 7.0 Hz, 1H), 7.24 (dd, J=7.0, 0.7, 1H), 5.44 (s, 2H), 4.16 (s, 3H); ES-MS [M+H]⁺=452.4.

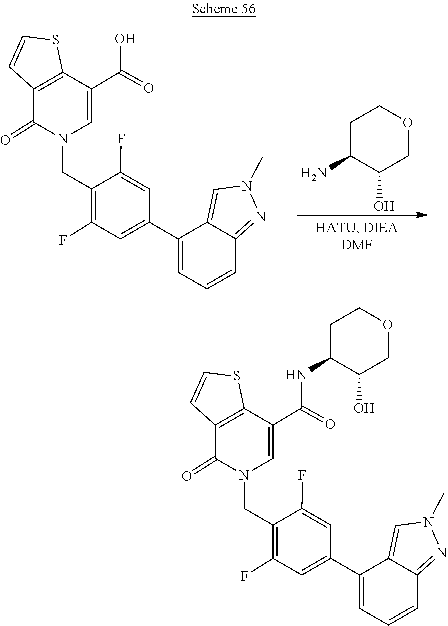 [SMILES: Cn1cc2c(-c3cc(F)c(Cn4cc(C(=O)O)c5sccc5c4=O)c(F)c3)cccc2n1 ; N[C@H]1CCOC[C@@H]1O ; Cn1cc2c(-c3cc(F)c(Cn4cc(C(=O)N[C@H]5CCOC[C@@H]5O)c5sccc5c4=O)c(F)c3)cccc2n1]

5-(2,6-Difluoro-4-(2-methyl-2H-indazol-4-yl)benzyl)-N-((3R,4S)-3-hydroxytetrahydro-2H-pyran-4-yl)-4-oxo-4,5-dihydrothieno[3,2-c]pyridine-7-carboxamide (Compound D30) is prepared in the similar manner as Compound A1. ¹H-NMR (400 MHz, DMSO-d₆) δ 8.70 (s, 1H), 8.61 (s, 1H), 8.34 (d, J=7.7, 1H), 7.68-7.63 (m, 2H), 7.48-7.43 (m, 3H), 7.32 (dd, J=8.5, 7.0 Hz, 1H), 7.26 (d, J=6.5, 1H), 7.02 (d, J=2.1, 1H), 5.30 (s, 2H), 5.02 (d, J=5.8 Hz, 1H), 4.17 (s, 3H), 3.92-3.80 (m, 4H), 3.54-3.45 (m, 1H), 3.05 (dd, J=10.7, 9.2 Hz, 1H), 1.97-1.92 (m, 1H), 1.64-1.54 (m, 1H); ES-MS [M+H]⁺=551.2.

The compounds presented in Table A, B, C and D may be prepared in an analogous manner to those described in the preceding schemes and examples using appropriate starting materials. Exemplary starting materials that may be used in the foregoing procedures include, but are not limited to, 4-(4-(chloromethyl)-3-fluorophenyl)-2-methyl-2H-indazole, (CAS #1478765-15-9; US 20140194471), 4-(4-(chloromethyl)-3-fluorophenyl)-1-methyl-1H-pyrazole, (CAS #1392081-37-6; WO 2013106795), 4-(4-(CHLOROMETHYL)PHENYL)-2-METHYLOXAZOLE (CAS #1859084-44-8; AstaTech, Inc.), 5-(chloromethyl)-2-(1-methyl-1H-pyrazol-4-yl)pyri dine, (CAS #1392081-39-8; ACS Med. Chem. Lett. 2018, 9, 917-922) and 5-(chloromethyl)-2-(1-methyl-1H-pyrazol-3-yl)pyri dine, (CAS #2247999-38-6; WO 2018235838).

TABLE A

| Cpd. | | | ES-MS |
|---|---|---|---|
| No. | Name | Structure | [M + H]⁺ |
| A1 | 6-(2-fluoro-4-(1-methyl-1H- pyrazol-3-yl)benzyl)-N- ((3R,4S)-3-hydroxytetrahydro- 2H-pyran-4-yl)-5-oxo-5,6- dihydro-1,6-naphthyridine-8- carboxamide | 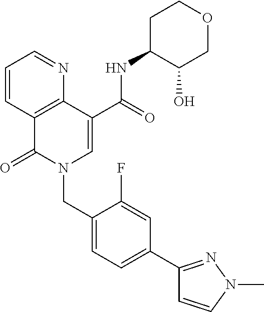 [SMILES: Cn1ccc(-c2ccc(Cn3cc(C(=O)N[C@H]4CCOC[C@@H]4O)c4ncccc4c3=O)c(F)c2)n1] | 478.4 |
| A2 | 6-(2-fluoro-4-(1-methyl-1H- pyrazol-3-yl)benzyl)-N- ((1S,2S)-2-hydroxycyclohexyl)- 5-oxo-5,6-dihydro-1,6- naphthyridine-8-carboxamide | 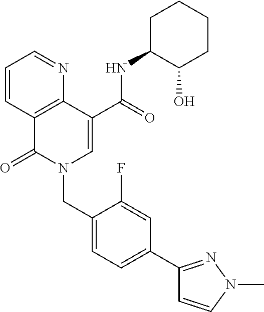 [SMILES: Cn1ccc(-c2ccc(Cn3cc(C(=O)N[C@H]4CCCC[C@@H]4O)c4ncccc4c3=O)c(F)c2)n1] | 476.4 |
| A3 | 6-(2-fluoro-4-(1-methyl-1H- pyrazol-3-yl)benzyl)-N- ((1S,2S)-2-hydroxycyclopentyl)- 5-oxo-5,6-dihydro-1,6- naphthyridine-8-carboxamide | 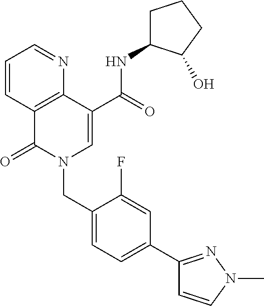 [SMILES: Cn1ccc(-c2ccc(Cn3cc(C(=O)N[C@H]4CCC[C@@H]4O)c4ncccc4c3=O)c(F)c2)n1] | 462.4 |
| A4 | 6-(2-fluoro-4-(1-methyl-1H- pyrazol-3-yl)benzyl)-N- ((1R,2R)-2-hydroxycyclobutyl)- 5-oxo-5,6-dihydro-1,6- naphthyridine-8-carboxamide | 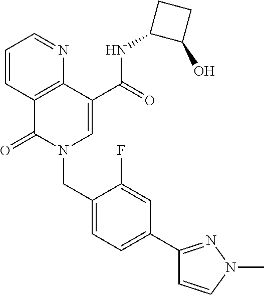 [SMILES: Cn1ccc(-c2ccc(Cn3cc(C(=O)N[C@@H]4CC[C@H]4O)c4ncccc4c3=O)c(F)c2)n1] | 448.4 |
| A5 | 6-(2-fluoro-4-(1-methyl-1H- pyrazol-3-yl)benzyl)-5-oxo-N- (2-oxaspiro[3.3]heptan-6-yl)- 5,6-dihydro-1,6-naphthyridine- 8-carboxamide | 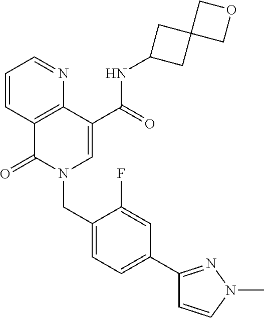 [SMILES: Cn1ccc(-c2ccc(Cn3cc(C(=O)NC4CC5(COC5)C4)c4ncccc4c3=O)c(F)c2)n1] | 474.4 |
| A6 | 6-(2-fluoro-4-(1-methyl-1H- pyrazol-4-yl)benzyl)-N- ((3R,4S)-3-hydroxytetrahydro- 2H-pyran-4-yl)-5-oxo-5,6- dihydro-1,6-naphthyridine-8- carboxamide | 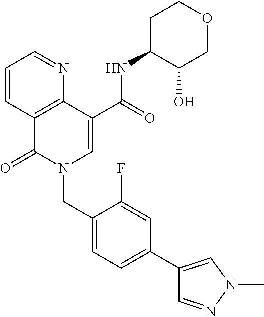 [SMILES: Cn1cc(-c2ccc(Cn3cc(C(=O)N[C@H]4CCOC[C@@H]4O)c4ncccc4c3=O)c(F)c2)cn1] | 478.4 |
| A7 | 6-(2-fluoro-4-(1-methyl-1H- pyrazol-4-yl)benzyl)-N- ((1S,2S)-2-hydroxycyclohexyl)- 5-oxo-5,6-dihydro-1,6- naphthyridine-8-carboxamide | 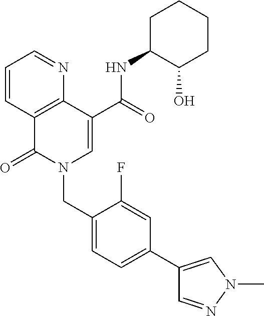 [SMILES: Cn1cc(-c2ccc(Cn3cc(C(=O)N[C@H]4CCCC[C@@H]4O)c4ncccc4c3=O)c(F)c2)cn1] | 476.4 |
| A8 | 6-(2-fluoro-4-(1-methyl-1H- pyrazol-4-yl)benzyl)-N- ((1S,2S)-2-hydroxycyclopentyl)- 5-oxo-5,6-dihydro-1,6- naphthyridine-8-carboxamide | 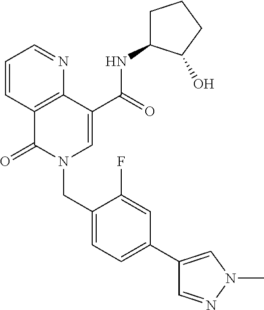 [SMILES: Cn1cc(-c2ccc(Cn3cc(C(=O)N[C@H]4CCC[C@@H]4O)c4ncccc4c3=O)c(F)c2)cn1] | 462.4 |
| A9 | 6-(2-fluoro-4-(1-methyl-1H- pyrazol-4-yl)benzyl)-N- ((1R,2R)-2-hydroxycyclobutyl)- 5-oxo-5,6-dihydro-1,6- naphthyridine-8-carboxamide | 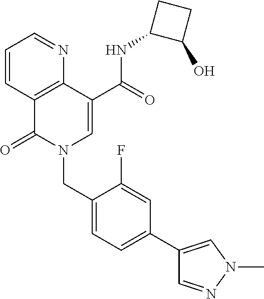 [SMILES: Cn1cc(-c2ccc(Cn3cc(C(=O)N[C@@H]4CC[C@H]4O)c4ncccc4c3=O)c(F)c2)cn1] | 448.4 |
| A10 | 6-(2-fluoro-4-(1-methyl-1H- pyrazol-4-yl)benzyl)-5-oxo-N- (tetrahydro-2H-pyran-4-yl)-5,6- dihydro-1,6-naphthyridine-8- carboxamide | 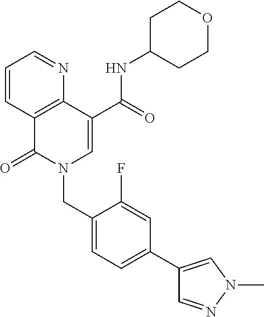 [SMILES: Cn1cc(-c2ccc(Cn3cc(C(=O)NC4CCOCC4)c4ncccc4c3=O)c(F)c2)cn1] | 462.4 |
| A11 | 6-(2-fluoro-4-(1-methyl-1H- pyrazol-4-yl)benzyl)-5-oxo-N- (2-oxaspiro[3.3]heptan-6-yl)- 5,6-dihydro-1,6-naphthyridine- 8-carboxamide | 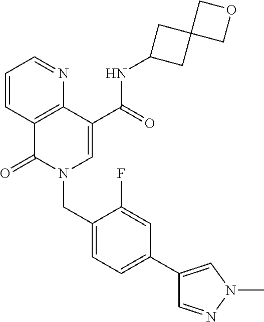 [SMILES: Cn1cc(-c2ccc(Cn3cc(C(=O)NC4CC5(COC5)C4)c4ncccc4c3=O)c(F)c2)cn1] | 474.4 |
| A12 | 6-(4-(1H-pyrazol-1-yl)benzyl)- N-((3R,4S)-3- hydroxytetrahydro-2H-pyran-4- yl)-5-oxo-5,6-dihydro-1,6- naphthyridine-8-carboxamide | 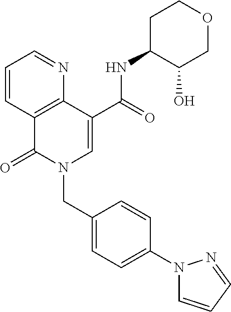 [SMILES: O=C(N[C@H]1CCOC[C@@H]1O)c1cn(Cc2ccc(-n3cccn3)cc2)c(=O)c2cccnc12] | 446.4 |
| A13 | 6-(4-(1H-pyrazol-1-yl)benzyl)- N-((1S,2S)-2- hydroxycyclohexyl)-5-oxo-5,6- dihydro-1,6-naphthyridine-8- carboxamide | 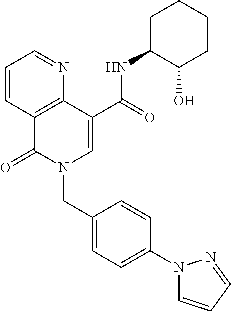 [SMILES: O=C(N[C@H]1CCCC[C@@H]1O)c1cn(Cc2ccc(-n3cccn3)cc2)c(=O)c2cccnc12] | 444.4 |
| A14 | 6-(4-(1H-pyrazol-1-yl)benzyl)- N-((1S,2S)-2- hydroxycyclopentyl)-5-oxo-5,6- dihydro-1,6-naphthyridine-8- carboxamide | 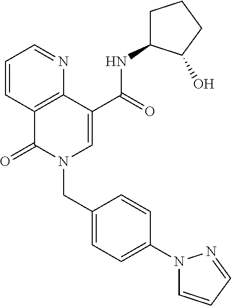 [SMILES: O=C(N[C@H]1CCC[C@@H]1O)c1cn(Cc2ccc(-n3cccn3)cc2)c(=O)c2cccnc12] | 430.4 |
| A15 | N-((3R,4S)-3- hydroxytetrahydro-2H-pyran-4- yl)-6-((6-(1-methyl-1H-pyrazol- 4-yl)pyridin-3-yl)methyl)-5-oxo- 5,6-dihydro-1,6-naphthyridine- 8-carboxamide | 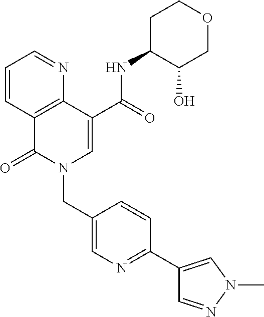 [SMILES: Cn1cc(-c2ccc(Cn3cc(C(=O)N[C@H]4CCOC[C@@H]4O)c4ncccc4c3=O)cn2)cn1] | 461.2 |
| A16 | N-((1S,2S)-2- hydroxycyclohexyl)-6-((6-(1- methyl-1H-pyrazol-4-yl)pyridin- 3-yl)methyl)-5-oxo-5,6-dihydro- 1,6-naphthyridine-8- carboxamide | 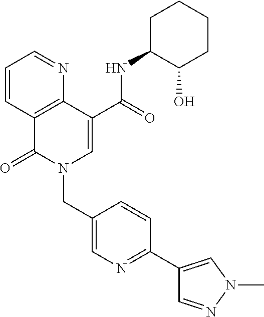 [SMILES: Cn1cc(-c2ccc(Cn3cc(C(=O)N[C@H]4CCCC[C@@H]4O)c4ncccc4c3=O)cn2)cn1] | 459.2 |
| A17 | N-((1S,2S)-2- hydroxycyclopentyl)-6-((6-(1- methyl-1H-pyrazol-4-yl)pyridin- 3-yl)methyl)-5-oxo-5,6-dihydro- 1,6-naphthyridine-8- carboxamide | 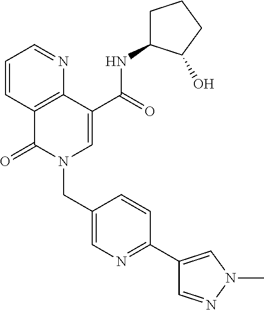 [SMILES: Cn1cc(-c2ccc(Cn3cc(C(=O)N[C@H]4CCC[C@@H]4O)c4ncccc4c3=O)cn2)cn1] | 445.2 |
| A18 | N-((1S,2S)-2- hydroxycycloheptyl)-6-((6-(1- methyl-1H-pyrazol-4-yl)pyridin- 3-yl)methyl)-5-oxo-5,6-dihydro- 1,6-naphthyridine-8- carboxamide | 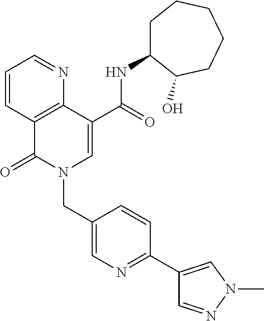 [SMILES: Cn1cc(-c2ccc(Cn3cc(C(=O)N[C@H]4CCCCC[C@@H]4O)c4ncccc4c3=O)cn2)cn1] | 473.2 |
| A19 | N-((3R,4S)-3- hydroxytetrahydro-2H-pyran-4- yl)-6-(4-(2-methyloxazol-4- yl)benzyl)-5-oxo-5,6-dihydro- 1,6-naphthyridine-8- carboxamide | 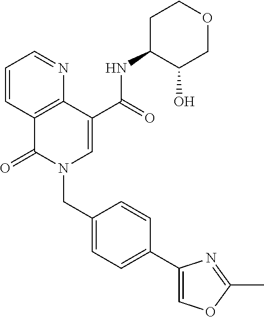 [SMILES: Cc1nc(-c2ccc(Cn3cc(C(=O)N[C@H]4CCOC[C@@H]4O)c4ncccc4c3=O)cc2)co1] | 461.2 |
| A20 | N-((1S,2S)-2- hydroxycyclohexyl)-6-(4-(2- methyloxazol-4-yl)benzyl)-5- oxo-5,6-dihydro-1,6- naphthyridine-8-carboxamide | 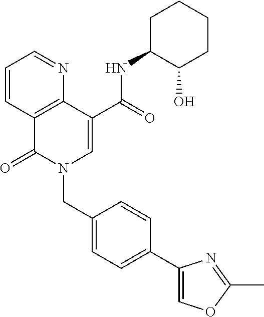 [SMILES: Cc1nc(-c2ccc(Cn3cc(C(=O)N[C@H]4CCCC[C@@H]4O)c4ncccc4c3=O)cc2)co1] | 459.2 |
| A21 | 6-(2,6-difluoro-4-(2-methyl-2H- indazol-4-yl)benzyl)-N- ((3R,4S)-3-hydroxytetrahydro- 2H-pyran-4-yl)-5-oxo-5,6- dihydro-1,6-naphthyridine-8- carboxamide | 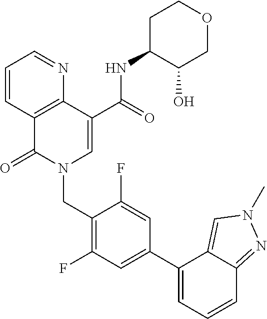 [SMILES: Cn1cc2c(-c3cc(F)c(Cn4cc(C(=O)N[C@H]5CCOC[C@@H]5O)c5ncccc5c4=O)c(F)c3)cccc2n1] | 546.2 |
| A22 | 6-(2,6-difluoro-4-(2-methyl-2H- indazol-4-yl)benzyl)-N-((1S,2S)- 2-hydroxycyclohexyl)-5-oxo- 5,6-dihydro-1,6-naphthyridine- 8-carboxamide | 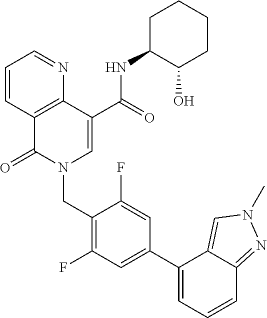 [SMILES: Cn1cc2c(-c3cc(F)c(Cn4cc(C(=O)N[C@H]5CCCC[C@@H]5O)c5ncccc5c4=O)c(F)c3)cccc2n1] | 544.2 |
| A23 | 6-(2,6-difluoro-4-(2-methyl-2H- indazol-4-yl)benzyl)-N-((1S,2S)- 2-hydroxycycloheptyl)-5-oxo- 5,6-dihydro-1,6-naphthyridine- 8-carboxamide | 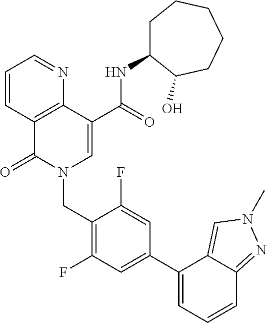 [SMILES: Cn1cc2c(-c3cc(F)c(Cn4cc(C(=O)N[C@H]5CCCCC[C@@H]5O)c5ncccc5c4=O)c(F)c3)cccc2n1] | 558.2 |
| A24 | 6-(2-fluoro-4-(1-methyl-1H- pyrazol-4-yl)benzyl)-N- ((1S,2S)-2-hydroxycycloheptyl)- 5-oxo-5,6-dihydro-1,6- naphthyridine-8-carboxamide | 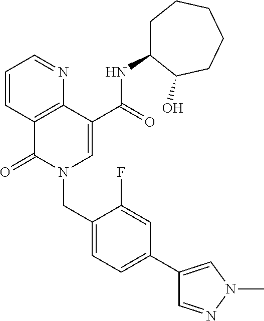 [SMILES: Cn1cc(-c2ccc(Cn3cc(C(=O)N[C@H]4CCCCC[C@@H]4O)c4ncccc4c3=O)c(F)c2)cn1] | 490.2 |
| A25 | 6-(4-(1H-pyrazol-1-yl)benzyl)- N-((1S,2S)-2- hydroxycycloheptyl)-5-oxo-5,6- dihydro-1,6-naphthyridine-8- carboxamide | 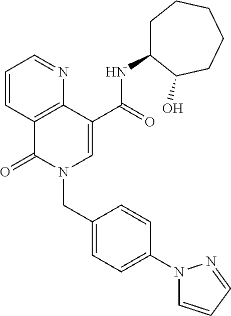 [SMILES: O=C(N[C@H]1CCCCC[C@@H]1O)c1cn(Cc2ccc(-n3cccn3)cc2)c(=O)c2cccnc12] | 458.2 |
| A26 | 6-(2-fluoro-4-(2-methylpyridin- 4-yl)benzyl)-N-((3R,4S)-3- hydroxytetrahydro-2H-pyran-4- yl)-5-oxo-5,6-dihydro-1,6- naphthyridine-8-carboxamide | 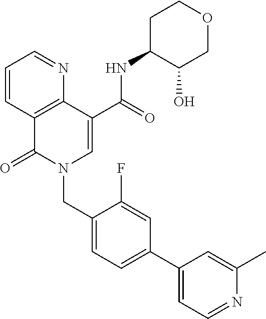 [SMILES: Cc1cc(-c2ccc(Cn3cc(C(=O)N[C@H]4CCOC[C@@H]4O)c4ncccc4c3=O)c(F)c2)ccn1] | 489.4 |
| A27 | 6-(2-fluoro-4-(2-methylpyridin- 4-yl)benzyl)-N-((1S,2S)-2- hydroxycyclohexyl)-5-oxo-5,6- dihydro-1,6-naphthyridine-8- carboxamide | 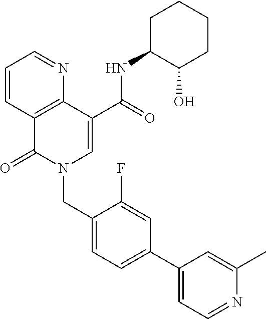 [SMILES: Cc1cc(-c2ccc(Cn3cc(C(=O)N[C@H]4CCCC[C@@H]4O)c4ncccc4c3=O)c(F)c2)ccn1] | 487.4 |
| A28 | 6-(2-fluoro-4-(2-methylpyridin- 4-yl)benzyl)-N-((1S,2S)-2- hydroxycyclopentyl)-5-oxo-5,6- dihydro-1,6-naphthyridine-8- carboxamide | 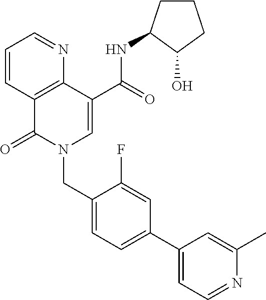 [SMILES: Cc1cc(-c2ccc(Cn3cc(C(=O)N[C@H]4CCC[C@@H]4O)c4ncccc4c3=O)c(F)c2)ccn1] | 473.4 |
| A29 | 6-((3-fluoro-3′-methyl-[1,1′- biphenyl]-4-yl)methyl)-N- ((3R,4S)-3-hydroxytetrahydro- 2H-pyran-4-yl)-5-oxo-5,6- dihydro-1,6-naphthyridine-8- carboxamide | 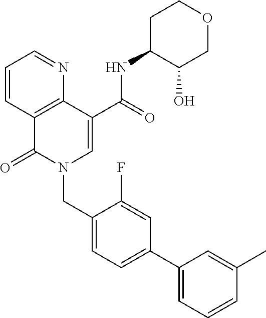 [SMILES: Cc1cccc(-c2ccc(Cn3cc(C(=O)N[C@H]4CCOC[C@@H]4O)c4ncccc4c3=O)c(F)c2)c1] | 488.4 |
| A30 | 6-((3-fluoro-3′,5′-dimethyl-[1,1′- biphenyl]-4-yl)methyl)-N- ((3R,4S)-3-hydroxytetrahydro- 2H-pyran-4-yl)-5-oxo-5,6- dihydro-1,6-naphthyridine-8- carboxamide | 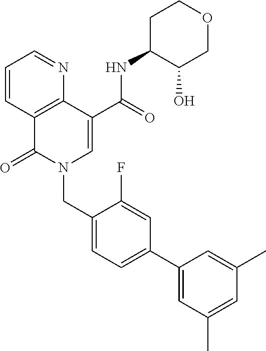 [SMILES: Cc1cc(C)cc(-c2ccc(Cn3cc(C(=O)N[C@H]4CCOC[C@@H]4O)c4ncccc4c3=O)c(F)c2)c1] | 502.4 |
| A31 | 6-((3,5-difluoro-3′-methyl-[1,1′- biphenyl]-4-yl)methyl)-N- ((3R,4S)-3-hydroxytetrahydro- 2H-pyran-4-yl)-5-oxo-5,6- dihydro-1,6-naphthyridine-8- carboxamide | 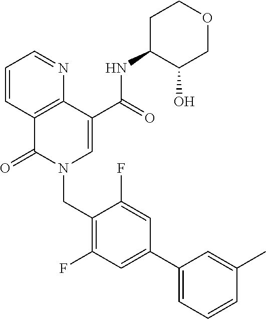 [SMILES: Cc1cccc(-c2cc(F)c(Cn3cc(C(=O)N[C@H]4CCOC[C@@H]4O)c4ncccc4c3=O)c(F)c2)c1] | 506.4 |
| A32 | 6-((3,5-difluoro-3′,5′-dimethyl- [1,1′-biphenyl]-4-yl)methyl)-N- ((3R,4S)-3-hydroxytetrahydro- 2H-pyran-4-yl)-5-oxo-5,6- dihydro-1,6-naphthyridine-8- carboxamide | 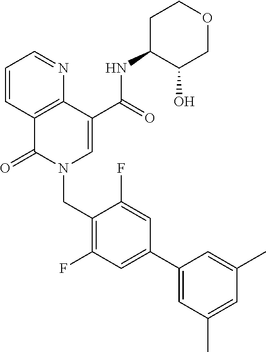 [SMILES: Cc1cc(C)cc(-c2cc(F)c(Cn3cc(C(=O)N[C@H]4CCOC[C@@H]4O)c4ncccc4c3=O)c(F)c2)c1] | 520.4 |
| A33 | 6-(2,6-difluoro-4-(5- methylpyridin-3-yl)benzyl)-N- ((3R,4S)-3-hydroxytetrahydro- 2H-pyran-4-yl)-5-oxo-5,6- dihydro-1,6-naphthyridine-8- carboxamide | 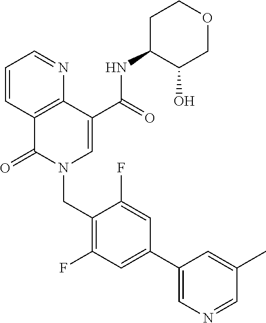 [SMILES: Cc1cncc(-c2cc(F)c(Cn3cc(C(=O)N[C@H]4CCOC[C@@H]4O)c4ncccc4c3=O)c(F)c2)c1] | 507.3 |
| A34 | 6-(2,6-difluoro-4-(6- methylpyridin-3-yl)benzyl)-N- ((3R,4S)-3-hydroxytetrahydro- 2H-pyran-4-yl)-5-oxo-5,6- dihydro-1,6-naphthyridine-8- carboxamide | 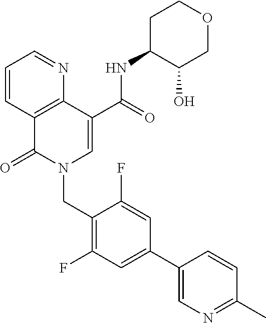 [SMILES: Cc1ccc(-c2cc(F)c(Cn3cc(C(=O)N[C@H]4CCOC[C@@H]4O)c4ncccc4c3=O)c(F)c2)cn1] | 507.4 |
| A35 | 6-(2,6-difluoro-4-(6- methylpyridin-3-yl)benzyl)-N- ((1S,2S)-2-hydroxycyclohexyl)- 5-oxo-5,6-dihydro-1,6- naphthyridine-8-carboxamide | 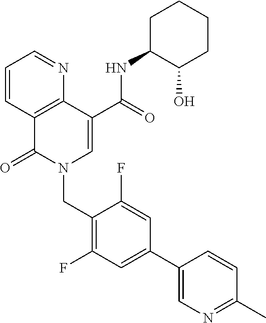 [SMILES: Cc1ccc(-c2cc(F)c(Cn3cc(C(=O)N[C@H]4CCCC[C@@H]4O)c4ncccc4c3=O)c(F)c2)cn1] | 505.4 |
| A36 | 6-(2,6-difluoro-4-(2- methylpyridin-4-yl)benzyl)-N- ((3R,4S)-3-hydroxytetrahydro- 2H-pyran-4-yl)-5-oxo-5,6- dihydro-1,6-naphthyridine-8- carboxamide | 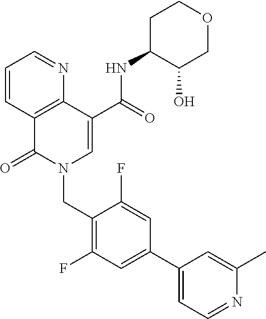 [SMILES: Cc1cc(-c2cc(F)c(Cn3cc(C(=O)N[C@H]4CCOC[C@@H]4O)c4ncccc4c3=O)c(F)c2)ccn1] | 507.4 |
| A37 | 6-(2,6-difluoro-4-(2- methylpyridin-4-yl)benzyl)-N- ((1S,2S)-2-hydroxycyclohexyl)- 5-oxo-5,6-dihydro-1,6- naphthyridine-8-carboxamide | 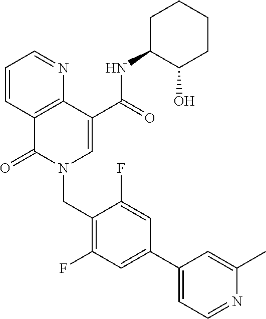 [SMILES: Cc1cc(-c2cc(F)c(Cn3cc(C(=O)N[C@H]4CCCC[C@@H]4O)c4ncccc4c3=O)c(F)c2)ccn1] | 505.4 |
| A38 | 6-(2,6-difluoro-4-(2- methylpyridin-4-yl)benzyl)-N- ((1S,2S)-2-hydroxycyclopentyl)- 5-oxo-5,6-dihydro-1,6- naphthyridine-8-carboxamide | 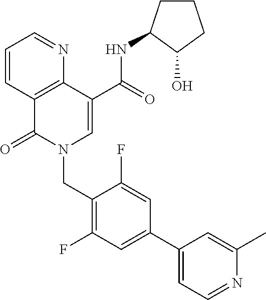 [SMILES: Cc1cc(-c2cc(F)c(Cn3cc(C(=O)N[C@H]4CCC[C@@H]4O)c4ncccc4c3=O)c(F)c2)ccn1] | 491.3 |
| A39 | 6-((4′-chloro-3,5-difluoro-3′- methoxy-[1,1′-biphenyl]-4- yl)methyl)-N-((3R,4S)-3- hydroxytetrahydro-2H-pyran-4- yl)-5-oxo-5,6-dihydro-1,6- naphthyridine-8-carboxamide | 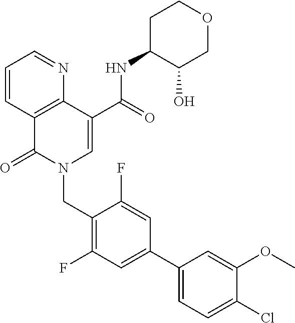 [SMILES: COc1cc(-c2cc(F)c(Cn3cc(C(=O)N[C@H]4CCOC[C@@H]4O)c4ncccc4c3=O)c(F)c2)ccc1Cl] | 556.3 |
| A40 | 6-(4-(1-cyclopropyl-1H-pyrazol- 4-yl)-2,6-difluorobenzyl)-N- ((3R,4S)-3-hydroxytetrahydro- 2H-pyran-4-yl)-5-oxo-5,6- dihydro-1,6-naphthyridine-8- carboxamide | 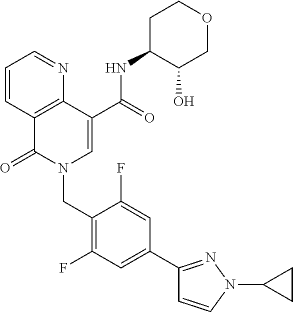 [SMILES: O=C(N[C@H]1CCOC[C@@H]1O)c1cn(Cc2c(F)cc(-c3ccn(C4CC4)n3)cc2F)c(=O)c2cccnc12] | 522.4 |
| A41 | 6-(4-(1-cyclopropyl-1H-pyrazol- 4-yl)-2,6-difluorobenzyl)-N- ((1S,2S)-2-hydroxycyclohexyl)- 5-oxo-5,6-dihydro-1,6- naphthyridine-8-carboxamide | 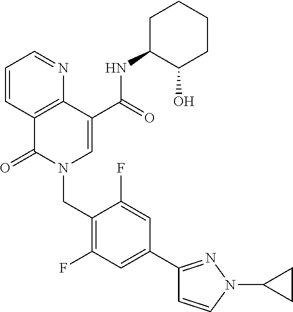 [SMILES: O=C(N[C@H]1CCCC[C@@H]1O)c1cn(Cc2c(F)cc(-c3ccn(C4CC4)n3)cc2F)c(=O)c2cccnc12] | 520 |
| A42 | 6-(4-(1-cyclopropyl-1H-pyrazol- 4-yl)-2,6-difluorobenzyl)-N- ((1S,2S)-2-hydroxycyclopentyl)- 5-oxo-5,6-dihydro-1,6- naphthyridine-8-carboxamide | 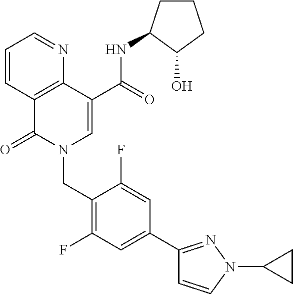 [SMILES: O=C(N[C@H]1CCC[C@@H]1O)c1cn(Cc2c(F)cc(-c3ccn(C4CC4)n3)cc2F)c(=O)c2cccnc12] | 506.4 |
| A43 | 6-(2,6-difluoro-4-(5- methylpyridin-3-yl)benzyl)-N- ((1S,2S)-2-hydroxycyclohexyl)- 5-oxo-5,6-dihydro-1,6- naphthyridine-8-carboxamide | 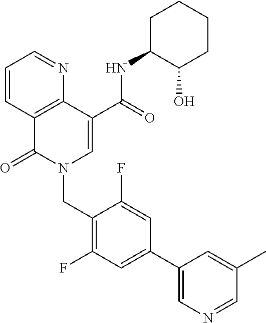 [SMILES: Cc1cncc(-c2cc(F)c(Cn3cc(C(=O)N[C@H]4CCCC[C@@H]4O)c4ncccc4c3=O)c(F)c2)c1] | 505.4 |
| A44 | 6-(2,6-difluoro-4-(5- methylpyridin-3-yl)benzyl)-N- ((1S,2S)-2-hydroxycyclopentyl)- 5-oxo-5,6-dihydro-1,6- naphthyridine-8-carboxamide | 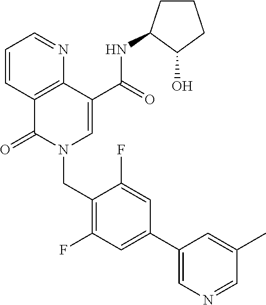 [SMILES: Cc1cncc(-c2cc(F)c(Cn3cc(C(=O)N[C@H]4CCC[C@@H]4O)c4ncccc4c3=O)c(F)c2)c1] | 491.4 |
| A45 | 6-(2-fluoro-4-(1-methyl-1H- pyrazol-5-yl)benzyl)-N- ((3R,4S)-3-hydroxytetrahydro- 2H-pyran-4-yl)-5-oxo-5,6- dihydro-1,6-naphthyridine-8- carboxamide | 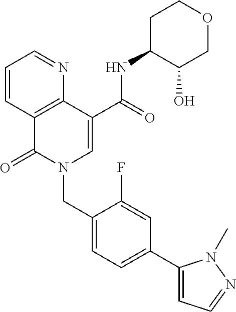 [SMILES: Cn1nccc1-c1ccc(Cn2cc(C(=O)N[C@H]3CCOC[C@@H]3O)c3ncccc3c2=O)c(F)c1] | 478.3 |
| A46 | 6-(2-fluoro-4-(1-methyl-1H- pyrazol-5-yl)benzyl)-N- ((1S,2S)-2-hydroxycyclohexyl)- 5-oxo-5,6-dihydro-1,6- naphthyridine-8-carboxamide | 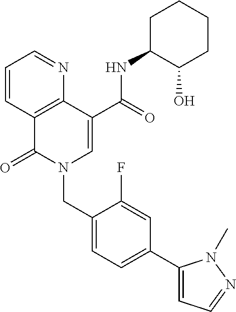 [SMILES: Cn1nccc1-c1ccc(Cn2cc(C(=O)N[C@H]3CCCC[C@@H]3O)c3ncccc3c2=O)c(F)c1] | 476.4 |
| A47 | 6-(2,6-difluoro-4-(1-methyl-1H- pyrazol-5-yl)benzyl)-N- ((3R,4S)-3-hydroxytetrahydro- 2H-pyran-4-yl)-5-oxo-5,6- dihydro-1,6-naphthyridine-8- carboxamide | 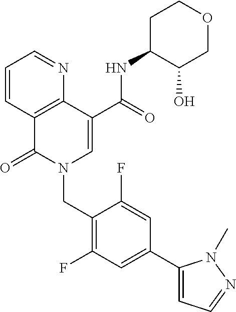 [SMILES: Cn1nccc1-c1cc(F)c(Cn2cc(C(=O)N[C@H]3CCOC[C@@H]3O)c3ncccc3c2=O)c(F)c1] | 496.4 |
| A48 | 6-(2,6-difluoro-4-(1-methyl-1H- pyrazol-5-yl)benzyl)-N- ((1S,2S)-2-hydroxycyclohexyl)- 5-oxo-5,6-dihydro-1,6- naphthyridine-8-carboxamide | 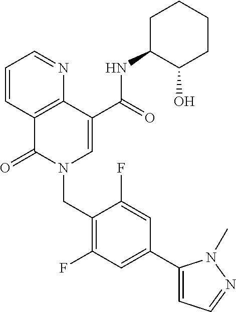 [SMILES: Cn1nccc1-c1cc(F)c(Cn2cc(C(=O)N[C@H]3CCCC[C@@H]3O)c3ncccc3c2=O)c(F)c1] | 494.4 |
| A49 | 6-(2,6-difluoro-4-(thiazol-5- yl)benzyl)-N-((3R,4S)-3- hydroxytetrahydro-2H-pyran-4- yl)-5-oxo-5,6-dihydro-1,6- naphthyridine-8-carboxamide | 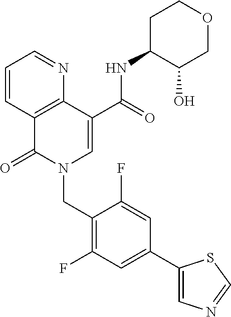 [SMILES: O=C(N[C@H]1CCOC[C@@H]1O)c1cn(Cc2c(F)cc(-c3cncs3)cc2F)c(=O)c2cccnc12] | 499.3 |
| A50 | 6-(2,6-difluoro-4-(isothiazol-5- yl)benzyl)-N-((3R,4S)-3- hydroxytetrahydro-2H-pyran-4- yl)-5-oxo-5,6-dihydro-1,6- naphthyridine-8-carboxamide | 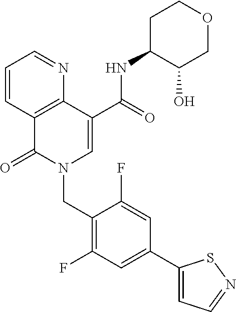 [SMILES: O=C(N[C@H]1CCOC[C@@H]1O)c1cn(Cc2c(F)cc(-c3ccns3)cc2F)c(=O)c2cccnc12] | 499.3 |
| A51 | 6-(2,6-difluoro-4-(2- methylthiazol-5-yl)benzyl)-N- ((3R,4S)-3-hydroxytetrahydro- 2H-pyran-4-yl)-5-oxo-5,6- dihydro-1,6-naphthyridine-8- carboxamide | 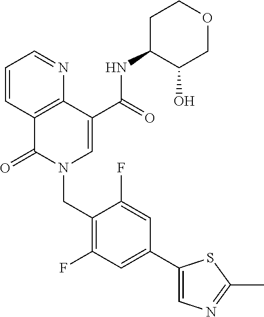 [SMILES: Cc1ncc(-c2cc(F)c(Cn3cc(C(=O)N[C@H]4CCOC[C@@H]4O)c4ncccc4c3=O)c(F)c2)s1] | 513.2 |
| A52 | 6-(2,6-difluoro-4-(2-methyl-2H- indazol-4-yl)benzyl)-N-((1S,2S)- 2-hydroxycyclohexyl)-5-oxo- 5,6-dihydropyrido[3,4- b]pyrazine-8-carboxamide | 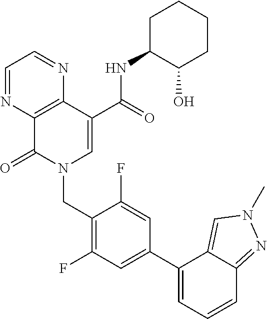 [SMILES: Cn1cc2c(-c3cc(F)c(Cn4cc(C(=O)N[C@H]5CCCC[C@@H]5O)c5nccnc5c4=O)c(F)c3)cccc2n1] | 545 |
| A53 | 6-(4-(1H-pyrazol-1-yl)benzyl)- N-((1S,2S)-2- hydroxycyclohexyl)-5-oxo-5,6- dihydropyrido[3,4-b]pyrazine-8- carboxamide | 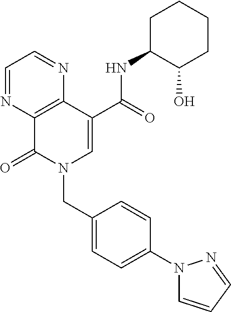 [SMILES: O=C(N[C@H]1CCCC[C@@H]1O)c1cn(Cc2ccc(-n3cccn3)cc2)c(=O)c2nccnc12] | 445 |
| A54 | 6-(2-fluoro-4-(1-methyl-1H- pyrazol-4-yl)benzyl)-N- ((1S,2S)-2-hydroxycyclohexyl)- 5-oxo-5,6-dihydropyrido[3,4- b]pyrazine-8-carboxamide | 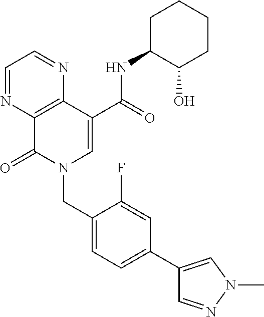 [SMILES: Cn1cc(-c2ccc(Cn3cc(C(=O)N[C@H]4CCCC[C@@H]4O)c4nccnc4c3=O)c(F)c2)cn1] | 477 |
| A55 | 6-(2-fluoro-4-(1-methyl-1H- pyrazol-3-yl)benzyl)-N- ((1S,2S)-2-hydroxycyclohexyl)- 5-oxo-5,6-dihydropyrido[3,4- b]pyrazine-8-carboxamide | 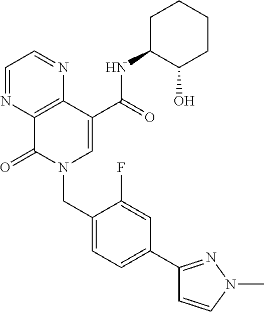 [SMILES: Cn1ccc(-c2ccc(Cn3cc(C(=O)N[C@H]4CCCC[C@@H]4O)c4nccnc4c3=O)c(F)c2)n1] | 477 |
| A56 | 6-(4-(1H-pyrazol-1-yl)benzyl)- N-((3R,4S)-3- hydroxytetrahydro-2H-pyran-4- yl)-5-oxo-5,6- dihydropyrido[3,4-b]pyrazine-8- carboxamide | 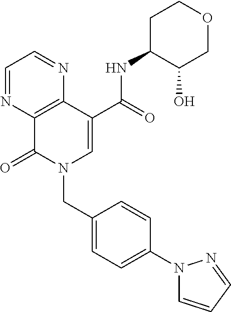 [SMILES: O=C(N[C@H]1CCOC[C@@H]1O)c1cn(Cc2ccc(-n3cccn3)cc2)c(=O)c2nccnc12] | 447 |
| A57 | N-((3R,4S)-3- hydroxytetrahydro-2H-pyran-4- yl)-5-oxo-6-(4-(thiazol-4- yl)benzyl)-5,6-dihydro-1,6- naphthyridine-8-carboxamide | 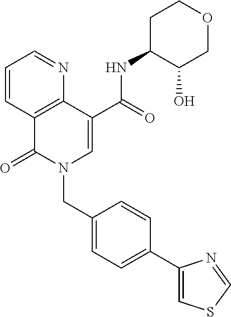 [SMILES: O=C(N[C@H]1CCOC[C@@H]1O)c1cn(Cc2ccc(-c3cscn3)cc2)c(=O)c2cccnc12] | 463 |
| A58 | N-((3R,4S)-3- hydroxytetrahydro-2H-pyran-4- yl)-6-(4-(1-methyl-1H-pyrazol- 4-yl)benzyl)-5-oxo-5,6-dihydro- 1,6-naphthyridine-8- carboxamide | 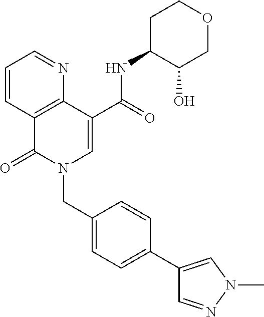 [SMILES: Cn1cc(-c2ccc(Cn3cc(C(=O)N[C@H]4CCOC[C@@H]4O)c4ncccc4c3=O)cc2)cn1] | 460.2 |
| A59 | N-((1S,2S)-2- hydroxycyclohexyl)-5-oxo-6-(4- (thiazol-4-yl)benzyl)-5,6- dihydro-1,6-naphthyridine-8- carboxamide | 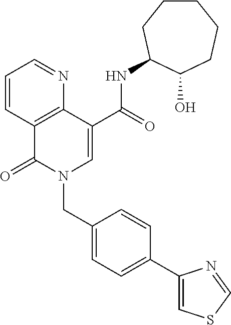 [SMILES: O=C(N[C@H]1CCCCC[C@@H]1O)c1cn(Cc2ccc(-c3cscn3)cc2)c(=O)c2cccnc12] | 461.2 |
| A60 | N-((1S,2S)-2- hydroxycycloheptyl)-5-oxo-6-(4- (thiazol-4-yl)benzyl)-5,6- dihydro-1,6-naphthyridine-8- carboxamide | 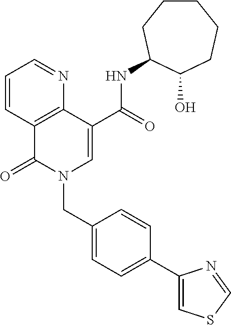 [SMILES: O=C(N[C@H]1CCCCC[C@@H]1O)c1cn(Cc2ccc(-c3cscn3)cc2)c(=O)c2cccnc12] | 475.2 |
| A61 | 6-(4-(1H-pyrazol-1-yl)benzyl)- N-((1S,2S)-2- hydroxycyclopentyl)-5-oxo-5,6- dihydropyrido[3,4-b]pyrazine-8- carboxamide | 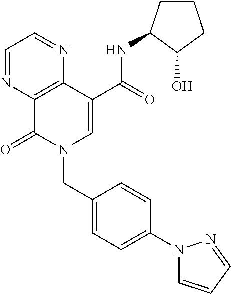 [SMILES: O=C(N[C@H]1CCC[C@@H]1O)c1cn(Cc2ccc(-n3cccn3)cc2)c(=O)c2nccnc12] | 431.2 |
| A62 | 6-(4-(1H-pyrazol-1-yl)benzyl)- N-((1S,2S)-2- hydroxycycloheptyl)-5-oxo-5,6- dihydropyrido[3,4-b]pyrazine-8- carboxamide | 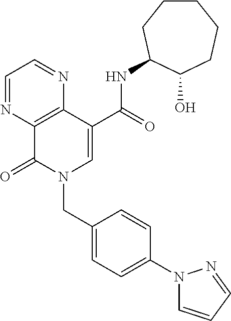 [SMILES: O=C(N[C@H]1CCCCC[C@@H]1O)c1cn(Cc2ccc(-n3cccn3)cc2)c(=O)c2nccnc12] | 459.2 |
| A63 | 6-(2-fluoro-4-(1-methyl-1H- pyrazol-3-yl)benzyl)-N- ((3R,4S)-3-hydroxytetrahydro- 2H-pyran-4-yl)-5-oxo-5,6- dihydropyrido[3,4-b]pyrazine-8- carboxamide | 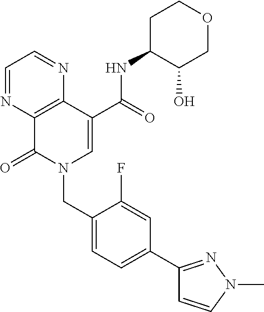 [SMILES: Cn1ccc(-c2ccc(Cn3cc(C(=O)N[C@H]4CCOC[C@@H]4O)c4nccnc4c3=O)c(F)c2)n1] | 479.2 |
| A64 | 6-(2-fluoro-4-(1-methyl-1H- pyrazol-3-yl)benzyl)-N- ((1S,2S)-2-hydroxycyclopentyl)- 5-oxo-5,6-dihydropyrido[3,4- b]pyrazine-8-carboxamide | 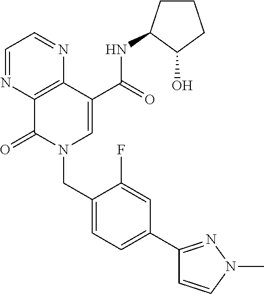 [SMILES: Cn1ccc(-c2ccc(Cn3cc(C(=O)N[C@H]4CCC[C@@H]4O)c4nccnc4c3=O)c(F)c2)n1] | 463.2 |
| A65 | 6-(2-fluoro-4-(1-methyl-1H- pyrazol-3-yl)benzyl)-N- ((1S,2S)-2-hydroxycycloheptyl)- 5-oxo-5,6-dihydropyrido[3,4- b]pyrazine-8-carboxamide | 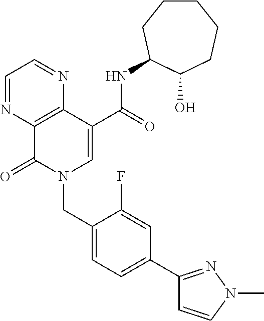 [SMILES: Cn1ccc(-c2ccc(Cn3cc(C(=O)N[C@H]4CCCCC[C@@H]4O)c4nccnc4c3=O)c(F)c2)n1] | 491.2 |
| A66 | 6-(2,6-difluoro-4-(2-methyl-2H- indazol-4-yl)benzyl)-N- ((3R,4S)-3-hydroxytetrahydro- 2H-pyran-4-yl)-5-oxo-5,6- dihydropyrido[3,4-b]pyrazine-8- carboxamide | 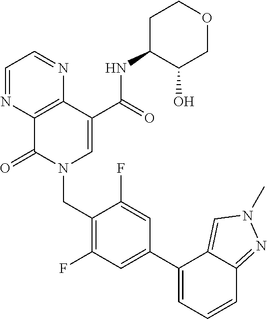 [SMILES: Cn1cc2c(-c3cc(F)c(Cn4cc(C(=O)N[C@H]5CCOC[C@@H]5O)c5nccnc5c4=O)c(F)c3)cccc2n1] | 547 |
| A67 | 6-(2,6-difluoro-4-(2-methyl-2H- indazol-4-yl)benzyl)-N-((1S,2S)- 2-hydroxycycloheptyl)-5-oxo- 5,6-dihydropyrido[3,4- b]pyrazine-8-carboxamide | 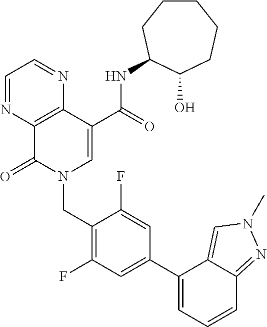 [SMILES: Cn1cc2c(-c3cc(F)c(Cn4cc(C(=O)N[C@H]5CCCCC[C@@H]5O)c5nccnc5c4=O)c(F)c3)cccc2n1] | 559 |
| A68 | 6-(2,6-difluoro-4-(2-methyl-2H- indazol-4-yl)benzyl)-N-((1S,2S)- 2-hydroxycyclopentyl)-5-oxo- 5,6-dihydropyrido[3,4- b]pyrazine-8-carboxamide | 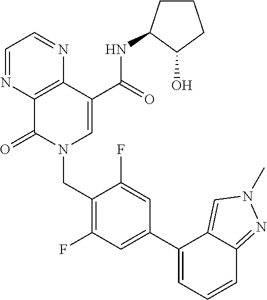 [SMILES: Cn1cc2c(-c3cc(F)c(Cn4cc(C(=O)N[C@H]5CCC[C@@H]5O)c5nccnc5c4=O)c(F)c3)cccc2n1] | 531 |
| A69 | 6-(2,6-difluoro-4-(2-methyl-2H- indazol-4-yl)benzyl)-N-((1S,2S)- 2-hydroxycyclobutyl)-5-oxo-5,6- dihydropyrido[3,4-b]pyrazine-8- carboxamide | 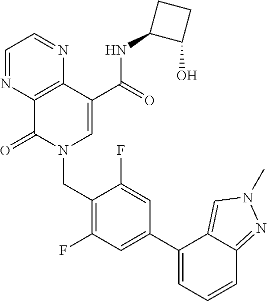 [SMILES: Cn1cc2c(-c3cc(F)c(Cn4cc(C(=O)N[C@H]5CC[C@@H]5O)c5nccnc5c4=O)c(F)c3)cccc2n1] | 517 |
| A70 | 6-(2,6-difluoro-4-(2-methyl-2H- indazol-4-yl)benzyl)-5-oxo-N- (tetrahydro-2H-pyran-4-yl)-5,6- dihydropyrido[3,4-b]pyrazine-8- carboxamide | 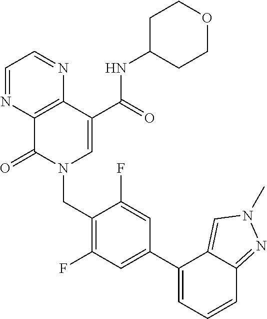 [SMILES: Cn1cc2c(-c3cc(F)c(Cn4cc(C(=O)NC5CCOCC5)c5nccnc5c4=O)c(F)c3)cccc2n1] | 531 |
| A71 | 6-(2,6-difluoro-4-(2-methyl-2H- indazol-4-yl)benzyl)-5-oxo-N- (2-oxaspiro[3.3]heptan-6-yl)- 5,6-dihydropyrido[3,4- b]pyrazine-8-carboxamide | 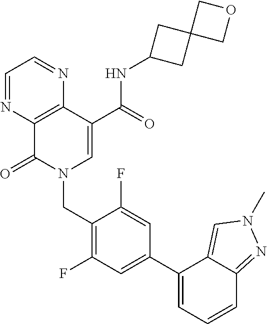 [SMILES: Cn1cc2c(-c3cc(F)c(Cn4cc(C(=O)NC5CC6(COC6)C5)c5nccnc5c4=O)c(F)c3)cccc2n1] | 543 |
| A72 | 6-(2,6-difluoro-4-(2-methyl-2H- indazol-4-yl)benzyl)-N-(3- (hydroxymethyl)tetrahydrofuran- 3-yl)-5-oxo-5,6- dihydropyrido[3,4-b]pyrazine-8- carboxamide | 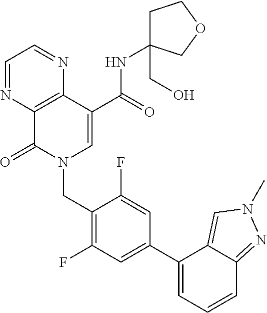 [SMILES: Cn1cc2c(-c3cc(F)c(Cn4cc(C(=O)NC5(CO)CCOC5)c5nccnc5c4=O)c(F)c3)cccc2n1] | 547 |
| A73 | 6-(2,6-difluoro-4-(2-methyl-2H- indazol-4-yl)benzyl)-N-(2- hydroxy-2-methylpropyl)-5-oxo- 5,6-dihydropyrido[3,4- b]pyrazine-8-carboxamide | 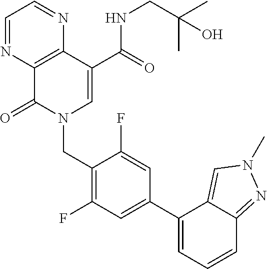 [SMILES: Cn1cc2c(-c3cc(F)c(Cn4cc(C(=O)NCC(C)(C)O)c5nccnc5c4=O)c(F)c3)cccc2n1] | 519 |
| A74 | 6-(2,6-difluoro-4-(2-methyl-2H- indazol-4-yl)benzyl)-5-oxo-N- (3,3,3-trifluoro-2- hydroxypropyl)-5,6- dihydropyrido[3,4-b]pyrazine-8- carboxamide | 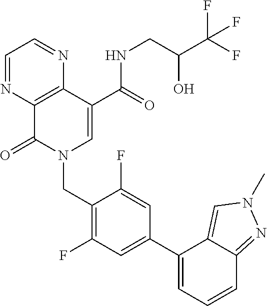 [SMILES: Cn1cc2c(-c3cc(F)c(Cn4cc(C(=O)NCC(O)C(F)(F)F)c5nccnc5c4=O)c(F)c3)cccc2n1] | 559 |
| A75 | (S)-6-(2,6-difluoro-4-(2-methyl- 2H-indazol-4-yl)benzyl)-N-(1- hydroxy-3-methylbutan-2-yl)-5- oxo-5,6-dihydropyrido[3,4- b]pyrazine-8-carboxamide | 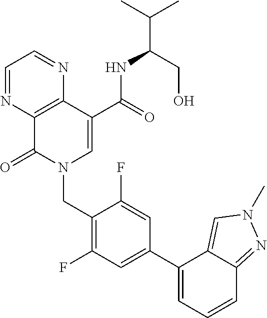 [SMILES: CC(C)[C@@H](CO)NC(=O)c1cn(Cc2c(F)cc(-c3cccc4nn(C)cc34)cc2F)c(=O)c2nccnc12] | 533 |
| A76 | 6-(2,6-difluoro-4-(2-methyl-2H- indazol-4-yl)benzyl)-N-((2S,3S)- 1-hydroxy-3-methylpentan-2- yl)-5-oxo-5,6- dihydropyrido[3,4-b]pyrazine-8- carboxamide | 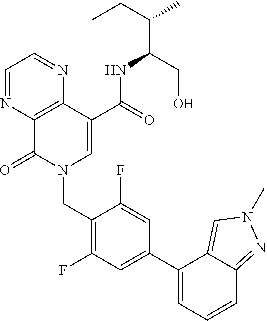 [SMILES: CC[C@H](C)[C@@H](CO)NC(=O)c1cn(Cc2c(F)cc(-c3cccc4nn(C)cc34)cc2F)c(=O)c2nccnc12] | 547 |
| A77 | 6-(2-fluoro-4-(1-methyl-1H- pyrazol-4-yl)benzyl)-N- ((3R,4S)-3-hydroxytetrahydro- 2H-pyran-4-yl)-5-oxo-5,6- dihydropyrido[3,4-b]pyrazine-8- carboxamide | 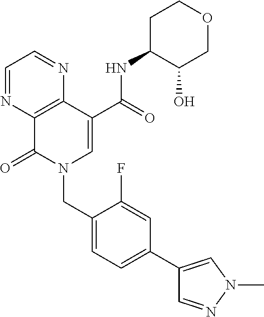 [SMILES: Cn1cc(-c2ccc(Cn3cc(C(=O)N[C@H]4CCOC[C@@H]4O)c4nccnc4c3=O)c(F)c2)cn1] | 479 |
| A78 | 6-(2-fluoro-4-(1-methyl-1H- pyrazol-4-yl)benzyl)-N- ((1S,2S)-2-hydroxycycloheptyl)- 5-oxo-5,6-dihydropyrido[3,4- b]pyrazine-8-carboxamide | 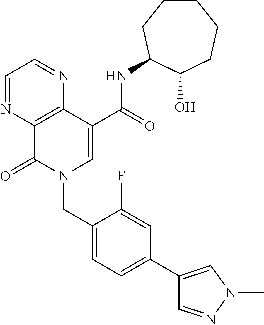 [SMILES: Cn1cc(-c2ccc(Cn3cc(C(=O)N[C@H]4CCCCC[C@@H]4O)c4nccnc4c3=O)c(F)c2)cn1] | 491 |
| A79 | 6-(2-fluoro-4-(1-methyl-1H- pyrazol-4-yl)benzyl)-N- ((1S,2S)-2-hydroxycyclopentyl)- 5-oxo-5,6-dihydropyrido[3,4- b]pyrazine-8-carboxamide | 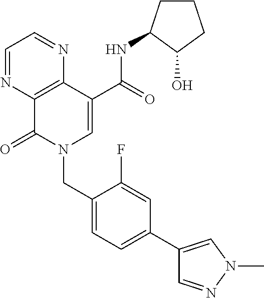 [SMILES: Cn1cc(-c2ccc(Cn3cc(C(=O)N[C@H]4CCC[C@@H]4O)c4nccnc4c3=O)c(F)c2)cn1] | 463 |
| A80 | 6-(2-fluoro-4-(1-methyl-1H- pyrazol-4-yl)benzyl)-N-(2- hydroxy-2-methylpropyl)-5-oxo- 5,6-dihydropyrido[3,4- b]pyrazine-8-carboxamide | 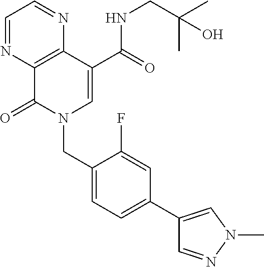 [SMILES: Cn1cc(-c2ccc(Cn3cc(C(=O)NCC(C)(C)O)c4nccnc4c3=O)c(F)c2)cn1] | 451 |
| A81 | 6-(2-fluoro-4-(1-methyl-1H- pyrazol-4-yl)benzyl)-5-oxo-N- (3,3,3-trifluoro-2- hydroxypropyl)-5,6- dihydropyrido[3,4-b]pyrazine-8- carboxamide | 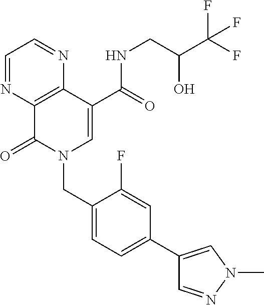 [SMILES: Cn1cc(-c2ccc(Cn3cc(C(=O)NCC(O)C(F)(F)F)c4nccnc4c3=O)c(F)c2)cn1] | 491 |
| A82 | (S)-6-(2-fluoro-4-(1-methyl-1H- pyrazol-4-yl)benzyl)-N-(1- hydroxy-3-methylbutan-2-yl)-5- oxo-5,6-dihydropyrido[3,4- b]pyrazine-8-carboxamide | 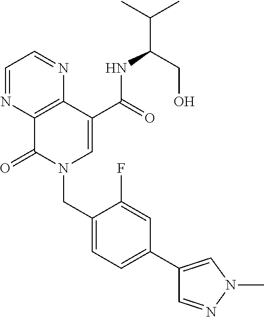 [SMILES: CC(C)[C@@H](CO)NC(=O)c1cn(Cc2ccc(-c3cnn(C)c3)cc2F)c(=O)c2nccnc12] | 465 |
| A83 | 6-(2-fluoro-4-(1-methyl-1H- pyrazol-4-yl)benzyl)-N- ((2S,3S)-1-hydroxy-3- methylpentan-2-yl)-5-oxo-5,6- dihydropyrido[3,4-b]pyrazine-8- carboxamide | 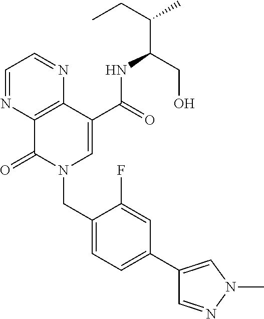 [SMILES: CC[C@H](C)[C@@H](CO)NC(=O)c1cn(Cc2ccc(-c3cnn(C)c3)cc2F)c(=O)c2nccnc12] | 479 |
| A84 | 6-(2,6-difluoro-4-(2-methyl-2H- indazol-4-yl)benzyl)-N- ((3R,4S)-3-hydroxytetrahydro- 2H-pyran-4-yl)-5-oxo-5,6- dihydropyrido[4,3-d]pyrimidine- 8-carboxamide | 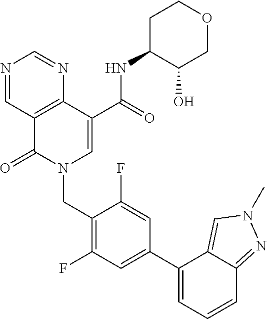 [SMILES: Cn1cc2c(-c3cc(F)c(Cn4cc(C(=O)N[C@H]5CCOC[C@@H]5O)c5ncncc5c4=O)c(F)c3)cccc2n1] | 547 |
| A85 | 6-(2,6-difluoro-4-(2-methyl-2H- indazol-4-yl)benzyl)-N-((1S,2S)- 2-hydroxycycloheptyl)-5-oxo- 5,6-dihydropyrido[4,3- d]pyrimidine-8-carboxamide | 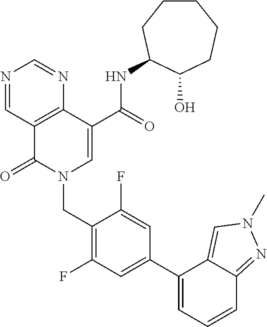 [SMILES: Cn1cc2c(-c3cc(F)c(Cn4cc(C(=O)N[C@H]5CCCCC[C@@H]5O)c5ncncc5c4=O)c(F)c3)cccc2n1] | 559 |
| A86 | N-((1S,2S)-2- hydroxycyclohexyl)-6-(4-(1- methyl-1H-pyrazol-4-yl)benzyl)- 5-oxo-5,6-dihydro-1,6- naphthyridine-8-carboxamide | 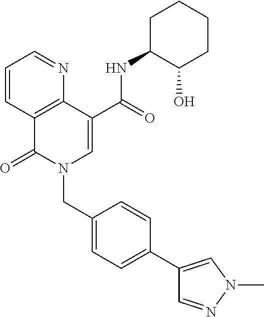 [SMILES: Cn1cc(-c2ccc(Cn3cc(C(=O)N[C@H]4CCCC[C@@H]4O)c4ncccc4c3=O)cc2)cn1] | 458.4 |
| A87 | N-((1S,2S)-2- hydroxycycloheptyl)-6-(4-(1- methyl-1H-pyrazol-4-yl)benzyl)- 5-oxo-5,6-dihydro-1,6- naphthyridine-8-carboxamide | 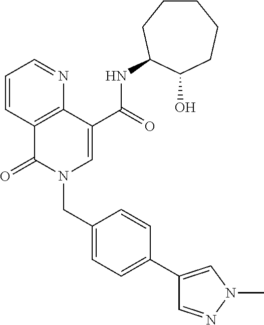 [SMILES: Cn1cc(-c2ccc(Cn3cc(C(=O)N[C@H]4CCCCC[C@@H]4O)c4ncccc4c3=O)cc2)cn1] | 472.4 |
| A88 | 6-(2,6-difluoro-4-(2-methyl-2H- indazol-4-yl)benzyl)-N-((1S,2S)- 2-hydroxycyclohexyl)-5-oxo- 5,6-dihydropyrido[4,3- d]pyrimidine-8-carboxamide | 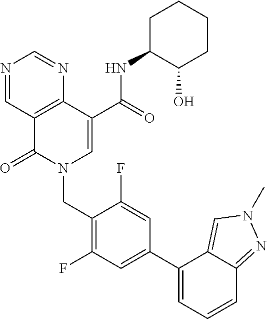 [SMILES: Cn1cc2c(-c3cc(F)c(Cn4cc(C(=O)N[C@H]5CCCC[C@@H]5O)c5ncncc5c4=O)c(F)c3)cccc2n1] | 545.4 |
| A89 | 6-(2-fluoro-4-(1-methyl-1H- pyrazol-4-yl)benzyl)-N- ((1S,2S)-2-hydroxycyclohexyl)- 5-oxo-5,6-dihydropyrido[4,3- d]pyrimidine-8-carboxamide | 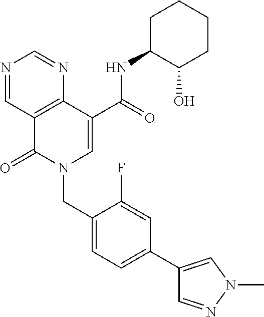 [SMILES: Cn1cc(-c2ccc(Cn3cc(C(=O)N[C@H]4CCCC[C@@H]4O)c4ncncc4c3=O)c(F)c2)cn1] | 477.3 |
| A90 | 6-(2-fluoro-4-(1-methyl-1H- pyrazol-4-yl)benzyl)-N- ((3R,4S)-3-hydroxytetrahydro- 2H-pyran-4-yl)-5-oxo-5,6- dihydropyrido[4,3-d]pyrimidine- 8-carboxamide | 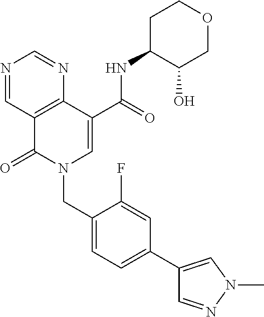 [SMILES: Cn1cc(-c2ccc(Cn3cc(C(=O)N[C@H]4CCOC[C@@H]4O)c4ncncc4c3=O)c(F)c2)cn1] | 479.2 |
| A91 | 6-(2-fluoro-4-(1-methyl-1H- pyrazol-4-yl)benzyl)-N- ((1S,2S)-2-hydroxycycloheptyl)- 5-oxo-5,6-dihydropyrido[4,3- d]pyrimidine-8-carboxamide | 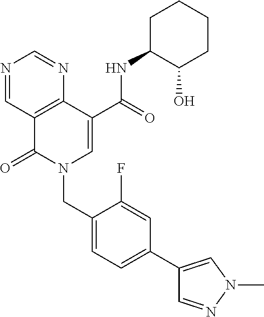 [SMILES: Cn1cc(-c2ccc(Cn3cc(C(=O)N[C@H]4CCCC[C@@H]4O)c4ncncc4c3=O)c(F)c2)cn1] | 491.4 |

TABLE B

| Cpd. | | | ES−MS |
|---|---|---|---|
| No. | Name | Structure | [M + 1]⁺ |
| B1 | 6-(2-fluoro-4-(1-methyl-1H-pyrazol- 3-yl)benzyl)-N-((3R,4S)-3- hydroxytetrahydro-2H-pyran-4-yl)- 5-oxo-5,6-dihydroimidazo[1,2- c]pyrimidine-8-carboxamide | 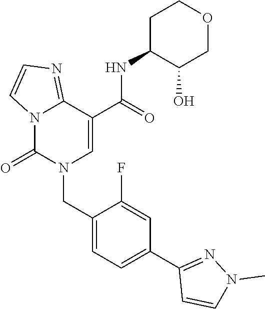 [SMILES: Cn1ccc(-c2ccc(Cn3cc(C(=O)N[C@H]4CCOC[C@@H]4O)c4nccn4c3=O)c(F)c2)n1] | 467.4 |
| B2 | 6-(2-fluoro-4-(1-methyl-1H-pyrazol- 3-yl)benzyl)-N-((1S,2S)-2- hydroxycyclohexyl)-5-oxo-5,6- dihydroimidazo[1,2-c]pyrimidine-8- carboxamide | 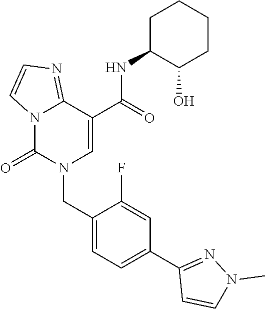 [SMILES: Cn1ccc(-c2ccc(Cn3cc(C(=O)N[C@H]4CCCC[C@@H]4O)c4nccn4c3=O)c(F)c2)n1] | 465.4 |
| B3 | 6-(2-fluoro-4-(1-methyl-1H-pyrazol- 3-yl)benzyl)-N-((1S,2S)-2- hydroxycyclopentyl)-5-oxo-5,6- dihydroimidazo[1,2-c]pyrimidine-8- carboxamide | 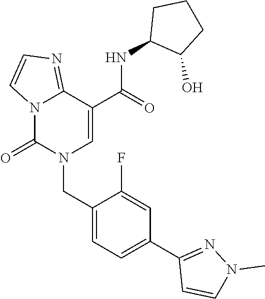 [SMILES: Cn1ccc(-c2ccc(Cn3cc(C(=O)N[C@H]4CCC[C@@H]4O)c4nccn4c3=O)c(F)c2)n1] | 451.4 |
| B4 | 6-(2-fluoro-4-(1-methyl-1H-pyrazol- 3-yl)benzyl)-N-((1R,2R)-2- hydroxycyclobutyl)-5-oxo-5,6- dihydroimidazo[1,2-c]pyrimidine-8- carboxamide | 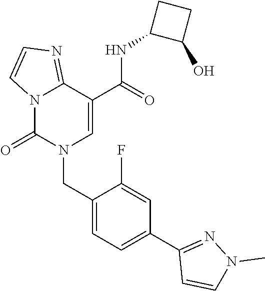 [SMILES: Cn1ccc(-c2ccc(Cn3cc(C(=O)N[C@@H]4CC[C@H]4O)c4nccn4c3=O)c(F)c2)n1] | 437.4 |
| B5 | 6-(2-fluoro-4-(1-methyl-1H-pyrazol- 4-yl)benzyl)-N-((3R,4S)-3- hydroxytetrahydro-2H-pyran-4-yl)- 5-oxo-5,6-dihydroimidazo[1,2- c]pyrimidine-8-carboxamide | 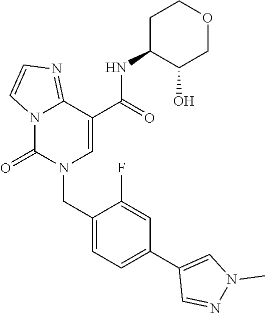 [SMILES: Cn1cc(-c2ccc(Cn3cc(C(=O)N[C@H]4CCOC[C@@H]4O)c4nccn4c3=O)c(F)c2)cn1] | 467.4 |
| B6 | 6-(2-fluoro-4-(1-methyl-1H-pyrazol- 4-yl)benzyl)-N-((1S,2S)-2- hydroxycyclohexyl)-5-oxo-5,6- dihydroimidazo[1,2-c]pyrimidine-8- carboxamide | 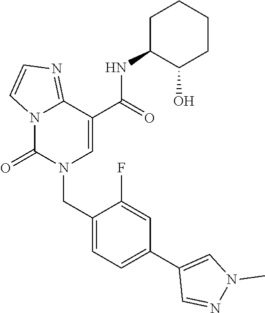 [SMILES: Cn1cc(-c2ccc(Cn3cc(C(=O)N[C@H]4CCCC[C@@H]4O)c4nccn4c3=O)c(F)c2)cn1] | 465.4 |
| B7 | 6-(2-fluoro-4-(1-methyl-1H-pyrazol- 4-yl)benzyl)-N-((1S,2S)-2- hydroxycyclopentyl)-5-oxo-5,6- dihydroimidazo[1,2-c]pyrimidine-8- carboxamide | 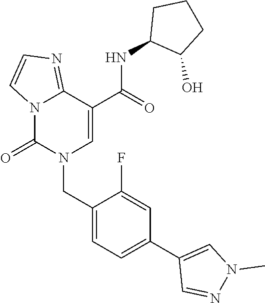 [SMILES: Cn1cc(-c2ccc(Cn3cc(C(=O)N[C@H]4CCC[C@@H]4O)c4nccn4c3=O)c(F)c2)cn1] | 451.4 |
| B8 | 6-(2-fluoro-4-(1-methyl-1H-pyrazol- 4-yl)benzyl)-N-((1R,2R)-2- hydroxycyclobutyl)-5-oxo-5,6- dihydroimidazo[1,2-c]pyrimidine-8- carboxamide | 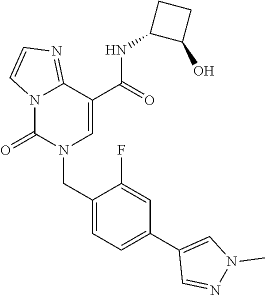 [SMILES: Cn1cc(-c2ccc(Cn3cc(C(=O)N[C@@H]4CC[C@H]4O)c4nccn4c3=O)c(F)c2)cn1] | 437.4 |
| B9 | 6-(2-fluoro-4-(1-methyl-1H-pyrazol- 4-yl)benzyl)-5-oxo-N-(2- oxaspiro[3.3]heptan-6-yl)-5,6- dihydroimidazo[1,2-c]pyrimidine-8- carboxamide | 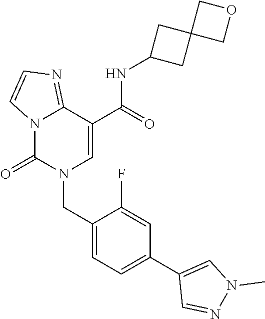 [SMILES: Cn1cc(-c2ccc(Cn3cc(C(=O)NC4CC5(COC5)C4)c4nccn4c3=O)c(F)c2)cn1] | 463.4 |
| B10 | N-((3R,4S)-3-hydroxytetrahydro- 2H-pyran-4-yl)-6-((6-(1-methyl-1H- pyrazol-4-yl)pyridin-3-yl)methyl)-5- oxo-5,6-dihydroimidazo[1,2- c]pyrimidine-8-carboxamide | 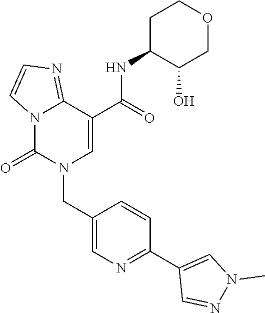 [SMILES: Cn1cc(-c2ccc(Cn3cc(C(=O)N[C@H]4CCOC[C@@H]4O)c4nccn4c3=O)cn2)cn1] | 450.5 |
| B11 | N-((1S,2S)-2-hydroxycyclohexyl)-6- ((6-(1-methyl-1H-pyrazol-4- yl)pyridin-3-yl)methyl)-5-oxo-5,6- dihydroimidazo[1,2-c]pyrimidine-8- carboxamide | 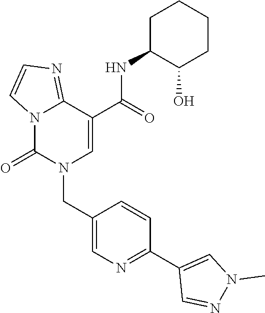 [SMILES: Cn1cc(-c2ccc(Cn3cc(C(=O)N[C@H]4CCCC[C@@H]4O)c4nccn4c3=O)cn2)cn1] | 448.4 |
| B12 | 6-(4-(1H-pyrazol-1-yl)benzyl)-N- ((3R,4S)-3-hydroxytetrahydro-2H- pyran-4-yl)-5-oxo-5,6- dihydroimidazo[1,2-c]pyrimidine-8- carboxamide | 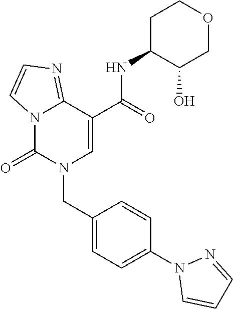 [SMILES: O=C(N[C@H]1CCOC[C@@H]1O)c1cn(Cc2ccc(-n3cccn3)cc2)c(=O)n2ccnc12] | 435.4 |
| B13 | 6-(4-(1H-pyrazol-1-yl)benzyl)-N- ((1S,2S)-2-hydroxycyclohexyl)-5- oxo-5,6-dihydroimidazo[1,2- c]pyrimidine-8-carboxamide | 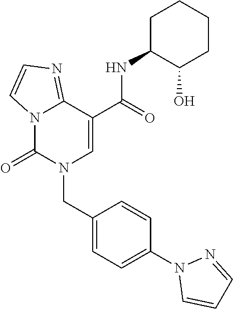 [SMILES: O=C(N[C@H]1CCCC[C@@H]1O)c1cn(Cc2ccc(-n3cccn3)cc2)c(=O)n2ccnc12] | 433.4 |
| B14 | 6-(4-(1H-pyrazol-1-yl)benzyl)-N- ((1S,2S)-2-hydroxycyclopentyl)-5- oxo-5,6-dihydroimidazo[1,2- c]pyrimidine-8-carboxamide | 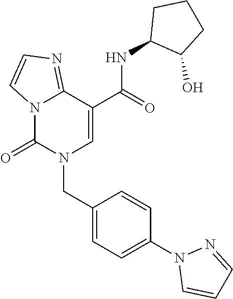 [SMILES: O=C(N[C@H]1CCC[C@@H]1O)c1cn(Cc2ccc(-n3cccn3)cc2)c(=O)n2ccnc12] | 419.4 |
| B15 | 6-(2-fluoro-4-(1-methyl-1H-pyrazol- 4-yl)benzyl)-N-((1S,2S)-2- hydroxycycloheptyl)-5-oxo-5,6- dihydroimidazo[1,2-c]pyrimidine-8- carboxamide | 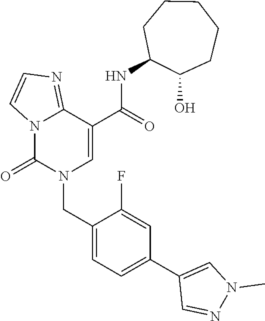 [SMILES: Cn1cc(-c2ccc(Cn3cc(C(=O)N[C@H]4CCCCC[C@@H]4O)c4nccn4c3=O)c(F)c2)cn1] | 479.2 |
| B16 | 6-(4-(1H-pyrazol-1-yl)benzyl)-N- ((1S,2S)-2-hydroxycycloheptyl)-5- oxo-5,6-dihydroimidazo[1,2- c]pyrimidine-8-carboxamide | 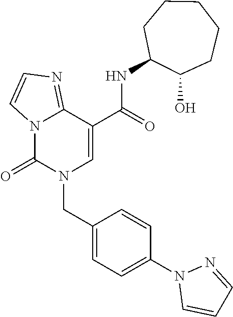 [SMILES: O=C(N[C@H]1CCCCC[C@@H]1O)c1cn(Cc2ccc(-n3cccn3)cc2)c(=O)n2ccnc12] | 447.5 |
| B17 | N-((3R,4S)-3-hydroxytetrahydro- 2H-pyran-4-yl)-5-oxo-6-(4-(thiazol- 4-yl)benzyl)-5,6- dihydroimidazo[1,2-c]pyrimidine-8- carboxamide | 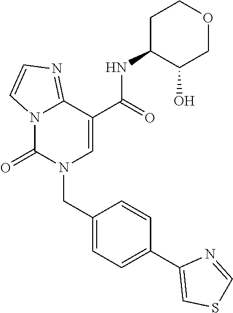 [SMILES: O=C(N[C@H]1CCOC[C@@H]1O)c1cn(Cc2ccc(-c3cscn3)cc2)c(=O)n2ccnc12] | 452.3 |
| B18 | 6-(2,6-difluoro-4-(2-methyl-2H- indazol-4-yl)benzyl)-N-((3R,4S)-3- hydroxytetrahydro-2H-pyran-4-yl)- 5-oxo-5,6-dihydroimidazo[1,2-c] pyrimidine-8-carboxamide | 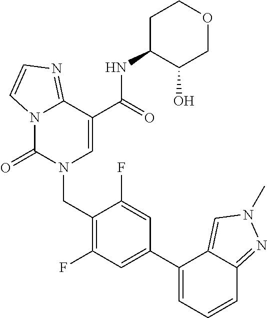 [SMILES: Cn1cc2c(-c3cc(F)c(Cn4cc(C(=O)N[C@H]5CCOC[C@@H]5O)c5nccn5c4=O)c(F)c3)cccc2n1] | 535.4 |
| B19 | 6-(2,6-difluoro-4-(2-methyl-2H- indazol-4-yl)benzyl)-N-((1S,2S)-2- hydroxycycloheptyl)-5-oxo-5,6- dihydroimidazo[1,2-c]pyrimidine-8- carboxamide | 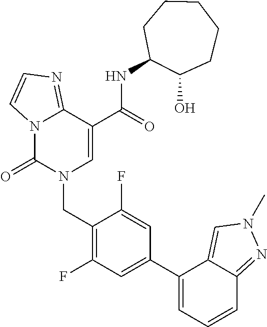 [SMILES: Cn1cc2c(-c3cc(F)c(Cn4cc(C(=O)N[C@H]5CCCCC[C@@H]5O)c5nccn5c4=O)c(F)c3)cccc2n1] | 547.2 |
| B20 | 6-(2,6-difluoro-4-(2-methyl-2H- indazol-4-yl)benzyl)-N-((1S,2S)-2- hydroxycyclohexyl)-5-oxo-5,6- dihydroimidazo[1,2-c]pyrimidine-8- carboxamide | 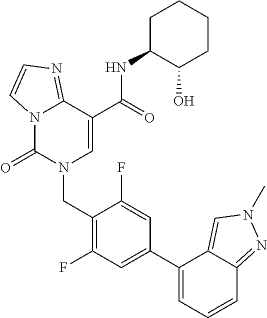 [SMILES: Cn1cc2c(-c3cc(F)c(Cn4cc(C(=O)N[C@H]5CCCC[C@@H]5O)c5nccn5c4=O)c(F)c3)cccc2n1] | 533.2 |
| B21 | 6-(2,6-difluoro-4-(2-methyl-2H- indazol-4-yl)benzyl)-N-((1S,2S)-2- hydroxycyclopentyl)-5-oxo-5,6- dihydroimidazo[1,2-c]pyrimidine-8- carboxamide | 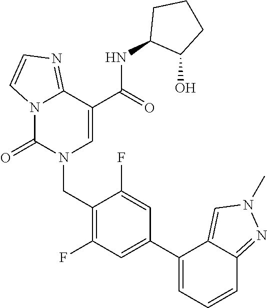 [SMILES: Cn1cc2c(-c3cc(F)c(Cn4cc(C(=O)N[C@H]5CCC[C@@H]5O)c5nccn5c4=O)c(F)c3)cccc2n1] | 519.2 |
| B22 | 6-(2,6-difluoro-4-(2-methyl-2H- indazol-4-yl)benzyl)-N-((1S,2S)-2- hydroxycyclobutyl)-5-oxo-5,6- dihydroimidazo[1,2-c]pyrimidine-8- carboxamide | 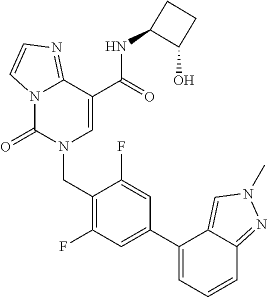 [SMILES: Cn1cc2c(-c3cc(F)c(Cn4cc(C(=O)N[C@H]5CC[C@@H]5O)c5nccn5c4=O)c(F)c3)cccc2n1] | 505.2 |
| B23 | 6-(2-fluoro-4-(1-methyl-1H-pyrazol- 4-yl)benzyl)-N-((3R,4S)-3- hydroxytetrahydro-2H-pyran-4-yl)- 2-methyl-5-oxo-5,6- dihydroimidazo[1,2-c]pyrimidine-8- carboxamide | 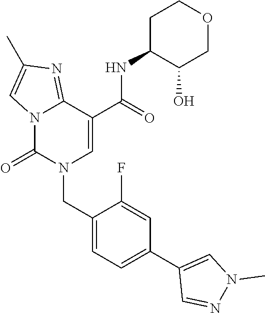 [SMILES: Cc1cn2c(=O)n(Cc3ccc(-c4cnn(C)c4)cc3F)cc(C(=O)N[C@H]3CCOC[C@@H]3O)c2n1] | 481.4 |
| B24 | 6-(2-fluoro-4-(1-methyl-1H-pyrazol- 4-yl)benzyl)-N-((1S,2S)-2- hydroxycyclohexyl)-2-methyl-5- oxo-5,6-dihydroimidazo[1,2- c]pyrimidine-8-carboxamide | 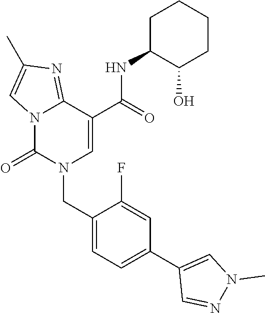 [SMILES: Cc1cn2c(=O)n(Cc3ccc(-c4cnn(C)c4)cc3F)cc(C(=O)N[C@H]3CCCC[C@@H]3O)c2n1] | 479.4 |
| B25 | 6-(2-fluoro-4-(1-methyl-1H-pyrazol- 3-yl)benzyl)-N-((3R,4S)-3- hydroxytetrahydro-2H-pyran-4-yl)- 2-methyl-5-oxo-5,6- dihydroimidazo[1,2-c]pyrimidine-8- carboxamide | 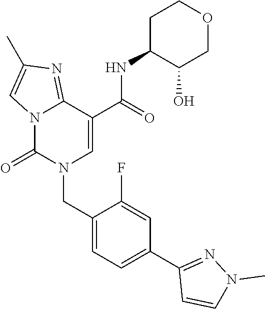 [SMILES: Cc1cn2c(=O)n(Cc3ccc(-c4ccn(C)n4)cc3F)cc(C(=O)N[C@H]3CCOC[C@@H]3O)c2n1] | 481.4 |
| B26 | 6-(2-fluoro-4-(1-methyl-1H-pyrazol- 3-yl)benzyl)-N-((1S,2S)-2- hydroxycyclohexyl)-2-methyl-5- oxo-5,6-dihydroimidazo[1,2- c]pyrimidine-8-carboxamide | 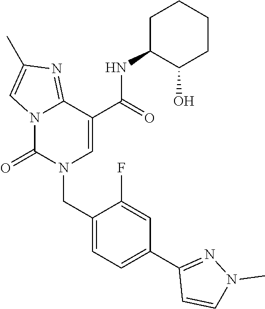 [SMILES: Cc1cn2c(=O)n(Cc3ccc(-c4ccn(C)n4)cc3F)cc(C(=O)N[C@H]3CCCC[C@@H]3O)c2n1] | 479.4 |
| B27 | 6-(4-(1H-pyrazol-1-yl)benzyl)-N- ((3R,4S)-3-hydroxytetrahydro-2H- pyran-4-yl)-2-methyl-5-oxo-5,6- dihydroimidazo[1,2-c]pyrimidine-8- carboxamide | 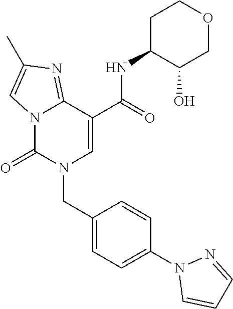 [SMILES: Cc1cn2c(=O)n(Cc3ccc(-n4cccn4)cc3)cc(C(=O)N[C@H]3CCOC[C@@H]3O)c2n1] | 449.4 |
| B28 | 6-(4-(1H-pyrazol-1-yl)benzyl)-N- ((1S,2S)-2-hydroxycyclohexyl)-2- methyl-5-oxo-5,6- dihydroimidazo[1,2-c]pyrimidine-8- carboxamide | 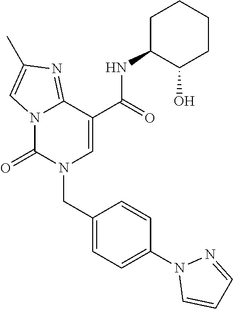 [SMILES: Cc1cn2c(=O)n(Cc3ccc(-n4cccn4)cc3)cc(C(=O)N[C@H]3CCCC[C@@H]3O)c2n1] | 447.4 |
| B29 | 6-(2,6-difluoro-4-(2-methyl-2H- indazol-4-yl)benzyl)-N-((3R,4S)-3- hydroxytetrahydro-2H-pyran-4-yl)- 2-methyl-5-oxo-5,6- dihydroimidazo[1,2-c]pyrimidine-8- carboxamide | 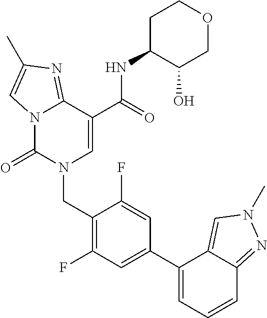 [SMILES: Cc1cn2c(=O)n(Cc3c(F)cc(-c4cccc5nn(C)cc45)cc3F)cc(C(=O)N[C@H]3CCOC[C@@H]3O)c2n1] | 549.4 |
| B30 | 6-(2,6-difluoro-4-(2-methyl-2H- indazol-4-yl)benzyl)-N-((1S,2S)-2- hydroxycyclohexyl)-2-methyl-5- oxo-5,6-dihydroimidazo[1,2- c]pyrimidine-8-carboxamide | 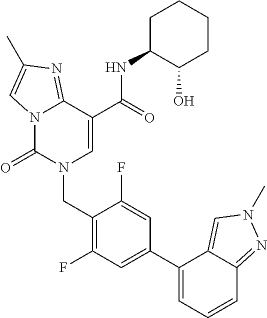 [SMILES: Cc1cn2c(=O)n(Cc3c(F)cc(-c4cccc5nn(C)cc45)cc3F)cc(C(=O)N[C@H]3CCCC[C@@H]3O)c2n1] | 547.4 |
| B31 | 6-(2-fluoro-4-(1-methyl-1H-pyrazol- 5-yl)benzyl)-N-((3R,4S)-3- hydroxytetrahydro-2H-pyran-4-yl)- 5-oxo-5,6-dihydroimidazo[1,2- c]pyrimidine-8-carboxamide | 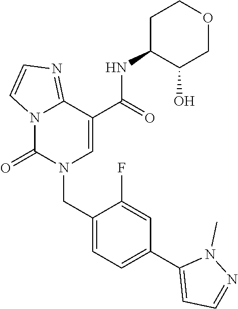 [SMILES: Cn1nccc1-c1ccc(Cn2cc(C(=O)N[C@H]3CCOC[C@@H]3O)c3nccn3c2=O)c(F)c1] | 467.4 |
| B32 | 6-(2-fluoro-4-(1-methyl-1H-pyrazol- 5-yl)benzyl)-N-((1S,2S)-2- hydroxycyclohexyl)-5-oxo-5,6- dihydroimidazo[1,2-c]pyrimidine-8- carboxamide | 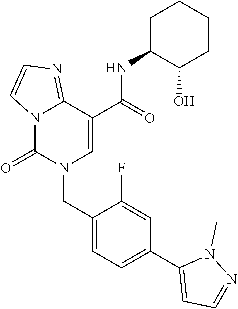 [SMILES: Cn1nccc1-c1ccc(Cn2cc(C(=O)N[C@H]3CCCC[C@@H]3O)c3nccn3c2=O)c(F)c1] | 465.4 |
| B33 | 6-(2-fluoro-4-(1-methyl-1H-pyrazol- 5-yl)benzyl)-N-((1S,2S)-2- hydroxycyclopentyl)-5-oxo-5,6- dihydroimidazo[1,2-c]pyrimidine-8- carboxamide | 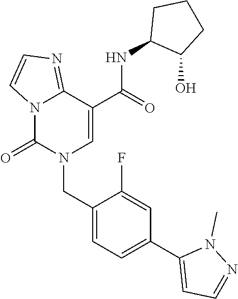 [SMILES: Cn1nccc1-c1ccc(Cn2cc(C(=O)N[C@H]3CCC[C@@H]3O)c3nccn3c2=O)c(F)c1] | 451.4 |
| B34 | 6-(4-(1,4-dimethyl-1H-pyrazol-5- yl)-2-fluorobenzyl)-N-((3R,4S)-3- hydroxytetrahydro-2H-pyran-4-yl)- 5-oxo-5,6-dihydroimidazo[1,2- c]pyrimidine-8-carboxamide | 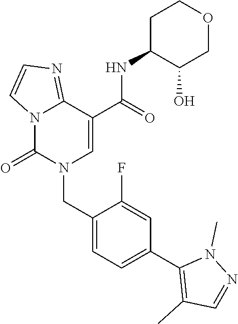 [SMILES: Cc1cnn(C)c1-c1ccc(Cn2cc(C(=O)N[C@H]3CCOC[C@@H]3O)c3nccn3c2=O)c(F)c1] | 481.4 |
| B35 | 6-(4-(1,4-dimethyl-1H-pyrazol-5- yl)-2-fluorobenzyl)-N-((1S,2S)-2- hydroxycyclohexyl)-5-oxo-5,6- dihydroimidazo[1,2-c]pyrimidine-8- carboxamide | 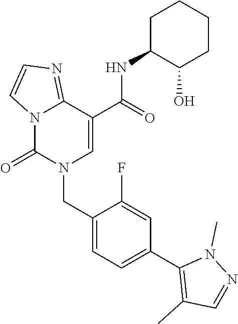 [SMILES: Cc1cnn(C)c1-c1ccc(Cn2cc(C(=O)N[C@H]3CCCC[C@@H]3O)c3nccn3c2=O)c(F)c1] | 479.4 |
| B36 | 6-(2,6-difluoro-4-(1-methyl-1H- pyrazol-5-yl)benzyl)-N-((3R,4S)-3- hydroxytetrahydro-2H-pyran-4-yl)- 5-oxo-5,6-dihydroimidazo[1,2- c]pyrimidine-8-carboxamide | 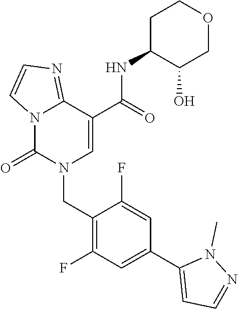 [SMILES: Cn1nccc1-c1cc(F)c(Cn2cc(C(=O)N[C@H]3CCOC[C@@H]3O)c3nccn3c2=O)c(F)c1] | 485.4 |

TABLE C

| | | | |
|---|---|---|---|
| C1 | 5-(2-fluoro-4-(1-methyl-1H-pyrazol- 3-yl)benzyl)-N-((1S,2S)-2- hydroxycyclohexyl)-3-methyl-4- oxo-4,5-dihydro-3H-imidazo[4,5- c]pyridine-7-carboxamide | 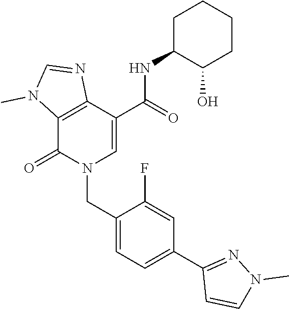 | 479.3 |
| C2 | 5-(2-fluoro-4-(1-methyl-1H-pyrazol- 3-yl)benzyl)-N-((1S,2S)-2- hydroxycyclopentyl)-3-methyl-4- oxo-4,5-dihydro-3H-imidazo[4,5- c]pyridine-7-carboxamide | 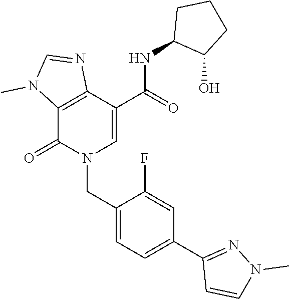 | 465.4 |
| C3 | 5-(2-fluoro-4-(1-methyl-1H-pyrazol- 3-yl)benzyl)-N-((1S,2S)-2- hydroxycycloheptyl)-3-methyl-4- oxo-4,5-dihydro-3H-imidazo[4,5- c]pyridine-7-carboxamide | 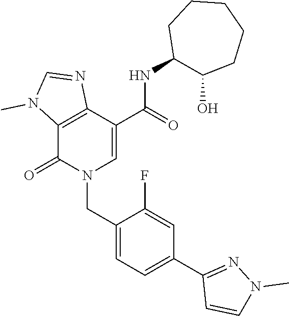 | 493.4 |
| C4 | 5-(2,6-difluoro-4-(2-methyl-2H- indazol-4-yl)benzyl)-N-((3R,4S)-3- hydroxytetrahydro-2H-pyran-4-yl)- 3-methyl-4-oxo-4,5-dihydro-3H- imidazo[4,5-c]pyridine-7- carboxamide | 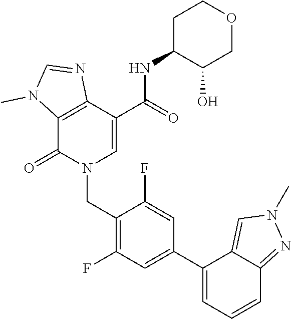 | 549.2 |
| C5 | 5-(4-(1H-pyrazol-1-yl)benzyl)-N- ((3S,4S)-4-hydroxytetrahydro-2H- pyran-3-yl)-3-methyl-4-oxo-4,5- dihydro-3H-imidazo[4,5-c]pyridine- 7-carboxamide | 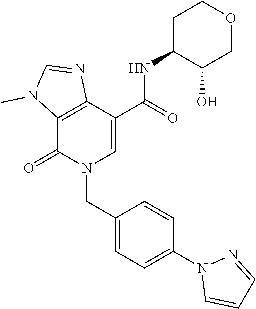 [SMILES: Cn1cnc2c(C(=O)N[C@H]3CCOC[C@@H]3O)cn(Cc3ccc(-n4cccn4)cc3)c(=O)c21] | 449.3 |
| C6 | 5-(4-(1H-pyrazol-1-yl)benzyl)-N- ((1S,2S)-2-hydroxycyclohexyl)-3- methyl-4-oxo-4,5-dihydro-3H- imidazo[4,5-c]pyridine-7- carboxamide | 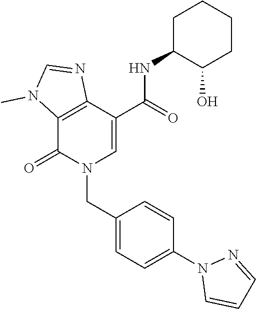 [SMILES: Cn1cnc2c(C(=O)N[C@H]3CCCC[C@@H]3O)cn(Cc3ccc(-n4cccn4)cc3)c(=O)c21] | 447.4 |

TABLE D

| | | | |
|---|---|---|---|
| D1 | 5-(4-(1H-pyrazol-1-yl)benzyl)-N- (3,3-dimethyltetrahydro-2H-pyran-4- yl)-4-oxo-4,5-dihydrofuro[3,2- c]pyridine-7-carboxamide | 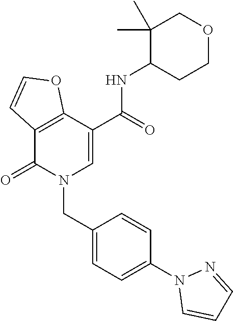 [SMILES: CC1(C)COCCC1NC(=O)c1cn(Cc2ccc(-n3cccn3)cc2)c(=O)c2ccoc12] | 447.4 |
| D2 | 5-(4-(1H-pyrazol-1-yl)benzyl)-N- ((3R,4S)-3-hydroxytetrahydro-2H- pyran-4-yl)-4-oxo-4,5- dihydrofuro[3,2-c]pyridine-7- carboxamide | 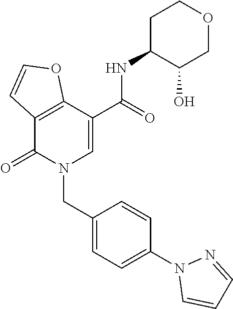 [SMILES: O=C(N[C@H]1CCOC[C@@H]1O)c1cn(Cc2ccc(-n3cccn3)cc2)c(=O)c2ccoc12] | 435.2 |
| D3 | 5-(4-(1H-pyrazol-1-yl)benzyl)-N- ((1S,2S)-2-hydroxycyclohexyl)-4- oxo-4,5-dihydrofuro[3,2-c]pyridine- 7-carboxamide | 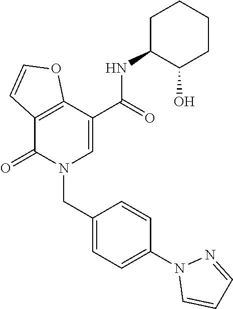 [SMILES: O=C(N[C@H]1CCCC[C@@H]1O)c1cn(Cc2ccc(-n3cccn3)cc2)c(=O)c2ccoc12] | 433.4 |
| D4 | 5-(4-(1H-pyrazol-1-yl)benzyl)-N- ((1S,2S)-2-hydroxycyclopentyl)-4- oxo-4,5-dihydrofuro[3,2-c]pyridine- 7-carboxamide | 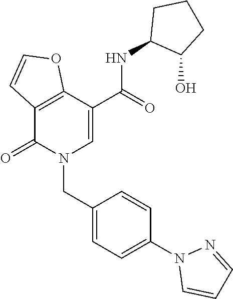 [SMILES: O=C(N[C@H]1CCC[C@@H]1O)c1cn(Cc2ccc(-n3cccn3)cc2)c(=O)c2ccoc12] | 419.3 |
| D5 | 5-(4-(1H-pyrazol-1-yl)benzyl)-N- ((1S,2S)-2-hydroxycycloheptyl)-4- oxo-4,5-dihydrofuro[3,2-c]pyridine- 7-carboxamide | 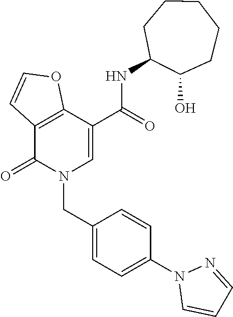 [SMILES: O=C(N[C@H]1CCCCC[C@@H]1O)c1cn(Cc2ccc(-n3cccn3)cc2)c(=O)c2ccoc12] | 447.4 |
| D6 | 5-(2-fluoro-4-(1-methyl-1H-pyrazol- 3-yl)benzyl)-N-((3R,4S)-3- hydroxytetrahydro-2H-pyran-4-yl)- 4-oxo-4,5-dihydrofuro[3,2- c]pyridine-7-carboxamide | 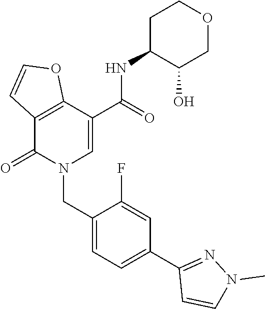 [SMILES: Cn1ccc(-c2ccc(Cn3cc(C(=O)N[C@H]4CCOC[C@@H]4O)c4occc4c3=O)c(F)c2)n1] | 467.2 |
| D7 | 5-(2-fluoro-4-(1-methyl-1H-pyrazol- 3-yl)benzyl)-N-((1S,2S)-2- hydroxycyclohexyl)-4-oxo-4,5- dihydrofuro[3,2-c]pyridine-7- carboxamide | 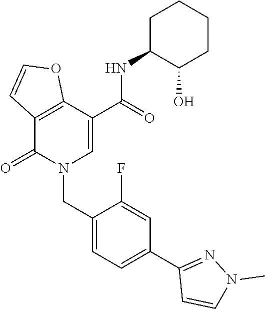 [SMILES: Cn1ccc(-c2ccc(Cn3cc(C(=O)N[C@H]4CCCC[C@@H]4O)c4occc4c3=O)c(F)c2)n1] | 465.4 |
| D8 | 5-(2-fluoro-4-(1-methyl-1H-pyrazol- 3-yl)benzyl)-N-((1S,2S)-2- hydroxycyclopentyl)-4-oxo-4,5- dihydrofuro[3,2-c]pyridine-7- carboxamide | 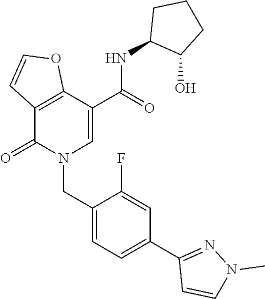 [SMILES: Cn1ccc(-c2ccc(Cn3cc(C(=O)N[C@H]4CCC[C@@H]4O)c4occc4c3=O)c(F)c2)n1] | 451.3 |
| D9 | 5-(2-fluoro-4-(1-methyl-1H-pyrazol- 3-yl)benzyl)-N-((1S,2S)-2- hydroxycycloheptyl)-4-oxo-4,5- dihydrofuro[3,2-c]pyridine-7- carboxamide | 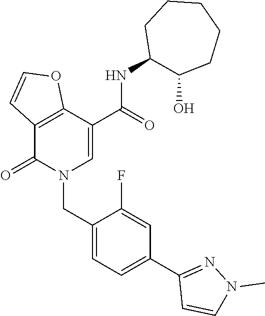 [SMILES: Cn1ccc(-c2ccc(Cn3cc(C(=O)N[C@H]4CCCCC[C@@H]4O)c4occc4c3=O)c(F)c2)n1] | 479.4 |
| D10 | 5-(2-fluoro-4-(1-methyl-1H-pyrazol- 3-yl)benzyl)-4-oxo-N-(2- oxaspiro[3.3]heptan-6-yl)-4,5- dihydrofuro[3,2-c]pyridine-7- carboxamide | 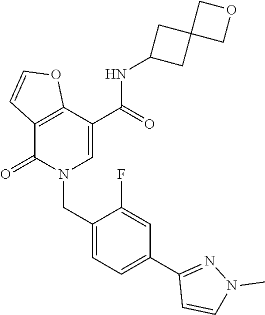 [SMILES: Cn1ccc(-c2ccc(Cn3cc(C(=O)NC4CC5(COC5)C4)c4occc4c3=O)c(F)c2)n1] | 463.4 |
| D11 | N-(2,2-dimethyltetrahydro-2H- pyran-4-yl)-5-(2-fluoro-4-(1-methyl- 1H-pyrazol-3-yl)benzyl)-4-oxo-4,5- dihydrofuro[3,2-c]pyridine-7- carboxamide | 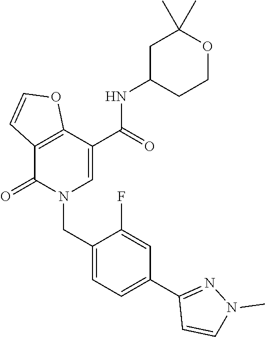 [SMILES: Cn1ccc(-c2ccc(Cn3cc(C(=O)NC4CCOC(C)(C)C4)c4occc4c3=O)c(F)c2)n1] | 479.4 |
| D12 | 5-(2-fluoro-4-(1-methyl-1H-pyrazol- 4-yl)benzyl)-N-((3R,4S)-3- hydroxytetrahydro-2H-pyran-4-yl)- 4-oxo-4,5-dihydrofuro[3,2- c]pyridine-7-carboxamide | 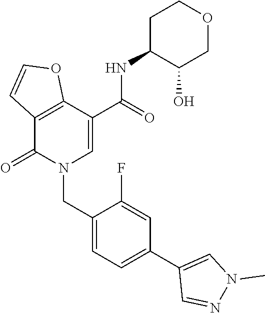 [SMILES: Cn1cc(-c2ccc(Cn3cc(C(=O)N[C@H]4CCOC[C@@H]4O)c4occc4c3=O)c(F)c2)cn1] | 467.2 |
| D13 | 5-(2-fluoro-4-(1-methyl-1H-pyrazol- 4-yl)benzyl)-N-((1S,2S)-2- hydroxycyclohexyl)-4-oxo-4,5- dihydrofuro[3,2-c]pyridine-7- carboxamide | 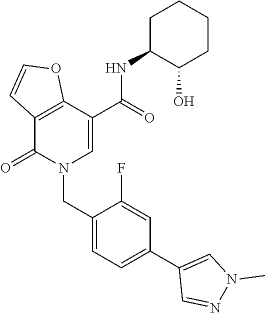 [SMILES: Cn1cc(-c2ccc(Cn3cc(C(=O)N[C@H]4CCCC[C@@H]4O)c4occc4c3=O)c(F)c2)cn1] | 465.4 |
| D14 | 5-(2-fluoro-4-(1-methyl-1H-pyrazol- 4-yl)benzyl)-N-((1S,2S)-2- hydroxycyclopentyl)-4-oxo-4,5- dihydrofuro[3,2-c]pyridine-7- carboxamide | 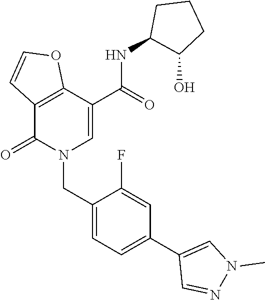 [SMILES: Cn1cc(-c2ccc(Cn3cc(C(=O)N[C@H]4CCC[C@@H]4O)c4occc4c3=O)c(F)c2)cn1] | 451.4 |
| D15 | 5-(2-fluoro-4-(1-methyl-1H-pyrazol- 4-yl)benzyl)-N-((1R,2R)-2- hydroxycyclobutyl)-4-oxo-4,5- dihydrofuro[3,2-c]pyridine-7- carboxamide | 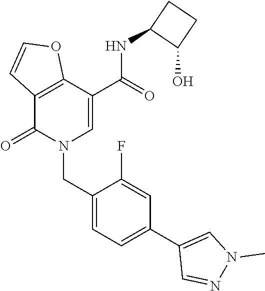 [SMILES: Cn1cc(-c2ccc(Cn3cc(C(=O)N[C@H]4CC[C@@H]4O)c4occc4c3=O)c(F)c2)cn1] | 437.3 |
| D16 | 5-(2-fluoro-4-(1-methyl-1H-pyrazol- 4-yl)benzyl)-N-((1S,2S)-2- hydroxycycloheptyl)-4-oxo-4,5- dihydrofuro[3,2-c]pyridine-7- carboxamide | 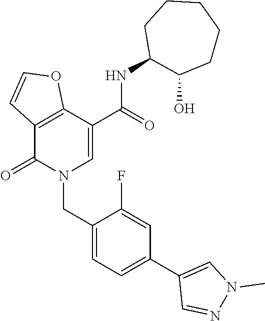 [SMILES: Cn1cc(-c2ccc(Cn3cc(C(=O)N[C@H]4CCCCC[C@@H]4O)c4occc4c3=O)c(F)c2)cn1] | 479.4 |
| D17 | 5-(2-fluoro-4-(1-methyl-1H-pyrazol- 4-yl)benzyl)-4-oxo-N-(2- oxaspiro[3.3]heptan-6-yl)-4,5- dihydrofuro[3,2-c]pyridine-7- carboxamide | 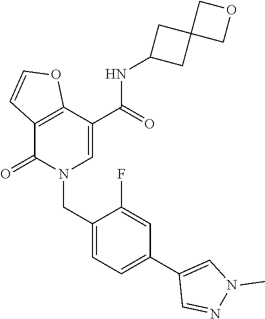 [SMILES: Cn1cc(-c2ccc(Cn3cc(C(=O)NC4CC5(COC5)C4)c4occc4c3=O)c(F)c2)cn1] | 463.4 |
| D18 | 5-(2,6-difluoro-4-(2-methyl-2H- indazol-4-yl)benzyl)-N-((3R,4S)-3- hydroxytetrahydro-2H-pyran-4-yl)- 4-oxo-4,5-dihydrofuro[3,2- c]pyridine-7-carboxamide | 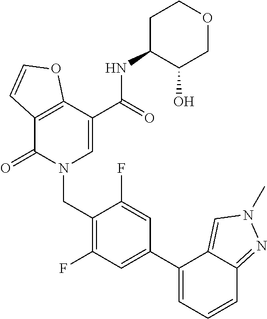 [SMILES: Cn1cc2c(-c3cc(F)c(Cn4cc(C(=O)N[C@H]5CCOC[C@@H]5O)c5occc5c4=O)c(F)c3)cccc2n1] | 535.3 |
| D19 | 5-(2,6-difluoro-4-(2-methyl-2H- indazol-4-yl)benzyl)-N-((1S,2S)-2- hydroxycyclohexyl)-4-oxo-4,5- dihydrofuro[3,2-c]pyridine-7- carboxamide | 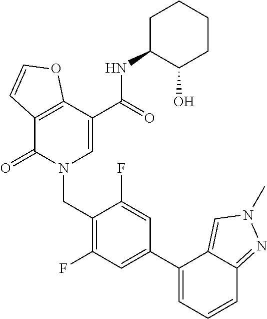 [SMILES: Cn1cc2c(-c3cc(F)c(Cn4cc(C(=O)N[C@H]5CCCC[C@@H]5O)c5occc5c4=O)c(F)c3)cccc2n1] | 533.4 |
| D20 | 5-(2,6-difluoro-4-(2-methyl-2H- indazol-4-yl)benzyl)-N-((1S,2S)-2- hydroxycyclopentyl)-4-oxo-4,5- dihydrofuran[3,2-c]pyridine-7- carboxamide | 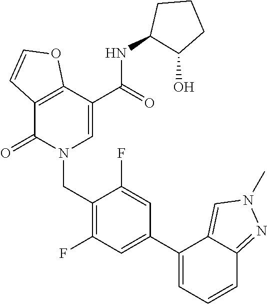 [SMILES: Cn1cc2c(-c3cc(F)c(Cn4cc(C(=O)N[C@H]5CCC[C@@H]5O)c5occc5c4=O)c(F)c3)cccc2n1] | 518.5 |
| D21 | 5-(2,6-difluoro-4-(2-methyl-2H- indazol-4-yl)benzyl)-N-((1R,2R)-2- hydroxycyclobutyl)-4-oxo-4,5- dihydrofuro[3,2-c]pyridine-7- carboxamide | 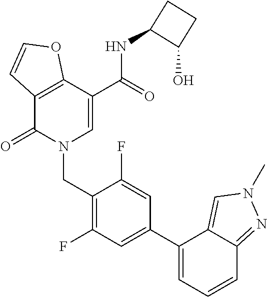 [SMILES: Cn1cc2c(-c3cc(F)c(Cn4cc(C(=O)N[C@H]5CC[C@@H]5O)c5occc5c4=O)c(F)c3)cccc2n1] | 504.4 |
| D22 | 5-(2,6-difluoro-4-(2-methyl-2H- indazol-4-yl)benzyl)-N-((1S,2S)-2- hydroxycycloheptyl)-4-oxo-4,5- dihydrofuro[3,2-c]pyridine-7- carboxamide | 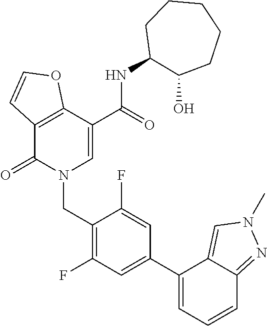 [SMILES: Cn1cc2c(-c3cc(F)c(Cn4cc(C(=O)N[C@H]5CCCCC[C@@H]5O)c5occc5c4=O)c(F)c3)cccc2n1] | 547.4 |
| D23 | 5-(2,6-difluoro-4-(2-methyl-2H- indazol-4-yl)benzyl)-4-oxo-N- (tetrahydro-2H-pyran-4-yl)-4,5- dihydrofuro[3,2-c]pyridine-7- carboxamide | 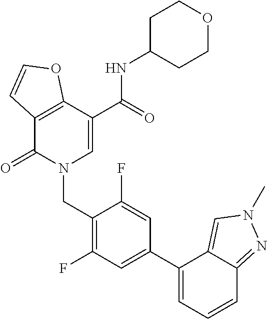 [SMILES: Cn1cc2c(-c3cc(F)c(Cn4cc(C(=O)NC5CCOCC5)c5occc5c4=O)c(F)c3)cccc2n1] | 518.5 |
| D24 | 5-(2,6-difluoro-4-(2-methyl-2H- indazol-4-yl)benzyl)-4-oxo-N-(2- oxaspiro[3.3]heptan-6-yl)-4,5- dihydrofuro[3,2-c]pyridine-7- carboxamide | 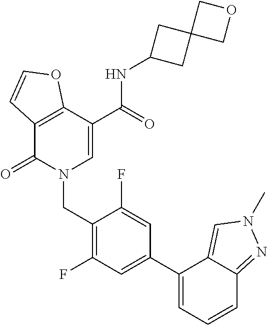 [SMILES: Cn1cc2c(-c3cc(F)c(Cn4cc(C(=O)NC5CC6(COC6)C5)c5occc5c4=O)c(F)c3)cccc2n1] | 531.2 |
| D25 | 5-(2,6-difluoro-4-(2-methyl-2H- indazol-4-yl)benzyl)-4-oxo-N- ((tetrahydrofuran-2-yl)methyl)-4,5- dihydrofuro[3,2-c]pyridine-7- carboxamide | 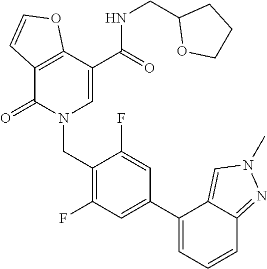 [SMILES: Cn1cc2c(-c3cc(F)c(Cn4cc(C(=O)NCC5CCCO5)c5occc5c4=O)c(F)c3)cccc2n1] | 519.3 |
| D26 | 5-(2-fluoro-4-(1-methyl-1H-pyrazol- 3-yl)benzyl)-N-((3R,4S)-3- hydroxytetrahydro-2H-pyran-4-yl)- 4-oxo-4,5-dihydrothieno[3,2- c]pyridine-7-carboxamide | 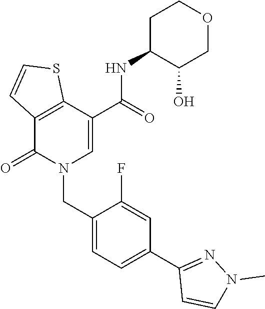 [SMILES: Cn1ccc(-c2ccc(Cn3cc(C(=O)N[C@H]4CCOC[C@@H]4O)c4sccc4c3=O)c(F)c2)n1] | 483.2 |
| D27 | 5-(2-fluoro-4-(1-methyl-1H-pyrazol- 3-yl)benzyl)-N-((1S,2S)-2- hydroxycyclohexyl)-4-oxo-4,5- dihydrothieno[3,2-c]pyridine-7- carboxamide | 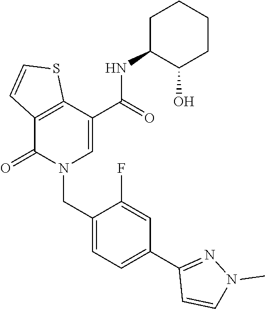 [SMILES: Cn1ccc(-c2ccc(Cn3cc(C(=O)N[C@H]4CCCC[C@@H]4O)c4sccc4c3=O)c(F)c2)n1] | 481.3 |
| D28 | 5-(2-fluoro-4-(1-methyl-1H-pyrazol- 3-yl)benzyl)-N-((1S,2S)-2- hydroxycycloheptyl)-4-oxo-4,5- dihydrothieno[3,2-c]pyridine-7- carboxamide | 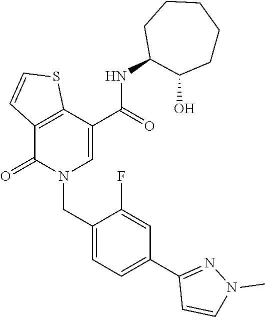 [SMILES: Cn1ccc(-c2ccc(Cn3cc(C(=O)N[C@H]4CCCCC[C@@H]4O)c4sccc4c3=O)c(F)c2)n1] | 495.4 |
| D29 | 5-(2-fluoro-4-(1-methyl-1H-pyrazol- 4-yl)benzyl)-N-((3R,4S)-3- hydroxytetrahydro-2H-pyran-4-yl)- 4-oxo-4,5-dihydrothieno[3,2- c]pyridine-7-carboxamide | 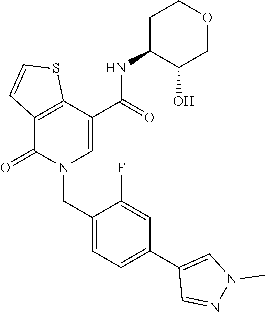 [SMILES: Cn1cc(-c2ccc(Cn3cc(C(=O)N[C@H]4CCOC[C@@H]4O)c4sccc4c3=O)c(F)c2)cn1] | 483.3 |
| D30 | 5-(2,6-difluoro-4-(2-methyl-2H- indazol-4-yl)benzyl)-N-((3R,4S)-3- hydroxytetrahydro-2H-pyran-4-yl)- 4-oxo-4,5-dihydrothieno[3,2- c]pyridine-7-carboxamide | 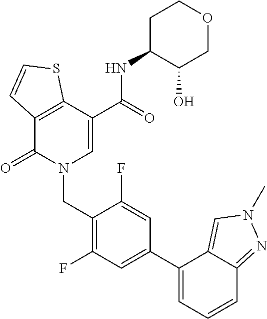 [SMILES: Cn1cc2c(-c3cc(F)c(Cn4cc(C(=O)N[C@H]5CCOC[C@@H]5O)c5sccc5c4=O)c(F)c3)cccc2n1] | 551.2 |
| D31 | 5-(2,6-difluoro-4-(2-methyl-2H- indazol-4-yl)benzyl)-N-((1S,2S)-2- hydroxycyclohexyl)-4-oxo-4,5- dihydrothieno[3,2-c]pyridine-7- carboxamide | 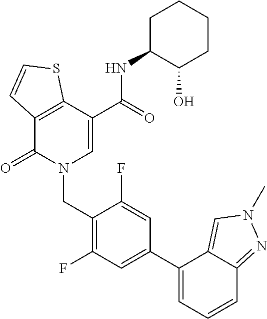 [SMILES: Cn1cc2c(-c3cc(F)c(Cn4cc(C(=O)N[C@H]5CCCC[C@@H]5O)c5sccc5c4=O)c(F)c3)cccc2n1] | 549.3 |
| D32 | 5-(2,6-difluoro-4-(2-methyl-2H- indazol-4-yl)benzyl)-N-((1S,2S)-2- hydroxycyclopentyl)-4-oxo-4,5- dihydrothieno[3,2-c]pyridine-7- carboxamide | 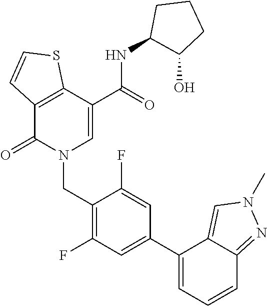 [SMILES: Cn1cc2c(-c3cc(F)c(Cn4cc(C(=O)N[C@H]5CCC[C@@H]5O)c5sccc5c4=O)c(F)c3)cccc2n1] | 535.3 |
| D33 | 5-(2,6-difluoro-4-(2-methyl-2H- indazol-4-yl)benzyl)-N-((1S,2S)-2- hydroxycycloheptyl)-4-oxo-4,5- dihydrothieno[3,2-c]pyridine-7- carboxamide | 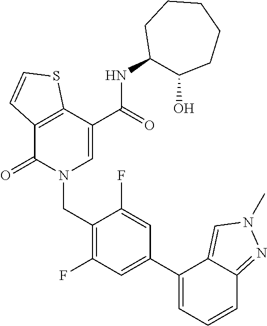 [SMILES: Cn1cc2c(-c3cc(F)c(Cn4cc(C(=O)N[C@H]5CCCCC[C@@H]5O)c5sccc5c4=O)c(F)c3)cccc2n1] | 563.4 |

### Biological Activity

a. Cell Lines Expressing Muscarinic Acetylcholine Receptors

Chinese hamster ovary (CHO-K1) cells stably expressing rat (r)M₁ were purchased from the American Type Culture Collection and cultured according to their indicated protocol. CHO cells stably expressing human (h)M₂, hM₃, and hM₅ were described previously (Levey, et al., 1991); hM₁ and hM₄ cDNAs were purchased from Missouri S&T cDNA Resource; rM₄ cDNA was provided by T. L. Bonner (National Institutes of Health, Bethesda, MID). rM₂ and rM₃ were cloned from a rat brain cDNA library and sequence verified. hM₁, rM₂, rM₃, hM₄, and rM₄ cDNAs were used to stably transfect CHO-K1 cells purchased from the American Type Culture Collection using Lipofectamine2000. To make stable rM₂, hM₂, rM₃, hM₄, and rM₄ cell lines for use in calcium mobilization assays, these cells also were stably transfected with a chimeric G-protein (G_{qi5}) (provided by B. R. Conklin, University of California, San Francisco) using Lipofectamine 2000. rM₁, hM₁, rM₃, hM₃, rM₅, and hM₅ cells were grown in Ham's F-12 medium containing 10% heat-inactivated fetal bovine serum (FBS), 20 mM HEPES, and 50 μg/mL G418 sulfate. rM₂-G_{q15}, hM₂-G_{qi5}, and hM₄-G_{qi5} cells were grown in the same medium also containing 500 μg/mL Hygromycin B. Stable rM₄-G_{qi5} cells were grown in DMEM containing 10% heat-inactivated FBS, 20 mM HEPES, 400 μg/mL G418 sulfate, and 500 μg/mL Hygromycin B.

b. Cell-Based Functional Assay of Muscarinic Acetylcholine Receptor Activity

For high throughput measurement of agonist-evoked increases in intracellular calcium, CHO-K1 cells stably expressing muscarinic receptors were plated in growth medium lacking G418 and hygromycin at 15,000 cells/20 μL/well in Greiner 384-well black-walled, tissue culture (TC)-treated, clear-bottom plates (VWR). Cells were incubated overnight at 37° C. and 5% CO₂. The next day, cells were washed using an ELX 405 (BioTek) with four washes (80 μL) of assay buffer then aspirated to 20 μL. Next, 20 μL of 16 μM Fluo-4/acetoxymethyl ester (Invitrogen, Carlsbad, Calif.) prepared as a 2.3 mM stock in DMSO and mixed in a 1:1 ratio with 10% (w/v) Pluronic F-127 and diluted in assay buffer was added to the wells and the cell plates were incubated for 50 min at 37° C. and 5% CO₂. Dye was removed by washing with the ELX 405 (four 80 μL washes of assay buffer) then aspirated to 20 μL. Compound master plates were formatted in an 11 point CRC format (1:3 dilutions) in 100% DMSO with a starting concentration of 10 mM using the BRAVO liquid handler (Agilent). Test compound CRCs were then transferred to daughter plates (240 nL) using the Echo acoustic plate reformatter (Labcyte, Sunnyvale, Calif.) and then diluted into assay buffer (40 μL) to a 2× stock using a Thermo Fisher Combi (Thermo Fisher Scientific, Waltham, Mass.).

Calcium flux was measured using the Functional Drug Screening System (FDSS) 6000 (Hamamatsu Corporation, Tokyo, Japan) as an increase in the fluorescent static ratio. Compounds were applied to cells (20 μL, 2×) using the automated system of the FDSS 6000 at 4 s into the 300 s protocol and the data were collected at 1 Hz. At 144 s into the 300s protocol, 10 μL of an EC₂₀ concentration of the muscarinic receptor agonist acetylcholine was added (5×), followed by the addition of 12 μL an EC₈₀ concentration of acetylcholine at the 230 s time point (5×). Agonist activity was analyzed as a concentration-dependent increase in calcium mobilization upon compound addition. Eₘₐₓ values for agonist activity are expressed relative to the maximum for acetylcholine. Positive allosteric modulator activity was analyzed as a concentration-dependent increase in the EC₂₀ acetylcholine response. Antagonist activity was analyzed as a concentration-dependent decrease in the EC₈₀ acetylcholine response. Concentration-response curves were generated using a four-parameter logistical equation in XLfit curve fitting software (IDBS, Bridgewater, N.J.) for Excel (Microsoft, Redmond, Wash.) or Prism (GraphPad Software, Inc., San Diego, Calif.).

The above described assay was also operated in a second mode where an appropriate fixed concentration of the present compounds were added to the cells after establishment of a fluorescence baseline for about 3 seconds, and the response in cells was measured. 140 s later the appropriate concentration of agonist was added and readings taken for an additional 106 s. Data were reduced as described above and the EC₅₀ values for the agonist in the presence of test compound were determined by nonlinear curve fitting. A decrease in the EC₅₀ value of the agonist with increasing concentrations of the present compounds (a leftward shift of the agonist concentration-response curve) is an indication of the degree of muscarinic positive allosteric modulation at a given concentration of the present compound. An increase in the EC₅₀ value of the agonist with increasing concentrations of the present compounds (a rightward shift of the agonist concentration response curve) is an indication of the degree of muscarinic antagonism at a given concentration of the present compound. The second mode also indicates whether the present compounds also affect the maximum response of the muscarinic receptor to agonists.

c. Results and Discussion of Biological Activity Data

Activity (EC₈₀ and Eₘₐₓ) was determined in the mAChR M₁ cell-based functional assay as described above and the data are shown in Table 1. The compound numbers correspond to the compound numbers used in Table A, B, C and D. The data in Table 1 demonstrate that the disclosed compounds are positive allosteric modulators of human mAChR M₁ and show high PAM activity for the human mAChR M₁ receptor(s).

TABLE 1
Biological Activity Data

| | PAM Activity | | Agonist Activity | |
|---|---|---|---|---|
| | Human M₁ | | Human M₁ | |
| Cpd. No. | EC₅₀ (μM) | Eₘₐₓ (%) | EC₅₀ (μM) | Eₘₐₓ (%) |
| A1 | 0.48 | 90 | 7.90 | 20 |
| A2 | 0.73 | 91 | >10 | <5 |
| A3 | 1.66 | 83 | >10 | <5 |
| A4 | 3.45 | 42 | >10 | <5 |
| A5 | 2.74 | 44 | >10 | <5 |
| A6 | 0.22 | 89 | 8.20 | 23 |
| A7 | 0.29 | 89 | 10 | 12 |
| A8 | 1.29 | 83 | >10 | <5 |
| A9 | 5.87 | 65 | >10 | <5 |
| A10 | 10 | 59 | >10 | <5 |
| A11 | 6.75 | 58 | >10 | <5 |
| A12 | 1.39 | 70 | 10 | 28 |
| A13 | 1.13 | 71 | 8.95 | 21 |
| A14 | 7.78 | 64 | >10 | <5 |
| A15 | 1.37 | 68 | 10 | 34 |
| A16 | 0.98 | 68 | 10 | 27 |
| A17 | 10 | 55 | >10 | <5 |
| A18 | 1.66 | 60 | >10 | <5 |
| A19 | 2.49 | 67 | 10 | 20 |
| A20 | 0.78 | 63 | >10 | <5 |
| A21 | 0.019 | 72 | 1.23 | 31 |
| A22 | 0.052 | 67 | >10 | <5 |
| A23 | 0.21 | 67 | >10 | <5 |
| A24 | 0.35 | 69 | 6.12 | 16 |
| A25 | 0.97 | 68 | >10 | <5 |
| A26 | 0.49 | 75 | 10 | 10 |
| A27 | 0.86 | 74 | >10 | 6 |
| A28 | 1.14 | 54 | >10 | <5 |
| A29 | 4.84 | 61 | >10 | <5 |
| A30 | 4.61 | 47 | >10 | <5 |
| A31 | 2.60 | 70 | >10 | <5 |
| A32 | 2.24 | 70 | >10 | <5 |
| A33 | 0.86 | 64 | 3.22 | 13 |
| A34 | 0.62 | 65 | 2.88 | 12 |
| A35 | 0.55 | 48 | >10 | <5 |
| A36 | 0.61 | 65 | 1.77 | 13 |
| A37 | 0.78 | 46 | >10 | <5 |
| A38 | 0.57 | 25 | >10 | <5 |
| A39 | 10 | 41 | >10 | <5 |
| A40 | 0.52 | 72 | 9.11 | 30 |
| A41 | 0.88 | 65 | >10 | 20 |
| A42 | 2.13 | 37 | >10 | <5 |
| A43 | 0.64 | 51 | >10 | <5 |
| A44 | 10 | 26 | >10 | <5 |
| A45 | 4.37 | 70 | >10 | <5 |
| A46 | 1.13 | 41 | >10 | <5 |
| A47 | 1.09 | 57 | >10 | <5 |
| A48 | 0.66 | 38 | >10 | <5 |
| A49 | 0.98 | 65 | >10 | <5 |
| A50 | 1.79 | 69 | >10 | <5 |
| A51 | 1.68 | 75 | >10 | <5 |
| A52 | 0.40 | 64 | 4.36 | 50 |
| A53 | 9.64 | 48 | >10 | <5 |
| A54 | 2.37 | 62 | >10 | 26 |
| A55 | 7.90 | 67 | >10 | 12 |
| A56 | 10 | 61 | >10 | <5 |
| A57 | 1.15 | 68 | >10 | 25 |
| A58 | 0.37 | 68 | 5.27 | 40 |
| A59 | 0.42 | 45 | >10 | <5 |
| A60 | 0.61 | 34 | >10 | <5 |
| A61 | 10 | 33 | >10 | <5 |
| A62 | 10 | 53 | >10 | <5 |
| A63 | 10 | 55 | >10 | 7 |
| A64 | 10 | 41 | >10 | 41 |
| A65 | 5.25 | 63 | >10 | 6 |
| A66 | 0.28 | 72 | 5.00 | 46 |
| A67 | 0.62 | 77 | 5.00 | 46 |
| A68 | 1.20 | 74 | 10 | 8 |
| A69 | 3.89 | 57 | >10 | <5 |
| A70 | 5.50 | 45 | >10 | <5 |
| A71 | 3.10 | 57 | >10 | <5 |
| A72 | 10 | 32 | >10 | <5 |
| A73 | 4.04 | 67 | >10 | <5 |
| A74 | 3.81 | 53 | >10 | <5 |
| A75 | 3.54 | 56 | >10 | <5 |
| A76 | 4.33 | 53 | >10 | <5 |
| A77 | 4.21 | 72 | 10 | 18 |
| A78 | 2.98 | 67 | 10 | 5 |
| A79 | 10 | 51 | >10 | <5 |
| A80 | 10 | 30 | >10 | <5 |
| A81 | 10 | 33 | >10 | <5 |
| A82 | 10 | 26 | >10 | <5 |
| A83 | 10 | 26 | >10 | <5 |
| A84 | 10 | 40 | >10 | <5 |
| A85 | 10 | 41 | >10 | <5 |
| A86 | 0.37 | 52 | >10 | <5 |
| A87 | 0.82 | 44 | >10 | <5 |
| A88 | 10 | 40 | >10 | <5 |
| A89 | 10 | 51 | >10 | <5 |
| A90 | 10 | 47 | >10 | <5 |
| A91 | 10 | 34 | >10 | <5 |
| B1 | 0.13 | 67 | 3.00 | 55 |
| B2 | 0.096 | 68 | 3.70 | 60 |
| B3 | 0.54 | 67 | 10 | 33 |
| B4 | 3.22 | 52 | >10 | <5 |
| B5 | 0.046 | 68 | 0.15 | 62 |
| B6 | 0.037 | 74 | 1.54 | 63 |
| B7 | 0.24 | 73 | 10 | 48 |
| B8 | 2.00 | 72 | 10 | 29 |
| B9 | 2.06 | 61 | 10 | 17 |
| B10 | 0.48 | 60 | 10 | 41 |
| B11 | 0.21 | 63 | 5.00 | 45 |
| B12 | 0.38 | 67 | 6.02 | 55 |
| B13 | 0.10 | 70 | 3.17 | 55 |
| B14 | 1.33 | 70 | 10 | 28 |
| B15 | 0.027 | 70 | 2.96 | 64 |
| B16 | 0.52 | 81 | >10 | <5 |
| B17 | 0.47 | 83 | >10 | 24 |
| B18 | 0.093 | 84 | 5.27 | 24 |
| B19 | 0.47 | 42 | 5.24 | 51 |
| B20 | 0.24 | 51 | 2.79 | 63 |
| B21 | 0.90 | 59 | >10 | <5 |
| B22 | 1.27 | 59 | >10 | 19 |
| B23 | 7.85 | 39 | >10 | <5 |
| B24 | 10 | 41 | >10 | <5 |
| B25 | 9.29 | 33 | >10 | <5 |
| B26 | 5.53 | 28 | >10 | <5 |
| B27 | 10 | 30 | >10 | <5 |
| B28 | 10 | 30 | >10 | <5 |
| B29 | 4.31 | 40 | >10 | <5 |
| B30 | 5.89 | 27 | >10 | <5 |
| B31 | 1.13 | 82 | >10 | 6 |
| B32 | 0.89 | 80 | >10 | <5 |
| B33 | 2.23 | 57 | >10 | <5 |
| B34 | 10 | 44 | >10 | <5 |
| B35 | 7.02 | 44 | >10 | <5 |
| B36 | 1.25 | 78 | >10 | <5 |
| C1 | 0.25 | 65 | 10 | 8 |
| C2 | 0.60 | 45 | >10 | <5 |
| C3 | 0.31 | 60 | >10 | <5 |
| C4 | 0.31 | 58 | >10 | <5 |
| C5 | 0.88 | 69 | 2.75 | 15 |
| C6 | 0.17 | 43 | >10 | <5 |
| D1 | >10 | 25 | >10 | <5 |
| D2 | >10 | 57 | >10 | <5 |
| D3 | 5.12 | 62 | >10 | <5 |
| D4 | >10 | 34 | >10 | <5 |
| D5 | >10 | 58 | >10 | <5 |
| D6 | >10 | 68 | >10 | <5 |
| D7 | 6.05 | 82 | >10 | <5 |
| D8 | 5.13 | 32 | >10 | <5 |
| D9 | 3.09 | 80 | >10 | <5 |
| D10 | >10 | 25 | >10 | <5 |
| D11 | >10 | 24 | >10 | <5 |
| D12 | 4.02 | 67 | >10 | <5 |
| D13 | 2.12 | 81 | >10 | 11 |
| D14 | >10 | 51 | >10 | <5 |
| D15 | >10 | 29 | >10 | <5 |
| D16 | 2.02 | 75 | >10 | 8 |
| D17 | >10 | 40 | >10 | <5 |
| D18 | 0.28 | 85 | 2.27 | 11 |
| D19 | 0.43 | 82 | >10 | 9 |
| D20 | 0.81 | 77 | >10 | <5 |
| D21 | 2.19 | 75 | >10 | <5 |
| D22 | 0.95 | 81 | >10 | 9 |
| D23 | 4.12 | 64 | >10 | <5 |
| D24 | 2.48 | 70 | >10 | <5 |
| D25 | >10 | 53 | >10 | <5 |
| D26 | >10 | 34 | >10 | <5 |
| D27 | >10 | 37 | >10 | <5 |
| D28 | >10 | 40 | >10 | <5 |
| D29 | >10 | 37 | >10 | <5 |
| D30 | 4.52 | 69 | >10 | <5 |
| D31 | 7.98 | 54 | >10 | <5 |
| D32 | 7.73 | 40 | >10 | <5 |
| D33 | 5.29 | 42 | >10 | <5 |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A compound of formula (I), or a pharmaceutically acceptable salt thereof, 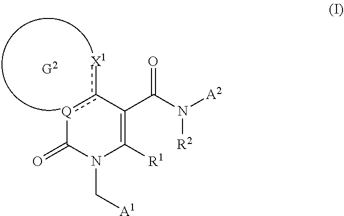
wherein
Q is C or N;
G² is a 5- to 6-membered monocyclic heteroarene containing X¹ and, in addition, 0, 1, or 2 nitrogen atoms, the heteroarene being unsubstituted or substituted with 1-3 substituents independently selected from the group consisting of halogen, C₁₋₄alkyl, and C₁₋₄haloalkyl;
X¹ is an unsubstituted heteroatom selected from the group consisting of nitrogen, oxygen, and sulfur;
A¹ is 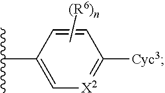
X² is N or CR^{6a};
Cyc³ is a phenyl or 5- to 12-membered heteroaryl, wherein the phenyl and 5- to 12-membered heteroaryl are unsubstituted or substituted with 1-5 substituents independently selected from the group consisting of halogen, C₁₋₄alkyl, C₁₋₄haloalkyl, —OC₁₋₄alkyl, —OC₁₋₄haloalkyl, —OC₃₋₆cycloalkyl, —O—C₁₋₃alkylene-C₃₋₆cycloalkyl, —OH, oxo, cyano, C₃₋₆cycloalkyl, and —C₁₋₃alkylene-C₃₋₆cycloalkyl, wherein each cycloalkyl is unsubstituted or substituted with 1-4 substituents independently selected from the group consisting of halogen and C₁₋₄alkyl;
A² is C₁₋₆alkyl, C₁₋₆haloalkyl, or -L¹-G¹, wherein the C₁₋₆alkyl and C₁₋₆haloalkyl are unsubstituted or substituted with 1-2 substituents independently selected from the group consisting of cyano, oxo, —OH, and —OC₁₋₄alkyl;
L¹ is a bond, C₂₋₆alkenylene, or C₁₋₆alkylene, wherein the C₂₋₆alkenylene and C₁₋₆alkylene are unsubstituted or substituted with 1-4 substituents independently selected from the group consisting of halogen, cyano, —OH, oxo, —OC₁₋₄alkyl, and C₃₋₆cycloalkyl;
G¹ is C₃₋₁₂cycloalkyl or 4- to 12-membered heterocyclyl, wherein G¹ is unsubstituted or substituted with 1-4 substituents independently selected from the group consisting of C₁₋₄alkyl, C₁₋₄haloalkyl, —OH, —OC₁₋₄alkyl, cyano, oxo, C₃₋₆cycloalkyl, —C₁₋₃alkylene-OH, and —C₁₋₃alkylene-OC₁₋₄alkyl;
R¹ is hydrogen, halogen, cyano, C₁₋₄alkyl, C₁₋₄haloalkyl, —OC₁₋₄alkyl, or —C₁₋₃alkylene-OC₁₋₄ alkyl;
R² is hydrogen, C₁₋₆alkyl, or C₁₋₆haloalkyl;
R⁶, at each occurrence, is independently selected from the group consisting of halogen, C₁₋₄alkyl, C₁₋₄haloalkyl, —OC₁₋₄alkyl, —OC₁₋₄haloalkyl, —OH, cyano, C₃₋₆cycloalkyl, and —C₁₋₃alkylene-C₃₋₆cycloalkyl;
R^{6a} is selected from the group consisting of hydrogen, halogen, C₁₋₄alkyl, C₁₋₄haloalkyl, —OC₁₋₄alkyl, —OC₁₋₄haloalkyl, —OH, cyano, C₃₋₆cycloalkyl, and —C₁₋₃alkylene-C₃₋₆cycloalkyl; and
n is 0, 1, 2, or 3.

2. The compound of claim 1, or a pharmaceutically acceptable salt thereof, wherein G² is the 5-membered monocyclic heteroarene, wherein X¹ is unsubstituted nitrogen.

3. The compound of claim 2 of formula (I-B), or a pharmaceutically acceptable salt thereof, 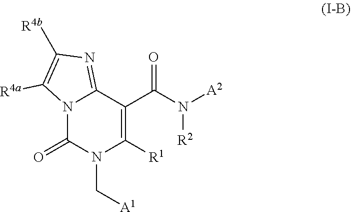
wherein R^{4a} and R^{4b} are independently selected from the group consisting of hydrogen, halogen, C₁₋₄alkyl, and C₁₋₄haloalkyl.

4. The compound of claim 3, or a pharmaceutically acceptable salt thereof, wherein R^{4a} is hydrogen and R^{4b} is hydrogen or methyl.

5. The compound of claim 2 of formula (I-C), or a pharmaceutically acceptable salt thereof, 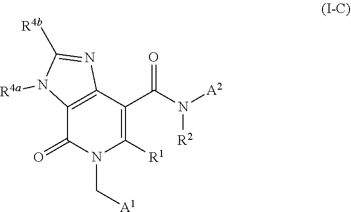
wherein R^{4a} and R^{4b} are independently selected from the group consisting of hydrogen, C₁₋₄alkyl, and C₁₋₄haloalkyl.

6. The compound of claim 5, or a pharmaceutically acceptable salt thereof, wherein R^{4a} is methyl and R^{4b} is hydrogen.

7. The compound of claim 1, or a pharmaceutically acceptable salt thereof, wherein G² is the 5-membered monocyclic heteroarene, wherein X¹ is oxygen or unsubstituted sulfur; and Q is C.

8. The compound of claim 7 of formula (I-D), or a pharmaceutically acceptable salt thereof, 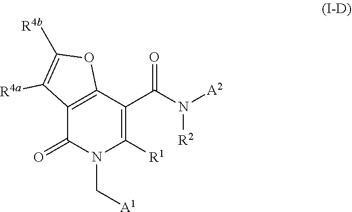
wherein R^{4a} and R^{4b} are independently selected from the group consisting of hydrogen, halogen, C₁₋₄alkyl, and C₁₋₄haloalkyl.

9. The compound of claim 7 of formula (I-E), or a pharmaceutically acceptable salt thereof, 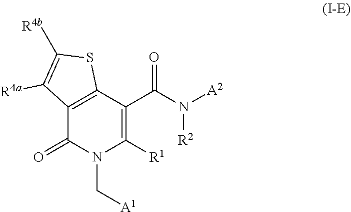
wherein R^{4a} and R^{4b} are independently selected from the group consisting of hydrogen, halogen, C₁₋₄alkyl, and C₁₋₄haloalkyl.

10. The compound of claim 8 or 9, or a pharmaceutically acceptable salt thereof, wherein R^{4a} and R^{4b} are hydrogen.

11. The compound of claim 1, or a pharmaceutically acceptable salt thereof, wherein G² is the 6-membered heteroarene; X¹ is unsubstituted nitrogen; and Q is C.

12. The compound of claim 11 of formula (I-F), or a pharmaceutically acceptable salt thereof, 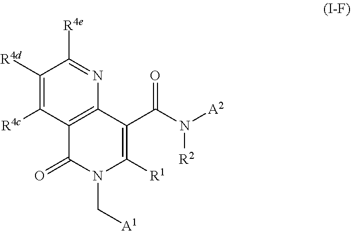
wherein R^{4c}, R^{4d}, and R^{4e} are independently selected from the group consisting of hydrogen, halogen, C₁₋₄alkyl, and C₁₋₄haloalkyl.

13. The compound of claim 11 of formula (I-G), or a pharmaceutically acceptable salt thereof, 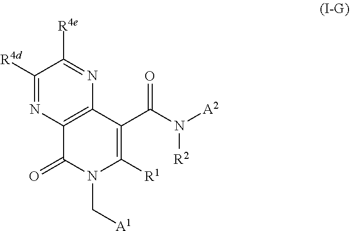
wherein R^{4d} and R^{4e} are independently selected from the group consisting of hydrogen, halogen, C₁₋₄alkyl, and C₁₋₄haloalkyl.

14. The compound of claim 11 of formula (I-H), or a pharmaceutically acceptable salt thereof, wherein 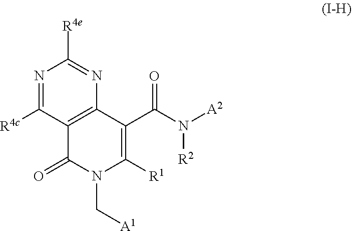
wherein R^{4c} and R^{4e} are independently selected from the group consisting of hydrogen, halogen, C₁₋₄alkyl, and C₁₋₄haloalkyl.

15. The compound of any of claims 12-14, or a pharmaceutically acceptable salt thereof, wherein R^{4c}, R^{4d}, and R^{4e} are hydrogen.

16. The compound of any of claims 1-15, or a pharmaceutically acceptable salt thereof, wherein X² is CR^{6a}.

17. The compound of any of claims 1-16, or a pharmaceutically acceptable salt thereof, wherein when X² is CR^{6a} then R^{6a} is hydrogen.

18. The compound of any of claims 1-15, or a pharmaceutically acceptable salt thereof, wherein X² is N.

19. The compound of any of claims 1-18, or a pharmaceutically acceptable salt thereof, wherein n is 0, 1, or 2.

20. The compound of any of claims 1-19, or a pharmaceutically acceptable salt thereof, wherein R⁶ is halogen; and when X² is CR^{6a} then R^{6a} is hydrogen or halogen.

21. The compound of any of claims 1-20, or a pharmaceutically acceptable salt thereof, halogen wherein A¹ is

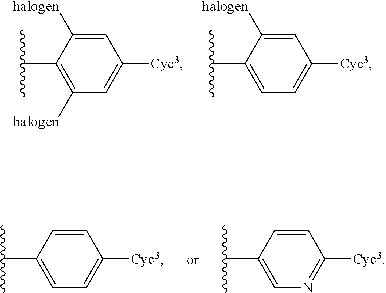

22. The compound of claim 21, or a pharmaceutically acceptable salt thereof, wherein A¹ is

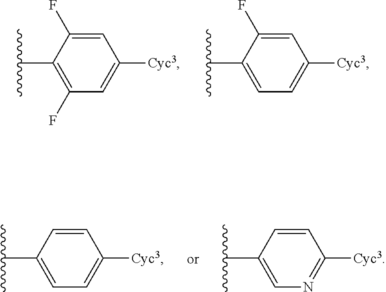

23. The compound of any of claims 1-22, or a pharmaceutically acceptable salt thereof, wherein Cyc³ is the unsubstituted or substituted 5- to 12-membered heteroaryl.

24. The compound of any of claims 1-23, or a pharmaceutically acceptable salt thereof, wherein the ring system of the unsubstituted or substituted 5- to 12-membered heteroaryl of Cyc³ is a 5- to 6-membered monocyclic heteroaryl containing 1-3 heteroatoms independently selected from the group consisting of O, N, and S.

25. The compound of claim 24, or a pharmaceutically acceptable salt thereof, wherein the ring system of the unsubstituted or substituted 5- to 6-membered heteroaryl of Cyc³ is pyrazolyl, oxazolyl, thiazolyl, isothiazolyl, or pyridinyl.

26. The compound of any of claims 1-23, or a pharmaceutically acceptable salt thereof, wherein the ring system of the unsubstituted or substituted 5- to 12-membered heteroaryl of Cyc³ is a 9- to 10-membered fused bicyclic heteroaryl containing 1-3 nitrogen atoms.

27. The compound of claim 26, or a pharmaceutically acceptable salt thereof, wherein the ring system of the unsubstituted or substituted 9- to 10-membered fused bicyclic heteroaryl is a phenyl fused to a 5-membered heteroarene containing 1-3 nitrogen atoms.

28. The compound of claim 26, or a pharmaceutically acceptable salt thereof, wherein the ring system of the unsubstituted or substituted 9- to 10-membered fused bicyclic heteroaryl of Cyc³ is indazolyl.

29. The compound of any of claims 1-28, or a pharmaceutically acceptable salt thereof, wherein Cyc³ is unsubstituted or substituted with 1-3 substituents independently selected from the group consisting of C₁₋₄alkyl and C₃₋₄cycloalkyl.

30. The compound of any of claims 1-25, or a pharmaceutically acceptable salt thereof, wherein Cyc³ is

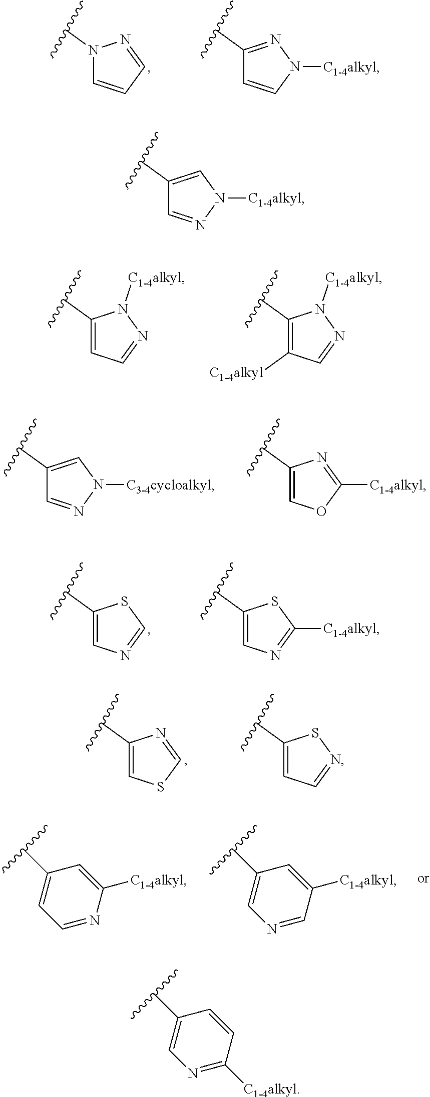

31. The compound of claim 30, or a pharmaceutically acceptable salt thereof, wherein Cyc³ is

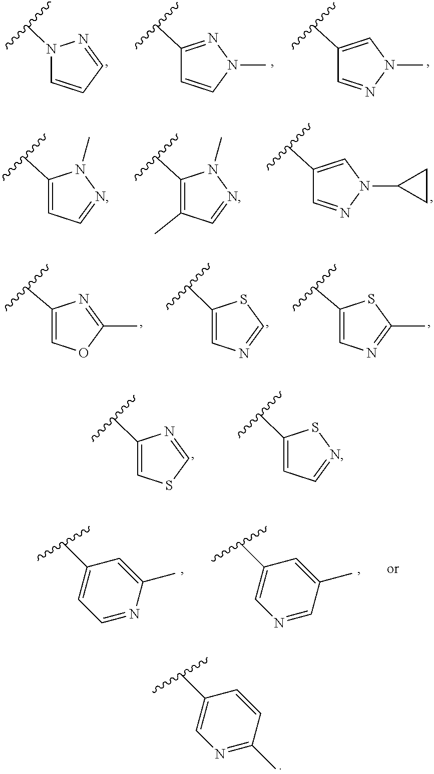

32. The compound of claim 28, or a pharmaceutically acceptable salt thereof, wherein Cyc³ is

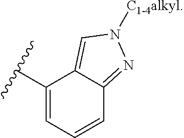

33. The compound of claim 32, or a pharmaceutically acceptable salt thereof, wherein Cyc³ is

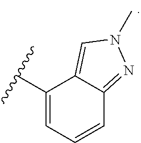 [SMILES: *c1cccc2nn(C)cc12]

34. The compound of any of claims 1-22, or a pharmaceutically acceptable salt thereof, wherein Cyc³ is the unsubstituted or substituted phenyl.

35. The compound of claim 34, or a pharmaceutically acceptable salt thereof, wherein Cyc³ is phenyl unsubstituted or substituted with 1-3 substituents independently selected from the group consisting of C₁₋₄alkyl, halogen, and —OC₁₋₄alkyl.

36. The compound of claim 35, or a pharmaceutically acceptable salt thereof, wherein Cyc³ is

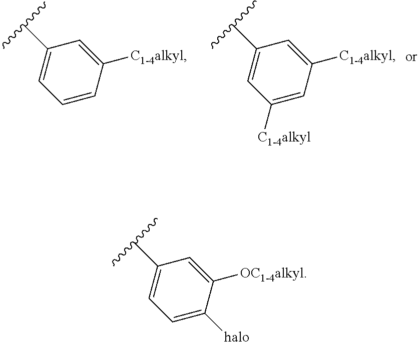

37. The compound of claim 36, or a pharmaceutically acceptable salt thereof, wherein Cyc³ is

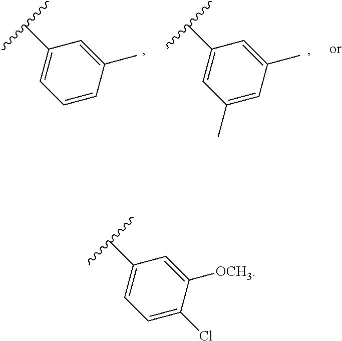

38. The compound of any of claims 1-15, or a pharmaceutically acceptable salt thereof, wherein A¹ is

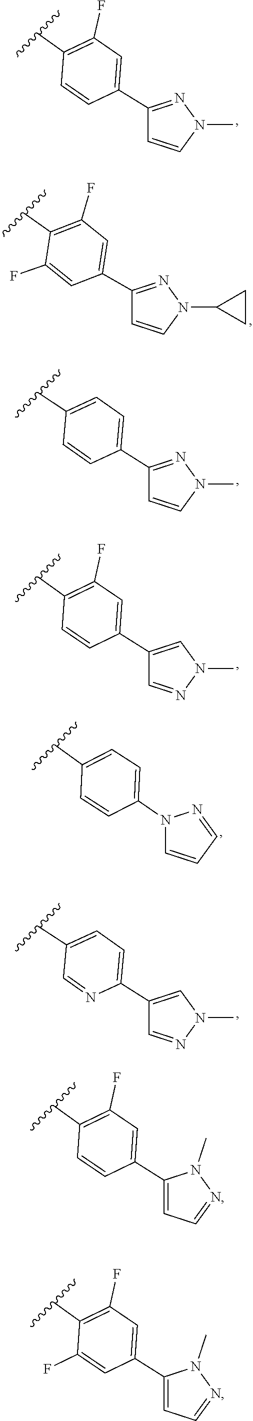

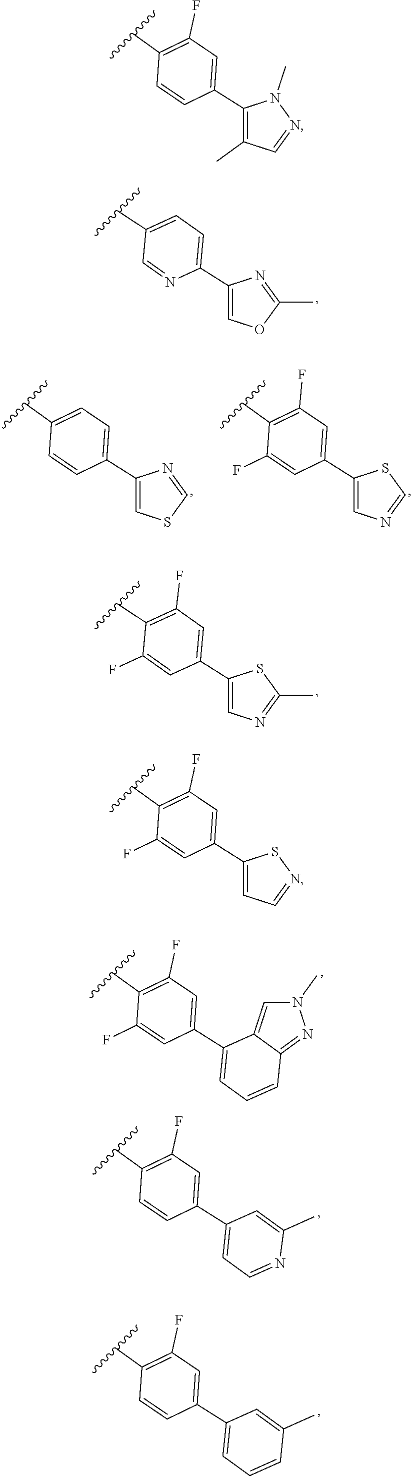

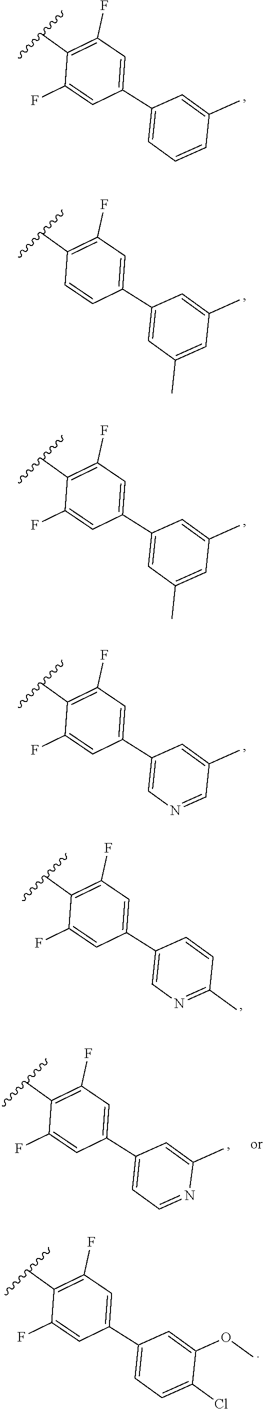

39. The compound of any of claims 1-38, or a pharmaceutically acceptable salt thereof, wherein A² is -L¹-G¹.

40. The compound of any of claims 1-39, or a pharmaceutically acceptable salt thereof, wherein the 4- to 12-membered heterocyclyl of G¹ contains one oxygen ring atom.

41. The compound of any of claims 1-40, or a pharmaceutically acceptable salt thereof, wherein G¹ is a monocyclic C₃₋₈cycloalkyl, a monocyclic 4- to 8-membered heterocyclyl containing one oxygen atom, or a 7- to 12-membered spirocyclic heterocyclyl containing one oxygen atom, wherein G¹ is unsubstituted or substituted with 1-2 substituents selected from the group consisting of —OH, —C₁₋₃alkylene-OH, and C₁₋₄alkyl.

42. The compound of any of claims 1-41, or a pharmaceutically acceptable salt thereof, wherein -L¹-G¹ is

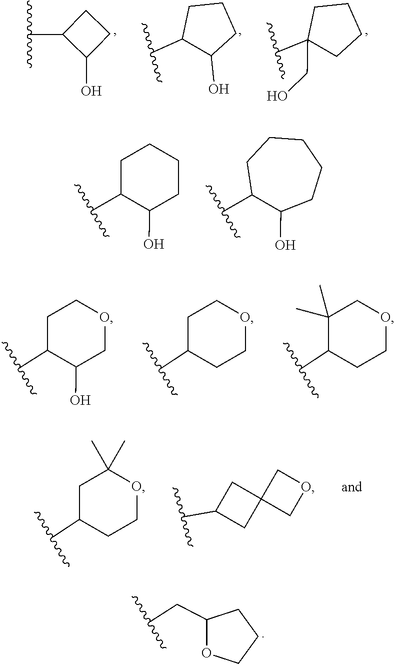

43. The compound of any of claims 1-42, or a pharmaceutically acceptable salt thereof, wherein L¹ is a bond.

44. The compound of any of claims 1-42, or a pharmaceutically acceptable salt thereof, wherein L¹ is a —CH₂—.

45. The compound of any of claims 1-38, or a pharmaceutically acceptable salt thereof, wherein A² is C₁₋₆alkyl optionally substituted with OH.

46. The compound of any of claims 1-38, or a pharmaceutically acceptable salt thereof, wherein A² is C₁₋₆haloalkyl optionally substituted with OH.

47. The compound of any of claims 1-46, or a pharmaceutically acceptable salt thereof, wherein R¹ is hydrogen.

48. The compound of any of claims 1-47, or a pharmaceutically acceptable salt thereof, wherein R² is hydrogen.

49. The compound of claim 1 selected from the group consisting of
6-(2-fluoro-4-(1-methyl-1H-pyrazol-3-yl)benzyl)-N-((3R,4S)-3-hydroxytetrahydro-2H-pyran-4-yl)-5-oxo-5,6-dihydro-1,6-naphthyridine-8-carboxamide;
6-(2-fluoro-4-(1-methyl-1H-pyrazol-3-yl)benzyl)-N-((1S,2S)-2-hydroxycyclohexyl)-5-oxo-5,6-dihydro-1,6-naphthyridine-8-carboxamide;
6-(2-fluoro-4-(1-methyl-1H-pyrazol-3-yl)benzyl)-N-((1S,2S)-2-hydroxycyclopentyl)-5-oxo-5,6-dihydro-1,6-naphthyridine-8-carboxamide;
6-(2-fluoro-4-(1-methyl-1H-pyrazol-3-yl)benzyl)-N-((1R,2R)-2-hydroxycyclobutyl)-5-oxo-5,6-dihydro-1,6-naphthyridine-8-carboxamide;
6-(2-fluoro-4-(1-methyl-1H-pyrazol-3-yl)benzyl)-5-oxo-N-(2-oxaspiro[3.3]heptan-6-yl)-5,6-dihydro-1,6-naphthyridine-8-carboxamide;
6-(2-fluoro-4-(1-methyl-1H-pyrazol-4-yl)benzyl)-N-((3R,4S)-3-hydroxytetrahydro-2H-pyran-4-yl)-5-oxo-5,6-dihydro-1,6-naphthyridine-8-carboxamide;
6-(2-fluoro-4-(1-methyl-1H-pyrazol-4-yl)benzyl)-N-((1S,2S)-2-hydroxycyclohexyl)-5-oxo-5,6-dihydro-1,6-naphthyridine-8-carboxamide;
6-(2-fluoro-4-(1-methyl-1H-pyrazol-4-yl)benzyl)-N-((1S,2S)-2-hydroxycyclopentyl)-5-oxo-5,6-dihydro-1,6-naphthyridine-8-carboxamide;
6-(2-fluoro-4-(1-methyl-1H-pyrazol-4-yl)benzyl)-N-((1R,2R)-2-hydroxycyclobutyl)-5-oxo-5,6-dihydro-1,6-naphthyridine-8-carboxamide;
6-(2-fluoro-4-(1-methyl-1H-pyrazol-4-yl)benzyl)-5-oxo-N-(tetrahydro-2H-pyran-4-yl)-5,6-dihydro-1,6-naphthyridine-8-carboxamide;
6-(2-fluoro-4-(1-methyl-1H-pyrazol-4-yl)benzyl)-5-oxo-N-(2-oxaspiro[3.3]heptan-6-yl)-5,6-dihydro-1,6-naphthyridine-8-carboxamide;
6-(4-(1H-pyrazol-1-yl)benzyl)-N-((3R,4S)-3-hydroxytetrahydro-2H-pyran-4-yl)-5-oxo-5,6-dihydro-1,6-naphthyridine-8-carboxamide;
6-(4-(1H-pyrazol-1-yl)benzyl)-N-((1S,2S)-2-hydroxycyclohexyl)-5-oxo-5,6-dihydro-1,6-naphthyridine-8-carboxamide;
6-(4-(1H-pyrazol-1-yl)benzyl)-N-((1S,2S)-2-hydroxycyclopentyl)-5-oxo-5,6-dihydro-1,6-naphthyridine-8-carboxamide;
N-((3R,4S)-3-hydroxytetrahydro-2H-pyran-4-yl)-6-((6-(1-methyl-1H-pyrazol-4-yl)pyridin-3-yl)methyl)-5-oxo-5,6-dihydro-1,6-naphthyridine-8-carboxamide;
N-((1S,2S)-2-hydroxycyclohexyl)-6-((6-(1-methyl-1H-pyrazol-4-yl)pyridin-3-yl)methyl)-5-oxo-5,6-dihydro-1,6-naphthyridine-8-carboxamide;
N-((1S,2S)-2-hydroxycyclopentyl)-6-((6-(1-methyl-1H-pyrazol-4-yl)pyridin-3-yl)methyl)-5-oxo-5,6-dihydro-1,6-naphthyridine-8-carboxamide;
N-((1S,2S)-2-hydroxycycloheptyl)-6-((6-(1-methyl-1H-pyrazol-4-yl)pyridin-3-yl)methyl)-5-oxo-5,6-dihydro-1,6-naphthyridine-8-carboxamide;
N-((3R,4S)-3-hydroxytetrahydro-2H-pyran-4-yl)-6-(4-(2-methyloxazol-4-yl)benzyl)-5-oxo-5,6-dihydro-1,6-naphthyridine-8-carboxamide;
N-((1S,2S)-2-hydroxycyclohexyl)-6-(4-(2-methyloxazol-4-yl)benzyl)-5-oxo-5,6-dihydro-1,6-naphthyridine-8-carboxamide;
6-(2,6-difluoro-4-(2-methyl-2H-indazol-4-yl)benzyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(2-methyl-2H-indazol-4-yl)benzyl)-N-((1S,2S)-2-hydroxycyclohexyl)-5-oxo-5,6-dihydro-1,6-naphthyridine-8-carboxamide;
6-(2,6-difluoro-4-(2-methyl-2H-indazol-4-yl)benzyl)-N-((1S,2S)-2-hydroxycycloheptyl)-5-oxo-5,6-dihydro-1,6-naphthyridine-8-carboxamide;
6-(2-fluoro-4-(1-methyl-1H-pyrazol-4-yl)benzyl)-N-((1S,2S)-2-hydroxycycloheptyl)-5-oxo-5,6-dihydro-1,6-naphthyridine-8-carboxamide;
6-(4-(1H-pyrazol-1-yl)benzyl)-N-((1S,2S)-2-hydroxycycloheptyl)-5-oxo-5,6-dihydro-1,6-naphthyridine-8-carboxamide;
6-(2-fluoro-4-(2-methylpyridin-4-yl)benzyl)-N-((3R,4S)-3-hydroxytetrahydro-2H-pyran-4-yl)-5-oxo-5,6-dihydro-1,6-naphthyridine-8-carboxamide;
6-(2-fluoro-4-(2-methylpyridin-4-yl)benzyl)-N-((1S,2S)-2-hydroxycyclohexyl)-5-oxo-5,6-dihydro-1,6-naphthyridine-8-carboxamide;
6-(2-fluoro-4-(2-methylpyridin-4-yl)benzyl)-N-((1S,2S)-2-hydroxycyclopentyl)-5-oxo-5,6-dihydro-1,6-naphthyridine-8-carboxamide;
6-((3-fluoro-3′-methyl-[1,1′-biphenyl]-4-yl)methyl)-N-((3R,4S)-3-hydroxytetrahydro-2H-pyran-4-yl)-5-oxo-5,6-dihydro-1,6-naphthyridine-8-carboxamide;
6-((3-fluoro-3′,5′-dimethyl-[1,1′-biphenyl]-4-yl)methyl)-N-((3R,4S)-3-hydroxytetrahydro-2H-pyran-4-yl)-5-oxo-5,6-dihydro-1,6-naphthyridine-8-carboxamide;
6-((3,5-difluoro-3′-methyl-[1,1′-biphenyl]-4-yl)methyl)-N-((3R,4S)-3-hydroxytetrahydro-2H-pyran-4-yl)-5-oxo-5,6-dihydro-1,6-naphthyridine-8-carboxamide;
6-((3,5-difluoro-3′,5′-dimethyl-[1,1′-biphenyl]-4-yl)methyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(5-methylpyridin-3-yl)benzyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(6-methylpyridin-3-yl)benzyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(6-methylpyridin-3-yl)benzyl)-N-((1S,2S)-2-hydroxycyclohexyl)-5-oxo-5,6-dihydro-1,6-naphthyridine-8-carboxamide;
6-(2,6-difluoro-4-(2-methylpyridin-4-yl)benzyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(2-methylpyridin-4-yl)benzyl)-N-((1S,2S)-2-hydroxycyclohexyl)-5-oxo-5,6-dihydro-1,6-naphthyridine-8-carboxamide;
6-(2,6-difluoro-4-(2-methylpyridin-4-yl)benzyl)-N-((1S,2S)-2-hydroxycyclopentyl)-5-oxo-5,6-dihydro-1,6-naphthyridine-8-carboxamide;
6-((4′-chloro-3,5-difluoro-3′-methoxy-[1,1′-biphenyl]-4-yl)methyl)-N-((3R,4S)-3-hydroxytetrahydro-2H-pyran-4-yl)-5-oxo-5,6-dihydro-1,6-naphthyridine-8-carboxamide;
6-(4-(1-cyclopropyl-1H-pyrazol-4-yl)-2,6-difluorobenzyl)-N-((3R,4S)-3-hydroxytetrahydro-2H-pyran-4-yl)-5-oxo-5,6-dihydro-1,6-naphthyridine-8-carboxamide;
6-(4-(1-cyclopropyl-1H-pyrazol-4-yl)-2,6-difluorobenzyl)-N-((1S,2S)-2-hydroxycyclohexyl)-5-oxo-5,6-dihydro-1,6-naphthyridine-8-carboxamide;
6-(4-(1-cyclopropyl-1H-pyrazol-4-yl)-2,6-difluorobenzyl)-N-((1S,2S)-2-hydroxycyclopentyl)-5-oxo-5,6-dihydro-1,6-naphthyridine-8-carboxamide;
6-(2,6-difluoro-4-(5-methylpyridin-3-yl)benzyl)-N-((1S,2S)-2-hydroxycyclohexyl)-5-oxo-5,6-dihydro-1,6-naphthyridine-8-carboxamide;
6-(2,6-difluoro-4-(5-methylpyridin-3-yl)benzyl)-N-((1S,2S)-2-hydroxycyclopentyl)-5-oxo-5,6-dihydro-1,6-naphthyridine-8-carboxamide;
6-(2-fluoro-4-(1-methyl-1H-pyrazol-5-yl)benzyl)-N-((3R,4S)-3-hydroxytetrahydro-2H-pyran-4-yl)-5-oxo-5,6-dihydro-1,6-naphthyridine-8-carboxamide;
6-(2-fluoro-4-(1-methyl-1H-pyrazol-5-yl)benzyl)-N-((1S,2S)-2-hydroxycyclohexyl)-5-oxo-5,6-dihydro-1,6-naphthyridine-8-carboxamide;
6-(2,6-difluoro-4-(1-methyl-1H-pyrazol-5-yl)benzyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(1-methyl-1H-pyrazol-5-yl)benzyl)-N-((1S,2S)-2-hydroxycyclohexyl)-5-oxo-5,6-dihydro-1,6-naphthyridine-8-carboxamide;
6-(2,6-difluoro-4-(thiazol-5-yl)benzyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(isothiazol-5-yl)benzyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(2-methylthiazol-5-yl)benzyl)-N-((3R,4S)-3-hydroxytetrahydro-2H-pyran-4-yl)-5-oxo-5,6-dihydro-1,6-naphthyridine-8-carboxamide;
6-(2,6-difluoro-4-(2-methyl-2H-indazol-4-yl)benzyl)-N-((1S,2S)-2-hydroxycyclohexyl)-5-oxo-5,6-dihydropyrido[3,4-b]pyrazine-8-carboxamide;
6-(4-(1H-pyrazol-1-yl)benzyl)-N-((1S,2S)-2-hydroxycyclohexyl)-5-oxo-5,6-dihydropyrido[3,4-b]pyrazine-8-carboxamide;
6-(2-fluoro-4-(1-methyl-1H-pyrazol-4-yl)benzyl)-N-((1S,2S)-2-hydroxycyclohexyl)-5-oxo-5,6-dihydropyrido[3,4-b]pyrazine-8-carboxamide;
6-(2-fluoro-4-(1-methyl-1H-pyrazol-3-yl)benzyl)-N-((1S,2S)-2-hydroxycyclohexyl)-5-oxo-5,6-dihydropyrido[3,4-b]pyrazine-8-carboxamide;
6-(4-(1H-pyrazol-1-yl)benzyl)-N-((3R,4S)-3-hydroxytetrahydro-2H-pyran-4-yl)-5-oxo-5,6-dihydropyrido[3,4-b]pyrazine-8-carboxamide;
N-((3R,4S)-3-hydroxytetrahydro-2H-pyran-4-yl)-5-oxo-6-(4-(thiazol-4-yl)benzyl)-5,6-dihydro-1,6-naphthyridine-8-carboxamide;
N-((3R,4S)-3-hydroxytetrahydro-2H-pyran-4-yl)-6-(4-(1-methyl-1H-pyrazol-4-yl)benzyl)-5-oxo-5,6-dihydro-1,6-naphthyridine-8-carboxamide;
N-((1S,2S)-2-hydroxycyclohexyl)-5-oxo-6-(4-(thiazol-4-yl)benzyl)-5,6-dihydro-1,6-naphthyridine-8-carboxamide;
N-((1S,2S)-2-hydroxycycloheptyl)-5-oxo-6-(4-(thiazol-4-yl)benzyl)-5,6-dihydro-1,6-naphthyridine-8-carboxamide;
6-(4-(1H-pyrazol-1-yl)benzyl)-N-((1S,2S)-2-hydroxycyclopentyl)-5-oxo-5,6-dihydropyrido[3,4-b]pyrazine-8-carboxamide;
6-(4-(1H-pyrazol-1-yl)benzyl)-N-((1S,2S)-2-hydroxycycloheptyl)-5-oxo-5,6-dihydropyrido[3,4-b]pyrazine-8-carboxamide;
6-(2-fluoro-4-(1-methyl-1H-pyrazol-3-yl)benzyl)-N-((3R,4S)-3-hydroxytetrahydro-2H-pyran-4-yl)-5-oxo-5,6-dihydropyrido[3,4-b]pyrazine-8-carboxamide;
6-(2-fluoro-4-(1-methyl-1H-pyrazol-3-yl)benzyl)-N-((1S,2S)-2-hydroxycyclopentyl)-5-oxo-5,6-dihydropyrido[3,4-b]pyrazine-8-carboxamide;
6-(2-fluoro-4-(1-methyl-1H-pyrazol-3-yl)benzyl)-N-((1S,2S)-2-hydroxycycloheptyl)-5-oxo-5,6-dihydropyrido[3,4-b]pyrazine-8-carboxamide;
6-(2,6-difluoro-4-(2-methyl-2H-indazol-4-yl)benzyl)-N-((3R,4S)-3-hydroxytetrahydro-2H-pyran-4-yl)-5-oxo-5,6-dihydropyrido[3,4-b]pyrazine-8-carboxamide;
6-(2,6-difluoro-4-(2-methyl-2H-indazol-4-yl)benzyl)-N-((1S,2S)-2-hydroxycycloheptyl)-5-oxo-5,6-dihydropyrido[3,4-b]pyrazine-8-carboxamide;
6-(2,6-difluoro-4-(2-methyl-2H-indazol-4-yl)benzyl)-N-((1S,2S)-2-hydroxycyclopentyl)-5-oxo-5,6-dihydropyrido[3,4-b]pyrazine-8-carboxamide;
6-(2,6-difluoro-4-(2-methyl-2H-indazol-4-yl)benzyl)-N-((1S,2S)-2-hydroxycyclobutyl)-5-oxo-5,6-dihydropyrido[3,4-b]pyrazine-8-carboxamide;
6-(2,6-difluoro-4-(2-methyl-2H-indazol-4-yl)benzyl)-5-oxo-N-(tetrahydro-2H-pyran-4-yl)-5,6-dihydropyrido[3,4-b]pyrazine-8-carboxamide;
6-(2,6-difluoro-4-(2-methyl-2H-indazol-4-yl)benzyl)-5-oxo-N-(2-oxaspiro[3.3]heptan-6-yl)-5,6-dihydropyrido[3,4-b]pyrazine-8-carboxamide
6-(2,6-difluoro-4-(2-methyl-2H-indazol-4-yl)benzyl)-N-(3-(hydroxymethyl)tetrahydrofuran-3-yl)-5-oxo-5,6-dihydropyrido[3,4-b]pyrazine-8-carboxamide;
6-(2,6-difluoro-4-(2-methyl-2H-indazol-4-yl)benzyl)-N-(2-hydroxy-2-methylpropyl)-5-oxo-5,6-dihydropyrido[3,4-b]pyrazine-8-carboxamide;
6-(2,6-difluoro-4-(2-methyl-2H-indazol-4-yl)benzyl)-5-oxo-N-(3,3,3-trifluoro-2-hydroxypropyl)-5,6-dihydropyrido[3,4-b]pyrazine-8-carboxamide;
(S)-6-(2,6-difluoro-4-(2-methyl-2H-indazol-4-yl)benzyl)-N-(1-hydroxy-3-methylbutan-2-yl)-5-oxo-5,6-dihydropyrido[3,4-b]pyrazine-8-carboxamide;
6-(2,6-difluoro-4-(2-methyl-2H-indazol-4-yl)benzyl)-N-((2S,3S)-1-hydroxy-3-methylpentan-2-yl)-5-oxo-5,6-dihydropyrido[3,4-b]pyrazine-8-carboxamide;
6-(2-fluoro-4-(1-methyl-1H-pyrazol-4-yl)benzyl)-N-((3R,4S)-3-hydroxytetrahydro-2H-pyran-4-yl)-5-oxo-5,6-dihydropyrido[3,4-b]pyrazine-8-carboxamide;
6-(2-fluoro-4-(1-methyl-1H-pyrazol-4-yl)benzyl)-N-((1S,2S)-2-hydroxycycloheptyl)-5-oxo-5,6-dihydropyrido[3,4-b]pyrazine-8-carboxamide;
6-(2-fluoro-4-(1-methyl-1H-pyrazol-4-yl)benzyl)-N-((1S,2S)-2-hydroxycyclopentyl)-5-oxo-5,6-dihydropyrido[3,4-b]pyrazine-8-carboxamide;
6-(2-fluoro-4-(1-methyl-1H-pyrazol-4-yl)benzyl)-N-(2-hydroxy-2-methylpropyl)-5-oxo-5,6-dihydropyrido[3,4-b]pyrazine-8-carboxamide;
6-(2-fluoro-4-(1-methyl-1H-pyrazol-4-yl)benzyl)-5-oxo-N-(3,3,3-trifluoro-2-hydroxypropyl)-5,6-dihydropyrido[3,4-b]pyrazine-8-carboxamide;
(S)-6-(2-fluoro-4-(1-methyl-1H-pyrazol-4-yl)benzyl)-N-(1-hydroxy-3-methylbutan-2-yl)-5-oxo-5,6-dihydropyrido[3,4-b]pyrazine-8-carboxamide;
6-(2-fluoro-4-(1-methyl-1H-pyrazol-4-yl)benzyl)-N-((2S,3S)-1-hydroxy-3-methylpentan-2-yl)-5-oxo-5,6-dihydropyrido[3,4-b]pyrazine-8-carboxamide;
6-(2,6-difluoro-4-(2-methyl-2H-indazol-4-yl)benzyl)-N-((3R,4S)-3-hydroxytetrahydro-2H-pyran-4-yl)-5-oxo-5,6-dihydropyrido[4,3-d]pyrimidine-8-carboxamide;
6-(2,6-difluoro-4-(2-methyl-2H-indazol-4-yl)benzyl)-N-((1S,2S)-2-hydroxycycloheptyl)-5-oxo-5,6-dihydropyrido[4,3-d]pyrimidine-8-carboxamide;
N-((1S,2S)-2-hydroxycyclohexyl)-6-(4-(1-methyl-1H-pyrazol-4-yl)benzyl)-5-oxo-5,6-dihydro-1,6-naphthyridine-8-carboxamide;
N-((1S,2S)-2-hydroxycycloheptyl)-6-(4-(1-methyl-1H-pyrazol-4-yl)benzyl)-5-oxo-5,6-dihydro-1,6-naphthyridine-8-carboxamide;
6-(2,6-difluoro-4-(2-methyl-2H-indazol-4-yl)benzyl)-N-((1S,2S)-2-hydroxycyclohexyl)-5-oxo-5,6-dihydropyrido[4,3-d]pyrimidine-8-carboxamide;
6-(2-fluoro-4-(1-methyl-1H-pyrazol-4-yl)benzyl)-N-((1S,2S)-2-hydroxycyclohexyl)-5-oxo-5,6-dihydropyrido[4,3-d]pyrimidine-8-carboxamide;
6-(2-fluoro-4-(1-methyl-1H-pyrazol-4-yl)benzyl)-N-((3R,4S)-3-hydroxytetrahydro-2H-pyran-4-yl)-5-oxo-5,6-dihydropyrido[4,3-d]pyrimidine-8-carboxamide;
6-(2-fluoro-4-(1-methyl-1H-pyrazol-4-yl)benzyl)-N-((1S,2S)-2-hydroxycycloheptyl)-5-oxo-5,6-dihydropyrido[4,3-d]pyrimidine-8-carboxamide;
6-(2-fluoro-4-(1-methyl-1H-pyrazol-3-yl)benzyl)-N-((3R,4S)-3-hydroxytetrahydro-2H-pyran-4-yl)-5-oxo-5,6-dihydroimidazo[1,2-c]pyrimidine-8-carboxamide;
6-(2-fluoro-4-(1-methyl-1H-pyrazol-3-yl)benzyl)-N-((1S,2S)-2-hydroxycyclohexyl)-5-oxo-5,6-dihydroimidazo[1,2-c]pyrimidine-8-carboxamide;
6-(2-fluoro-4-(1-methyl-1H-pyrazol-3-yl)benzyl)-N-((1S,2S)-2-hydroxycyclopentyl)-5-oxo-5,6-dihydroimidazo[1,2-c]pyrimidine-8-carboxamide;
6-(2-fluoro-4-(1-methyl-1H-pyrazol-3-yl)benzyl)-N-((1R,2R)-2-hydroxycyclobutyl)-5-oxo-5,6-dihydroimidazo[1,2-c]pyrimidine-8-carboxamide;
6-(2-fluoro-4-(1-methyl-1H-pyrazol-4-yl)benzyl)-N-((3R,4S)-3-hydroxytetrahydro-2H-pyran-4-yl)-5-oxo-5,6-dihydroimidazo[1,2-c]pyrimidine-8-carboxamide;
6-(2-fluoro-4-(1-methyl-1H-pyrazol-4-yl)benzyl)-N-((1S,2S)-2-hydroxycyclohexyl)-5-oxo-5,6-dihydroimidazo[1,2-c]pyrimidine-8-carboxamide;
6-(2-fluoro-4-(1-methyl-1H-pyrazol-4-yl)benzyl)-N-((1S,2S)-2-hydroxycyclopentyl)-5-oxo-5,6-dihydroimidazo[1,2-c]pyrimidine-8-carboxamide;
6-(2-fluoro-4-(1-methyl-1H-pyrazol-4-yl)benzyl)-N-((1R,2R)-2-hydroxycyclobutyl)-5-oxo-5,6-dihydroimidazo[1,2-c]pyrimidine-8-carboxamide;
6-(2-fluoro-4-(1-methyl-1H-pyrazol-4-yl)benzyl)-5-oxo-N-(2-oxaspiro[3.3]heptan-6-yl)-5,6-dihydroimidazo[1,2-c]pyrimidine-8-carboxamide;
N-((3R,4S)-3-hydroxytetrahydro-2H-pyran-4-yl)-6-((6-(1-methyl-1H-pyrazol-4-yl)pyridin-3-yl)methyl)-5-oxo-5,6-dihydroimidazo[1,2-c]pyrimidine-8-carboxamide;
N-((1S,2S)-2-hydroxycyclohexyl)-6-((6-(1-methyl-1H-pyrazol-4-yl)pyridin-3-yl)methyl)-5-oxo-5,6-dihydroimidazo[1,2-c]pyrimidine-8-carboxamide;
6-(4-(1H-pyrazol-1-yl)benzyl)-N-((3R,4S)-3-hydroxytetrahydro-2H-pyran-4-yl)-5-oxo-5,6-dihydroimidazo[1,2-c]pyrimidine-8-carboxamide;
6-(4-(1H-pyrazol-1-yl)benzyl)-N-((1S,2S)-2-hydroxycyclohexyl)-5-oxo-5,6-dihydroimidazo[1,2-c]pyrimidine-8-carboxamide;
6-(4-(1H-pyrazol-1-yl)benzyl)-N-((1S,2S)-2-hydroxycyclopentyl)-5-oxo-5,6-dihydroimidazo[1,2-c]pyrimidine-8-carboxamide;
6-(2-fluoro-4-(1-methyl-1H-pyrazol-4-yl)benzyl)-N-((1S,2S)-2-hydroxycycloheptyl)-5-oxo-5,6-dihydroimidazo[1,2-c]pyrimidine-8-carboxamide;
6-(4-(1H-pyrazol-1-yl)benzyl)-N-((1S,2S)-2-hydroxycycloheptyl)-5-oxo-5,6-dihydroimidazo[1,2-c]pyrimidine-8-carboxamide;
N-((3R,4S)-3-hydroxytetrahydro-2H-pyran-4-yl)-5-oxo-6-(4-(thiazol-4-yl)benzyl)-5,6-dihydroimidazo[1,2-c]pyrimidine-8-carboxamide;
6-(2,6-difluoro-4-(2-methyl-2H-indazol-4-yl)benzyl)-N-((3R,4S)-3-hydroxytetrahydro-2H-pyran-4-yl)-5-oxo-5,6-dihydroimidazo[1,2-c]pyrimidine-8-carboxamide;
6-(2,6-difluoro-4-(2-methyl-2H-indazol-4-yl)benzyl)-N-((1S,2S)-2-hydroxycycloheptyl)-5-oxo-5,6-dihydroimidazo[1,2-c]pyrimidine-8-carboxamide;
6-(2,6-difluoro-4-(2-methyl-2H-indazol-4-yl)benzyl)-N-((1S,2S)-2-hydroxycyclohexyl)-5-oxo-5,6-dihydroimidazo[1,2-c]pyrimidine-8-carboxamide;
6-(2,6-difluoro-4-(2-methyl-2H-indazol-4-yl)benzyl)-N-((1S,2S)-2-hydroxycyclopentyl)-5-oxo-5,6-dihydroimidazo[1,2-c]pyrimidine-8-carboxamide;
6-(2,6-difluoro-4-(2-methyl-2H-indazol-4-yl)benzyl)-N-((1S,2S)-2-hydroxycyclobutyl)-5-oxo-5,6-dihydroimidazo[1,2-c]pyrimidine-8-carboxamide;
6-(2-fluoro-4-(1-methyl-1H-pyrazol-4-yl)benzyl)-N-((3R,4S)-3-hydroxytetrahydro-2H-pyran-4-yl)-2-methyl-5-oxo-5,6-dihydroimidazo[1,2-c]pyrimidine-8-carboxamide;
6-(2-fluoro-4-(1-methyl-1H-pyrazol-4-yl)benzyl)-N-((1S,2S)-2-hydroxycyclohexyl)-2-methyl-5-oxo-5,6-dihydroimidazo[1,2-c]pyrimidine-8-carboxamide;
6-(2-fluoro-4-(1-methyl-1H-pyrazol-3-yl)benzyl)-N-((3R,4S)-3-hydroxytetrahydro-2H-pyran-4-yl)-2-methyl-5-oxo-5,6-dihydroimidazo[1,2-c]pyrimidine-8-carboxamide;
6-(2-fluoro-4-(1-methyl-1H-pyrazol-3-yl)benzyl)-N-((1S,2S)-2-hydroxycyclohexyl)-2-methyl-5-oxo-5,6-dihydroimidazo[1,2-c]pyrimidine-8-carboxamide;
6-(4-(1H-pyrazol-1-yl)benzyl)-N-((3R,4S)-3-hydroxytetrahydro-2H-pyran-4-yl)-2-methyl-5-oxo-5,6-dihydroimidazo[1,2-c]pyrimidine-8-carboxamide;
6-(4-(1H-pyrazol-1-yl)benzyl)-N-((1S,2S)-2-hydroxycyclohexyl)-2-methyl-5-oxo-5,6-dihydroimidazo[1,2-c]pyrimidine-8-carboxamide;
6-(2,6-difluoro-4-(2-methyl-2H-indazol-4-yl)benzyl)-N-((3R,4S)-3-hydroxytetrahydro-2H-pyran-4-yl)-2-methyl-5-oxo-5,6-dihydroimidazo[1,2-c]pyrimidine-8-carboxamide;
6-(2,6-difluoro-4-(2-methyl-2H-indazol-4-yl)benzyl)-N-((1S,2S)-2-hydroxycyclohexyl)-2-methyl-5-oxo-5,6-dihydroimidazo[1,2-c]pyrimidine-8-carboxamide;
6-(2-fluoro-4-(1-methyl-1H-pyrazol-5-yl)benzyl)-N-((3R,4S)-3-hydroxytetrahydro-2H-pyran-4-yl)-5-oxo-5,6-dihydroimidazo[1,2-c]pyrimidine-8-carboxamide;
6-(2-fluoro-4-(1-methyl-1H-pyrazol-5-yl)benzyl)-N-((1S,2S)-2-hydroxycyclohexyl)-5-oxo-5,6-dihydroimidazo[1,2-c]pyrimidine-8-carboxamide;
6-(2-fluoro-4-(1-methyl-1H-pyrazol-5-yl)benzyl)-N-((1S,2S)-2-hydroxycyclopentyl)-5-oxo-5,6-dihydroimidazo[1,2-c]pyrimidine-8-carboxamide;
6-(4-(1,4-dimethyl-1H-pyrazol-5-yl)-2-fluorobenzyl)-N-((3R,4S)-3-hydroxytetrahydro-2H-pyran-4-yl)-5-oxo-5,6-dihydroimidazo[1,2-c]pyrimidine-8-carboxamide;
6-(4-(1,4-dimethyl-1H-pyrazol-5-yl)-2-fluorobenzyl)-N-((1S,2S)-2-hydroxycyclohexyl)-5-oxo-5,6-dihydroimidazo[1,2-c]pyrimidine-8-carboxamide;
6-(2,6-difluoro-4-(1-methyl-1H-pyrazol-5-yl)benzyl)-N-((3R,4S)-3-hydroxytetrahydro-2H-pyran-4-yl)-5-oxo-5,6-dihydroimidazo[1,2-c]pyrimidine-8-carboxamide;
5-(2-fluoro-4-(1-methyl-1H-pyrazol-3-yl)benzyl)-N-((1S,2S)-2-hydroxycyclohexyl)-3-methyl-4-oxo-4,5-dihydro-3H-imidazo[4,5-c]pyridine-7-carboxamide;
5-(2-fluoro-4-(1-methyl-1H-pyrazol-3-yl)benzyl)-N-((1S,2S)-2-hydroxycyclopentyl)-3-methyl-4-oxo-4,5-dihydro-3H-imidazo[4,5-c]pyridine-7-carboxamide;
5-(2-fluoro-4-(1-methyl-1H-pyrazol-3-yl)benzyl)-N-((1S,2S)-2-hydroxycycloheptyl)-3-methyl-4-oxo-4,5-dihydro-3H-imidazo[4,5-c]pyridine-7-carboxamide;
5-(2,6-difluoro-4-(2-methyl-2H-indazol-4-yl)benzyl)-N-((3R,4S)-3-hydroxytetrahydro-2H-pyran-4-yl)-3-methyl-4-oxo-4,5-dihydro-3H-imidazo[4,5-c]pyridine-7-carboxamide;
5-(4-(1H-pyrazol-1-yl)benzyl)-N-((3S,4S)-4-hydroxytetrahydro-2H-pyran-3-yl)-3-methyl-4-oxo-4,5-dihydro-3H-imidazo[4,5-c]pyridine-7-carboxamide;
5-(4-(1H-pyrazol-1-yl)benzyl)-N-((1S,2S)-2-hydroxycyclohexyl)-3-methyl-4-oxo-4,5-dihydro-3H-imidazo[4,5-c]pyridine-7-carboxamide;
5-(4-(1H-pyrazol-1-yl)benzyl)-N-(3,3-dimethyltetrahydro-2H-pyran-4-yl)-4-oxo-4,5-dihydrofuro[3,2-c]pyridine-7-carboxamide;
5-(4-(1H-pyrazol-1-yl)benzyl)-N-((3R,4S)-3-hydroxytetrahydro-2H-pyran-4-yl)-4-oxo-4,5-dihydrofuro[3,2-c]pyridine-7-carboxamide;
5-(4-(1H-pyrazol-1-yl)benzyl)-N-((1S,2S)-2-hydroxycyclohexyl)-4-oxo-4,5-dihydrofuro[3,2-c]pyridine-7-carboxamide;
5-(4-(1H-pyrazol-1-yl)benzyl)-N-((1S,2S)-2-hydroxycyclopentyl)-4-oxo-4,5-dihydrofuro[3,2-c]pyridine-7-carboxamide;
5-(4-(1H-pyrazol-1-yl)benzyl)-N-((1S,2S)-2-hydroxycycloheptyl)-4-oxo-4,5-dihydrofuro[3,2-c]pyridine-7-carboxamide;
5-(2-fluoro-4-(1-methyl-1H-pyrazol-3-yl)benzyl)-N-((3R,4S)-3-hydroxytetrahydro-2H-pyran-4-yl)-4-oxo-4,5-dihydrofuro[3,2-c]pyridine-7-carboxamide;
5-(2-fluoro-4-(1-methyl-1H-pyrazol-3-yl)benzyl)-N-((1S,2S)-2-hydroxycyclohexyl)-4-oxo-4,5-dihydrofuro[3,2-c]pyridine-7-carboxamide;
5-(2-fluoro-4-(1-methyl-1H-pyrazol-3-yl)benzyl)-N-((1S,2S)-2-hydroxycyclopentyl)-4-oxo-4,5-dihydrofuro[3,2-c]pyridine-7-carboxamide;
5-(2-fluoro-4-(1-methyl-1H-pyrazol-3-yl)benzyl)-N-((1S,2S)-2-hydroxycycloheptyl)-4-oxo-4,5-dihydrofuro[3,2-c]pyridine-7-carboxamide;
5-(2-fluoro-4-(1-methyl-1H-pyrazol-3-yl)benzyl)-4-oxo-N-(2-oxaspiro[3.3]heptan-6-yl)-4,5-dihydrofuro[3,2-c]pyridine-7-carboxamide;
N-(2,2-dimethyltetrahydro-2H-pyran-4-yl)-5-(2-fluoro-4-(1-methyl-1H-pyrazol-3-yl)benzyl)-4-oxo-4,5-dihydrofuro[3,2-c]pyridine-7-carboxamide;
5-(2-fluoro-4-(1-methyl-1H-pyrazol-4-yl)benzyl)-N-((3R,4S)-3-hydroxytetrahydro-2H-pyran-4-yl)-4-oxo-4,5-dihydrofuro[3,2-c]pyridine-7-carboxamide;
5-(2-fluoro-4-(1-methyl-1H-pyrazol-4-yl)benzyl)-N-((1S,2S)-2-hydroxycyclohexyl)-4-oxo-4,5-dihydrofuro[3,2-c]pyridine-7-carboxamide;
5-(2-fluoro-4-(1-methyl-1H-pyrazol-4-yl)benzyl)-N-((1S,2S)-2-hydroxycyclopentyl)-4-oxo-4,5-dihydrofuro[3,2-c]pyridine-7-carboxamide;
5-(2-fluoro-4-(1-methyl-1H-pyrazol-4-yl)benzyl)-N-((1R,2R)-2-hydroxycyclobutyl)-4-oxo-4,5-dihydrofuro[3,2-c]pyridine-7-carboxamide;
5-(2-fluoro-4-(1-methyl-1H-pyrazol-4-yl)benzyl)-N-((1S,2S)-2-hydroxycycloheptyl)-4-oxo-4,5-dihydrofuro[3,2-c]pyridine-7-carboxamide;
5-(2-fluoro-4-(1-methyl-1H-pyrazol-4-yl)benzyl)-4-oxo-N-(2-oxaspiro[3.3]heptan-6-yl)-4,5-dihydrofuro[3,2-c]pyridine-7-carboxamide;
5-(2,6-difluoro-4-(2-methyl-2H-indazol-4-yl)benzyl)-N-((3R,4S)-3-hydroxytetrahydro-2H-pyran-4-yl)-4-oxo-4,5-dihydrofuro[3,2-c]pyridine-7-carboxamide;
5-(2,6-difluoro-4-(2-methyl-2H-indazol-4-yl)benzyl)-N-((1S,2S)-2-hydroxycyclohexyl)-4-oxo-4,5-dihydrofuro[3,2-c]pyridine-7-carboxamide;
5-(2,6-difluoro-4-(2-methyl-2H-indazol-4-yl)benzyl)-N-((1S,2S)-2-hydroxycyclopentyl)-4-oxo-4,5-dihydrofuro[3,2-c]pyridine-7-carboxamide;
5-(2,6-difluoro-4-(2-methyl-2H-indazol-4-yl)benzyl)-N-((1R,2R)-2-hydroxycyclobutyl)-4-oxo-4,5-dihydrofuro[3,2-c]pyridine-7-carboxamide;
5-(2,6-difluoro-4-(2-methyl-2H-indazol-4-yl)benzyl)-N-((1S,2S)-2-hydroxycycloheptyl)-4-oxo-4,5-dihydrofuro[3,2-c]pyridine-7-carboxamide;
5-(2,6-difluoro-4-(2-methyl-2H-indazol-4-yl)benzyl)-4-oxo-N-(tetrahydro-2H-pyran-4-yl)-4,5-dihydrofuro[3,2-c]pyridine-7-carboxamide;
5-(2,6-difluoro-4-(2-methyl-2H-indazol-4-yl)benzyl)-4-oxo-N-(2-oxaspiro[3.3]heptan-6-yl)-4,5-dihydrofuro[3,2-c]pyridine-7-carboxamide;
5-(2,6-difluoro-4-(2-methyl-2H-indazol-4-yl)benzyl)-4-oxo-N-((tetrahydrofuran-2-yl)methyl)-4,5-dihydrofuro[3,2-c]pyridine-7-carboxamide;
5-(2-fluoro-4-(1-methyl-1H-pyrazol-3-yl)benzyl)-N-((3R,4S)-3-hydroxytetrahydro-2H-pyran-4-yl)-4-oxo-4,5-dihydrothieno[3,2-c]pyridine-7-carboxamide;
5-(2-fluoro-4-(1-methyl-1H-pyrazol-3-yl)benzyl)-N-((1S,2S)-2-hydroxycyclohexyl)-4-oxo-4,5-dihydrothieno[3,2-c]pyridine-7-carboxamide;
5-(2-fluoro-4-(1-methyl-1H-pyrazol-3-yl)benzyl)-N-((1S,2S)-2-hydroxycycloheptyl)-4-oxo-4,5-dihydrothieno[3,2-c]pyridine-7-carboxamide;
5-(2-fluoro-4-(1-methyl-1H-pyrazol-4-yl)benzyl)-N-((3R,4S)-3-hydroxytetrahydro-2H-pyran-4-yl)-4-oxo-4,5-dihydrothieno[3,2-c]pyridine-7-carboxamide;
5-(2,6-difluoro-4-(2-methyl-2H-indazol-4-yl)benzyl)-N-((3R,4S)-3-hydroxytetrahydro-2H-pyran-4-yl)-4-oxo-4,5-dihydrothieno[3,2-c]pyridine-7-carboxamide;
5-(2,6-difluoro-4-(2-methyl-2H-indazol-4-yl)benzyl)-N-((1S,2S)-2-hydroxycyclohexyl)-4-oxo-4,5-dihydrothieno[3,2-c]pyridine-7-carboxamide;
5-(2,6-difluoro-4-(2-methyl-2H-indazol-4-yl)benzyl)-N-((1S,2S)-2-hydroxycyclopentyl)-4-oxo-4,5-dihydrothieno[3,2-c]pyridine-7-carboxamide; and
5-(2,6-difluoro-4-(2-methyl-2H-indazol-4-yl)benzyl)-N-((1S,2S)-2-hydroxycycloheptyl)-4-oxo-4,5-dihydrothieno[3,2-c]pyridine-7-carboxamide;
or a pharmaceutically acceptable salt thereof.

50. A pharmaceutical composition comprising the compound of any of claims 1-49, or pharmaceutically acceptable salt thereof, and a pharmaceutically acceptable carrier.

51. A method for the treatment of a disorder associated with muscarinic acetylcholine receptor activity in a mammal, comprising administering to the mammal an effective amount of the compound of any of claims 1-49, or a pharmaceutically acceptable salt thereof, or the pharmaceutical composition of claim 50, or the hydrate, solvate, polymorph or prodrug of claim 62.

52. The method of claim 51, wherein the mammal is human.

53. The method of claim 51 or 52, wherein the muscarinic acetylcholine receptor is mAChR M1.

54. The method of any of claims 51-53, wherein the mammal has been diagnosed with a need for treatment of the disorder prior to the administering step.

55. The method of any of claims 51-54, further comprising the step of identifying a mammal in need of treatment of the disorder.

56. The method of any of claims 51-55, wherein the disorder is a neurological disorder or psychiatric disorder, or a combination thereof.

57. The method of any of claims 51-55, wherein the disorder is psychosis, schizophrenia, conduct disorder, disruptive behavior disorder, bipolar disorder, psychotic episodes of anxiety, anxiety associated with psychosis, psychotic mood disorders, severe major depressive disorder, mood disorders associated with psychotic disorders, acute mania, depression associated with bipolar disorder, mood disorders associated with schizophrenia, behavioral manifestations of mental retardation, conduct disorder, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, or memory disorders, or a combination thereof.

58. The method of any of claims 51-55, wherein the disorder is Alzheimer's disease, schizophrenia, a sleep disorder, a pain disorder, or a cognitive disorder, or a combination thereof.

59. The method of claim 58, wherein the pain disorder is neuropathic pain, central pain syndrome, postsurgical pain syndrome, bone and joint pain, repetitive motion pain, dental pain, cancer pain, myofascial pain, perioperative pain, chronic pain, dysmennorhea, inflammatory pain, headache, migraine headache, cluster headache, headache, primary hyperalgesia, secondary hyperalgesis, primary allodynia, secondary allodynia, or a combination thereof.

60. The compound of any of claims 1-49, or a pharmaceutically acceptable salt thereof, or the pharmaceutical composition of claim 50, or the hydrate, solvate, polymorph or prodrug of claim 62, for use in the treatment of a disorder associated with muscarinic acetylcholine receptor activity in a mammal.

61. Use of the compound of any of claims 1-49, or a pharmaceutically acceptable salt thereof, or the pharmaceutical composition of claim 50, or the hydrate, solvate, polymorph or prodrug of claim 62, for the preparation of a medicament for the treatment of a disorder associated with muscarinic acetylcholine receptor activity in a mammal.

62. A hydrate, solvate, polymorph, or prodrug of the compound of any of claims 1-49, or a pharmaceutically acceptable salt thereof.
